(19) United States
(12) Patent Application Publication
(10) Pub. No.: US 2025/0381298 A1
(43) Pub. Date: Dec. 18, 2025

(54) DUAL AAV-MYO7A VECTORS WITH IMPROVED SAFETY FOR THE TREATMENT OF USH1B

(71) Applicant: University of Florida Research Foundation, Incorporated, Gainesville, FL (US)

(72) Inventors: Shannon E. Boye, Gainesville, FL (US); Sanford L. Boye, Gainesville, FL (US); H. Lee Sweeney, Gainesville, FL (US); Kaitlyn Calabro, Gainesville, FL (US)

(73) Assignee: University of Florida Research Foundation, Incorporated, Gainesville, FL (US)

(21) Appl. No.: 19/245,523

(22) Filed: Jun. 23, 2025

Related U.S. Application Data
(62) Division of application No. 17/916,308, filed on Sep. 30, 2022, now Pat. No. 12,377,169.
(60) Provisional application No. 63/003,774, filed on Apr. 1, 2020.

(51) Int. Cl. A61K 48/00; A61K 38/00; C07K 14/47; C12N 15/86

(52) U.S. Cl. CPC A61K 48/005; A61K 48/0075; C07K 14/4716; C12N 15/86; A61K 38/00; C12N 2750/14132; C12N 2750/14143; C12N 2800/40

(57) ABSTRACT

Disclosed are compositions and methods for treating diseases of the mammalian eye, and in particular, complications of the retina associated with Usher syndrome 1B (USH1B). Further disclosed are compositions and methods for treating diseases of the mammalian inner ear, and in particular, complications of ear hair cells associated with Usher syndrome 1B (USH1B). The disclosure provides improved AAV-based, dual vector systems that facilitate the expression of full-length proteins whose coding sequences exceed that of the polynucleotide packaging capacity of an individual AAV vector. Described herein are modified hybrid dual vector systems that shift the coding sequence for the MYO7A tail domain from the front-half vector to the back-half vector by altering the split point (e.g., from between exons 23 and 24, to between exons 21 and 22), in order to eliminate the production of truncated MYO7A protein. Further described herein are improved, codon-modified hybrid and overlap vector systems in which putative stop codons and residual sequences in non-coding sequences are removed.

### RELATED APPLICATIONS

This application claims the benefit of the filing date of U.S. Provisional Application No. 63/003,774, filed Apr. 1, 2020, the entire contents of which are incorporated by reference.

### NON-FEDERAL SUPPORT

This invention was made in whole or in part from funding under grant award number TA-GT-0419-0774-UFL-GH received from the Foundation for Fighting Blindness, and under agreement number AGR00018211, received from Atsena Therapeutics, Inc.

### BACKGROUND OF THE DISCLOSURE

Recombinant AAV has emerged as a useful gene delivery vehicle to treat retinal disease. However, one limitation of AAV is its relatively small DNA packaging capacity-approximately 4.7 kilobases (KB). Thus, standard AAV vector systems are unsuitable for addressing diseases in which large genes are mutated or otherwise dysfunctional, such as Usher syndrome. A solution is needed in order to package large genes into AAV vector systems and safely deliver gene therapy treatment to patients.

### SUMMARY OF THE DISCLOSURE

The disclosure relates generally to the fields of molecular biology and virology, and in particular, to the development of gene delivery vehicles. Disclosed are improved rAAV dual vector and polynucleotide vector systems, and compositions useful in delivering a variety of nucleic acid segments, including those encoding therapeutic proteins, polypeptides, peptides, antisense oligonucleotides, or ribozyme constructs to selected host cells for use in various gene-therapy regimens. Further disclosed are recombinant viral particles, isolated host cells, and pharmaceutical compositions comprising any of these rAAV dual vector and polynucleotide vector systems. Methods are also provided for preparing and using the improved rAAV dual vector systems disclosed herein in a variety of viral-based gene therapies, and in particular, for the treatment and/or amelioration of symptoms of Myosin VII-deficiency, including, without limitation, the treatment of human Usher syndrome type IB. Further provided herein are methods of treatment or amelioration of a disease or condition involving the administration of rAAV dual vector systems that encode the MYO7A protein and result in reduced cytotoxicities than previously available vector systems. In some aspects, provided are methods of administering a vector system, whereby an amount of truncated MYO7A protein and/or associated cytotoxicity is minimized. In some embodiments, the therapeutic polypeptide is not a myosin polypeptide.

In various aspects, the methods of treatment and pharmaceutical compositions provided herein are intended for administration to one or both eyes of a subject, e.g., a human or animal subject. In further various aspects, the methods of treatment and pharmaceutical compositions provided herein are intended for administration to one or both ears of a subject, e.g., a human or animal subject.

The disclosure provides materials and methods for gene therapy of diseases, such as Usher syndrome. Usher syndrome, including types I (e.g., USH1B), II, and III, is a condition that results in sensory impairment, specifically in the visual, auditory, and vestibular systems. The sensory loss that accompanies Usher syndrome can be present even at birth, and gets progressively worse with age.

The most common form of Usher syndrome, USH1B, is a severe autosomal-recessive, deaf-blindness disorder caused by mutations in the MyosinVIIa gene. Patients are born deaf due to insufficient expression of human Myosin VII protein (MYO7A) and/or mutations in the gene causing protein malfunction. Blindness occurs from a progressive retinal degeneration that begins within the first decade of life. MYO7A protein is expressed in photoreceptors and retinal pigment epithelium (RPE), and is involved in opsin transport through photoreceptor cilia and the movement of RPE melanosomes. A study showed that photoreceptors (PRs) may be the initial site of disease, and that defects in an adhesion belt structure that sits around the photoreceptor outer segment in humans may cause the retinal degeneration seen in USH1B patients (Sahly, et al., 2012). The coding region for the MYO7A protein, however, is 6534 or 6648 nucleotides in length (depending on the isoform), making traditional AAV vector systems unsuitable for gene therapy of USH1B.

While there are currently no treatments available for this condition, gene therapy offers promise for recovering/maintaining function within the visual, auditory, and vestibular systems. Previously, Allocca et al. (2008) published results suggesting that AAV5 serotype vectors were capable of packaging genomes of up to 8.9 KB in size, and that these vectors expressed full-length proteins when delivered in vivo. In Allocca et al. (2008), the authors expressed full-length MYO7A protein from an AAV5 vector containing the CMV promoter driving hMYO7A. Subsequent studies confirmed that these ‘oversized’ AAV5 vectors did indeed drive full-length protein expression, however the genetic content of each vector capsid was found to be limited only to ˜5 KB of DNA, and not the 8.7 KB originally reported by Allocca et al. (2008) (Lai et al., 2010; Dong et al., 2010; Wu et al., 2010). These vector capsids were shown to contain a “heterogeneous mixture” of truncated vector genomes (e.g., the 5′ end of the gene, the 3′ end of the gene, or a mixture of the two with an internal sequence deletion). Additionally, these oversized/heterogeneous vectors exhibited poor packaging efficiency (for example, resulting in low-vector titers) and low transduction efficiency when compared to matched reporter vectors of standard size (<5 KB) (Wu et al., 2010).

Using the ‘heterogeneous’ system as described in Lai et al. (2010), Dong et al. (2010) and Wu et al. (2010), vectors containing portions of the MYO7A transgene were packaged despite the observed poor packaging efficiency, and proof-of-concept results were demonstrated in the shaker-1 mouse model of USH1B. The therapeutic results achieved with the heterogeneous AAV-hMYO7A vectors were comparable to previous gene replacement results using a lentivirus-based hMYO7A vector (Hashimoto et al., 2007). This lentivirus-MYO7A vector is under development by Oxford BioMedica in collaboration with Sanofi-Aventis for a phase I/HI clinical trial of USH1B, marketed under the name UshStat® LentiVector®. Lentivirus is regarded as a vector platform that is not well-suited for infecting post-mitotic (for example, non-dividing) cells. Furthermore, although the vector is suitable for transducing RPE, many studies have shown it to be ineffective at transducing adult photoreceptors. Because photoreceptors (PRs) may be the initial site of disease (Sahly, et al., 2012), the exclusive targeting by UshStat® of RPE cells may not bring about a complete or effective therapy, although this remains to be seen in human clinical trials.

Because of the excellent safety profile and encouraging reports of efficacy in the AAV gene therapy trials for LCA2/RPE65, there has been continuing interest in creating an AAV-based system for treating USH1B patients. The inventors have previously characterized AAV dual vector platforms for use in treating USH1B patients, also described herein. The original dual vector systems designed by the inventors (e.g., the “first generation” dual vector systems) have successfully demonstrated that mRNA arising from the system is 100% accurate relative to what would be predicted by correct homologous recombination of the front and back vector pairs, making them useful as gene therapy delivery vector systems. These vectors are described in US Patent Publication Nos. 2019/0153050 and 2014/0256802, each of which is incorporated herein by reference in its entirety.

This disclosure is based, at least in part, on the observation that some of the previous dual vector platforms resulted in the production of truncated MYO7A protein that was correlated with production of a truncated fragment of the MYO7A protein within the cell. Specifically, loss of retinal structure/function was observed following injection of a previous, first-generation dual vector hybrid system into mouse retina, which may have been attributable to the gain of function exerted by truncated MYO7A protein containing a portion of the tail domain. Hybrid vector systems contain both recombinogenic and spliceosome-recognition sequences that enable two paths through which the two halves of the polynucleotide vector system can combine in a cell to make a full-length polynucleotide. Hybrid vector systems are thus modular and versatile alternatives to simple overlap and simple trans-splicing dual vector systems. Described herein are modified dual hybrid vector systems that shift (all of, or a portion of) the coding sequence for the MYO7A tail domain from the front-half vector to the back-half vector by altering the split point (e.g., from between exons 23 and 24, to between exons 21 and 22) in order to eliminate the production of a truncated MYO7A protein and any associated cytotoxicity (for example, a gain of function toxicity observed in the retina). Further described herein are modified dual overlap vector systems that shift the coding sequence for the MYO7A tail domain from the front-half vector to the back-half vector by altering the overlapping coding sequence among the two vector halves.

Further described herein are codon-modified hybrid and overlap vector systems in which putative stop codons in non-coding sequences are removed. Further described herein are modified overlap vector systems that contain altered and/or reduced lengths of the overlapping coding sequence between the two vectors. Further described herein are modified hybrid vector systems that contain reductions in the lengths of the back half vector.

This disclosure is also based, at least in part, on the improvement of a previous, first-generation dual vector overlap system to increase transduction efficiency in the retina. In some embodiments, the disclosed improvements encompass the shortening of 5′ (front) and/or 3′ (back) AAV vectors in the system to increase rAAV particle packaging efficiencies.

In some embodiments, the disclosed rAAV vectors comprise a transgene encoding a MYO7A protein, e.g., human MYO7A protein. In some embodiments, the disclosed rAAV vectors comprise transgenes that encode other proteins relevant to Usher syndrome. In some embodiments, the disclosed rAAV vectors comprise transgenes that encode other proteins relevant to other ocular or aural diseases, disorders, or conditions.

Accordingly, aspects of the disclosure provide modified dual AAV vector systems that permit expression of full-length proteins, whose coding sequence exceeds the polynucleotide packaging capacity of an individual AAV vector.

Thus, in some aspects, provided herein are hybrid dual vector systems. Provided herein are polynucleotide vector systems comprising: i) a first AAV vector polynucleotide comprising an inverted terminal repeat at each end of the polynucleotide, and between the inverted terminal repeats a promoter followed by a partial coding sequence that encodes an N-terminal part of a myosin polypeptide followed by a splice donor (SD) site and an intron, and ii) a second AAV vector polynucleotide comprising an inverted terminal repeat at each end of the polynucleotide, and between the inverted terminal repeats an intron and a splice acceptor (SA) site for the intron, wherein the intron sequence in the first and second AAV vectors comprises a polynucleotide sequence that overlaps, and wherein the split point between the first and second AAV vector polynucleotide sequences is between exon 21 and exon 22 of the hMYO7A gene (see FIGS. 22D and 22E). Provided herein are hybrid polynucleotide systems in which the N-terminal part of the myosin polypeptide does not comprise the single-alpha helix (SAH) domain of the myosin polypeptide (e.g., in which the first AAV vector polynucleotide comprises a partial coding sequence that does not encode the SAH domain of the myosin polypeptide). In some embodiments, the intron sequence that overlaps comprises an alkaline phosphatase intron. Further provided herein are polynucleotide vector systems wherein the first AAV vector polynucleotide comprises the nucleotide sequence of SEQ ID NO: 33 or 34, and the second AAV vector polynucleotide comprises the nucleotide sequence of SEQ ID NO: 32, 35, or 44.

In other aspects, provided herein are overlap dual vector systems. Provided herein are polynucleotide vector systems comprising: i) a first AAV vector polynucleotide comprising an inverted terminal repeat at each end of the polynucleotide, and between the inverted terminal repeats a promoter followed by a partial coding sequence that encodes an N-terminal part of a myosin polypeptide, and ii) a second AAV vector polynucleotide comprising an inverted terminal repeat at each end of the polynucleotide, and between the inverted terminal repeats a partial coding sequence that encodes a C-terminal part of the myosin polypeptide, wherein the polynucleotide sequence encoding the polypeptide sequence in the first and second AAV vectors comprises a polynucleotide sequence that overlaps, and wherein the C-terminal part of the myosin polypeptide comprises the single-alpha helix (SAH) domain of the myosin polypeptide. Further provided herein are polynucleotide vector systems wherein the first AAV vector polynucleotide comprises a nucleic acid sequence at least about 80%, at least 85%, at least 90%, at least 95%, at least 98%, or at least 99% identical to the nucleotide sequence of SEQ ID NO: 36, and the second AAV vector polynucleotide comprises a nucleic acid sequence at least about 80%, at least 85%, at least 90%, at least 95%, at least 98%, or at least 99% identical to the nucleotide sequence of SEQ ID NO: 38.

In some aspects, provided herein are polynucleotide vector systems comprising: i) a first AAV vector polynucleotide comprising an inverted terminal repeat at each end of the polynucleotide, and between the inverted terminal repeats a promoter followed by a partial coding sequence that encodes an N-terminal part of a myosin polypeptide, and ii) a second AAV vector polynucleotide comprising an inverted terminal repeat at each end of the polynucleotide, and between the inverted terminal repeats a partial coding sequence that encodes a C-terminal part of the myosin polypeptide, wherein the polynucleotide sequence encoding the polypeptide sequence in the first and second AAV vectors comprises a polynucleotide sequence that overlaps, and wherein (i) the first AAV vector polynucleotide comprises a nucleic acid sequence at least about 80%, at least 85%, at least 90%, at least 95%, at least 98%, or at least 99% identical to SEQ ID NO: 63, 90, or 66, and (ii) the second AAV vector polynucleotide comprises a nucleic acid sequence at least about 80% at least 85%, at least 90%, at least 95%, at least 98%, or at least 99% identical to SEQ ID NO: 77 or 80.

Provided herein are polynucleotide vector systems comprising: i) a first AAV vector polynucleotide comprising an inverted terminal repeat at each end of the polynucleotide, and between the inverted terminal repeats a promoter followed by a partial coding sequence that encodes an N-terminal part of a myosin polypeptide, and ii) a second AAV vector polynucleotide comprising an inverted terminal repeat at each end of the polynucleotide, and between the inverted terminal repeats a partial coding sequence that encodes a C-terminal part of the myosin polypeptide, wherein the polynucleotide sequence encoding the polypeptide sequence in the first and second AAV vectors comprises a polynucleotide sequence that overlaps, and wherein (i) the first AAV vector polynucleotide comprises a nucleotide sequence selected from SEQ ID NOs: 63, 90, and 66, and (ii) the second AAV vector polynucleotide comprises a nucleotide sequence selected from SEQ ID NOs: 77 and 80.

Provided herein are polynucleotide vector systems comprising: i) a first AAV vector polynucleotide comprising an inverted terminal repeat at each end of the polynucleotide, and between the inverted terminal repeats a promoter followed by a partial coding sequence that encodes an N-terminal part of a myosin polypeptide, and ii) a second AAV vector polynucleotide comprising an inverted terminal repeat at each end of the polynucleotide, and between the inverted terminal repeats a partial coding sequence that encodes a C-terminal part of the myosin polypeptide, wherein the polynucleotide sequence encoding the polypeptide sequence in the first and second AAV vectors comprises a polynucleotide sequence that overlaps, and wherein (i) the first AAV vector polynucleotide encodes an amino acid sequence at least about 80%, at least 85%, at least 90%, at least 95%, at least 98%, or at least 99% identical to the amino acid sequence of SEQ ID NO: 62, 91, or 65, and (ii) the second AAV vector polynucleotide encodes an amino acid sequence at least about 80%, at least 85%, at least 90%, at least 95%, at least 98%, or at least 99% identical to the amino acid sequence of SEQ ID NO: 78 or 81.

Provided herein are polynucleotide vector systems comprising: i) a first AAV vector polynucleotide comprising an inverted terminal repeat at each end of the polynucleotide, and between the inverted terminal repeats a promoter followed by a partial coding sequence that encodes an N-terminal part of a myosin polypeptide, and ii) a second AAV vector polynucleotide comprising an inverted terminal repeat at each end of the polynucleotide, and between the inverted terminal repeats a partial coding sequence that encodes a C-terminal part of the myosin polypeptide, wherein the polynucleotide sequence encoding the polypeptide sequence in the first and second AAV vectors comprises a polynucleotide sequence that overlaps, and wherein the polynucleotide sequence that overlaps comprises a nucleotide sequence at least about 80%, at least 85%, at least 90%, at least 95%, at least 98%, or at least 99% identical to a sequence selected from any one of SEQ ID NOs: 39 and 52-59.

Provided herein are polynucleotide vector systems comprising: i) a first AAV vector polynucleotide comprising an inverted terminal repeat at each end of the polynucleotide, and between the inverted terminal repeats a promoter followed by a partial coding sequence that encodes an N-terminal part of a myosin polypeptide, and ii) a second AAV vector polynucleotide comprising an inverted terminal repeat at each end of the polynucleotide, and between the inverted terminal repeats a partial coding sequence that encodes a C-terminal part of the myosin polypeptide, wherein the polynucleotide sequence encoding the polypeptide sequence in the first and second AAV vectors comprises a polynucleotide sequence that overlaps, and wherein the polynucleotide sequence that overlaps comprises a sequence encoding any one of SEQ ID NOs: 79 and 82-89.

Provided herein are polynucleotide vector systems comprising: i) a first AAV vector polynucleotide comprising an inverted terminal repeat at each end of the polynucleotide, and between the inverted terminal repeats a promoter followed by a partial coding sequence that encodes an N-terminal part of a myosin polypeptide followed by a splice donor site and a first intron, and ii) a second AAV vector polynucleotide comprising an inverted terminal repeat at each end of the polynucleotide, and between the inverted terminal repeats a second intron and a splice acceptor site for the first intron, wherein the nucleotide sequences of the first and second introns (collectively referred to herein as “the intron sequence”) comprise a polynucleotide sequence that overlaps, and wherein the first and/or second intron sequence comprises a nucleic acid sequence at least about 80% at least 85%, at least 90%, at least 95%, at least 98%, or at least 99% identical to SEQ ID NO: 69 or SEQ ID NO: 70.

Provided herein are polynucleotide vector systems comprising: i) a first AAV vector polynucleotide comprising an inverted terminal repeat at each end of the polynucleotide, and between the inverted terminal repeats a promoter followed by a partial coding sequence that encodes an N-terminal part of a myosin polypeptide followed by a splice donor site and a first intron, and ii) a second AAV vector polynucleotide comprising an inverted terminal repeat at each end of the polynucleotide, and between the inverted terminal repeats a second intron and a splice acceptor site for the first intron, wherein the nucleotide sequences of the first and second introns comprise a polynucleotide sequence that overlaps, and wherein the split point between the first and second AAV vector polynucleotide sequences is between two exons of the gene encoding the therapeutic protein.

Provided herein are polynucleotide vector systems comprising: i) a first AAV vector polynucleotide comprising an inverted terminal repeat at each end of the polynucleotide, and between the inverted terminal repeats a promoter followed by a partial coding sequence that encodes an N-terminal part of a myosin polypeptide followed by a splice donor site and a first intron, and ii) a second AAV vector polynucleotide comprising an inverted terminal repeat at each end of the polynucleotide, and between the inverted terminal repeats a second intron and a splice acceptor site for the first intron, wherein the nucleotide sequences of the first and second introns comprise a polynucleotide sequence that overlaps, and wherein (i) the first AAV vector polynucleotide comprises a nucleic acid sequence at least about 80%, at least 85%, at least 90%, at least 95%, at least 98%, or at least 99% identical to SEQ ID NOs: 31, 33, 34, and 46, and (ii) the second AAV vector polynucleotide comprises a nucleic acid sequence at least about 80%, at least 85%, at least 90%, at least 95%, at least 98%, or at least 99% identical to SEQ ID NOs: 32, 35, 44, and 47-49.

Provided herein are polynucleotide vector systems comprising: i) a first AAV vector polynucleotide comprising an inverted terminal repeat at each end of the polynucleotide, and between the inverted terminal repeats a promoter followed by a partial coding sequence that encodes an N-terminal part of a myosin polypeptide followed by a splice donor site and a first intron, and ii) a second AAV vector polynucleotide comprising an inverted terminal repeat at each end of the polynucleotide, and between the inverted terminal repeats a second intron and a splice acceptor site for the first intron, wherein the nucleotide sequences of the first and second introns comprise a polynucleotide sequence that overlaps, and wherein (i) the first AAV vector polynucleotide comprises a nucleic acid sequence at least about 80%, at least 85%, at least 90%, at least 95%, at least 98%, or at least 99% identical to the nucleotide sequence of SEQ ID NO: 33, and (ii) the second AAV vector polynucleotide comprises a nucleic acid sequence at least about 80%, at least 85%, at least 90%, at least 95%, at least 98%, or at least 99% identical to the nucleotide sequence of SEQ ID NO: 32.

Provided herein are polynucleotide vector systems comprising: i) a first AAV vector polynucleotide comprising an inverted terminal repeat at each end of the polynucleotide, and between the inverted terminal repeats a promoter followed by a partial coding sequence that encodes an N-terminal part of a myosin polypeptide followed by a splice donor site and a first intron, and ii) a second AAV vector polynucleotide comprising an inverted terminal repeat at each end of the polynucleotide, and between the inverted terminal repeats a second intron and a splice acceptor site for the first intron, wherein the nucleotide sequences of the first and second introns comprise a polynucleotide sequence that overlaps, and wherein (i) the first AAV vector polynucleotide comprises a nucleic acid sequence at least about 80%, at least 85%, at least 90%, at least 95%, at least 98%, or at least 99% identical to the nucleotide sequence of SEQ ID NO: 34, and (ii) the second AAV vector polynucleotide comprises a nucleic acid sequence at least about 80%, at least 85%, at least 90%, at least 95%, at least 98%, or at least 99% identical to the nucleotide sequence of SEQ ID NO: 35.

Provided herein are polynucleotide vector systems comprising: i) a first AAV vector polynucleotide comprising an inverted terminal repeat at each end of the polynucleotide, and between the inverted terminal repeats a promoter followed by a partial coding sequence that encodes an N-terminal part of a myosin polypeptide followed by a splice donor site and a first intron, and ii) a second AAV vector polynucleotide comprising an inverted terminal repeat at each end of the polynucleotide, and between the inverted terminal repeats a second intron and a splice acceptor site for the first intron, wherein the nucleotide sequences of the first and second introns comprise a polynucleotide sequence that overlaps, and wherein (i) the first AAV vector polynucleotide comprises a nucleic acid sequence at least about 80%, at least 85%, at least 90%, at least 95%, at least 98%, or at least 99% identical to the nucleotide sequence of SEQ ID NO: 34, and (ii) the second AAV vector polynucleotide comprises a nucleic acid sequence at least about 80%, at least 85%, at least 90%, at least 95%, at least 98%, or at least 99% identical to the nucleotide sequence of SEQ ID NO: 44.

### BRIEF DESCRIPTION OF THE DRAWINGS

For promoting an understanding of the principles of the disclosure, reference will now be made to the embodiments, or examples, illustrated in the drawings and specific language will be used to describe the same. It will nevertheless be understood that no limitation of the scope of the disclosure is thereby intended. Any alterations and further modifications in the described embodiments, and any further applications of the principles of the disclosure as described herein are contemplated as would normally occur to one of ordinary skill in the art to which the disclosure relates.

FIG. 1 shows the formation of complete gene cassette from dual AAV vectors via homologous recombination.

FIG. 2 shows a schematic of the two vector components that make up the Overlap Dual Vector System in accordance with one aspect of the disclosure.

FIG. 3 shows a schematic of the two vector components that make up an exemplary Hybrid Dual Vector System containing a native intron. Native hMYO7A intron 23 in shown in light shading; splice donor and splice acceptor sequences are shown in darker shading and indicated with a (•).

FIG. 4 shows the schematic of the two vector components that make up an exemplary Hybrid Dual Vector System containing a synthetic intron. This is an exemplary standard trans splicing dual vector system, with the “intron” referring to the synthetic alkaline phosphatase splice donor and acceptor sites. Synthetic alkaline phosphatase (AP) intron is shown in light shading; AP splice donor and splice acceptor sequences are shown in darker shading and indicated with a (•).

FIG. 5 shows an immunoblot to detect the presence of MYO7A in infected or transfected HEK293 cells. Heterogeneous vectors are compared to all three dual vector systems. Dual vectors were packaged either in AAV2 or in AAV2 (triple mutant) capsids. The triple mutant contains three tyrosine-to-phenylalanine mutations on the capsid surface. For all three dual vector systems, infections were performed with either a) the front-half (N-terminal) and back-half (C-terminal) vectors; or b) the front-half vectors alone (to confirm the presence or absence of a truncated protein product expressed from the promoter-containing N-terminal vectors).

FIG. 6A and FIG. 6B show immunoblot to detect the presence of MYO7A in HEK293 cells infected with an exemplary Overlap Dual Vector System. Results are presented as a time course from 3-7 days post infection (lanes 3-7) and are compared to cells transfected with MYO7A plasmid (lane 1) and uninfected control (lane 2). An area of interest in FIG. 6A is magnified and presented at higher contrast in FIG. 6B. Starting at 3 days post-infection, full-length human MYO7A protein was visible, with peak expression occurring around day 5.

FIG. 7A and FIG. 7B show retinas from untreated mice and mice treated subretinally with an exemplary Overlap Dual Vector System. Immunohistochemistry (IHC) was performed using an antibody directed against MYO7A. MYO7A stains and nuclear (DAPI) stains are indicated.

FIGS. 8A-8D show differences in RPE melanosome localization in wild type vs. shaker-1 mice. In wild type mice, RPE melanosome apically migrate towards photoreceptor outer segments (FIG. 8A) whereas this phenomenon fails to occur in mice lacking MYO7A (shaker-1), as seen in (FIG. 8B). To the right is a high magnification image of single RPE cells from either a wild type (FIG. 8C) or shaker-1 (FIG. 8D) mouse showing this phenomenon up close.

FIGS. 9A-9C show that apical migration of RPE melanosomes is restored in shaker-1 mice injected with an exemplary Overlap Dual Vector System. Electron microscopy reveals that melanosomes of untreated shaker-1 mice do not apically migrate (FIG. 9A). In shaker-1 mice injected with exemplary overlap vectors (packaged in AAV2), RPE melanosomes migrate apically towards photoreceptors, which can be seen here in both low- and high-magnification images (FIGS. 9B and 9C).

FIGS. 10A-10F illustrate the expression of MYO7A from single AAV2 and AAV5 vectors in cultured cells. FIG. 10A is a diagram of the viral vector encoding human MYO7A cDNA. FIG. 10B is a western blot of WT eyecup (lane 1), primary RPE cultures derived from MYO7A-null mice and infected with AAV2-MYO7A (lane 2) or AAV5-MYO7A (lane 3), or not infected (lane 4), and primary RPE cultures derived from MYO7A^{+/-} mice (lane 5). All lanes were immunolabeled with antibodies against actin (as a loading indicator of relative protein loading) and MYO7A. FIGS. 10C-10F are immunofluorescence images of primary RPE cell cultures. Cells derived from MYO7A-null mice that were not infected (FIG. 10C), from MYO7A^{+/-} mice (FIG. 10D), or from MYO7A-null mice infected with either 1×AAV2-MYO7A (FIG. 10E) or 1×AAV5-MYO7A (FIG. 10F). Scale=10 μm.

FIGS. 11A-11M show the expression of MYO7A from AAV2 and AAV5 dual vectors in vivo. FIGS. 11A-11E show EM images of MYO7A immunogold labelling of the connecting cilium and pericilium from rod photoreceptors in a MYO7A-null retina. FIG. 11A is a longitudinal section from an untreated MYO7A-null retina (background label only). FIG. 11B and FIG. 11C are longitudinal sections from MYO7A-null retinas treated with AAV2-MYO7A (FIG. 11B) or AAV5-MYO7A (FIG. 11C). Scale=50 nm. FIG. 11D and FIG. 11E are transverse sections of connecting cilia from rod photoreceptors in MYO7A-null retinas treated with AAV2-MYO7A (FIG. 11D) or AAV5-MYO7A (FIG. 11E). Scale=50 nm. FIG. 11F and FIG. 11G show EM images of RPE cells from MYO7A-null retinas treated with AAV2-MYO7A (FIG. 11F) or AAV5-MYO7A (FIG. 11G). Scale=500 nm. Areas indicated by rectangles are enlarged in FIG. 11F-1 and FIG. 11G-1 to show MYO7A immunogold labeling (indicated by circles). Scale=50 nm. FIG. 11H and FIG. 11I show EM image of a longitudinal section of the connecting cilium and pericilium from a rod (FIG. 11H) and a cone (FIG. 11I) photoreceptor in a MYO7A-null retina, treated with AAV2-MYO7A. The section was double-labeled with MYO7A (12-nm gold) and rod opsin (15-nm gold) antibodies. Rod outer segments were labeled with the opsin antibody, while cones were identified by lack of rod opsin labeling in their outer segments. The sections show just the base of the outer segments. Nearly all the label in the connecting cilium is MYO7A, even in the rod. Scale=50 nm. FIGS. 11J-11M are bar graphs indicating MYO7A immunogold particle density in the rod photoreceptor cilium and pericilium (FIG. 11J and FIG. 11L) and in the RPE (FIG. 11K and FIG. 11M), following treatment with AAV2-MYO7A (FIG. 11J and FIG. 11K) or AAV5-MYO7A (FIG. 11L and FIG. 11M) at different concentrations. n=3 animals per condition. Bars indicate SEM.

FIGS. 12A-12F show correction of melanosome localization, following subretinal injections with AAV2-MYO7A or AAV5-MYO7A. Light micrographs showing the presence of melanosomes in the apical processes of the RPE in a WT retina (FIG. 12A) and retinas injected with AAV2-MYO7A (FIG. 12B) or AAV5-MYO7A (FIG. 12C). Further away from the injection site (FIG. 12D), melanosomes are present in the apical processes of some RPE cells, but not in others (arrows indicate apical melanosomes; white lines indicate regions where melanosomes are absent from the apical processes). FIG. 12E illustrates a region distant from injection site, where all RPE cells lacked melanosomes in their apical processes. Brackets on left side indicate RPE apical processes. Scale=25 μm. FIG. 12F is a diagram of an eyecup, indicating the relative locations of the images shown in FIGS. 12A-12E. ONH indicates the optic nerve head.

FIG. 13 shows the correction of abnormal levels of opsin in the connecting cilium and pericilium of rod photoreceptors, following subretinal injections with AAV2-MYO7A or AAV5-MYO7A. The bar graph shows opsin immunogold gold particle density along the length of the connecting cilium. Ultrathin sections of retinas from MYO7A-null and WT mice were stained with rod opsin antibody. The MYO7A-null retinas had been untreated, or treated with either 1× or 1:100 AAV2-MYO7A or AAV5-MYO7A. n=3 animals per condition. Bars indicate SEM.

FIGS. 14A-14G show the expression of MYO7A from the overlapping AAV2-MYO7A dual vectors. FIG. 14A-1 and FIG. 14A-2 illustrate a diagram of the overlapping AAV2-MYO7A dual vectors. The overlapping region contains 1365 bases. FIG. 14B is a Western blot of proteins from primary RPE cultures derived from MYO7A-null mice that were either not infected (lane 1), or infected with AAV2-MYO7A (overlap dual) (lane 2); primary RPE cultures derived from MYO7A^{+/-} mice (lane 3); WT eyecup (lane 4); HEK293 cells transfected with pTR-smCBA-MYO7A (lane 5). All lanes were immunolabeled with anti-MYO7A and anti-actin. FIGS. 14C-14F show immunofluorescence of cultured RPE cells transduced with AAV2-MYO7A (overlap dual). FIGS. 14C-14E show primary RPE cultures derived from MYO7A-null mice and ARPE19 (FIG. 14F) cells. Scale=10 μm. FIG. 14G is a bar graph indicating the distribution of MYO7A immunogold particle density among RPE cells from retinas of MYO7A-null mice, injected with AAV2-MYO7A(dual). n=3 animals.

FIGS. 15A-15G illustrate correction of mutant phenotypes, following subretinal injections with AAV2-MYO7A (overlap dual). FIG. 15A shows light microscopy of a semi-thin section from a treated MYO7A-null mouse retina. The region shown is near the injection site. Arrows indicate melanosomes in the apical processes. White lines indicate cells that still show the MYO7A-null phenotype, with an absence of melanosomes in the apical processes. Scale=50 μm. FIG. 15B is a low-magnification immunoEM image of RPE from a retina treated with AAV2-MYO7A (overlap dual). As in FIG. 15A, the white line indicated a region that still showed the MYO7A-null phenotype. Rectangle ‘c’, includes melanosomes in the apical region, indicating a corrected RPE cell. Scale=500 nm. FIGS. 15C-15E show higher-magnification images of regions outlined by the rectangles shown in FIG. 15B. MYO7A immunogold particles are indicated by circles. Scale=50 nm. FIG. 15F is a bar graph illustrating MYO7A immunogold particle density measured in RPE cells from MYO7A-null retinas, WT retinas, or from MYO7A-null retinas treated with AAV2-MYO7A (overlap dual) and determined to be corrected or not corrected by the location of their apical melanosomes. n=3 animals per condition. Bars indicate SEM. FIG. 15G is an immunoEM image of a rod photoreceptor cilium double-labeled with antibodies against MYO7A (small gold particles) and against rod opsin (large gold particles). MYO7A labeling is associated with the connecting cilium and periciliary membrane, indicating expression and correct localization of MYO7A. While this region is devoid of opsin labeling, which is restricted to the disk membranes, it is consistent with the wild type (WT) phenotype, thus indicating correction of the mutant phenotype. Scale=300 nm.

FIG. 16 shows the validation of dual AAV vectors for delivery of full-length MYO7A in vivo. Immunoblot showing expression of MYO7A in retinas of wild type (C57BL/6) mice (lane 1), heterozygous shaker-1^{+/- mice (lane} 2) and shaker-1^{-/-} mice injected with ‘simple overlap’ MYO7A vectors packaged in AAV8(733) vectors. Both N-terminal and C-terminal vectors of the ‘simple overlap’ system were injected at a concentration of 3×10¹⁰ vector genomes/μL. Dual AAV vectors mediated expression of a MYO7A that was identical in size to that found in WT and shaker-1^{+/-} mice. β-actin (visualized here in red) was used as a loading control to validate that equal amounts of protein were loaded in each well.

FIGS. 17A-17F show AAV-mediated MYO7A expression in ARPE-19 with fragmented vectors and the simple overlap dual-vector system. Cells were transduced with fragmented vectors: 1×AAV2-MYO7A (FIG. 17A), AAV5-MYO7A (FIG. 17B), 1/100 dilutions thereof (FIG. 17C and FIG. 17D), and the simple overlap dual-vector system: AAV2-MYO7A (dual) (FIG. 17F). Non-transduced cells were used as a control (FIG. 17E); lighter color, MYO7A; darker color, DAPI. Scale=10 μm.

FIGS. 18A-18D show MYO7A expression in the connecting cilium and pericilium of rod photoreceptors from MYO7A-null retinas injected with diluted AAV2-MYO7A (FIG. 18A and FIG. 18B) or AAV5-MYO7A (FIG. 18C and FIG. 18D); (FIG. 18A and FIG. 18C) 1:10, (FIG. 18B and FIG. 18D) 1:100. Scale=200 nm.

FIG. 19 shows the structural preservation of injected MYO7A-null retinas. Light microscopy of the photoreceptor layer 3 weeks after injection with 10×AAV5-MYO7A. Scale=15 μm.

FIG. 20 shows structural preservation of injected MYO7A-null retinas. Light microscopy of photoreceptor layer 3 months after injection with 1×AAV2-MYO7A. Scale=10 μm.

FIGS. 21A-21D show correction of abnormal levels of opsin in the connecting cilium and pericilium of rod photoreceptors following subretinal injections with AAV2-MYO7A or AAV5-MYO7A. ImmunoEMs from WT retina (FIG. 21A), MYO7A-null retinas treated with 1×AAV2-MYO7A (FIG. 21B) or 1×AAV5-MYO7A (FIG. 21C), and from an untreated MYO7A-null retina (FIG. 21D) labeled with anti-rod opsin and 12-nm gold-conjugated secondary antibody. Scale=200 nm.

FIGS. 22A-22E show a schematic representation of the dual-AAV-vector pairs created for this study. FIG. 22A is a fragmented AAV (fAAV) vector. FIG. 22B shows simple overlap: the 1365-bp shared between the two vectors is shaded gray. FIG. 22C is a trans-splicing vector. FIG. 22D shows an AP hybrid vector: the 270-bp element shared between the two vectors is marked with diagonal gradient shading (1/3 APhead as described by Ghosh et al., 2011). FIG. 22E shows the native intron hybrid vectors utilizing a 250-bp sequence of MYO7A intron 23. 3′ MYO7A is the 3′-portion of MYO7A; 5′ MYO7A is the 5′-portion of MYO7A; AAV is adeno-associated virus; AP is alkaline phosphatase; intron=intron 23 of MYO7A; pA=polyadenylation signal; SA=splice-acceptor site; SD=splice-donor site; and smCBA refers to a (truncated) chimeric cytomegalovirus immediate early/chicken 3-actin chimeric promoter.

FIGS. 23A-23C show cells expressing human MYO7A after infection with the simple overlap vectors of the present disclosure. FIG. 23A shows human embryonic kidney (HEK293) cells expressing human MYO7A after infection with simple overlap vectors (MOI of 10,000 for both vectors) packaged in AAV2 (tripleY-F). Equal amounts of protein were separated on 7.5% sodium dodecyl sulfate (SDS) polyacrylamide gel electrophoresis (PAGE) and stained for MYO7A. FIG. 23B shows HEK293 cells infected with AAV2 (tripleY-F) at MOIs of 10,000, 2,000, and 400. FIG. 23C is a time-course assay of MYO7A expressed in HEK293 cells. Cells were harvested 3-7 days after infection. MOI=multiplicity of infection; T=HEK293 cells transfected with full-length MYO7A plasmid; U=untreated HEK293 cells.

FIG. 24 shows the comparison of AAV2 and AAV2 (tripleY-F mutant capsid)-based vectors in HEK293 cells. Cells were infected with AP hybrid and simple overlap MYO7A dual vector platforms packaged in AAV2 or AAV2(tripleY-F) at an MOI of 10,000 for each vector.

FIGS. 25A-25C show human MYO7A expressed in HEK293 cells. Cells were infected with AAV2-based vector platforms. For each of the dual vector systems, the corresponding 5′ and 3′ vectors (or the 5′ vector alone) were used for infection. HEK293 cells transfected with MYO7A plasmid were used as a positive control. Cells were infected with the MYO7A dual vector pairs at an MOI of 10,000 for each vector. Protein samples were analyzed on Western blot with an antibody against MYO7A (FIG. 25A). Each dual vector platform's relative ability to promote reconstitution was compared by quantifying the amount of 5′ vector-mediated truncated protein product in the presence or absence of the respective 3′ vector (FIG. 25B). Full-length MYO7A expression mediated by dual vectors was quantified relative to transfection control (FIG. 25C).

FIGS. 26A-26C show the ability of MYO7A dual vectors to recombine and properly restore coding sequence. The experimental plan is shown in FIG. 26A. HEK293 cells were infected with AAV2-based dual vector platforms, RNA was extracted, and gene-specific primers amplified the sequences using PCR. Control digests with BglII (B) and PpuMI (P) revealed the predicted banding pattern shown in FIGS. 26B-26C. Undigested (U) PCR product is shown as control and a DNA size marker for reference (M). Separately, products were digested with KpnI and AgeI, and then cloned into pUC57 for sequencing of the entire overlap region. Ten clones per vector platform were analyzed. M13 forward- and reverse-primers specific for the subclone vector were used to obtain sense and antisense reads (each 1,000 bp) resulting in 140 bp for which the sense and antisense reads overlapped (FIG. 26C; Table). PCR=polymerase chain reaction.

FIGS. 27A-27H show the dual vector-mediated MYO7A expression in vivo. C57BL/6J mice were injected subretinally with AAV2-based dual vectors containing a C-terminal hemagglutinin (HA) tag. Retinal protein expression was analyzed four weeks post-injection by immunohistochemistry and western blot. Ten-micron frozen retinal cross sections were stained with an antibody for HA and imaged at 1Ox (FIG. 27A, FIG. 27C, and FIG. 27E) and 60× (FIG. 27B, FIG. 27D, and FIG. 27F). An untreated C57BL/6J retina was also stained with an antibody against HA (FIG. 27G). Equal amounts of protein were separated on a 4-15% polyacrylamide gel and stained with an HA antibody (FIG. 27H). For comparison, endogenous MYO7A from C57BL/6J retina was probed with an antibody against MYO7A to confirm that HA-tagged MYO7A migrated at the appropriate size. RPE—retinal pigment epithelium, IS—inner segments, OS—outer segments, ONL—outer nuclear layer, INL—inner nuclear layer, GCL—ganglion cell layer, PR—photoreceptors. Nuclear (DAPI) stains are indicated with brackets (}).

FIG. 28 is a representative schematic of how the dual vector systems deliver, recombine, and produce full-length transgene. MYO7A cDNA is split into two parts, or “halves,” and each half is delivered via a separate AAV vector. Following co-infection, gene halves recombine via their shared/overlapping sequence to form full-length MYO7A. The recombined transgene can then be transcribed and translated into the desired protein product.

FIGS. 29A-29B show the dual vector-mediated MYO7A expression Myo7a^{-/-} mice. FIG. 29A shows the resultant protein expression following simple overlap or AP hybrid dual vector injections. Myo7a^{-/-} mice were injected with 5.0×10⁸ vector genomes (vg) total (2.5×10⁸ vg each) of either the simple overlap or AP hybrid dual vectors. All vector expression was driven by the smCBA promoter. Retinas were collected and analyzed at 6 weeks post-injection. FIG. 29B shows the quantification of the full-length MYO7A expression normalized to vinculin (VCL) for multiple treatment groups.

FIG. 30 shows two bar graphs illustrating the scotopic b-wave responses and average ONL thickness by treatment at 6 weeks post-injection, in order to evaluate the safety of dual AAV-MYO7A vectors. All treatments were delivered at 5.0×10⁸ vg total (2.5×10⁸ vg each).

FIGS. 31A-31B show that only the front half hybrid vectors produced truncated protein. FIG. 31A is a bar graph showing the average MYO7A front half transcript expression at 6 weeks post-injection. FIG. 31B shows the Western Blot results at 6 weeks post-injection, using VCL as a loading control. All vectors were delivered at 5.0×10⁸ vg.

FIG. 32 shows two bar graphs illustrating the scotopic b-wave responses in C57BL/6J and Myo7a^{-/-} mice at 6 weeks post-injection. These results show that only the front half hybrid vectors led to a loss of retinal function. All vectors were delivered at 5.0×10⁸ vg.

FIG. 33 shows two bar graphs illustrating the average MYO7A back half transcript expression and vector genomes at 6 weeks post-injection. These results show that the back half vectors do not produce transcript or truncated protein. All vectors were delivered at 8.0×10⁸ vg.

FIG. 34 is a diagram showing the split site relocation from the original site, between exons 23 and 24, to a new split site, between exons 21 and 22, in order to prevent a loss of function observed following injection with front half hybrid vectors. The hybrid vector system with the new split site between exons 21 and 22 will be hereinafter referred to as the “second generation” hybrid.

FIGS. 35A-35B show in vitro results after administration of first and second generation hybrid vectors in HEK293 cells. FIG. 35A shows the Western Blot results of the original hybrid, with a split site between exons 23 and 24 (“Ex23/24”), and the second generation hybrid, with a split site between exons 21 and 22 (“Ex21/22”), using VCL as a loading control. FIG. 35B illustrates the normalized full-length MYO7A expression. NI indicates “not injected”.

FIGS. 36A-36C show in vivo results from the subretinal injection of second generation dual hybrid vectors in Myo7a^{-/-} mice. The injection dose was 5×10⁸ vg total. FIG. 36A shows the Western Blot results, using VCL as a loading control. FIG. 36B shows p-values for the comparison of full-length protein expression between exons 21/22 and exons 23/24 in the hybrid vector systems. FIG. 36C displays the normalized MYO7A expression relative to WT. Hybrid vectors were encapsidated and administered in AAV5 and AAV8(Y733F) virions, and the MYO7A expression associated with each serotype was measured and normalized to WT.

FIGS. 37A-37B show schematics of an exemplary second generation MYO7A polynucleotide vector systems of the disclosure. FIG. 37A shows the second generation hybrid vector system, with a split site between exons 21 and 22. AP sequences act as a polyA and/or splicing signal. FIG. 37B shows the overlap vectors with the potential in-frame stop codons from the 3′ end (downstream of the MYO7A N-terminal fragment) in the front hybrid vector plasmid removed.

FIG. 38 is a bar graph showing the results from in vivo hybrid AAV-MYO7A injections. The second generation Ex2l/22 hybrid vectors express equivalent amounts of full-length MYO7A compared to the first generation Ex23/24 hybrid vectors, but do not cause the production of a truncated protein fragment observed in the first generation Ex23/24 hybrid vectors.

FIGS. 39A-39B show in vitro results of expression of a truncated MYO7A protein in HEK293 cells. FIG. 39A shows the Western blot results of the original hybrid (exons 23/24) front, second generation hybrid (exons 21/22) front, and the hybrid CMv1 (exons 21/22) front. As used herein, “CMv1” (or “COv2”) refers to the human codon-modified version 1 hybrid vector. FIG. 39B is a bar graph showing the truncated MYO7A expression normalized to vinculin.

FIG. 40 shows the western blot results for the original hybrid front vector, second generation ex21/22 hybrid front vector, CMv1 ex21/22 hybrid front vector, and the CMv2 ex21/22 hybrid front vector. As used herein, “CMv2” (may be referred to herein as “COv2”) refers to the human codon-modified version 2 hybrid vector.

FIG. 41 shows the nonhuman primate data for the expression of AAV-mediated MYO7A transcript in macaque retina and tolerability of dual AAV5-MYO7A vectors in subretinally injected macaque retinas. Vectors were delivered at titers of 4×10⁸ vg each (8×10⁸ vg total).

FIG. 42 shows the procedure for removal of the stop codons from the second generation hybrid front-half vector (“Myo7a NT-Ex21”) to create the CMv1 hybrid front-half vector. The second generation hybrid front-half vector (relevant fragment of SEQ ID NO: 31 shown as SEQ ID NO: 40) contained three stop codons within the AP intron (potential stop codons shown in red). The restriction enzymes Bsu36I and NheI were used to excise the region containing the stop codons. Gibson primer sets (SEQ ID NO: 42, SEQ ID NO: 43) were then used to replace the excised sequence. The new sequence had all three of the potential in-frame stop codons removed (are indicated with asterisks (*)) (relevant fragment of SEQ ID NO: 33 shown as SEQ ID NO: 41). In addition to modifying the potential stop codons, two restriction sites, Hpal and Mfel, were added to the new sequence to simplify the screening of the resultant clones.

FIG. 43 shows the changes made in the second generation overlap front-half vector to create the CMv1 overlap front-half vector.

FIG. 44 is a schematic that shows the dual AAV vector approach for the delivery of MYO7A gene therapy. The MYO7A cDNA is split into two halves and each half is delivered via a separate AAV vector, delivered in separate AAV particles that are co-injected. The gene halves recombine in each cell to form full-length MYO7A.

FIGS. 45A-45C show two different dual AAV vector platforms that drive full-length MYO7A expression. FIG. 45A contains schematics depicting the overlap and hybrid dual AAV vector platforms. FIGS. 45B and 45C show the amount of MYO7A produced from the original hybrid and overlap vectors following encapsidation in AAV5 or AAV8(Y733F) virions. VCL marker was used as a control.

FIG. 46 shows improved (third generation) overlap dual vectors that show increased packaging efficiency.

FIG. 47 shows length and location of overlap. Shorter overlap lengths result in less shared sequence between front and back half vectors.

FIGS. 48A and 48B show overlap vectors compared using the Protein Simple Jess quantification system through use of a capillary-based Western blot tool. FIG. 48A shows the expression of MYO7A and truncated MYO7A from various overlap vectors. FIG. 48B shows a table describing the overlap length and the respective ITR-ITR length (bp).

FIGS. 49A-49B show overlap panel quantification. Overlap vectors containing 687 or 945 bp of overlapping MYO7A sequence produce as much or more full length MYO7A as original hybrid vectors. FIG. 49A shows MYO7A expression normalized to VCL relative to the original hybrid vectors. FIG. 49B shows the degree of expression of truncated MYO7A protein normalized to VCL relative to the original hybrid vectors.

FIGS. 50A-50D show improved hybrid dual vectors that provide reduced cytotoxicity and greater safety. FIG. 50A shows Hybrid-V2 with altered ‘split point’ such that no neck/tail domain is encoded by the front half vector. FIG. 50B shows a schematic of the hybrid vectors. Four potential in-frame stop codons were modified in the Hybrid-V2 front half vector sequence. All codon modifications were made on the Hybrid-V2 MIN background. FIG. 50C shows a table describing the modifications made to each vector, and the respective ITR to ITR (ITR-ITR) length (in bp). FIG. 50D shows a comparison of production of full-length MYO7A and truncated MYO7A fragment against the original hybrid vectors.

FIGS. 51A-51E show improved hybrid dual vectors for safety. FIG. 51A shows Hybrid-V2 back MIN, Hybrid-CMv2 back MIN, and an exemplary vector representing Hybrid-V2 back MIN HA and Hybrid-CMv2 back MIN HA. In the Hybrid back MIN vectors, an ‘unneeded legacy’ sequence was removed from the back half vector to ensure the vector size did not exceed packaging capacity. FIG. 51B shows a table describing the codon modifications made to each vector, and the respective ITR-ITR length (bp). FIGS. 51C-E show that reducing the size of back half vectors leads to increased expression of full length MYO7A from hybrid vectors.

### DETAILED DESCRIPTION OF THE DISCLOSURE

Illustrative embodiments of the disclosure are described below. The disclosure provides compositions and methods for genetic therapy of diseases and conditions, such as Usher syndrome 1B (USH1B). Aspects of the disclosure concern AAV-based dual vector systems that allow for expression of full-length proteins whose coding sequence exceeds the polynucleotide packaging capacity of individual AAV vectors. Aspects of this disclosure provide AAV-based dual vector systems for expression in the retina of the eyes of the subject, or the hair cells of the inner ear of a subject. Accordingly, provided herein are methods for treatment of ocular and aural symptoms associated with USHIB, as well as other diseases and disorders. The disclosure provides nucleic acid vectors of an overlap vector system and nucleic acid vectors of a hybrid vector system.

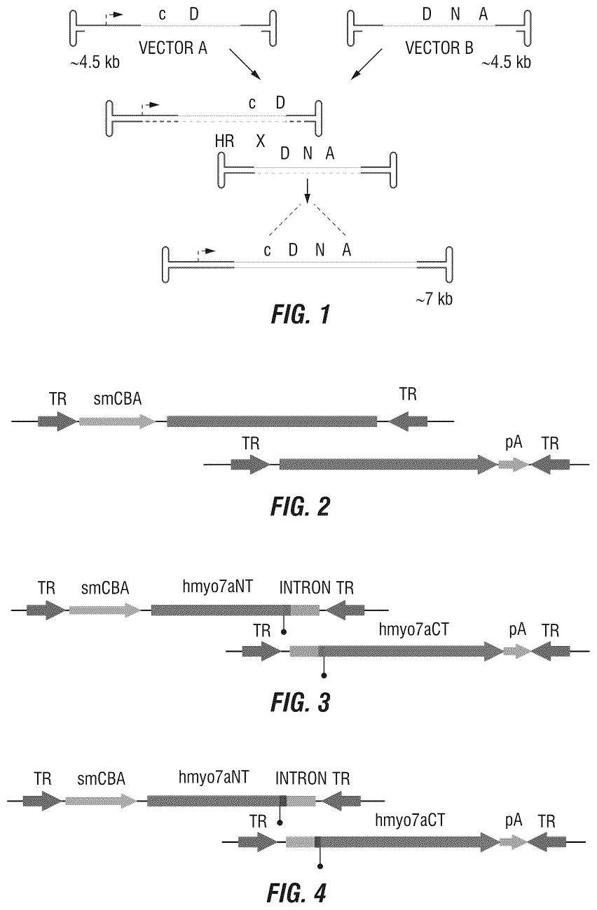

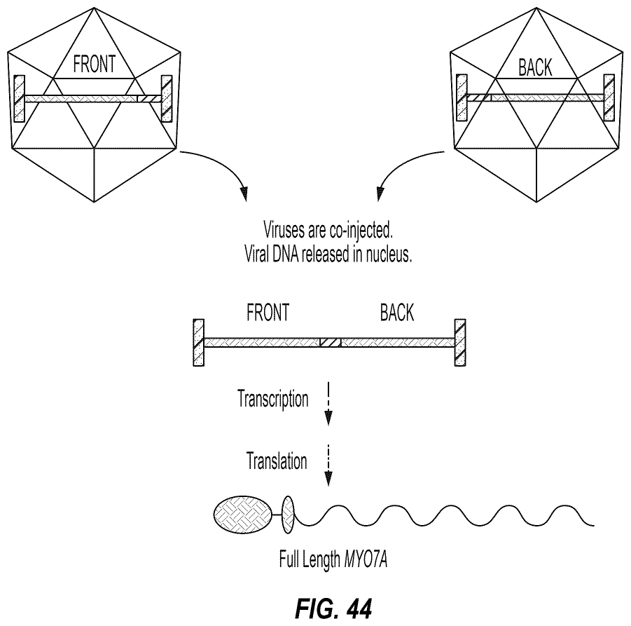

Multiple distinct, AAV-based, dual vector systems have been created and disclosed herein for use in gene replacement therapies, including, for example, in the treatment of USHIB in human patients. In particular embodiments, a vector system of the disclosure employs two discrete AAV vectors that each packages a maximal-size DNA molecule (for example, ˜4.5 to 4.8 Kb). The two vectors are co-administered to selected recipient cells to reconstitute the full-length, biologically-active, MYO7A polypeptide. In these constructs, a portion of overlapping nucleic acid sequence is common to each of the vector genomes (see FIG. 1). When co-delivered to suitable cells (FIG. 44), the overlapping sequence region facilitates the proper concatamerization of the two partial gene cassettes. These gene cassettes then undergo homologous recombination to produce a full-length gene cassette within the cells (see FIG. 1). Exemplary shared components of exemplary embodiments of the dual vector systems include the use of AAV inverted terminal repeats (ITR), the small (truncated) version of the chimeric CMV/chicken R-actin promoter (smCBA), human MYO7A (hMYO7A) cDNA sequence and the SV40 polyadenylation (pA) signal.

In some embodiments, the polynucleotide vector and vector systems provided herein do not comprise any of the nucleotide sequences of SEQ ID NOs: 1-4. In exemplary embodiments, the overlap vectors of the disclosure do not comprise any of SEQ ID NOs: 1 and 2. In exemplary embodiments, the hybrid vectors of the disclosure do not comprise any of the nucleotide sequences of SEQ ID NOs: 3 and 4. In some embodiments, the vectors of the disclosure do not comprise the nucleotide sequence of SEQ ID NO: 67 or NO: 71.

### Overlap Vector System

In some aspects, overlap dual AAV vector systems are provided. In some embodiments, the overlap vector systems of the disclosure do not produce a truncated MYO7A protein fragment following administration to a mouse or a subject.

In one aspect of the disclosure, an overlap vector system of the disclosure includes:
i) a first AAV vector polynucleotide comprising an inverted terminal repeat at each end (5′ and 3′ end) of the polynucleotide, and between the inverted terminal repeats a suitable promoter followed by (for example, 3′ to the promoter) a partial coding sequence that encodes an N-terminal part of a selected full-length polypeptide, and
ii) a second AAV vector polynucleotide comprising an inverted terminal repeat at each end (for example, the 5′- and 3′-ends) of the polynucleotide, and between the inverted terminal repeats a partial coding sequence that encodes a C-terminal portion of the selected full-length polypeptide, and optionally followed by a polyadenylation (pA) sequence. The coding sequences in the first and second vectors when combined encode the selected full-length polypeptide, or a functional fragment or variant thereof. The polypeptide encoding sequence in the first and second AAV vectors comprises sequence that overlaps.

In some embodiments of the provided overlap vector systems, the selected full-length polypeptide is a myosin polypeptide. In some embodiments, the myosin polypeptide is human myosin VII A (hMYO7A). In some embodiments, the myosin polypeptide is human myosin VII B (hMYO7B). In some embodiments, the myosin polypeptide is myosin 7 (VII) isoform II. Isoform II (2) of hMYO7A (NM_001127180) encodes a 2175-amino acid protein (250.2 kDa) and lacks an in-frame segment in the coding region (a portion of exon 35), relative to isoform I (see Chen et al., 1996; Weil et al., 1996). In some embodiments, the myosin polypeptide is another myosin isoform or a functional fragment thereof. In particular embodiments, full-length myosin 7A or isoform II is encoded in the provided vector systems. The peptide sequence of isoform II is set forth as SEQ ID NO: 8.

In some embodiments, the selected full-length polypeptide is selected from ABCA4 (Stargardt disease), CEP290 (LCA10), EYS (Retinitis Pigmentosa), RP1 (Retinitis Pigmentosa), ALMS1(Alstrom syndrome), CDH23 (Usher syndrome 1D), PCDH15 (Usher syndrome 1F), and USHERIN (USH2A) Usher syndrome 2A). In some embodiments, the selected full-length polypeptide is selected from DMD (Duchenne muscular dystrophy), CFTR (Cystic fibrosis), GDE (Glycogen storage disease III), DYSF (dysferlinopathies), OTOF (neurosensory nonsyndromic recessive deafness) and F8 (Hemophilia A). The diseases and disorders associated with each of these genes are provided in parentheses. In some embodiments, the selected full-length polypeptide is encoded by a gene of about 6 Kb to about 9 Kb in length. In some embodiments, the selected full-length polypeptide is encoded by a gene of about 7 Kb to about 8 Kb in length.

The inventors have also discovered that hMYO7A overlapping regions, e.g., SEQ ID NOs: 39 and 53-59, may be used as the polynucleotide sequence that overlaps in additional overlap dual vectors expressing large genes (other than MYO7A). Accordingly, in some embodiments, overlap dual vectors expressing portions (or halves) of a large gene selected from ABCA4, CEP290, EYS, RP1, ALMS1, CDH23, PCDH15, USH1C, USH1G, USH2A, DNFB31, DMD, CFTR, GDE, DYSF, F8, and DFNB2, contain an overlap region that comprises a part of the hMYO7A gene in the polynucleotide sequence that overlaps. These overlap vectors express a large gene other than MYO7A and that comprises a nucleotide sequence having at least 80%, 85%, 90%, 95%, 98%, or 99% identity to any one of SEQ ID NOs: 39 and 52-59. Such overlap vectors may comprise an overlapping region that contains the nucleotide sequence of any one of SEQ ID NOs: 39 and 53-59, e.g., SEQ ID NO: 56 or 57.

In some embodiments, the selected full-length polypeptide is expressed in one or more photoreceptor cells. In some embodiments, the selected full-length polypeptide is expressed in one or more cells that do not comprise photoreceptor cells. In some embodiments, the selected full-length polypeptide is expressed in one or more hair cells, e.g., hair cells of the auditory system or the vestibular system.

In some embodiments, the C-terminal part of the selected full-length polypeptide (e.g., the myosin polypeptide) comprises the single-alpha helix (SAH) domain of the selected full-length polypeptide.

In some embodiments, the first AAV vector polynucleotide comprises the nucleotide sequence of SEQ ID NO: 1, or a functional fragment and/or variant thereof, and the second AAV vector polynucleotide comprises the nucleotide sequence of SEQ ID NO: 2, or a functional fragment and/or variant thereof.

In some embodiments, the first generation overlap vector (for example, the AAV vector polynucleotide comprising the nucleotide sequence of SEQ ID NO: 1, or a functional fragment and/or variant thereof, and/or the second AAV vector polynucleotide comprising the nucleotide sequence of SEQ ID NO: 2, or a functional fragment and/or variant thereof) contains nucleotides 1 through 3644 of MYO7A cDNA from the ATG in the 5′ vector and/or nucleotides 2279 through 6534 in the 3′ vector. In some embodiments, the fragments are amplified with P1 and P3 by polymerase chain reaction (PCR) and cloned into the 5′ vector via NotI and NheI and the 3′ vector with P3 (AflII) and P4 (KpnI), respectively. The resulting two vector plasmids share 1365 bp of overlapping MYO7A sequence (FIG. 2), and the overlap between the sequences ends at the split point between exons 23 and 24.

In some embodiments, a portion of the coding sequence present at the 3′-end of the coding sequence of the first generation overlap vector is identical or substantially identical with a portion of the coding sequence present at the 5′-end of the coding sequence of the first generation overlap vector. In particular embodiments, the sequence overlap between the first and second AAV (first generation) overlap vectors of the disclosure is between about 500 and about 3,000 nucleotides; between about 1,000 and about 2,000 nucleotides; between about 1,200 and about 1,800 nucleotides; or between about 1,300 and about 1,400 nucleotides.

In particular embodiments, the sequence overlap between the first and second AAV overlap vectors of the disclosure is 1284 bp, 1027 bp, 1026 bp, 945 bp, 687 bp, 361 bp, 279 bp, or 20 bp in length. In particular embodiments, the sequence overlap between the first and second AAV overlap vectors of the disclosure has a length that is within 1, 2, 3, 4, 5, 6, 7, 8, 9, or 10 nucleotides different from 1284 bp, 1027 bp, 1026 bp, 945 bp, 687 bp, 361 bp, 279 bp, or 20 bp. In some embodiments, the sequence overlap is 945 bp, 687 bp, or 361 bp.

In particular embodiments, the sequence overlap of the first generation overlap vector system is about 1,350 nucleotides. In an exemplary embodiment, the sequence overlap of the first generation overlap vector system is 1,365 nucleotides. In particular embodiments, the polynucleotide sequence that overlaps comprises SEQ ID NO: 45. In particular embodiments, the polypeptide encoded is wild type or functional human myosin VIIa (hMYO7A). Amino acid sequences of wild type and functional hMYO7A polypeptides, and polynucleotides encoding them, are known in the art (see, e.g., GenBank accession numbers NP_000251 and U39226.1). In particular embodiments, a hMYO7A polypeptide comprises the amino acid sequence shown in SEQ ID NO: 6 or SEQ ID NO: 8, or a functional fragment or a variant thereof. In particular embodiments, the hMYO7A polypeptide is encoded by the nucleotide sequence shown in SEQ ID NO: 5 or SEQ ID NO: 7.

### Codon-Modified Overlap Vectors

In some embodiments of the disclosed overlap vector systems, a codon-modified overlap vector is provided. In some embodiments, the first generation overlap front-half vector (“AAV-smCBA-hMYO7A-NT”) is shortened. All coding sequences corresponding to the tail domain of MYO7A was removed from the front half vector, thus reducing the size of the overlap region to 361 bp (SEQ ID NO: 39). (This vector does not generate a truncated MYO7A fragment containing a tail, or SAH, domain.) The vector was also altered so that all potential (or putative) stop codons were removed. (See FIG. 37B.) The resultant vector is the CMv1 overlap front-half vector (“AAV-smCBA-hMYO7A-noDimNT-CMv1”). Accordingly, the overlap vector system of the disclosure may comprise a CMv1 overlap vector system.

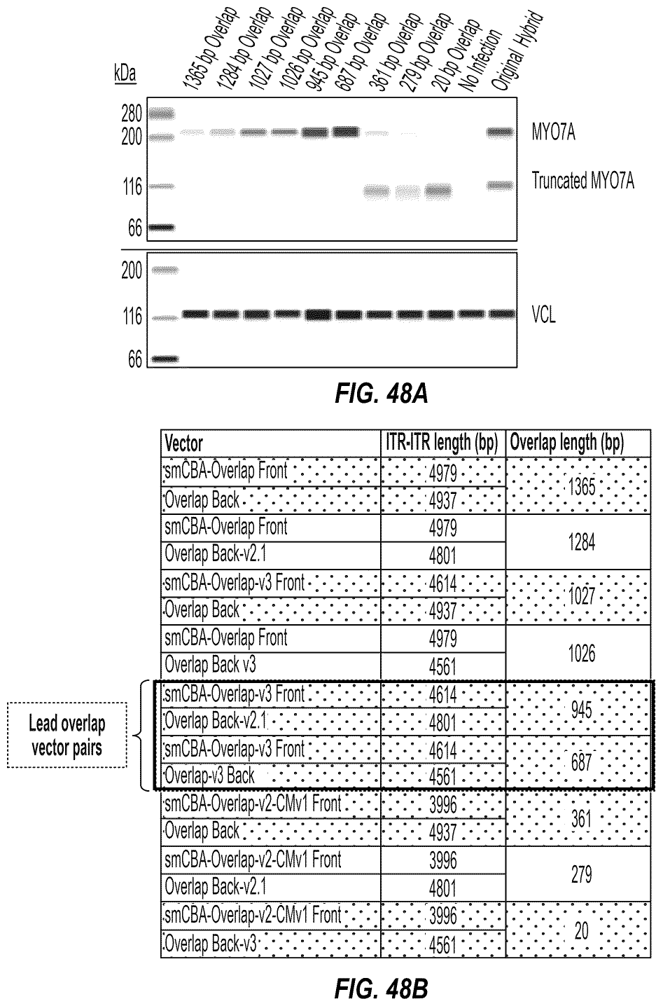

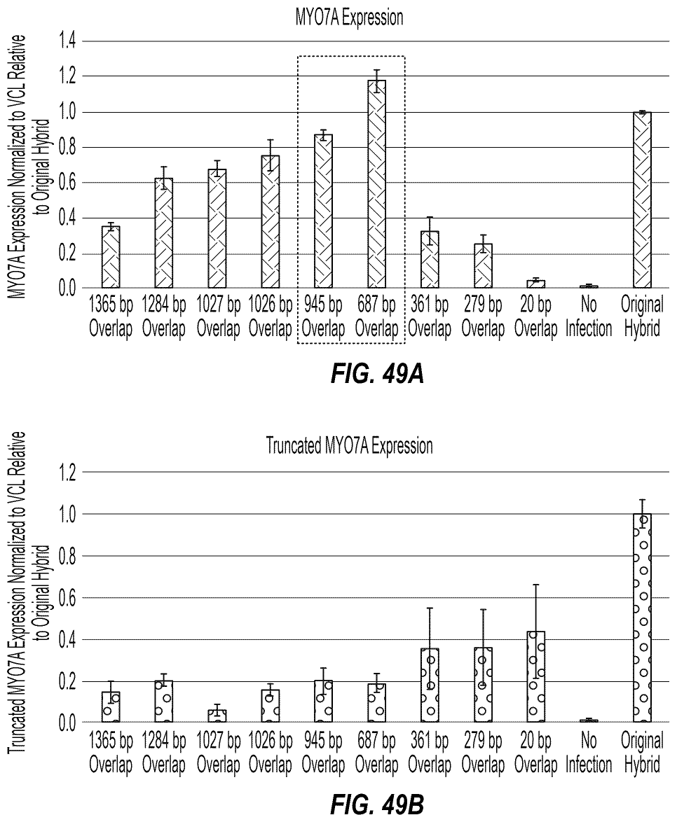

In some embodiments, an overlap vector having an altered (e.g., a shortened) overlapping coding sequence is provided. In such embodiments, an overlap vector containing an overlap sequence in the MYO7A gene or another gene that is less than 1365 bp in length is provided. In these systems, the length of the overlapping sequence is reduced to a certain point, therefore ensuring neither vector genome is pushing the packaging capacity of AAV capsid (4.7-4.9 kb), leads to increased expression of full length MYO7A. If the overlap length is too small (≤361 bp), full length MYO7A expression is reduced, and truncated protein appears. Overlap vectors containing 687 or 945 bp of overlapping MYO7A sequence produce as much or more full-length MYO7A as original hybrid vectors. (See FIGS. 48A, 48B, 49A, and 49B.) Such vectors may be referred to herein as “V3” or 3^{rd} generation overlap vectors. In exemplary embodiments, overlap vectors containing 687 or 945 bp of overlapping MYO7A sequence are exemplified.

Accordingly, provided herein are polynucleotide vector systems wherein the polynucleotide sequence that overlaps comprises a nucleotide sequence selected from any one of SEQ ID NOs: 39 and 52-59. In some embodiments, the polynucleotide sequence that overlaps comprises a nucleotide sequence selected from any one of SEQ ID NOs: 39, 56, and 57. In exemplary embodiments, the polynucleotide sequence that overlaps comprises the sequence of SEQ ID NO: 56 or 57. In some embodiments, the length between the inverted terminal repeats at each end of the first AAV vector polynucleotide is about 4615 nucleotides (nt) or fewer. In some embodiments, the length between the inverted terminal repeats at each end of the second AAV vector polynucleotide is about 4800 nt or fewer. In some embodiments, the length between the inverted terminal repeats at each end of the second AAV vector polynucleotide is about 4560 nt.

Thus, in some embodiments, the polynucleotide vector system of the disclosure is a CMv1 overlap system. In some embodiments, the vector system is an overlap V2 (2^{nd} generation) system. In some embodiments, the vector system is a V3 overlap (3^{rd} generation) system. Any of the disclosed front-half overlap vectors may be combined with any of the disclosed back-half overlap vectors in the compositions of the disclosure. The resulting third generation overlap front half vector (“AAV-smCBA-hMYO7A-NTlong-v3”) is set forth as SEQ ID NO: 50. The resulting third generation overlap back half vector, inclusive of an HA tag, is set forth as SEQ ID NO: 51.

Accordingly, in some aspects, provided herein are polynucleotide vector systems comprising:
i) a first AAV vector polynucleotide comprising an inverted terminal repeat at each end of the polynucleotide, and between the inverted terminal repeats a promoter followed by a partial coding sequence that encodes an N-terminal part of a myosin polypeptide, and
ii) a second AAV vector polynucleotide comprising an inverted terminal repeat at each end of the polynucleotide, and between the inverted terminal repeats a partial coding sequence that encodes a C-terminal part of the myosin polypeptide,
wherein the polynucleotide sequence encoding the polypeptide sequence in the first and second AAV vectors comprises a polynucleotide sequence that overlaps, and
wherein the first AAV vector polynucleotide comprises a nucleotide sequence selected from SEQ ID NOs: 36, 37, and 50;
and the second AAV vector polynucleotide comprises a nucleotide sequence selected from SEQ ID NOs: 38 and 51. In exemplary embodiments, the first AAV vector polynucleotide comprises the nucleotide sequence of SEQ ID NO: 50, and the second AAV vector polynucleotide comprises the nucleotide sequence of SEQ ID NO: 51. In some embodiments, the first AAV vector polynucleotide comprises the nucleotide sequence of SEQ ID NO: 50, and the second AAV vector polynucleotide comprises the nucleotide sequence of SEQ ID NO: 38. In some embodiments, the first AAV vector polynucleotide comprises the nucleotide sequence of SEQ ID NO: 36, and the second AAV vector polynucleotide comprises the nucleotide sequence of SEQ ID NO: 38.

In some embodiments, the first AAV vector polynucleotide comprises a partial coding sequence that does not encode the single-alpha helix (SAH) domain of the selected full-length polypeptide. In some embodiments, the first AAV vector polynucleotide of the second generation overlap vector comprises the nucleotide sequence of SEQ ID NO: 37, or a functional fragment and/or variant thereof, and the second AAV vector polynucleotide of the second generation overlap vector comprises the nucleotide sequence of SEQ ID NO: 38, or a functional fragment and/or variant thereof.

In some embodiments, the second generation overlap vector (for example, the AAV vector polynucleotide comprising the nucleotide sequence of SEQ ID NO: 37, or a functional fragment and/or variant thereof, and/or the second AAV vector polynucleotide comprising the nucleotide sequence of SEQ ID NO: 38, or a functional fragment and/or variant thereof) contains nucleotides 1 through 2640 of MYO7A cDNA from the ATG in the 5′ vector and/or nucleotides 2279 through 6534 in the 3′ vector. In some embodiments, the fragments are amplified with P1 and P3 by polymerase chain reaction (PCR) and cloned into the 5′ vector via NotI and NheI and the 3′ vector with P3 (AflII) and P4 (KpnI), respectively. The resulting two vector plasmids share 361 bp of overlapping MYO7A sequence (FIG. 37), and the overlap between the sequences ends at the split point between exons 21 and 22.

In some embodiments, the polynucleotide sequence that overlaps does not comprise any portion of exon 23 of the hMYO7A gene. In some embodiments, the polynucleotide sequence that overlaps does not comprise exon 23 in full (e.g., 100% of exon 23). In some embodiments, the polynucleotide sequence that overlaps comprises a portion of exon 17, exon 18 in full, exon 19 in full, exon 20 in full, and a portion of exon 21 of the hMYO7A gene. In some embodiments, the polynucleotide sequence that overlaps comprises a portion of exon 17, a portion of exon 18, a portion of exon 19, a portion of exon 20, and/or a portion of exon 21 of the hMYO7A gene. As used herein, a “portion” may comprise e.g. at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, etc. of the exon and/or intron sequence.

In some embodiments, a portion of the coding sequence present at the 3′-end of the coding sequence of the first vector of the second generation overlap vector is identical or substantially identical with a portion of the coding sequence present at the 5′-end of the coding sequence of the second vector of the second generation overlap vector. In particular embodiments, the sequence overlap between the first and second AAV vectors is between about 1 and about 500 nucleotides; between about 100 and about 200 nucleotides; between about 200 and about 300 nucleotides; or between about 300 and about 400 nucleotides.

In particular embodiments, the sequence overlap of the second generation overlap vector system is about 350 nucleotides. In an exemplary embodiment, the sequence overlap of the second generation overlap vector system is 361 nucleotides. In particular embodiments, the polynucleotide sequence that overlaps comprises SEQ ID NO: 39. In particular embodiments, the polypeptide encoded is wild type or functional human myosin VIIa (hMYO7A). Amino acid sequences of wild type and functional hMYO7A polypeptides, and polynucleotides encoding them, are known in the art (see, e.g., GenBank accession numbers NP_000251 and U39226.1). In particular embodiments, a hMYO7A polypeptide comprises the amino acid sequence shown in SEQ ID NO: 6 or SEQ ID NO: 8, or a functional fragment or a variant thereof. In particular embodiments, the hMYO7A polypeptide is encoded by the nucleotide sequence shown in SEQ ID NO: 5 or SEQ ID NO: 7.

SEQ ID NO: 5 is a nucleotide sequence encoding a human myosin VIIa polypeptide
(protein coding sequence is nucleotides 273-6920);
GCTCTGGGCAGGAGAGAGAGTGAGAGACAAGAGACACACACAGAGAGACGGCG
AGGAAGGGAAAGACCCAGAGGGACGCCTAGAACGAGACTTGGAGCCAGACAGA
GGAAGAGGGGACGTGTGTTTGCAGACTGGCTGGGCCCGTGACCCAGCTTCCTGAG
TCCTCCGTGCAGGTGGCAGCTGTACCAGGCTGGCAGGTCACTGAGAGTGGGCAGC
TGGGCCCCAGAACTGTGCCTGGCCCAGTGGGCAGCAGGAGCTCCTGACTTGGGAC
CATGGTGATTCTTCAGCAGGGGGACCATGTGTGGATGGACCTGAGATTGGGGCAG
GAGTTCGACGTGCCCATCGGGGCGGTGGTGAAGCTCTGCGACTCTGGGCAGGTCC
AGGTGGTGGATGATGAAGACAATGAACACTGGATCTCTCCGCAGAACGCAACGC
ACATCAAGCCTATGCACCCCACGTCGGTCCACGGCGTGGAGGACATGATCCGCCT
GGGGGACCTCAACGAGGCGGGCATCTTGCGCAACCTGCTTATCCGCTACCGGGAC
CACCTCATCTACACGTATACGGGCTCCATCCTGGTGGCTGTGAACCCCTACCAGCT
GCTCTCCATCTACTCGCCAGAGCACATCCGCCAGTATACCAACAAGAAGATTGGG
GAGATGCCCCCCCACATCTTTGCCATTGCTGACAACTGCTACTTCAACATGAAAC
GCAACAGCCGAGACCAGTGCTGCATCATCAGTGGGGAATCTGGGGCCGGGAAGA
CGGAGAGCACAAAGCTGATCCTGCAGTTCCTGGCAGCCATCAGTGGGCAGCACTC
GTGGATTGAGCAGCAGGTCTTGGAGGCCACCCCCATTCTGGAAGCATTTGGGAAT
GCCAAGACCATCCGCAATGACAACTCAAGCCGTTTCGGAAAGTACATCGACATCC
ACTTCAACAAGCGGGGCGCCATCGAGGGCGCGAAGATTGAGCAGTACCTGCTGG
AAAAGTCACGTGTCTGTCGCCAGGCCCTGGATGAAAGGAACTACCACGTGTTCTA
CTGCATGCTGGAGGGTATGAGTGAGGATCAGAAGAAGAAGCTGGGCTTGGGCCA
GGCCTCTGACTACAACTACTTGGCCATGGGTAACTGCATAACCTGTGAGGGCCGG
GTGGACAGCCAGGAGTACGCCAACATCCGCTCCGCCATGAAGGTGCTCATGTTCA
CTGACACCGAGAACTGGGAGATCTCGAAGCTCCTGGCTGCCATCCTGCACCTGGG
CAACCTGCAGTATGAGGCACGCACATTTGAAAACCTGGATGCCTGTGAGGTTCTC
TTCTCCCCATCGCTGGCCACAGCTGCATCCCTGCTTGAGGTGAACCCCCCAGACCT
GATGAGCTGCCTGACTAGCCGCACCCTCATCACCCGCGGGGAGACGGTGTCCACC
CCACTGAGCAGGGAACAGGCACTGGACGTGCGCGACGCCTTCGTAAAGGGGATC
TACGGGCGGCTGTTCGTGTGGATTGTGGACAAGATCAACGCAGCAATTTACAAGC
CTCCCTCCCAGGATGTGAAGAACTCTCGCAGGTCCATCGGCCTCCTGGACATCTTT
GGGTTTGAGAACTTTGCTGTGAACAGCTTTGAGCAGCTCTGCATCAACTTCGCCA
ATGAGCACCTGCAGCAGTTCTTTGTGCGGCACGTGTTCAAGCTGGAGCAGGAGGA
ATATGACCTGGAGAGCATTGACTGGCTGCACATCGAGTTCACTGACAACCAGGAT
GCCCTGGACATGATTGCCAACAAGCCCATGAACATCATCTCCCTCATCGATGAGG
AGAGCAAGTTCCCCAAGGGCACAGACACCACCATGTTACACAAGCTGAACTCCC
AGCACAAGCTCAACGCCAACTACATCCCCCCCAAGAACAACCATGAGACCCAGTT
TGGCATCAACCATTTTGCAGGCATCGTCTACTATGAGACCCAAGGCTTCCTGGAG
AAGAACCGAGACACCCTGCATGGGGACATTATCCAGCTGGTCCACTCCTCCAGGA
ACAAGTTCATCAAGCAGATCTTCCAGGCCGATGTCGCCATGGGCGCCGAGACCAG
GAAGCGCTCGCCCACACTTAGCAGCCAGTTCAAGCGGTCACTGGAGCTGCTGATG
CGCACGCTGGGTGCCTGCCAGCCCTTCTTTGTGCGATGCATCAAGCCCAATGAGT
TCAAGAAGCCCATGCTGTTCGACCGGCACCTGTGCGTGCGCCAGCTGCGGTACTC
AGGAATGATGGAGACCATCCGAATCCGCCGAGCTGGCTACCCCATCCGCTACAGC
TTCGTAGAGTTTGTGGAGCGGTACCGTGTGCTGCTGCCAGGTGTGAAGCCGGCCT
ACAAGCAGGGCGACCTCCGCGGGACTTGCCAGCGCATGGCTGAGGCTGTGCTGG
GCACCCACGATGACTGGCAGATAGGCAAAACCAAGATCTTTCTGAAGGACCACC
ATGACATGCTGCTGGAAGTGGAGCGGGACAAAGCCATCACCGACAGAGTCATCC
TCCTTCAGAAAGTCATCCGGGGATTCAAAGACAGGTCTAACTTTCTGAAGCTGAA
GAACGCTGCCACACTGATCCAGAGGCACTGGCGGGGTCACAACTGTAGGAAGAA
CTACGGGCTGATGCGTCTGGGCTTCCTGCGGCTGCAGGCCCTGCACCGCTCCCGG
AAGCTGCACCAGCAGTACCGCCTGGCCCGCCAGCGCATCATCCAGTTCCAGGCCC
GCTGCCGCGCCTATCTGGTGCGCAAGGCCTTCCGCCACCGCCTCTGGGCTGTGCTC
ACCGTGCAGGCCTATGCCCGGGGCATGATCGCCCGCAGGCTGCACCAACGCCTCA
GGGCTGAGTATCTGTGGCGCCTCGAGGCTGAGAAAATGCGGCTGGCGGAGGAAG
AGAAGCTTCGGAAGGAGATGAGCGCCAAGAAGGCCAAGGAGGAGGCCGAGCGC
AAGCATCAGGAGCGCCTGGCCCAGCTGGCTCGTGAGGACGCTGAGCGGGAGCTG
AAGGAGAAGGAGGCCGCTCGGCGGAAGAAGGAGCTCCTGGAGCAGATGGAAAG
GGCCCGCCATGAGCCTGTCAATCACTCAGACATGGTGGACAAGATGTTTGGCTTC
CTGGGGACTTCAGGTGGCCTGCCAGGCCAGGAGGGCCAGGCACCTAGTGGCTTTG
AGGACCTGGAGCGAGGGCGGAGGGAGATGGTGGAGGAGGACCTGGATGCAGCC
CTGCCCCTGCCTGACGAGGATGAGGAGGACCTCTCTGAGTATAAATTTGCCAAGT
TCGCGGCCACCTACTTCCAGGGGACAACCACGCACTCCTACACCCGGCGGCCACT
CAAACAGCCACTGCTCTACCATGACGACGAGGGTGACCAGCTGGCAGCCCTGGC
GGTCTGGATCACCATCCTCCGCTTCATGGGGGACCTCCCTGAGCCCAAGTACCAC
ACAGCCATGAGTGATGGCAGTGAGAAGATCCCTGTGATGACCAAGATTTATGAG
ACCCTGGGCAAGAAGACGTACAAGAGGGAGCTGCAGGCCCTGCAGGGCGAGGGC
GAGGCCCAGCTCCCCGAGGGCCAGAAGAAGAGCAGTGTGAGGCACAAGCTGGTG
CATTTGACTCTGAAAAAGAAGTCCAAGCTCACAGAGGAGGTGACCAAGAGGCTG
CATGACGGGGAGTCCACAGTGCAGGGCAACAGCATGCTGGAGGACCGGCCCACC
TCCAACCTGGAGAAGCTGCACTTCATCATCGGCAATGGCATCCTGCGGCCAGCAC
TCCGGGACGAGATCTACTGCCAGATCAGCAAGCAGCTGACCCACAACCCCTCCAA
GAGCAGCTATGCCCGGGGCTGGATTCTCGTGTCTCTCTGCGTGGGCTGTTTCGCCC
CCTCCGAGAAGTTTGTCAAGTACCTGCGGAACTTCATCCACGGGGGCCCGCCCGG
CTACGCCCCGTACTGTGAGGAGCGCCTGAGAAGGACCTTTGTCAATGGGACACGG
ACACAGCCGCCCAGCTGGCTGGAGCTGCAGGCCACCAAGTCCAAGAAGCCAATC
ATGTTGCCCGTGACATTCATGGATGGGACCACCAAGACCCTGCTGACGGACTCGG
CAACCACGGCCAAGGAGCTCTGCAACGCGCTGGCCGACAAGATCTCTCTCAAGG
ACCGGTTCGGGTTCTCCCTCTACATTGCCCTGTTTGACAAGGTGTCCTCCCTGGGC
AGCGGCAGTGACCACGTCATGGACGCCATCTCCCAGTGCGAGCAGTACGCCAAG
GAGCAGGGCGCCCAGGAGCGCAACGCCCCCTGGAGGCTCTTCTTCCGCAAAGAG
GTCTTCACGCCCTGGCACAGCCCCTCCGAGGACAACGTGGCCACCAACCTCATCT
ACCAGCAGGTGGTGCGAGGAGTCAAGTTTGGGGAGTACAGGTGTGAGAAGGAGG
ACGACCTGGCTGAGCTGGCCTCCCAGCAGTACTTTGTAGACTATGGCTCTGAGAT
GATCCTGGAGCGCCTCCTGAACCTCGTGCCCACCTACATCCCCGACCGCGAGATC
ACGCCCCTGAAGACGCTGGAGAAGTGGGCCCAGCTGGCCATCGCCGCCCACAAG
AAGGGGATTTATGCCCAGAGGAGAACTGATGCCCAGAAGGTCAAAGAGGATGTG
GTCAGTTATGCCCGCTTCAAGTGGCCCTTGCTCTTCTCCAGGTTTTATGAAGCCTA
CAAATTCTCAGGCCCCAGTCTCCCCAAGAACGACGTCATCGTGGCCGTCAACTGG
ACGGGTGTGTACTTTGTGGATGAGCAGGAGCAGGTACTTCTGGAGCTGTCCTTCC
CAGAGATCATGGCCGTGTCCAGCAGCAGGGAGTGCCGTGTCTGGCTCTCACTGGG
CTGCTCTGATCTTGGCTGTGCTGCGCCTCACTCAGGCTGGGCAGGACTGACCCCG
GCGGGGCCCTGTTCTCCGTGTTGGTCCTGCAGGGGAGCGAAAACGACGGCCCCCA
GCTTCACGCTGGCCACCATCAAGGGGGACGAATACACCTTCACCTCCAGTAATGC
TGAGGACATTCGTGACCTGGTGGTCACCTTCCTAGAGGGGCTCCGGAAGAGATCT
AAGTATGTTGTGGCCCTGCAGGATAACCCCAACCCCGCAGGCGAGGAGTCAGGCT
TCCTCAGCTTTGCCAAGGGAGACCTCATCATCCTGGACCATGACACGGGCGAGCA
GGTCATGAACTCGGGCTGGGCCAACGGCATCAATGAGAGGACCAAGCAGCGTGG
GGACTTCCCCACCGACTGTGTGTACGTCATGCCCACTGTCACCATGCCACCGCGG
GAGATTGTGGCCCTGGTCACCATGACTCCCGATCAGAGGCAGGACGTTGTCCGGC
TCTTGCAGCTGCGAACGGCGGAGCCCGAGGTGCGTGCCAAGCCCTACACGCTGGA
GGAGTTTTCCTATGACTACTTCAGGCCCCCACCCAAGCACACGCTGAGCCGTGTC
ATGGTGTCCAAGGCCCGAGGCAAGGACCGGCTGTGGAGCCACACGCGGGAACCG
CTCAAGCAGGCGCTGCTCAAGAAGCTCCTGGGCAGTGAGGAGCTCTCGCAGGAG
GCCTGCCTGGCCTTCATTGCTGTGCTCAAGTACATGGGCGACTACCCGTCCAAGA
GGACACGCTCCGTCAACGAGCTCACCGACCAGATCTTTGAGGGTCCCCTGAAAGC
CGAGCCCCTGAAGGACGAGGCATATGTGCAGATCCTGAAGCAGCTGACCGACAA
CCACATCAGGTACAGCGAGGAGCGGGGTTGGGAGCTGCTCTGGCTGTGCACGGG
CCTTTTCCCACCCAGCAACATCCTCCTGCCCCACGTGCAGCGCTTCCTGCAGTCCC
GAAAGCACTGCCCACTCGCCATCGACTGCCTGCAACGGCTCCAGAAAGCCCTGAG
AAACGGGTCCCGGAAGTACCCTCCGCACCTGGTGGAGGTGGAGGCCATCCAGCA
CAAGACCACCCAGATTTTCCACAAGGTCTACTTCCCTGATGACACTGACGAGGCC
TTCGAAGTGGAGTCCAGCACCAAGGCCAAGGACTTCTGCCAGAACATCGCCACCA
GGCTGCTCCTCAAGTCCTCAGAGGGATTCAGCCTCTTTGTCAAAATTGCAGACAA
GGTCATCAGCGTTCCTGAGAATGACTTCTTCTTTGACTTTGTTCGACACTTGACAG
ACTGGATAAAGAAAGCTCGGCCCATCAAGGACGGAATTGTGCCCTCACTCACCTA
CCAGGTGTTCTTCATGAAGAAGCTGTGGACCACCACGGTGCCAGGGAAGGATCCC
ATGGCCGATTCCATCTTCCACTATTACCAGGAGTTGCCCAAGTATCTCCGAGGCTA
CCACAAGTGCACGCGGGAGGAGGTGCTGCAGCTGGGGGCGCTGATCTACAGGGT
CAAGTTCGAGGAGGACAAGTCCTACTTCCCCAGCATCCCCAAGCTGCTGCGGGAG
CTGGTGCCCCAGGACCTTATCCGGCAGGTCTCACCTGATGACTGGAAGCGGTCCA
TCGTCGCCTACTTCAACAAGCACGCAGGGAAGTCCAAGGAGGAGGCCAAGCTGG
CCTTCCTGAAGCTCATCTTCAAGTGGCCCACCTTTGGCTCAGCCTTCTTCGAGGTG
AAGCAAACTACGGAGCCAAACTTCCCTGAGATCCTCCTAATTGCCATCAACAAGT
ATGGGGTCAGCCTCATCGATCCCAAAACGAAGGATATCCTCACCACTCATCCCTT
CACCAAGATCTCCAACTGGAGCAGCGGCAACACCTACTTCCACATCACCATTGGG
AACTTGGTGCGCGGGAGCAAACTGCTCTGCGAGACGTCACTGGGCTACAAGATG
GATGACCTCCTGACTTCCTACATTAGCCAGATGCTCACAGCCATGAGCAAACAGC
GGGGCTCCAGGAGCGGCAAGTGAACAGTCACGGGGAGGTGCTGGTTCCATGCCT
GCTCTCGAGGCAGCAGTGGGTTCAGGCCCATCAGCTACCCCTGCAGCTGGGGAAG
ACTTATGCCATCCCGGCAGCGAGGCTGGGCTGGCCAGCCACCACTGACTATACCA
ACTGGGCCTCTGATGTTCTTCCAGTGAGGCATCTCTCTGGGATGCAGAACTTCCCT
CCATCCACCCCTCTGGCACCTGGGTTGGTCTAATCCTAGTTTGCTGTGGCCTTCCC
GGTTGTGAGAGCCTGTGATCCTTAGATGTGTCTCCTGTTTCAGACCAGCCCCACCA
TGCAACTTCCTTTGACTTTCTGTGTACCACTGGGATAGAGGAATCAAGAGGACAA
TCTAGCTCTCCATACTTTGAACAACCAAATGTGCATTGAATACTCTGAAACCGAA
GGGACTGGATCTGCAGGTGGGATGAGGGAGACAGACCACTTTTCTATATTGCAGT
GTGAATGCTGGGCCCCTGCTCAAGTCTACCCTGATCACCTCAGGGCATAAAGCAT
GTTTCATTCTCTGAAA
SEQ ID NO: 6 is the amino acid sequence of the human myosin VIIa polypeptide
encoded by nucleotides 273-6920 of SEQ ID NO: 5;
MVILQQGDHVWMDLRLGQEFDVPIGAVVKLCDSGQVQVVDDEDNEHWISPQNATH
IKPMHPTSVHGVEDMIRLGDLNEAGILRNLLIRYRDHLIYTYTGSILVAVNPYQLLSIY
SPEHIRQYTNKKIGEMPPHIFAIADNCYFNMKRNSRDQCCIISGESGAGKTESTKLILQ
FLAAISGQHSWIEQQVLEATPILEAFGNAKTIRNDNSSRFGKYIDIHFNKRGAIEGAKIE
QYLLEKSRVCRQALDERNYHVFYCMLEGMSEDQKKKLGLGQASDYNYLAMGNCIT
CEGRVDSQEYANIRSAMKVLMFTDTENWEISKLLAAILHLGNLQYEARTFENLDACE
VLFSPSLATAASLLEVNPPDLMSCLTSRTLITRGETVSTPLSREQALDVRDAFVKGIYG
RLFVWIVDKINAAIYKPPSQDVKNSRRSIGLLDIFGFENFAVNSFEQLCINFANEHLQQ
FFVRHVFKLEQEEYDLESIDWLHIEFTDNQDALDMIANKPMNIISLIDEESKFPKGTDT
TMLHKLNSQHKLNANYIPPKNNHETQFGINHFAGIVYYETQGFLEKNRDTLHGDIIQL
VHSSRNKFIKQIFQADVAMGAETRKRSPTLSSQFKRSLELLMRTLGACQPFFVRCIKP
NEFKKPMLFDRHLCVRQLRYSGMMETIRIRRAGYPIRYSFVEFVERYRVLLPGVKPA
YKQGDLRGTCQRMAEAVLGTHDDWQIGKTKIFLKDHHDMLLEVERDKAITDRVILL
QKVIRGFKDRSNFLKLKNAATLIQRHWRGHNCRKNYGLMRLGFLRLQALHRSRKLH
QQYRLARQRIIQFQARCRAYLVRKAFRHRLWAVLTVQAYARGMIARRLHQRLRAEY
LWRLEAEKMRLAEEEKLRKEMSAKKAKEEAERKHQERLAQLAREDAERELKEKEA
ARRKKELLEQMERARHEPVNHSDMVDKMFGFLGTSGGLPGQEGQAPSGFEDLERGR
REMVEEDLDAALPLPDEDEEDLSEYKFAKFAATYFQGTTTHSYTRRPLKQPLLYHDD
EGDQLAALAVWITILRFMGDLPEPKYHTAMSDGSEKIPVMTKIYETLGKKTYKRELQ
ALQGEGEAQLPEGQKKSSVRHKLVHLTLKKKSKLTEEVTKRLHDGESTVQGNSMLE
DRPTSNLEKLHFIIGNGILRPALRDEIYCQISKQLTHNPSKSSYARGWILVSLCVGCFAP
SEKFVKYLRNFIHGGPPGYAPYCEERLRRTFVNGTRTQPPSWLELQATKSKKPIMLPV
TFMDGTTKTLLTDSATTAKELCNALADKISLKDRFGFSLYIALFDKVSSLGSGSDHV
MDAISQCEQYAKEQGAQERNAPWRLFFRKEVFTPWHSPSEDNVATNLIYQQVVRGV
KFGEYRCEKEDDLAELASQQYFVDYGSEMILERLLNLVPTYIPDREITPLKTLEKWAQ
LAIAAHKKGIYAQRRTDAQKVKEDVVSYARFKWPLLFSRFYEAYKFSGPSLPKNDVI
VAVNWTGVYFVDEQEQVLLELSFPEIMAVSSSRECRVWLSLGCSDLGCAAPHSGWA
GLTPAGPCSPCWSCRGAKTTAPSFTLATIKGDEYTFTSSNAEDIRDLVVTFLEGLRKR
SKYVVALQDNPNPAGEESGFLSFAKGDLIILDHDTGEQVMNSGWANGINERTKQRG
DFPTDCVYVMPTVTMPPREIVALVTMTPDQRQDVVRLLQLRTAEPEVRAKPYTLEEF
SYDYFRPPPKHTLSRVMVSKARGKDRLWSHTREPLKQALLKKLLGSEELSQEACLAF
IAVLKYMGDYPSKRTRSVNELTDQIFEGPLKAEPLKDEAYVQILKQLTDNHIRYSEER
GWELLWLCTGLFPPSNILLPHVQRFLQSRKHCPLAIDCLQRLQKALRNGSRKYPPHL
VEVEAIQHKTTQIFHKVYFPDDTDEAFEVESSTKAKDFCQNIATRLLLKSSEGFSLFVK
IADKVISVPENDFFFDFVRHLTDWIKKARPIKDGIVPSLTYQVFFMKKLWTTTVPGKD
PMADSIFHYYQELPKYLRGYHKCTREEVLQLGALIYRVKFEEDKSYFPSIPKLLRELV
PQDLIRQVSPDDWKRSIVAYFNKHAGKSKEEAKLAFLKLIFKWPTFGSAFFEVKQTTE
PNFPEILLIAINKYGVSLIDPKTKDILTTHPFTKISNWSSGNTYFHITIGNLVRGSKLLCE
TSLGYKMDDLLTSYISQMLTAMSKQRGSRSGK
SEQ ID NO: 7 is a nucleotide sequence that encodes a human myosin VIIa
polypeptide;
ATGGTGATTCTTCAGCAGGGGGACCATGTGTGGATGGACCTGAGATTGGGGCAGG
AGTTCGACGTGCCCATCGGGGCGGTGGTGAAGCTCTGCGACTCTGGGCAGGTCCA
GGTGGTGGATGATGAAGACAATGAACACTGGATCTCTCCGCAGAACGCAACGCA
CATCAAGCCTATGCACCCCACGTCGGTCCACGGCGTGGAGGACATGATCCGCCTG
GGGGACCTCAACGAGGCGGGCATCTTGCGCAACCTGCTTATCCGCTACCGGGACC
ACCTCATCTACACGTATACGGGCTCCATCCTGGTGGCTGTGAACCCCTACCAGCT
GCTCTCCATCTACTCGCCAGAGCACATCCGCCAGTATACCAACAAGAAGATTGGG
GAGATGCCCCCCCACATCTTTGCCATTGCTGACAACTGCTACTTCAACATGAAAC
GCAACAGCCGAGACCAGTGCTGCATCATCAGTGGGGAATCTGGGGCCGGGAAGA
CGGAGAGCACAAAGCTGATCCTGCAGTTCCTGGCAGCCATCAGTGGGCAGCACTC
GTGGATTGAGCAGCAGGTCTTGGAGGCCACCCCCATTCTGGAAGCATTTGGGAAT
GCCAAGACCATCCGCAATGACAACTCAAGCCGTTTCGGAAAGTACATCGACATCC
ACTTCAACAAGCGGGGCGCCATCGAGGGCGCGAAGATTGAGCAGTACCTGCTGG
AAAAGTCACGTGTCTGTCGCCAGGCCCTGGATGAAAGGAACTACCACGTGTTCTA
CTGCATGCTGGAGGGTATGAGTGAGGATCAGAAGAAGAAGCTGGGCTTGGGCCA
GGCCTCTGACTACAACTACTTGGCCATGGGTAACTGCATAACCTGTGAGGGCCGG
GTGGACAGCCAGGAGTACGCCAACATCCGCTCCGCCATGAAGGTGCTCATGTTCA
CTGACACCGAGAACTGGGAGATCTCGAAGCTCCTGGCTGCCATCCTGCACCTGGG
CAACCTGCAGTATGAGGCACGCACATTTGAAAACCTGGATGCCTGTGAGGTTCTC
TTCTCCCCATCGCTGGCCACAGCTGCATCCCTGCTTGAGGTGAACCCCCCAGACCT
GATGAGCTGCCTGACTAGCCGCACCCTCATCACCCGCGGGGAGACGGTGTCCACC
CCACTGAGCAGGGAACAGGCACTGGACGTGCGCGACGCCTTCGTAAAGGGGATC
TACGGGCGGCTGTTCGTGTGGATTGTGGACAAGATCAACGCAGCAATTTACAAGC
CTCCCTCCCAGGATGTGAAGAACTCTCGCAGGTCCATCGGCCTCCTGGACATCTTT
GGGTTTGAGAACTTTGCTGTGAACAGCTTTGAGCAGCTCTGCATCAACTTCGCCA
ATGAGCACCTGCAGCAGTTCTTTGTGCGGCACGTGTTCAAGCTGGAGCAGGAGGA
ATATGACCTGGAGAGCATTGACTGGCTGCACATCGAGTTCACTGACAACCAGGAT
GCCCTGGACATGATTGCCAACAAGCCCATGAACATCATCTCCCTCATCGATGAGG
AGAGCAAGTTCCCCAAGGGCACAGACACCACCATGTTACACAAGCTGAACTCCC
AGCACAAGCTCAACGCCAACTACATCCCCCCCAAGAACAACCATGAGACCCAGTT
TGGCATCAACCATTTTGCAGGCATCGTCTACTATGAGACCCAAGGCTTCCTGGAG
AAGAACCGAGACACCCTGCATGGGGACATTATCCAGCTGGTCCACTCCTCCAGGA
ACAAGTTCATCAAGCAGATCTTCCAGGCCGATGTCGCCATGGGCGCCGAGACCAG
GAAGCGCTCGCCCACACTTAGCAGCCAGTTCAAGCGGTCACTGGAGCTGCTGATG
CGCACGCTGGGTGCCTGCCAGCCCTTCTTTGTGCGATGCATCAAGCCCAATGAGT
TCAAGAAGCCCATGCTGTTCGACCGGCACCTGTGCGTGCGCCAGCTGCGGTACTC
AGGAATGATGGAGACCATCCGAATCCGCCGAGCTGGCTACCCCATCCGCTACAGC
TTCGTAGAGTTTGTGGAGCGGTACCGTGTGCTGCTGCCAGGTGTGAAGCCGGCCT
ACAAGCAGGGCGACCTCCGCGGGACTTGCCAGCGCATGGCTGAGGCTGTGCTGG
GCACCCACGATGACTGGCAGATAGGCAAAACCAAGATCTTTCTGAAGGACCACC
ATGACATGCTGCTGGAAGTGGAGCGGGACAAAGCCATCACCGACAGAGTCATCC
TCCTTCAGAAAGTCATCCGGGGATTCAAAGACAGGTCTAACTTTCTGAAGCTGAA
GAACGCTGCCACACTGATCCAGAGGCACTGGCGGGGTCACAACTGTAGGAAGAA
CTACGGGCTGATGCGTCTGGGCTTCCTGCGGCTGCAGGCCCTGCACCGCTCCCGG
AAGCTGCACCAGCAGTACCGCCTGGCCCGCCAGCGCATCATCCAGTTCCAGGCCC
GCTGCCGCGCCTATCTGGTGCGCAAGGCCTTCCGCCACCGCCTCTGGGCTGTGCTC
ACCGTGCAGGCCTATGCCCGGGGCATGATCGCCCGCAGGCTGCACCAACGCCTCA
GGGCTGAGTATCTGTGGCGCCTCGAGGCTGAGAAAATGCGGCTGGCGGAGGAAG
AGAAGCTTCGGAAGGAGATGAGCGCCAAGAAGGCCAAGGAGGAGGCCGAGCGC
AAGCATCAGGAGCGCCTGGCCCAGCTGGCTCGTGAGGACGCTGAGCGGGAGCTG
AAGGAGAAGGAGGCCGCTCGGCGGAAGAAGGAGCTCCTGGAGCAGATGGAAAG
GGCCCGCCATGAGCCTGTCAATCACTCAGACATGGTGGACAAGATGTTTGGCTTC
CTGGGGACTTCAGGTGGCCTGCCAGGCCAGGAGGGCCAGGCACCTAGTGGCTTTG
AGGACCTGGAGCGAGGGCGGAGGGAGATGGTGGAGGAGGACCTGGATGCAGCC
CTGCCCCTGCCTGACGAGGATGAGGAGGACCTCTCTGAGTATAAATTTGCCAAGT
TCGCGGCCACCTACTTCCAGGGGACAACCACGCACTCCTACACCCGGCGGCCACT
CAAACAGCCACTGCTCTACCATGACGACGAGGGTGACCAGCTGGCAGCCCTGGC
GGTCTGGATCACCATCCTCCGCTTCATGGGGGACCTCCCTGAGCCCAAGTACCAC
ACAGCCATGAGTGATGGCAGTGAGAAGATCCCTGTGATGACCAAGATTTATGAG
ACCCTGGGCAAGAAGACGTACAAGAGGGAGCTGCAGGCCCTGCAGGGCGAGGGC
GAGGCCCAGCTCCCCGAGGGCCAGAAGAAGAGCAGTGTGAGGCACAAGCTGGTG
CATTTGACTCTGAAAAAGAAGTCCAAGCTCACAGAGGAGGTGACCAAGAGGCTG
CATGACGGGGAGTCCACAGTGCAGGGCAACAGCATGCTGGAGGACCGGCCCACC
TCCAACCTGGAGAAGCTGCACTTCATCATCGGCAATGGCATCCTGCGGCCAGCAC
TCCGGGACGAGATCTACTGCCAGATCAGCAAGCAGCTGACCCACAACCCCTCCAA
GAGCAGCTATGCCCGGGGCTGGATTCTCGTGTCTCTCTGCGTGGGCTGTTTCGCCC
CCTCCGAGAAGTTTGTCAAGTACCTGCGGAACTTCATCCACGGGGGCCCGCCCGG
CTACGCCCCGTACTGTGAGGAGCGCCTGAGAAGGACCTTTGTCAATGGGACACGG
ACACAGCCGCCCAGCTGGCTGGAGCTGCAGGCCACCAAGTCCAAGAAGCCAATC
ATGTTGCCCGTGACATTCATGGATGGGACCACCAAGACCCTGCTGACGGACTCGG
CAACCACGGCCAAGGAGCTCTGCAACGCGCTGGCCGACAAGATCTCTCTCAAGG
ACCGGTTCGGGTTCTCCCTCTACATTGCCCTGTTTGACAAGGTGTCCTCCCTGGGC
AGCGGCAGTGACCACGTCATGGACGCCATCTCCCAGTGCGAGCAGTACGCCAAG
GAGCAGGGCGCCCAGGAGCGCAACGCCCCCTGGAGGCTCTTCTTCCGCAAAGAG
GTCTTCACGCCCTGGCACAGCCCCTCCGAGGACAACGTGGCCACCAACCTCATCT
ACCAGCAGGTGGTGCGAGGAGTCAAGTTTGGGGAGTACAGGTGTGAGAAGGAGG
ACGACCTGGCTGAGCTGGCCTCCCAGCAGTACTTTGTAGACTATGGCTCTGAGAT
GATCCTGGAGCGCCTCCTGAACCTCGTGCCCACCTACATCCCCGACCGCGAGATC
ACGCCCCTGAAGACGCTGGAGAAGTGGGCCCAGCTGGCCATCGCCGCCCACAAG
AAGGGGATTTATGCCCAGAGGAGAACTGATGCCCAGAAGGTCAAAGAGGATGTG
GTCAGTTATGCCCGCTTCAAGTGGCCCTTGCTCTTCTCCAGGTTTTATGAAGCCTA
CAAATTCTCAGGCCCCAGTCTCCCCAAGAACGACGTCATCGTGGCCGTCAACTGG
ACGGGTGTGTACTTTGTGGATGAGCAGGAGCAGGTACTTCTGGAGCTGTCCTTCC
CAGAGATCATGGCCGTGTCCAGCAGCAGGGAGTGCCGTGTCTGGCTCTCACTGGG
CTGCTCTGATCTTGGCTGTGCTGCGCCTCACTCAGGCTGGGCAGGACTGACCCCG
GCGGGGCCCTGTTCTCCGTGTTGGTCCTGCAGGGGAGCGAAAACGACGGCCCCCA
GCTTCACGCTGGCCACCATCAAGGGGGACGAATACACCTTCACCTCCAGTAATGC
TGAGGACATTCGTGACCTGGTGGTCACCTTCCTAGAGGGGCTCCGGAAGAGATCT
AAGTATGTTGTGGCCCTGCAGGATAACCCCAACCCCGCAGGCGAGGAGTCAGGCT
TCCTCAGCTTTGCCAAGGGAGACCTCATCATCCTGGACCATGACACGGGCGAGCA
GGTCATGAACTCGGGCTGGGCCAACGGCATCAATGAGAGGACCAAGCAGCGTGG
GGACTTCCCCACCGACTGTGTGTACGTCATGCCCACTGTCACCATGCCACCGCGG
GAGATTGTGGCCCTGGTCACCATGACTCCCGATCAGAGGCAGGACGTTGTCCGGC
TCTTGCAGCTGCGAACGGCGGAGCCCGAGGTGCGTGCCAAGCCCTACACGCTGGA
GGAGTTTTCCTATGACTACTTCAGGCCCCCACCCAAGCACACGCTGAGCCGTGTC
ATGGTGTCCAAGGCCCGAGGCAAGGACCGGCTGTGGAGCCACACGCGGGAACCG
CTCAAGCAGGCGCTGCTCAAGAAGCTCCTGGGCAGTGAGGAGCTCTCGCAGGAG
GCCTGCCTGGCCTTCATTGCTGTGCTCAAGTACATGGGCGACTACCCGTCCAAGA
GGACACGCTCCGTCAACGAGCTCACCGACCAGATCTTTGAGGGTCCCCTGAAAGC
CGAGCCCCTGAAGGACGAGGCATATGTGCAGATCCTGAAGCAGCTGACCGACAA
CCACATCAGGTACAGCGAGGAGCGGGGTTGGGAGCTGCTCTGGCTGTGCACGGG
CCTTTTCCCACCCAGCAACATCCTCCTGCCCCACGTGCAGCGCTTCCTGCAGTCCC
GAAAGCACTGCCCACTCGCCATCGACTGCCTGCAACGGCTCCAGAAAGCCCTGAG
AAACGGGTCCCGGAAGTACCCTCCGCACCTGGTGGAGGTGGAGGCCATCCAGCA
CAAGACCACCCAGATTTTCCACAAGGTCTACTTCCCTGATGACACTGACGAGGCC
TTCGAAGTGGAGTCCAGCACCAAGGCCAAGGACTTCTGCCAGAACATCGCCACCA
GGCTGCTCCTCAAGTCCTCAGAGGGATTCAGCCTCTTTGTCAAAATTGCAGACAA
GGTCATCAGCGTTCCTGAGAATGACTTCTTCTTTGACTTTGTTCGACACTTGACAG
ACTGGATAAAGAAAGCTCGGCCCATCAAGGACGGAATTGTGCCCTCACTCACCTA
CCAGGTGTTCTTCATGAAGAAGCTGTGGACCACCACGGTGCCAGGGAAGGATCCC
ATGGCCGATTCCATCTTCCACTATTACCAGGAGTTGCCCAAGTATCTCCGAGGCTA
CCACAAGTGCACGCGGGAGGAGGTGCTGCAGCTGGGGGCGCTGATCTACAGGGT
CAAGTTCGAGGAGGACAAGTCCTACTTCCCCAGCATCCCCAAGCTGCTGCGGGAG
CTGGTGCCCCAGGACCTTATCCGGCAGGTCTCACCTGATGACTGGAAGCGGTCCA
TCGTCGCCTACTTCAACAAGCACGCAGGGAAGTCCAAGGAGGAGGCCAAGCTGG
CCTTCCTGAAGCTCATCTTCAAGTGGCCCACCTTTGGCTCAGCCTTCTTCGAGGTG
AAGCAAACTACGGAGCCAAACTTCCCTGAGATCCTCCTAATTGCCATCAACAAGT
ATGGGGTCAGCCTCATCGATCCCAAAACGAAGGATATCCTCACCACTCATCCCTT
CACCAAGATCTCCAACTGGAGCAGCGGCAACACCTACTTCCACATCACCATTGGG
AACTTGGTGCGCGGGAGCAAACTGCTCTGCGAGACGTCACTGGGCTACAAGATG
GATGACCTCCTGACTTCCTACATTAGCCAGATGCTCACAGCCATGAGCAAACAGC
GGGGCTCCAGGAGCGGCAAGTGA
SEQ ID NO: 8 is an amino acid sequence of a human myosin VIIa polypeptide
(isoform 2);
MVILQQGDHVWMDLRLGQEFDVPIGAVVKLCDSGQVQVVDDEDNEHWISPQNATH
IKPMHPTSVHGVEDMIRLGDLNEAGILRNLLIRYRDHLIYTYTGSILVAVNPYQLLSIY
SPEHIRQYTNKKIGEMPPHIFAIADNCYFNMKRNSRDQCCIISGESGAGKTESTKLILQ
FLAAISGQHSWIEQQVLEATPILEAFGNAKTIRNDNSSRFGKYIDIHFNKRGAIEGAKIE
QYLLEKSRVCRQALDERNYHVFYCMLEGMSEDQKKKLGLGQASDYNYLAMGNCIT
CEGRVDSQEYANIRSAMKVLMFTDTENWEISKLLAAILHLGNLQYEARTFENLDACE
VLFSPSLATAASLLEVNPPDLMSCLTSRTLITRGETVSTPLSREQALDVRDAFVKGIYG
RLFVWIVDKINAAIYKPPSQDVKNSRRSIGLLDIFGFENFAVNSFEQLCINFANEHLQQ
FFVRHVFKLEQEEYDLESIDWLHIEFTDNQDALDMIANKPMNIISLIDEESKFPKGTDT
TMLHKLNSQHKLNANYIPPKNNHETQFGINHFAGIVYYETQGFLEKNRDTLHGDIIQL
VHSSRNKFIKQIFQADVAMGAETRKRSPTLSSQFKRSLELLMRTLGACQPFFVRCIKP
NEFKKPMLFDRHLCVRQLRYSGMMETIRIRRAGYPIRYSFVEFVERYRVLLPGVKPA
YKQGDLRGTCQRMAEAVLGTHDDWQIGKTKIFLKDHHDMLLEVERDKAITDRVILL
QKVIRGFKDRSNFLKLKNAATLIQRHWRGHNCRKNYGLMRLGFLRLQALHRSRKLH
QQYRLARQRIIQFQARCRAYLVRKAFRHRLWAVLTVQAYARGMIARRLHQRLRAEY
LWRLEAEKMRLAEEEKLRKEMSAKKAKEEAERKHQERLAQLAREDAERELKEKEA
ARRKKELLEQMERARHEPVNHSDMVDKMFGFLGTSGGLPGQEGQAPSGFEDLERGR
REMVEEDLDAALPLPDEDEEDLSEYKFAKFAATYFQGTTTHSYTRRPLKQPLLYHDD
EGDQLAALAVWITILRFMGDLPEPKYHTAMSDGSEKIPVMTKIYETLGKKTYKRELQ
ALQGEGEAQLPEGQKKSSVRHKLVHLTLKKKSKLTEEVTKRLHDGESTVQGNSMLE
DRPTSNLEKLHFIIGNGILRPALRDEIYCQISKQLTHNPSKSSYARGWILVSLCVGCFAP
SEKFVKYLRNFIHGGPPGYAPYCEERLRRTFVNGTRTQPPSWLELQATKSKKPIMLPV
TFMDGTTKTLLTDSATTAKELCNALADKISLKDRFGFSLYIALFDKVSSLGSGSDHV
MDAISQCEQYAKEQGAQERNAPWRLFFRKEVFTPWHSPSEDNVATNLIYQQVVRGV
KFGEYRCEKEDDLAELASQQYFVDYGSEMILERLLNLVPTYIPDREITPLKTLEKWAQ
LAIAAHKKGIYAQRRTDAQKVKEDVVSYARFKWPLLFSRFYEAYKFSGPSLPKNDVI
VAVNWTGVYFVDEQEQVLLELSFPEIMAVSSSRGAKTTAPSFTLATIKGDEYTFTSSN
AEDIRDLVVTFLEGLRKRSKYVVALQDNPNPAGEESGFLSFAKGDLIILDHDTGEQV
MNSGWANGINERTKQRGDFPTDSVYVMPTVTMPPREIVALVTMTPDQRQDVVRLL
QLRTAEPEVRAKPYTLEEFSYDYFRPPPKHTLSRVMVSKARGKDRLWSHTREPLKQA
LLKKLLGSEELSQEACLAFIAVLKYMGDYPSKRTRSVNELTDQIFEGPLKAEPLKDEA
YVQILKQLTDNHIRYSEERGWELLWLCTGLFPPSNILLPHVQRFLQSRKHCPLAIDCL
QRLQKALRNGSRKYPPHLVEVEAIQHKTTQIFHKVYFPDDTDEAFEVESSTKAKDFC
QNIATRLLLKSSEGFSLFVKIADKVLSVPENDFFFDFVRHLTDWIKKARPIKDGIVPSL
TYQVFFMKKLWTTTVPGKDPMADSIFHYYQELPKYLRGYHKCTREEVLQLGALIYR
VKFEEDKSYFPSIPKLLRELVPQDLIRQVSPDDWKRSIVAYFNKHAGKSKEEAKLAFL
KLIFKWPTFGSAFFEQTTEPNFPEILLIAINKYGVSLIDPKTKDILTTHPFTKISNWSSGN
TYFHITIGNLVRGSKLLCETSLGYKMDDLLTSYISQMLTAMSKQRGSRSGK

### CMv1 Overlap Vector System

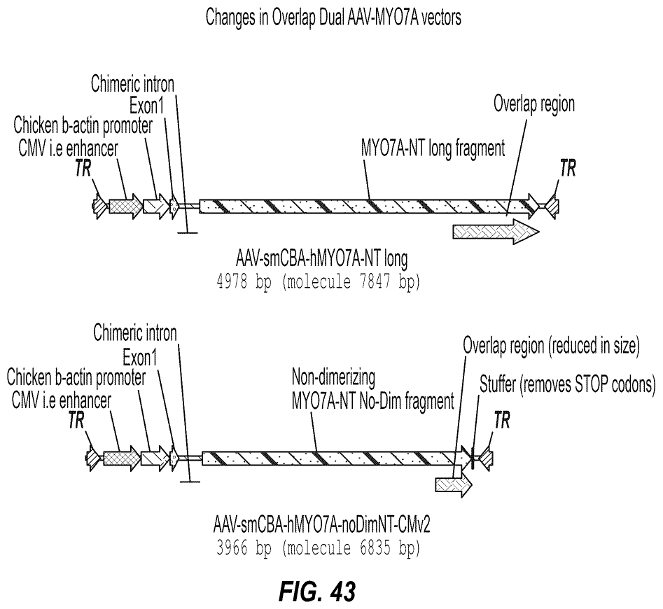

Some embodiments contemplate the overlapping vector system as described herein, with one or more substitutions made in the 3′ untranslated region downstream of the MYO7A partial coding sequence, and before the 3′ AAV inverted terminal repeat. In some embodiments, these substitutions are intended to remove potential (or putative) in-frame stop codons. In some embodiments, these substitutions remove one or more putative stop codons in a non-coding sequence. In particular embodiments, the substitutions remove one or more putative stop codons in the 3′ untranslated region between the partial coding sequence encoding the C-terminal part of the polypeptide and the 3′ AAV inverted terminal repeat of the second AAV vector polynucleotide (e.g., downstream of the MYO7A N-terminal fragment). In some embodiments, the one or more putative stop codons are removed and replaced with a “stuffer” sequence (see FIG. 43). In some embodiments, the first AAV vector polynucleotide thus comprises a partial coding sequence that does not encode the single-alpha helix (SAH) domain of the selected full-length polypeptide.

As a result of these substitutions, a front-half vector is created having the nucleotide sequence comprising SEQ ID NO: 36. In such embodiments, overlap vector systems of the disclosure may comprise:
(i) a first AAV vector polynucleotide comprising an inverted terminal repeat at each end of the polynucleotide, and between the inverted terminal repeats a promoter followed by a partial coding sequence that encodes an N-terminal part of a selected full-length polypeptide; and
(ii) a second AAV vector polynucleotide comprising an inverted terminal repeat at each end of the polynucleotide, and between the inverted terminal repeats a partial coding sequence that encodes a C-terminal part of the selected full-length polypeptide. In some embodiments, the second AAV vector polynucleotide is followed by a polyadenylation (pA) signal sequence. The coding sequences in the first and second vectors when combined encode the selected full-length polypeptide, or a functional fragment or variant thereof. The polypeptide encoding sequence in the first and second AAV vectors comprises sequence that overlaps.

In some embodiments, the C-terminal part of the selected full-length polypeptide (e.g., the myosin polypeptide) comprises the single-alpha helix (SAH) domain of the selected full-length polypeptide.

In some embodiments, the polynucleotide sequence that overlaps comprises SEQ ID NO: 39. In particular embodiments, the sequence overlap between the first and second AAV vectors is between about 1 and about 500 nucleotides; between about 100 and about 200 nucleotides; between about 200 and about 300 nucleotides; or between about 300 and about 400 nucleotides. In particular embodiments, the sequence overlap of the second generation overlap vector system is about 350 nucleotides. In an exemplary embodiment, the sequence overlap of the second generation overlap vector system is 361 nucleotides.

In some embodiments, the polynucleotide sequence that overlaps does not comprise any portion of exon 23 of the hMYO7A gene. In some embodiments, the polynucleotide sequence that overlaps does not comprise exon 23 in full (e.g., 100% of exon 23). In some embodiments, the polynucleotide sequence that overlaps comprises a portion of exon 17, exon 18 in full, exon 19 in full, exon 20 in full, and a portion of exon 21 of the hMYO7A gene. In some embodiments, the polynucleotide sequence that overlaps comprises a portion of exon 17, a portion of exon 18, a portion of exon 19, a portion of exon 20, and/or a portion of exon 21 of the hMYO7A gene. As used herein, a “portion” may comprise e.g. at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, etc. of the exon and/or intron.

In an exemplary embodiment, the first AAV vector polynucleotide comprises the nucleotide sequence of SEQ ID NO: 36, or a functional fragment and/or variant thereof, and the second AAV vector polynucleotide comprises the nucleotide sequence of SEQ ID NO: 38, or a functional fragment and/or variant thereof.

In some embodiments, first AAV vector polynucleotide comprises a partial coding sequence that does not encode the single-alpha helix (SAH) domain of the selected full-length (e.g. myosin) polypeptide. In some embodiments, the second AAV vector polynucleotide is followed by a polyadenylation (pA) signal sequence.

In some embodiments, any vector of the overlap polynucleotide vector systems described in the disclosure may be administered by parenteral administration, such as intravenous, intramuscular, intraocular, intranasal, etc. The vector can be administered in vivo, in vitro or ex vivo.

In some embodiments, any vector of the overlap polynucleotide vector systems described herein may be administered to the eye. In particular embodiments, a vector is administered to the eye of a subject by subretinal injection. In some embodiments, any vector of the hybrid polynucleotide vector systems described herein may be administered to the ear.

In some embodiments, any vector of the hybrid polynucleotide vector systems described herein the polynucleotide vector system is administered to the ear of a subject, e.g., by a round window injection or during cochlear implant surgery.

SEQ ID NO: 5 is a nucleotide sequence encoding a human myosin VIIa polypeptide (protein coding sequence is nucleotides 273-6920), the sequence of which is disclosed herein;

SEQ ID NO: 6 is the amino acid sequence of the human myosin VIIa polypeptide encoded by nucleotides 273-6920 of SEQ ID NO: 5, the sequence of which is disclosed herein;

SEQ ID NO: 7 is a nucleotide sequence that encodes a human myosin VIIa polypeptide, the sequence of which is disclosed herein;

SEQ ID NO: 8 is an amino acid sequence of a human myosin VIIa polypeptide (isoform 2), the sequence of which is disclosed herein.

Some aspects of the disclosed overlap vectors contemplate a virus or a recombinant viral particle comprising the first AAV vector polynucleotide or the second AAV vector polynucleotide as described herein. In particular embodiments, the first AAV vector polynucleotide comprises SEQ ID NO: 36, and the second AAV vector polynucleotide comprises SEQ ID NO: 38. In some embodiments, the virus or recombinant viral particle is characterized as an adeno-associated virus (AAV) or an infectious AAV viral particle. In some embodiments, the recombinant AAV viral particle includes one or more tyrosine-to-phenylalanine (Y-F) mutations in a capsid protein of the virus or virion. Tyrosine-to-phenylalanine (Y-F) mutations in a capsid protein of the virus or virion at amino acid position 733 are specifically contemplated herein (for example, AAV8 Y733F).

In some embodiments, the virus or virion is packaged in an AAV5, AAV7, AAV8, AAV9, AAV44.9, AAV44.9(E531D), AAV2(4pMut), AAVAnc80, AAVrh.8, AAVrh.8R, AAV9-PHP.B, AAV9-PHP.eB, AAVrh.10, or AAVrh.74 capsid. In some embodiments, the viral particle comprises an AAV1, AAV2, AAV3, AAV4, AAV5, AAV6, AAV7, AAV8, AAV9, AAV10, AAV7m8, AAV-DJ, AAV2/2-MAX, AAVSHh10, AAVSHh10Y, AAV3b, AAVLK03, AAV8PB2, AAV1(E531K), AAV6(D532N), AAV6-3pmut, AAV2G9, AAV44.9, AAV44.9(E531D), AAVrh.8, AAVrh.8R, AAV9-PHP.B, and/or AAVAnc80 capsid. In exemplary embodiments, the virion is packaged in an AAV44.9(E531D) capsid variant.

In some embodiments, the overlap polynucleotide vector systems described herein use a tissue-specific promoter. In some embodiments, the systems use a promoter that mediates expression in the eye. In some embodiments, the systems use a promoter that mediates expression in the ear.

In some embodiments, the overlap polynucleotide vector systems described herein use any one of the following promoters: a cytomegalovirus (CMV) promoter, an elongation factor-1 alpha (EF-1 alpha) promoter, a cone arrestin promoter, a chimeric CMV R actin promoter (CBA), a truncated chimeric CMV R actin (smCBA), a human myosin 7a gene-derived promoter, a cone transducin a (TaC) gene-derived promoter, a rhodopsin promoter, a cGMP-phosphodiesterase P-subunit promoter, human or mouse rhodopsin promoter, a human rhodopsin kinase (hGRK1) promoter, a synapsin promoter, a glial fibrillary acidic protein (GFAP) promoter, a rod specific IRBP promoter, a RPE-specific vitelliform macular dystrophy-2 [VMD2]promoter, and combinations thereof. In some embodiments, the polynucleotide vector system described herein uses a human rhodopsin kinase (hGRK1) promoter. In some embodiments, the polynucleotide vector system uses a cone arrestin promoter.

In some embodiments for delivery to the eyes (retina) of a subject, the disclosed overlap polynucleotide vector systems use a cytomegalovirus (CMV) promoter. In some embodiments, the polynucleotide vector system uses an EF-1 alpha promoter. In some embodiments for delivery to the ears (hair cells) of a subject, the polynucleotide vector system uses a synapsin or GFAP promoter (see Lee et al., Hearing Research).

### Hybrid Vector Systems

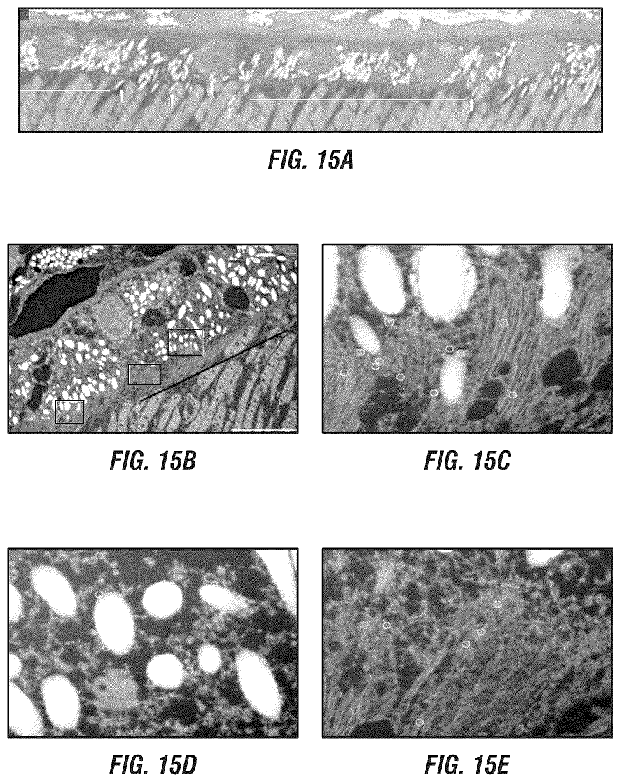

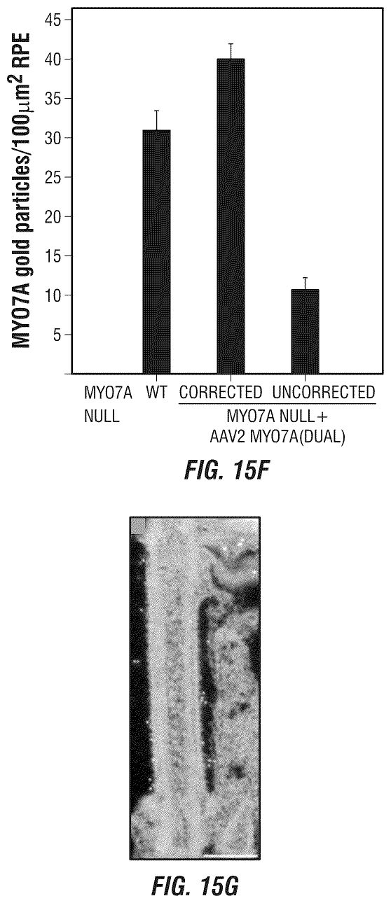

In some aspects, hybrid dual AAV vector systems are provided. These hybrid vector systems drive higher levels of full length MYO7A than overlap vectors and produce truncated protein from the front half vector. The hybrid front half vector leads to reduced retinal function in subretinally injected mice. (See FIGS. 15B-E.) In various embodiments, the hybrid vector systems of the disclosure do not produce a truncated MYO7A protein fragment following administration to a mouse or a subject.

Altering the split point, codon modifying the front half vector, and/or minimizing the length of the back half vector leads to production of full-length MYO7A at levels equal to or above that seen with the first generation hybrid vectors. The improved vectors provided herein generate far less undesired truncated protein side product. from the front half vector. In some embodiments, production of the truncated protein is eliminated, partially or completely.

In some aspects of the disclosure, a hybrid vector system of the disclosure includes:
(i) a first AAV vector polynucleotide comprising an inverted terminal repeat at each end (for example, the 5′-end and the 3′-end) of the polynucleotide, and between the inverted terminal repeats a suitable promoter followed by (for example, 3′ to the promoter) a partial coding sequence that encodes an N-terminal part of a selected full-length polypeptide followed by a splice donor site and an intron, and
(ii) a second AAV vector polynucleotide comprising an inverted terminal repeat at each end (5′-end and 3′-end) of the polynucleotide, and between the inverted terminal repeats an intron and a splice acceptor site for the intron, optionally followed by a partial coding sequence that encodes a C-terminal part of the selected full-length polypeptide, optionally followed by a polyadenylation (pA) signal sequence. The intron sequence in the first and second AAV vectors comprises sequence that overlaps.

In some embodiments, the split point between the first and second AAV vector polynucleotide sequences is between exon 21 and exon 22 of the hMYO7A gene.

In an exemplary embodiment, the first AAV vector polynucleotide comprises the nucleotide sequence of SEQ ID NO: 31, or a functional fragment and/or variant thereof, and the second AAV vector polynucleotide comprises the nucleotide sequence of SEQ ID NO: 32, or a functional fragment and/or variant thereof.

The coding sequences in the first and second vectors when combined encode the selected full-length polypeptide, or a functional fragment or variant thereof. In some embodiments, the selected full-length polypeptide is hMYO7A. In some embodiments, the selected full-length polypeptide is hMYO7B. In some embodiments, the selected full-length polypeptide is isoform II of hMYO7A. In some embodiments, the polynucleotide sequence corresponding to the tail domain of the MYO7A protein is removed from the first AAV vector polynucleotide.

It will be appreciated that this disclosure is not limited to delivery of full-length myosin 7A polypeptide or myosin 7A-encoding nucleotides. In some embodiments, the selected full-length polypeptide is selected from ABCA4 (Stargardt disease), CEP290 (LCA10), EYS (Retinitis Pigmentosa), RP1 (Retinitis Pigmentosa), ALMS1(Alstrom syndrome), CDH23 (Usher syndrome 1D), PCDH15 (Usher syndrome 1F), and USH2A (Usher syndrome 2A). In some embodiments, the selected full-length polypeptide is selected from DMD (Duchenne muscular dystrophy), CFTR (Cystic fibrosis), GDE (Glycogen storage disease III), DYSF (dysferlinopathies), OTOF (neurosensory nonsyndromic recessive deafness) and F8 (Hemophilia A). In some embodiments, the selected full-length polypeptide is not OTOF.

In some embodiments, all or part of the intron sequence present at the 3′-end of the coding sequence of the first vector is identical or substantially identical with all or part of the intron sequence present at the 5′-end of the coding sequence of the second vector. In some embodiments, intron sequence overlap between the first and second AAV vectors is several hundred nucleotides in length. In particular embodiments, the intron sequence overlap is about 50 to about 500 nucleotides or so in length; alternatively between about 200 and about 300 nucleotides or so in length.

In particular embodiments, the intron sequence utilized in any vector system of the disclosure is a sequence of an intron naturally present in the genomic sequence of the gene encoding the selected polypeptide. In some embodiments, characterized as the native intron hybrid vectors, the intron is intron 23 of the hMYO7A gene. In particular embodiments, the polypeptide encoded is hMYO7A, or a functional fragment thereof, and the intron is a partial sequence of full intron 23 of the hMYO7A gene. In particular embodiments, the polypeptide encoded is hMYO7A, or a functional fragment thereof, and the intron is the full intron 23 of the hMYO7A gene.

In some embodiments of the native intron hybrid vectors as described herein, the recombinogenic sequence of the dual vector system comprises partial sequences of exon 21, exon 22, and/or exon 23 of the hMYO7A gene. In some embodiments of the native intron hybrid vectors as described herein, the dual vector system comprising partial sequences of exon 21, exon 22, and/or exon 23 of the hMYO7A gene utilizes a partial sequence of a full native intron. In some embodiments, the native intron is intron 23 of the hMYO7A gene. Thus, in some embodiments, the intron sequence is a partial sequence of full intron 23 of the hMYO7A gene. As used herein, a “partial sequence” may comprise e.g. at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100%, etc. of the exon and/or intron sequence. In some embodiments, the first and/or second intron sequence comprises a sequence of an intron naturally present in the genomic sequence of the gene encoding a myosin polypeptide. In some embodiments, the intron sequence is a partial sequence of full intron 23 of the hMYO7A gene.

A number of strategies have been devised to overcome the issue of random concatemerization and thereby increase specificity as well as efficiency of dual vector platforms. First, the addition of a highly-recombinogenic sequence such as that used in the AP hybrid vector here has resulted in significantly increased protein expression compared with the trans-splicing system. Ghosh et al. (2011) provide a detailed analysis of the 270-bp AP sequence used in this study as well as other sequences derived from AP that direct recombination and lead to significant improvement over trans-splicing vectors. The finding that AP hybrid vectors are more efficient than trans-splicing vectors supports that the AP sequence directs at least some of the concatemerization events toward the proper orientation with recombination then occurring via this sequence or via the ITRs. The APhead domain in particular can mediate appropriate head-to-tail concatemerization following re-combination of the dual vectors in the cell. Regardless, with more concatemers properly aligned, the AP hybrid system mediates a more-efficient expression of MYO7A. (Another approach for directing concatemerization is the use of single-strand oligonucleotides that are capable of tethering the back end of the 5′ vector and the front end of the 3′ vector together (Hirsch et al., 2009); however, this strategy requires efficient delivery of the oligonucleotide to the nucleus of the target cells timed with the dual vectors.) Finally, dual vectors utilizing mismatched ITRs can be used to direct concatemerization in a head-to-tail orientation (Yan et al., 2005), although the process may require further optimization of the AAV packaging machinery.

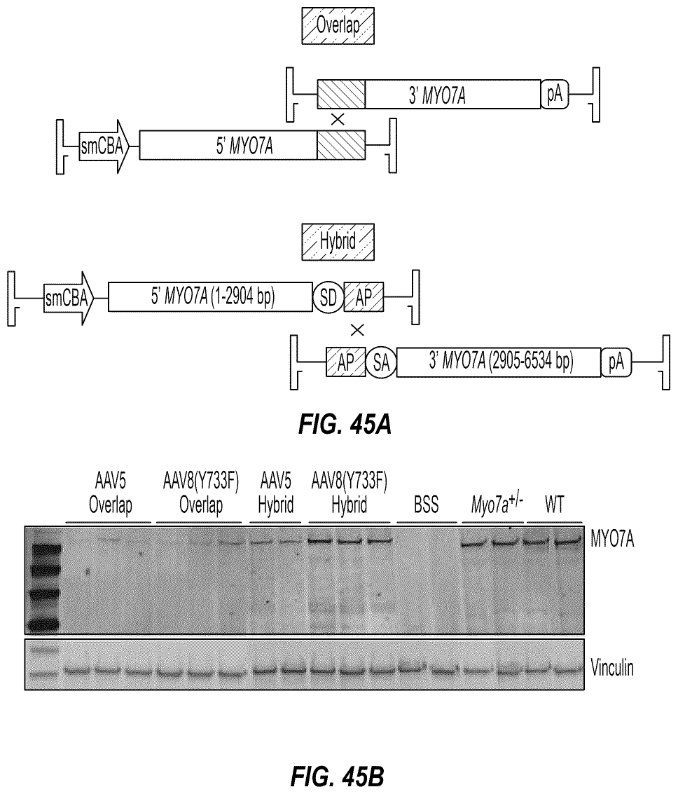

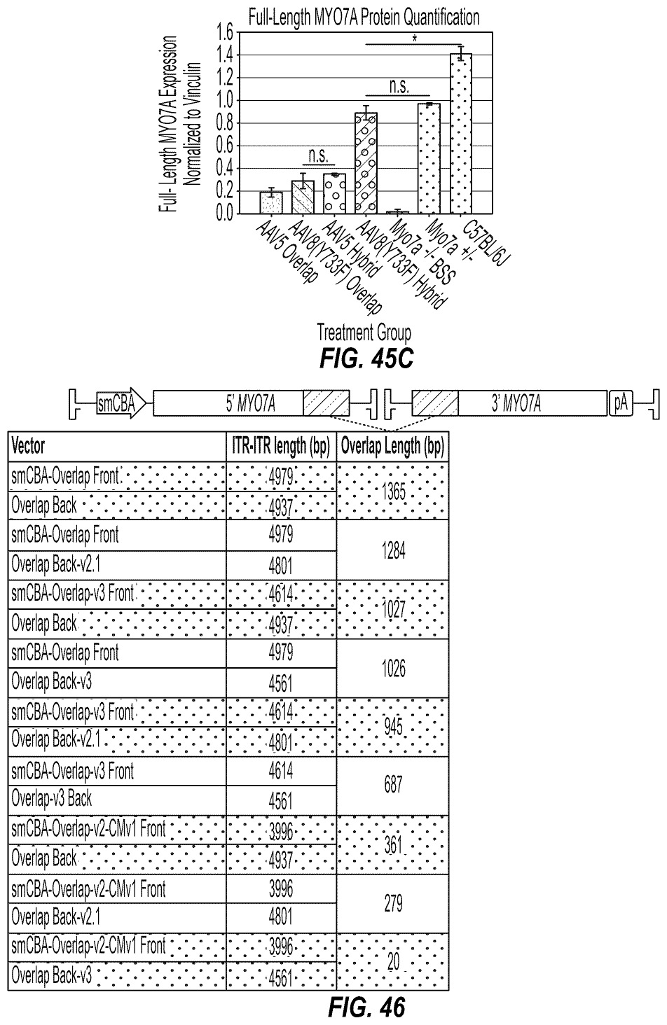

Accordingly, in some embodiments, the intron sequence utilized in the vector system of the disclosure is a sequence of an intron that is not naturally present in the genomic sequence of a gene encoding the selected polypeptide. In particular embodiments, the intron is a synthetic alkaline phosphatase (AP) intron. The intron sequences utilized in the vector system of the disclosure can comprise splice donor and splice acceptor sequences. In some embodiments, the intron sequence is a recombinogenic, intronic sequence (for example, the AK sequence of the F1 phage as shown in Trapani, et al. 2014). In these embodiments, the hybrid vectors, characterized as the second generation hybrid vectors described herein, rely on both ITR-mediated concatemerization and homologous recombination mediated by the AK sequence for the reconstitution of the full-length expression cassette. Thus, in some embodiments, the intron sequence is the AK sequence of the F1 phage. Accordingly, in some embodiments of the disclosed hybrid vectors, the vectors comprise one or more AP intronic spliceosome recognition sites, such as one or more AP splice acceptor (APSA) domains or AP splice donor (APSD) domains. In exemplary embodiments, these vectors comprise an APSA and an APSD. In some embodiments, the front half vector contains an APSA and the back half vector contains an APSD. In some embodiments, the front half vector contains an APSD and the back half vector contains an APSA. See FIGS. 37A and 45A.

Accordingly, in exemplary embodiments, the hybrid vector pairs contain an APhead-encoding sequence as part of the AP intron. In some embodiments of the disclosed hybrid vectors, the vectors comprise an intronic sequence comprising a nucleotide sequence having at least 85%, 90%, 92.5%, 95%, 98%, or 99% identity to either of SEQ ID NO: 69 or 70. In some embodiments of the disclosed hybrid vectors, the vectors comprise the nucleotide sequence of SEQ ID NO: 69 or 70 (APhead sequence).

Polypeptides other than hMYO7A that are contemplated for delivery using any of the disclosed hybrid vectors include, but are not limited to, harmonin (Uniprot Q9Y6N9), cadherin 23 (Uniprot Q9H251), protocadherin 15 (Uniprot Q96QU1), and usherin (USH2A) (Uniprot 075445). In some embodiments, the selected full-length polypeptide is encoded by a gene of about 5 Kb to about 10 Kb in length. In some embodiments, the selected full-length polypeptide is encoded by a gene of about 6 Kb to about 9 Kb in length. In some embodiments, the selected full-length polypeptide is encoded by a gene of about 7 Kb to about 8 Kb in length. In some embodiments, hybrid dual vectors expressing portions (or halves) of a large gene contain a sequence between the first intron and second intron of the first and second AAV vector polynucleotides, respectively; and a large gene other than MYO7A and that comprises a nucleotide sequence having at least 80%, 85%, 90%, 95%, 98%, or 99% identity to any one of SEQ ID NOs: 39 and 52-59. In some embodiments, these hybrid vectors an intronic sequence containing the nucleotide sequence of any one of SEQ ID NOs: 39 and 53-59, e.g., SEQ ID NO: 56 or 57. The large gene may be selected from ABCA4, CEP290, EYS, RP1, ALMS1, CDH23, PCDH15, USH1C, USH1G, USH2A, DNFB31, DMD, CFTR, GDE, DYSF, F8, and DFNB2. These hybrid vectors encoding non-MYO7A (e.g., ABCA4) genes may contain an overlapping region identified through the improved overlap vectors provided herein as the recombinogenic sequence, in place of the recombinogenic APhead sequence/domain. The overlapping regions of these hybrid vectors are flanked by splice acceptor and/or splice donor sequences, such that the overlapping region is spliced out, and does not code for any MYO7A protein.

In some embodiments, the hybrid vector system of the first generation contains a split point between exons 23 and 24, wherein the sequence corresponding to the tail domain of the MYO7A protein is contained within the front-half vector represented by SEQ ID NO: 3. The back-half vector of this exon 23/24 hybrid vector system is shown in SEQ ID NO: 4. In some embodiments, the second generation hybrid vector system contains a split point located between exons 21 and 22, wherein the sequence corresponding to the tail domain of the MYO7A protein is removed from the front-half vector of the exon 23/24 hybrid vector system, thereby generating the second generation hybrid front-half vector (SEQ ID NO: 31).

Thus, in an exemplified embodiment, the first AAV vector polynucleotide comprises the nucleotide sequence of SEQ ID NO: 31, or a functional fragment and/or variant thereof, and the second AAV vector polynucleotide comprises the nucleotide sequence of SEQ ID NO: 32, or a functional fragment and/or variant thereof, and the intronic sequence is the AK sequence of the F1 phage.

In some embodiments, the split point between the first and second AAV vector polynucleotide sequences is between exon 21 and exon 22 of the hMYO7A gene. In some embodiments, the split point between the first and second AAV vector polynucleotide sequences is between exon 22 and exon 23 of the hMYO7A gene.

In some embodiments, the split point between the first and second AAV vector polynucleotide sequences is not between exon 23 and exon 24 of the hMYO7A gene. In exemplary embodiments, the hybrid vectors of the disclosure do not comprise any of the nucleotide sequences of SEQ ID NOs: 3 and 4.

### CMv1 Hybrid Vector System

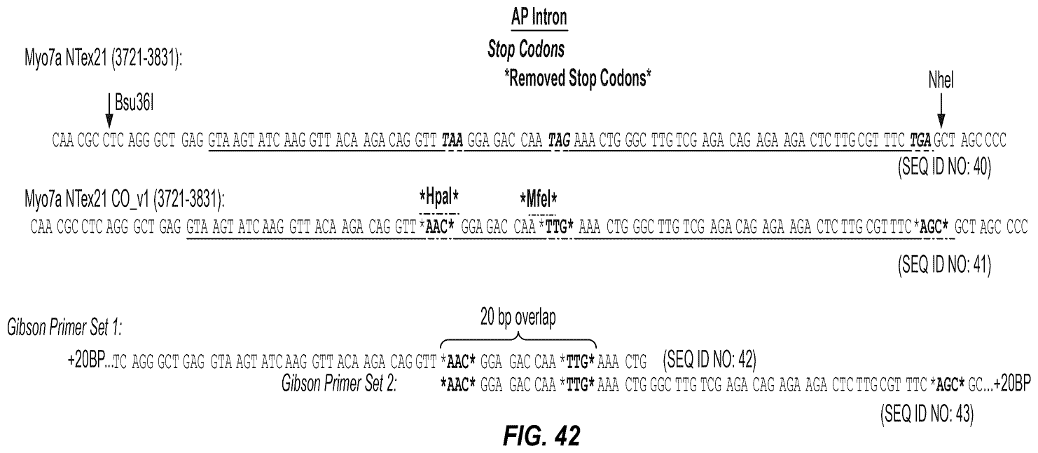

Some embodiments contemplate a hybrid vector system as described herein, wherein substitutions are made in a noncoding sequence of the vector (e.g., the 3′ untranslated region (3′ UTR) downstream of the MYO7A partial coding sequence, and before the 3′ AAV inverted terminal repeat of the inverted terminal repeat pairs of the first and/or second vector polynucleotide). In some embodiments, the substitutions are positioned in putative stop codons, such that these potential stop codons are removed. In some embodiments, one or more potential stop codons are removed by installing one or more nucleotide substitutions in the alkaline phosphatase (AP) intronic splice donor sequence (“AP intron”) of the front-half vector (for example, the front-half vector comprising SEQ ID NO: 31). In some embodiments, three potential stop codons are modified within the alkaline phosphatase intronic splice donor sequence of the front-half vector. As a result of the modification of these putative stop codons in the APhead sequence, a modified front-half vector is created comprising SEQ ID NO: 33. See FIG. 42.

Accordingly, in some embodiments, a hybrid vector system of the disclosure comprises:
i) a first AAV vector polynucleotide comprising an inverted terminal repeat at each end of the polynucleotide, and between the inverted terminal repeats a promoter followed by a partial coding sequence that encodes an N-terminal part of a selected full-length polypeptide followed by a splice donor site and an intron; and
ii) a second AAV vector polynucleotide comprising an inverted terminal repeat at each end of the polynucleotide, and between the inverted terminal repeats an intron and a splice acceptor site for the intron, and optionally followed by a partial coding sequence that encodes a C-terminal part of the selected full-length polypeptide, followed by a polyadenylation (pA) signal sequence. The intron sequence in the first and second AAV vectors comprises sequence that overlaps. In an exemplary embodiment, the first AAV vector polynucleotide comprises the nucleotide sequence of SEQ ID NO: 33, or a functional fragment and/or variant thereof, and the second AAV vector polynucleotide comprises the nucleotide sequence of SEQ ID NO: 32, or a functional fragment and/or variant thereof.

### CMv2 Hybrid Vector System

Still other embodiments contemplate the hybrid vector system as described herein, with one additional putative in-frame stop codon modified in the front-half vector (for example, the front-half vector comprising SEQ ID NO: 33). In some embodiments, the modification comprises the installation of a substitution into the APhead sequence of the front-half vector that removes a putative stop codon from this sequence. As a result of this modification and removal of this putative stop codon, a further modified front-half vector is created comprising SEQ ID NO: 34.

Upon modification of the additional putative in-frame stop codon as described herein, some embodiments consider making complementary changes to the back-half vector (for example, the back-half vector comprising SEQ ID NO: 32), where the identical codon is also modified in the back-half vector. Thus, in some embodiments, the modification comprises the installation of a substitution into the APhead sequence of the back-half vector that removes a putative stop codon from this sequence. As a result of the modification of this additional stop codon, a modified back-half vector is created comprising SEQ ID NO: 35. See FIG. 42.

As such, in the CMv1 vector, three potential in-frame stop codons in the AP intron are removed. In the CMv2 vector, these same three potential stop codons in the AP intron were removed, and one potential stop codon from the APhead coding sequence was removed.

Therefore, in particular embodiments, a hybrid vector system of the disclosure comprises:
i) a first AAV vector polynucleotide comprising an inverted terminal repeat at each end of the polynucleotide, and between the inverted terminal repeats a promoter followed by a partial coding sequence that encodes an N-terminal part of a selected full-length polypeptide followed by a splice donor site and an intron; and
ii) a second AAV vector polynucleotide comprising an inverted terminal repeat at each end of the polynucleotide, and between the inverted terminal repeats an intron and a splice acceptor site for the intron, and optionally followed by a partial coding sequence that encodes a C-terminal part of the selected full-length polypeptide, followed by a polyadenylation (pA) signal sequence. The intron sequence in the first and second AAV vectors comprises sequence that overlaps. In an exemplary embodiment, the first AAV vector polynucleotide comprises the nucleotide sequence of SEQ ID NO: 34, or a functional fragment and/or variant thereof, and the second AAV vector polynucleotide comprises the nucleotide sequence of SEQ ID NO: 35, or a functional fragment and/or variant thereof.

### V2 MIN and CMv2 MIN Hybrid Vector Systems (Also Known as CMv2.1 Hybrid Vector System)

When the split point is altered from the junction between exons 23 and 24, as in the first-generation hybrid vector system, to the junction between exons 21 and 22, as in the second-generation hybrid vector system, additional portions of the MYO7A sequence are shifted to the back-half second-generation hybrid vector. Because of this, in some embodiments the second-generation back-half hybrid vector is close to, but does not exceed, the AAV packaging limit. Accordingly, in some embodiments, the modified back-half vector comprising SEQ ID NO: 35 may be further modified to remove any residual (extra), non-essential sequences, such as restriction enzyme sites and tag sequences, from the 3′ end of the construct. These residual sequences are sometimes referred to as “unseeded legacy” sequences. The resulting modified back-half vector comprises SEQ ID NO: 49. This vector system is known as the “V2 MIN” or “V2-back MIN” hybrid system. In some embodiments, this system contains an HA tag (“V2 MIN HA”). The V2 MIN HA vector is set forth as SEQ ID NO: 48. The V2 MIN back-half vector is 122 bp shorter than the first-generation hybrid back-half vectors (4981 bp vs. 4861 bp).

In some embodiments, a vector is provided in which unseeded legacy sequences are removed, and one or more substitutions in non-coding sequences are installed. In some embodiments, these one or more substitutions are positioned in putative stop codons, such that these potential stop codons are removed. In some embodiments, one or more potential stop codons are removed by installing one or more nucleotide substitutions in the APhead sequence of the front-half vector. In some embodiments, the one or more putative stop codons are removed and replaced with a “stuffer” sequence (see FIG. 43).

In some embodiments, three potential stop codons are modified within the alkaline phosphatase intron sequence of the front-half vector. In some embodiments, one putative stop codon is modified (removed) by installing one or more substitutions in the APhead sequence of the front-half vector. As a result of the modification of these putative stop codons in the APhead sequence, the modified front-half vector of SEQ ID NO: 34 was generated.

In some embodiments, one putative stop codon is likewise modified in the APhead sequence of the back-half vector. As a result of the modification of this putative stop codon in the APhead sequence, the modified back-half vector of SEQ ID NO: 44 was generated.

This vector system is known as the CMv2 MIN system. In some embodiments, this system contains an HA tag (“CMv2 MIN HA”). The CMv2 MIN HA vector is set forth as SEQ ID NO: 47. The CMv2 MIN back-half vector is 121 bp shorter than the first-generation hybrid back-half vectors (4982 bp vs. 4861 bp).

Thus, in some embodiments, a hybrid vector system of the disclosure comprises:
i) a first AAV vector polynucleotide comprising an inverted terminal repeat at each end of the polynucleotide, and between the inverted terminal repeats a promoter followed by a partial coding sequence that encodes an N-terminal part of a selected full-length polypeptide followed by a splice donor site and an intron; and
ii) a second AAV vector polynucleotide comprising an inverted terminal repeat at each end of the polynucleotide, and between the inverted terminal repeats an intron and a splice acceptor site for the intron, and optionally followed by a partial coding sequence that encodes a C-terminal part of the selected full-length polypeptide, followed by a polyadenylation (pA) signal sequence. The intron sequence in the first and second AAV vectors comprises sequence that overlaps. In an exemplary embodiment, the first AAV vector polynucleotide comprises the nucleotide sequence of SEQ ID NO: 34, or a functional fragment and/or variant thereof, and the second AAV vector polynucleotide comprises the nucleotide sequence of SEQ ID NO: 44, or a functional fragment and/or variant thereof. In some embodiments, the first AAV vector polynucleotide comprises the nucleotide sequence of SEQ ID NO: 34, or a functional fragment and/or variant thereof, and the second AAV vector polynucleotide comprises the nucleotide sequence of SEQ ID NO: 47, or a functional fragment and/or variant thereof. In some embodiments, the first AAV vector polynucleotide comprises the nucleotide sequence of SEQ ID NO: 34, or a functional fragment and/or variant thereof, and the second AAV vector polynucleotide comprises the nucleotide sequence of SEQ ID NO: 48, or a functional fragment and/or variant thereof.

### CMv3 MIN Hybrid Vector Systems

Still other embodiments contemplate the hybrid vector system as described herein, with three additional in-frame stop codons modified in the front-half vector. In some embodiments, one or more nucleotide substitutions (e.g., three substitutions) are made in the 3′UTR or ITR downstream of the MYO7A coding sequence. Accordingly, in some embodiments, a front-half vector is provided in which one in-frame stop codon in the APhead sequence, three in-frame stop codons in the AP intron sequence, and three in-frame stop codons in the 3′ UTR sequence have been removed through the installation of substitutions. As a result of the modification of these putative stop codons, a further modified front-half vector is created comprising SEQ ID NO: 46 (“AAV-smCBA-hMYO7A-NT-Ex21-APSD-APhead-CMv3”, or simply “CMv3 hybrid system”). In exemplary embodiments, the CMv3 hybrid system is a CMv3 MIN system in that residual, unseeded legacy sequences (e.g., restriction enzyme sites) have been removed. In some embodiments, the CMv3 system has a HA tag.

Therefore, in an exemplary embodiment, the first AAV vector polynucleotide comprises the nucleotide sequence of SEQ ID NO: 46, or a functional fragment and/or variant thereof, and the second AAV vector polynucleotide comprises the nucleotide sequence of SEQ ID NO: 35, or a functional fragment and/or variant thereof.

Thus, in some embodiments, the polynucleotide vector system of the disclosure is a CMv3 hybrid system. In some embodiments, the vector system is a CMv3 MIN system. In some embodiments, the vector system is a CMv2 system. In some embodiments, the vector system is a CMv2 MIN system. In some embodiments, the vector system is a CMv1 or CMv1 MIN system. Any of the disclosed front-half hybrid vectors may be combined with any of the disclosed back-half hybrid vectors in the compositions of the disclosure.

In exemplary embodiments, polynucleotide vector systems are provided that comprise:
i) a first AAV vector polynucleotide comprising an inverted terminal repeat at each end of the polynucleotide, and between the inverted terminal repeats a promoter followed by a partial coding sequence that encodes an N-terminal part of a myosin polypeptide followed by a splice donor site and an intron, and
ii) a second AAV vector polynucleotide comprising an inverted terminal repeat at each end of the polynucleotide, and between the inverted terminal repeats an intron and a splice acceptor site for the intron,
wherein the intron sequence in the first and second AAV vectors comprises a polynucleotide sequence that overlaps, and
wherein the first AAV vector polynucleotide comprises a nucleotide sequence selected from SEQ ID NOs: 31, 33, 34, and 46;
and the second AAV vector polynucleotide comprises a nucleotide sequence selected from SEQ ID NOs: 32, 35, 44, and 47-49.

In some embodiments of the hybrid vector systems described herein, the selected full-length polypeptide is a myosin polypeptide. In some embodiments, the myosin polypeptide is human myosin VII A (hMYO7A). In some embodiments, the myosin polypeptide is human myosin VII B (hMYO7B). In some embodiments, the myosin polypeptide is myosin 7 (VII) isoform II. In some embodiments, the myosin polypeptide is another myosin isoform or a functional fragment thereof. In particular embodiments, full-length myosin 7A or isoform II is encoded in the provided vector systems.

In some embodiments, the C-terminal part of the selected full-length polypeptide (e.g., the myosin polypeptide) comprises the single-alpha helix (SAH) domain of the selected full-length polypeptide.

The coding sequences in the first and second vectors when combined encode the selected full-length polypeptide, or a functional fragment or variant thereof. Accordingly, in some embodiments, all or part of the intron sequence present at the 3′-end of the coding sequence of the first vector is identical or substantially identical with all or part of the intron sequence present at the 5′-end of the coding sequence of the second vector.

Some embodiments of the hybrid vectors described herein contemplate a virus or a recombinant viral particle comprising the first AAV vector polynucleotide or the second AAV vector polynucleotide as described herein. In particular embodiments, the first AAV vector polynucleotide comprises SEQ ID NO: 33, and the second AAV vector polynucleotide comprises SEQ ID NO: 32. In some embodiments, the virus or recombinant viral particle is characterized as an adeno-associated virus (AAV) or an infectious AAV viral particle. In some embodiments, the recombinant AAV viral particle includes one or more tyrosine-to-phenylalanine (Y-F) mutations in a capsid protein of the virus or virion. Tyrosine-to-phenylalanine (Y-F) mutations in a capsid protein of the virus or virion at amino acid position 733 are specifically contemplated herein (for example, AAV8 Y733F). Likewise, tyrosine-to-phenylalanine (Y-F) mutations in a capsid protein of the virus or virion at amino acid position 731 are specifically contemplated herein (for example, AAV44.9(Y731F)).

In some embodiments, the virus or virion is packaged in an AAV5, AAV7, AAV8, AAV9, AAV44.9, AAV44.9(E531D), AAV2(4pMut), AAVAnc80, AAVrh.8, AAVrh.8R, AAV9-PHP.B, AAV9-PHP.eB, AAVrh.10, or AAVrh.74 capsid. In some embodiments, the viral particle comprises an AAV1, AAV2, AAV3, AAV4, AAV5, AAV6, AAV7, AAV8, AAV9, AAV10, AAV7m8, AAV-DJ, AAV2/2-MAX, AAVSHh10, AAVSHh10Y, AAV3b, AAVLK03, AAV8PB2, AAV1(E531K), AAV6(D532N), AAV6-3pmut, AAV2G9, AAV44.9, AAV44.9(E531D), AAVrh.8, AAVrh.8R, AAV9-PHP.B, or an AAVAnc80 capsid. In exemplary embodiments for delivery to the retina, the virion is packaged in an AAV44.9(E531D) capsid variant. In exemplary embodiments for delivery to the hair cells of the ear, the virion is packaged in an AAV9-PHP.B capsid variant.

In some embodiments, the hybrid polynucleotide vector systems described herein use a tissue-specific promoter. In some embodiments, the systems use a promoter that mediates expression in the eye. In some embodiments, the systems use a promoter that mediates expression in the ear.

In some embodiments, the hybrid polynucleotide vector systems described herein use any one of the following promoters: a cytomegalovirus (CMV) promoter, an elongation factor-1 alpha (EF-1 alpha) promoter, a cone arrestin promoter, a chimeric CMV R actin (smCBA) promoter, a human myosin 7a gene-derived promoter, a cone transducin a (TaC) gene-derived promoter, a rhodopsin promoter, a cGMP-phosphodiesterase P-subunit promoter, human or mouse rhodopsin promoter, a human rhodopsin kinase (hGRK1) promoter, a synapsin promoter, a glial fibrillary acidic protein (GFAP) promoter, a rod specific IRBP promoter, a RPE-specific vitelliform macular dystrophy-2 [VMD2]promoter, and combinations thereof. In some embodiments, the polynucleotide vector system described herein uses a human rhodopsin kinase (hGRK1) promoter. In some embodiments, the polynucleotide vector system uses a cone arrestin promoter. In some embodiments, the polynucleotide vector system uses a cytomegalovirus (CMV) promoter. In some embodiments for delivery to the eyes (retina) of a subject, the disclosed overlap polynucleotide vector systems use a cytomegalovirus (CMV) promoter.

In some embodiments for delivery to the retina, the hybrid polynucleotide vector system uses an EF-1 alpha promoter. In some embodiments for delivery to the ears (hair cells) of a subject, the polynucleotide vector system uses a synapsin or GFAP promoter (see Lee et al., Hearing Research).

Each embodiment contained with the “hybrid vectors” section and as described herein is specifically contemplated for each hybrid vector system described, for example the vector system wherein the split point between the first and second AAV vector polynucleotide sequences is between exon 21 and exon 22 of the hMYO7A gene; the vector system wherein the split point between the first and second AAV vector polynucleotide sequences is between exon 22 and exon 23 of the hMYO7A gene; the vector system with a front-half vector comprising the nucleotide sequence of SEQ ID NO: 31 and a back-half vector comprising the nucleotide sequence of SEQ ID NO: 32; the vector system with a front-half vector comprising the nucleotide sequence of SEQ ID NO: 33 and a back-half vector comprising the nucleotide sequence of SEQ ID NO: 32; the vector system with a front-half vector comprising the nucleotide sequence of SEQ ID NO: 34 and a back-half vector comprising the nucleotide sequence of SEQ ID NO: 35; and/or the vector system with a front-half vector comprising the nucleotide sequence of SEQ ID NO: 34 and a back-half vector comprising the nucleotide sequence of SEQ ID NO: 44.

In some embodiments, any vector of the hybrid polynucleotide vector systems described in the disclosure may be administered by parenteral administration, such as intravenous, intramuscular, intraocular, intranasal, or (intra-)utricle injection, etc. The vector can be administered in vivo, in vitro or ex vivo.

In some embodiments, any vector of the hybrid polynucleotide vector systems described herein may be administered to the eye. In particular embodiments, a vector is administered to the eye of a subject by subretinal injection. In some embodiments, any vector of the hybrid polynucleotide vector systems described herein may be administered to the ear. In some embodiments, any vector of the hybrid polynucleotide vector systems described herein the polynucleotide vector system is administered to the ear of a subject, e.g., by a round window injection or during cochlear implant surgery.

The methods of the disclosure can be used with humans and other animals. Animals contemplated within the scope of the disclosure include, for example, dogs, cats, rabbits, ferrets, guinea pigs, hamsters, pigs, monkeys or other primates, mice, gerbils, horses, mules, donkeys, burros, cattle, cows, pigs, sheep, and alligators. As used herein, the terms “patient” and “subject” are used interchangeably and are intended to include such human and non-human species, including human and non-human cells. Likewise, in vitro methods of the disclosure may also be performed on cells of one or more human or non-human, mammalian species, including human and non-human cells.

### Components of Exemplary Dual AAV Vectors

Any of the dual polynucleotide vector systems of the disclosure may be used in conjunction with an AAV vector system known in the art. In treating some diseases, it may be preferable to administer the rAAV vector construct a single time, while in the management or treatment of other diseases or conditions, it may be desirable to provide two or more administrations of the vector constructs to the patient in one or more administration periods. In such circumstances, the AAV vector-based therapeutics may be provided successively in one or more daily, weekly, monthly, or less-frequent periods, as may be necessary to achieve treatment, or amelioration of one or more symptoms of the disease or disorder being treated. In some embodiments, the vector may be provided to one or both eyes by one or more administrations of an infectious adeno-associated viral particle, an rAAV virion, or a plurality of infectious rAAV particles in an amount and for a time sufficient to treat or ameliorate one or more symptoms of the disease or condition being treated.

In particular embodiments, the disclosure provides rAAV particles been derived from a number of different serotypes, including, for example, those selected from the group consisting of AAV1, AAV2, AAV3, AAV4, AAV5, AAV6, AAV7, AAV8, AAV9, and AAV10. In exemplary embodiments, particles derived from AAV2, AAV5 and AAV8 serotype vectors are utilized. In particular embodiments, particles having an AAV8(Y733F) or AAV2(tripleY-F) capsid are used. Accordingly, the disclosure provides recombinant AAV particles derived from, e.g., AAV8(Y733F) or AAV2(tripleY-F), that comprise overlap and hybrid polynucleotide vector systems. In some embodiments, the serotype of the AAV vector is not AAV6 or AAV2.

Additional exemplary capsids include AAV2, AAV6, and capsids derived from AAV2 and AAV6. Such capsids include AAV7m8, AAV-DJ, AAV2/2-MAX, AAVSHh10, AAVSHh10Y, AAV3b, AAVLK03, AAV8PB2, AAV1(E531K), AAV6(D532N), AAV6-3pmut, AAV2G9, AAV44.9, AAV44.9(E531D), AAVrh.8, AAVrh.8R, AAV9-PHP.B, and/or AAVAnc80. In some embodiments, the virus or virion is packaged in an AAV5, AAV7, AAV8, AAV9, AAV44.9, AAV44.9(E531D), AAV2(4pMut), AAVAnc80, AAVrh.8, AAVrh.8R, AAV9-PHP.B, AAVrh.10, or AAVrh.74 capsid.

The AAV-DJ capsid is described in Grimm et al., J. Virol., 2008, 5887-5911 and Katada et al., (2019), Evaluation of AAV-DJ vector for retinal gene therapy, Peer J 7:e6317, each of which is herein incorporated by reference. The AAV7m8 capsid, which is closely related to AAV-DJ, is described in Dalkara, et al. Sci Transl Med. 2013; 5(189):189ra76, herein incorporated by reference. The AAV2/2-MAX capsid is described in Reid, Ertel & Lipinski, Improvement of Photoreceptor Targeting via Intravitreal Delivery in Mouse and Human Retina Using Combinatory rAAV2 Capsid Mutant Vectors, Invest. Ophthalmol Vis Sci. 2017; 58:6429-6439, herein incorporated by reference. The AAV2/2-MAX capsid comprises five point mutations, Y272F, Y444F, Y500F, Y730F, T491V. The AAV1(E531K) capsid is described in Boye et al., Impact of Heparin Sulfate Binding on Transduction of Retina by Recombinant Adeno-Associated Virus Vectors, J. Virol. 90:4215-4231 (2016), herein incorporated by reference. The AAVSHh10 and AAV6(D532N) capsids, both derivatives of AAV6, are described in Klimczak et al., (2009) A Novel Adeno-Associated Viral Variant for Efficient and Selective Intravitreal Transduction of Rat Muller Cells, PLoS ONE 4(10): e746, herein incorporated by reference. The AAV6-3pmut (also known as AAV6(TM6) and AAV6(Y705+Y731F+T492V)) capsid is described in Rosario et al., Microglia-specific targeting by novel capsid-modified AAV6 vectors, Mol Ther Methods Clin Dev. (2016); 13(3):16026 and International Patent Publication No. 2016/126857, each of which are herein incorporated by reference.

Additional capsids suitable for use with the disclosed methods include the following: capsids comprising non-native amino acid substitutions at amino acid residues of a wild-type AAV2 capsid, wherein the non-native amino acid substitutions comprise one or more of Y272F, Y444F, T491V, Y500F, Y700F, Y704F and Y730F; capsids comprising non-native amino acid substitutions at amino acid residues of a wild-type AAV6 capsid, wherein the non-native amino acid substitutions comprise one or more of Y445F, Y705F, Y731F, T492V and S663V. In certain embodiments, the capsid comprises AAV2G9, a variant of AAV2.

In other embodiments, the capsid comprises a non-native amino acid substitution at amino acid residue 533 or 733 of a wild-type AAV8 capsid, wherein the non-native amino acid substitution is E533K, Y733F, or a combination thereof. The AAV8(Y733F) capsid is described in Doroudchi et al., Amer. Soc. of Gene & Cell Ther. 19(7): 1220-29 (2011). In certain embodiments of the disclosed methods, the capsid comprises AAV8PB2, a variant of AAV8.

In other embodiments, the capsid comprises non-native amino acid substitutions of a wild-type AAV2 capsid comprising one or more of the following mutations:

$$Y 444 F ; \qquad ( a )$$

$$Y 444 F + Y 500 F + Y 730 F ; \qquad ( b )$$

$$Y 272 F + Y 444 F + Y 500 F + Y 730 F ; \qquad ( c )$$

$$Y 444 F + Y 500 F + T 730 F + T 491 V ; or \qquad ( d )$$

$$Y 272 F + Y 444 F + Y 500 F + Y 730 F + T 491 V . \qquad ( e )$$

In other embodiments, the capsid comprises non-native amino acid substitutions of a wild-type AAV6 capsid, comprising one or more of the following mutations:

$$Y 445 F ; \qquad ( a )$$

$$Y 705 F + Y 731 F ; \qquad ( b )$$

$$T 492 V ; \qquad ( c )$$

$$Y 705 F + Y 731 F + T 492 V ; \qquad ( d )$$

$$S 663 V ; or \qquad ( e )$$

$$S 663 V + T 492 V . \qquad ( f )$$

Additional capsids suitable for use with the disclosed methods are described in International Patent Publication No. WO 2018/156654, published Aug. 30, 2018, herein incorporated by reference in its entirety. In particular embodiments, the rAAV particles of the disclosed invention comprise one of the following capsids: DGE-DF (also known as ‘V1V4 VR−V’), P2−V2, P2−V3, P2−V1 (also known as ME−B), and P2−V1(Y−F+T−V) (also known as ME−B(Y−F+T−V)). In still other embodiments, the rAAV particles may comprise a capsid selected from AAV6(3pMut) or AAV2(quadYF+T−V). In still other embodiments, the rAAV particles of the disclosed methods may comprise any of the capsid variants described in International Patent Publication No. WO 2018/156654.

In particular embodiments, disclosed herein are rAAV particles which may comprise a DGE−DF capsid, P2−V2 capsid, P2−V3 capsid, P2−V1 capsid (also known as ME−B), or P2−V1(Y−F+T−V) capsid for the enhanced transduction of said rAAV particles in retinal cells. In other embodiments, the disclosed rAAV particles may comprise a capsid selected from AAV2(Y444F), AAV2(Y444F+Y500F+Y730F), AAV2(Y272F+Y444F+Y500F+Y730F), AAV2(Y444F+Y500F+Y730F+T491V) and AAV2(Y272F+Y444F+Y500F+Y730F+T491V), AAV6(Y445F), AAV6(Y705F+Y731F), AAV6(Y705F+Y731F+T492V), AAV6(S663V), AAV6(T492V) or AAV6(S663V+T492V).

Exemplary inverted terminal repeat (ITR) sequences used in any AAV vector systems of the disclosure may comprise any AAV ITR. The ITRs used in an AAV vector can be the same or different. In particular embodiments, the ITR may be obtained from an AAV serotype 2 (AAV2), AAV serotype 5 (AAV5), AAV serotype 7 (AAV7), AAV serotype 8 (AAV8), AAV serotype 44.9 (AAV44.9), or a variant thereof, such as AAV serotype 44.9(E53ID) and 44.9(Y73IF) (see PCT Application No. PCT/US2020/14838, filed Jan. 23, 2020, herein incorporated by reference). An AAV vector of the disclosure can comprise different AAV ITRs. In a non-limiting example, a vector may comprise an ITR of AAV2 and an ITR of AAV5. AAV ITR sequences are well known in the art (see, e.g., GenBank Accession Nos. AF043303.1; NC_001401.2; J01901.1; JN898962.1; K01624.1; and K01625.1). The AAV dual vector systems disclosed herein are able to efficiently express a therapeutic gene that is larger than what may ordinarily be packaged within a single AAV vector.

Accordingly, in some aspects the disclosure provides a virus or virion comprising any of the polynucleotides or vectors of the disclosure. In particular embodiments, the virus or virion is an AAV virus. Methods for preparing viruses and virions comprising a heterologous polynucleotide or vector are known in the art. In the case of AAV, cells can be co-infected or transfected with adenovirus or polynucleotide vectors comprising adenovirus genes suitable for AAV helper function. Examples of materials and methods are described, for example, in U.S. Pat. Nos. 8,137,962 and 6,967,018 (each of which is incorporated herein by reference).

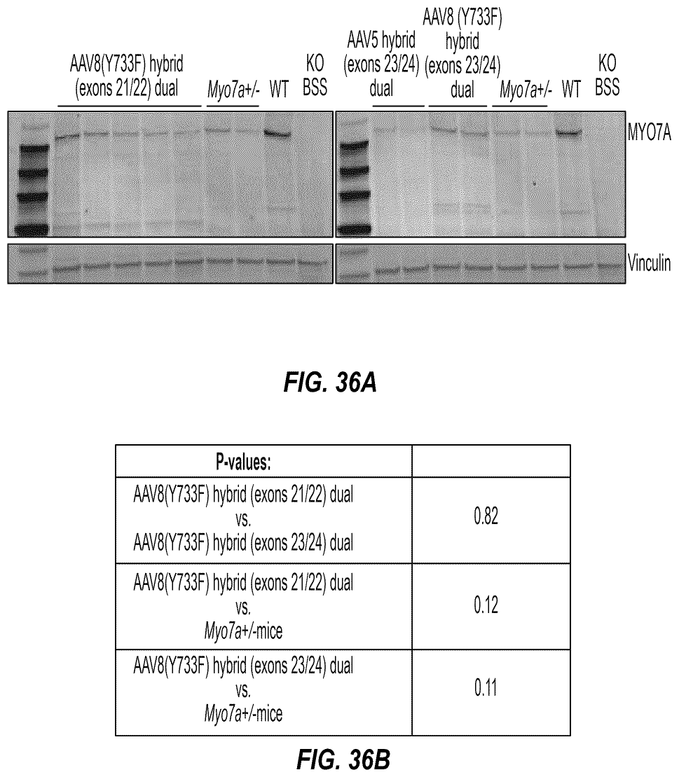

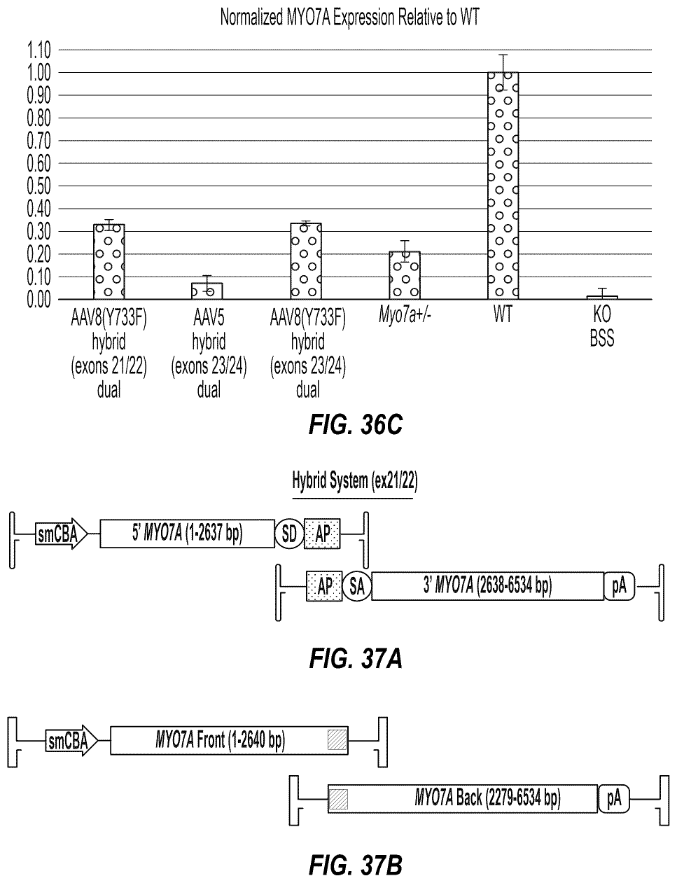

In particular embodiments, the AAV serotype provides for one or more tyrosine to phenylalanine (Y-F) mutations on the capsid surface. In particular embodiments, the AAV is an AAV8 serotype having a tyrosine-to-phenylalanine (Y-F) mutation at position 733 (Y733F). The abilities to produce full-length MYO7A protein for second-generation hybrid and overlap vectors encapsidated in AAV5 and AAV8(Y733F) virions is shown in FIGS. 36A-36C and 45B. AAV8(Y733F) virions outperformed AAV5 virions, as measured by Western blot (see FIG. 45B).

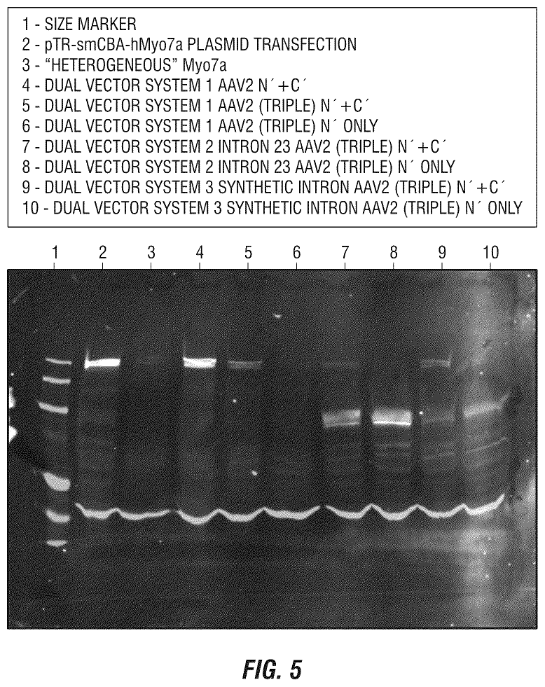

In some embodiments, a triple-mutant AAV8 vector, which contains tyrosine-to-phenylalanine Tyr-Phe mutations at positions Y733F, Y500F, and Y730F, respectively, is used (see FIG. 5). In other embodiments, a triple-mutant AAV8 vector, which contains tyrosine-to-phenylalanine Tyr-Phe mutations at positions Y447F, Y733F, and T494V (e.g. AAV8(Y447F+Y733F+T494F)) is used.

In exemplary embodiments, the rAAV particles of the disclosure may comprise a transgene, or heterologous nucleic acid, that is too large for delivery in standard AAV systems. Exemplary transgenes encode at least one diagnostic or therapeutic protein or polypeptide selected from the group consisting of a molecular marker, photosensitive opsins, including, without limitation, rhodopsin, melanopsin, cone opsins, channel rhodopsins, bacterial or archaea-associated opsins, an adrenergic agonist, an anti-apoptosis factor, an apoptosis inhibitor, a cytokine receptor, a cytokine, a cytotoxin, an erythropoietic agent, a glutamic acid decarboxylase, a glycoprotein, a growth factor, a growth factor receptor, a hormone, a hormone receptor, an interferon, an interleukin, an interleukin receptor, a kinase, a kinase inhibitor, a nerve growth factor, a netrin, a neuroactive peptide, a neuroactive peptide receptor, a neurogenic factor, a neurogenic factor receptor, a neurophilin, a neurotrophic factor, a neurotrophin, a neurotrophin receptor, an N-methyl-D-aspartate antagonist, a plexin, a protease, a protease inhibitor, a protein decarboxylase, a protein kinase, a protein kinase inhibitor, a proteolytic protein, a proteolytic protein inhibitor, a semaphorin, a semaphorin receptor, a serotonin transport protein, a serotonin uptake inhibitor, a serotonin receptor, a serpin, a serpin receptor, a tumor suppressor, and any combination thereof.

In some embodiments, the transgene is hMYO7A, which encodes a human myosin VIIa polypeptide. In particular embodiments, a hMYO7A polypeptide comprises the amino acid sequence shown in SEQ ID NO: 6 or SEQ ID NO: 8, or a functional fragment or a variant thereof. In particular embodiments, the hMYO7A polypeptide is encoded by the nucleotide sequence set forth in SEQ ID NO: 5 or SEQ ID NO: 7.

In some embodiments, the transgene is USHI C, CDH23, PCDH15 and USHI G, all of which are associated with Usher syndrome type I. In some embodiments, the transgene is USH2A or DFNB31, both of which are associated with Usher syndrome type II. In some embodiments, the transgene is ABCA4, CEP290, EYS, RP1, ALMS1, CDH23, PCDH15, DFNB2 or USHERIN.

In some embodiments, administration of any of the disclosed polynucleotide vectors to the eye of a subject in need thereof restores vision loss, partially or completely. The transgene may comprise a human MYO7A. These administrations may provide a partial or complete restoration of melanosome apical migration in retinal pigment epithelium (RPE) cells.

In some embodiments, the production of the therapeutic agent encoded by the transgene of any of the disclosed polynucleotide vector systems in cells of the eye (such as retinal cells or RPE cells) provides one or more of the following therapeutic endpoints: a) preserves one or more photoreceptor cells or one or more RPE cells, b) restores one or more rod- and/or cone-mediated functions, c) restores visual behavior in one or both eyes, or d) any combination thereof. In particular embodiments, production of the therapeutic agent in the disclosed methods preserves one or more PR cells, such as retinal ganglion cells, bipolar cells, Muller glial cells or astrocyte cells, or RPE cells.

In some embodiments, production of the therapeutic agent persists in the one or more photoreceptor cells or the one or more RPE cells substantially for a period of at least three months, at least six months, at least nine months, or at least a year or more, following an initial administration of any of the disclosed rAAV polynucleotide vector system into the one or both eyes of the mammal.

In some embodiments, administration of any of the disclosed polynucleotide vectors to the inner ear of a subject in need thereof restores hearing loss, partially or completely. In some embodiments, administration to the inner ear restores age-related hearing loss. The transgene may comprise a human MYO7A. These administrations may provide a partial or complete restoration of vestibular function in the inner ears. In some embodiments, any of the disclosed hybrid or overlap vectors may be administered to a vestibular hair cell, an inner ear hair cell, an outer ear hair cell, or a combination thereof.

In this manner, the polynucleotide vector systems and compositions thereof of the disclosure may be used to treat or ameliorate symptoms of USH1B (Usher Syndrome type 1B) in the eyes and/or inner ear of the subject. Likewise, administration of the vector systems and compositions of the disclosure may be used to treat or ameliorate symptoms of autosomal recessive isolated deafness (DFNB2), hearing loss, and/or vision loss. As an example, administration of the vector systems and compositions of the disclosure may be used to treat or ameliorate hearing loss associated with insufficiency of MYO7A protein expression (which may present in a USH1B patient). In some embodiments, administration of the vector systems and compositions of the disclosure may be used to treat or ameliorate age-related hearing loss presenting in carriers of a recessive defective MYO7A allele (i.e., USH1B carriers) or age-related hearing loss as the consequence of non-genetic deficiency or insufficiency in MYO7A expression.

As another example, administration of the vector systems and compositions of the disclosure may provide a restoration of melanosome migration in retinal pigment epithelium (RPE) cells.

In some embodiments, the disclosure provides rAAV nucleic acid vectors that include at least a first nucleic acid segment that encodes one or more diagnostic or therapeutic agents that alter, inhibit, reduce, prevent, eliminate, or impair the activity of one or more endogenous biological processes in a mammalian cell suitably transformed with the vector of interest. In certain embodiments, such diagnostic or therapeutic agents may include a molecule that selectively inhibits or reduces the effects of one or more metabolic processes, dysfunctions, disorders, or diseases. In certain embodiments, the defect may be caused by injury or trauma to the mammal for which treatment is desired. In other embodiments, the defect may be caused the over-expression of an endogenous biological compound, while in other embodiments still; the defect may be caused by the under-expression or even lack of one or more endogenous biological compounds.

Regulatory elements of rAAV vectors Any of the vector systems of the disclosure may include regulatory elements that are functional in the intended host cell in which the vector is to be expressed. A person of ordinary skill in the art can select regulatory elements for use in appropriate host cells, for example, mammalian or human host cells. Regulatory elements include, for example, promoters, transcription termination sequences, translation termination sequences, enhancers, and polyadenylation elements.

Any of the vector systems of the disclosure may include a promoter sequence operably linked to a nucleotide sequence encoding a desired polypeptide. Promoters contemplated for use in the disclosure include, but are not limited to, cytomegalovirus (CMV) promoter, SV40 promoter, human myosin 7a gene-derived promoter, Rous sarcoma virus (RSV) promoter, chimeric CMV/chicken β-actin promoter (CBA) and the truncated form of CBA (smCBA) (see, e.g., Haire et al. 2006 and U.S. Pat. No. 8,298,818, each of which is incorporated herein by reference). Additional photoreceptor-specific, human rhodopsin kinase (hGRK1) promoter, a synapsin promoter, a glial fibrillary acidic protein (GFAP) promoter, rod specific IRBP promoter, VMD2 (vitelliform macular dystrophy/Best disease) promoter, a RPE-specific vitelliform macular dystrophy-2 [VMD2]promoter, and EF1-alpha promoter sequences are also contemplated to be useful in the practice of various aspects of the disclosure. Exemplary photoreceptor-cell-specific promoters include, but are not limited to, hGRK1, IRBP, rod opsin, NRL, GNAT2e-IRBP, L/M opsin, and cone arrestin promoters.

In particular embodiments, the promoter is a chimeric CMV-β-actin promoter. In particular embodiments, the promoter is a tissue-specific promoter that shows selective activity in one or a group of tissues but is less active or not active in other tissue. In particular embodiments, the promoter is a photoreceptor-specific promoter. In a further embodiment, the promoter is preferably a cone cell-specific promoter or a rod cell-specific promoter, or any combination thereof. In particular embodiments, the promoter is the promoter for human MYO7A gene. In a further embodiment, the promoter comprises a cone transducin a (TaC) gene-derived promoter. In particular embodiments, the promoter is a human GNAT2-derived promoter. Other promoters contemplated within the scope of the disclosure include, without limitation, a rhodopsin promoter (human or mouse), a cGMP-phosphodiesterase β-subunit promoter, a retinitis pigmentosa-specific promoter, an RPE cell-specific promoter [such as a vitelliform macular dystrophy-2 (VMD2) promoter (Best1) (Esumi et al., 2004)], or any combination thereof.

Promoters can be incorporated into a vector using standard techniques known to those of ordinary skill in the molecular biology and/or virology arts. Multiple copies of promoters, and/or multiple distinct promoters can be used in the vectors of the disclosure. In one such embodiment, a promoter may be positioned about the same distance from the transcription start site as it is from the transcription start site in its natural genetic environment, although some variation in this distance is permitted, of course, without a substantial decrease in promoter activity. In the practice of the disclosure, one or more transcription start site(s) are typically included within the disclosed vectors.

The vectors of the disclosure may further include one or more transcription termination sequences, one or more translation termination sequences, one or more signal peptide sequences, one or more internal ribosome entry sites (IRES), and/or one or more enhancer elements, or any combination thereof. Transcription termination regions can typically be obtained from the 3′-untranslated region of a eukaryotic or viral gene sequence. Transcription termination sequences can be positioned downstream of a coding sequence to provide for efficient termination.

Any of the disclosed polynucleotide vectors may also further include one or more post-transcriptional regulatory sequences or one or more polyadenylation signals, including, for example, but not limited to, a woodchuck hepatitis virus post-transcription regulatory element (WRPE), a polyadenylation signal sequence, or an intron/exon junctions/splicing signals, or any combination thereof.

Signal peptide sequences are amino-terminal peptidic sequences that encode information responsible for the location of an operably-linked polypeptide to one or more post-translational cellular destinations, including, for example, specific organelle compartments, or to the sites of protein synthesis and/or activity, and even to the extracellular environment.

Enhancers—cis-acting regulatory elements that increase gene transcription—may also be included in one of the disclosed AAV-based vector systems. A variety of enhancer elements are known to those of ordinary skill in the relevant arts, and include, without limitation, a CaMV 35S enhancer element, a cytomegalovirus (CMV) early promoter enhancer element, an SV40 enhancer element, as well as combinations and/or derivatives thereof. One or more nucleic acid sequences that direct or regulate polyadenylation of the mRNA encoded by a structural gene of interest, may also be optionally included in one or more of the vectors of the disclosure.

### Host Cells and Methods for Transducing Cells

The disclosure provides host cells comprising vectors of the disclosed polynucleotide vector systems. In some embodiments, an isolated host cell comprising an overlap polynucleotide vector system is provided. In some embodiments, an isolated host cell comprising a hybrid polynucleotide vector system is provided. In particular embodiments, isolated host cells comprising second generation hybrid and isolated host cells comprising second generation overlap vectors are provided.

Examples of suitable host cells that comprise any of the disclosed dual vector systems include, but are not limited to, photoreceptor cells, cone cells, rod cells, retinal cells (e.g., ganglion cells, retinal pigment epithelium cells), or any combination thereof. Examples of retinal cells include retinal ganglion cells (RGCs), Muller cells, astrocytes, and bipolar cells.

Additional examples of suitable host cells are vestibular hair cells, inner ear hair cells, outer ear hair cells, or any combination thereof.

The disclosure also provides methods for expressing or transducing a selected polypeptide in a cell. In particular embodiments, the method comprises incorporating in the cell an AAV-based, dual vector system as disclosed herein, wherein the vector system includes a polynucleotide sequence that encodes a selected polypeptide and of interest, and expressing the polynucleotide sequences in the cell.

In certain embodiments, the selected polypeptide may be a polypeptide that is heterologous to the cell. In particular embodiments, the cell is a mammalian cell, and preferably, a human cell. In particular embodiments, the cell is a human photoreceptor cell, and preferably a human photoreceptor cone cell or a photoreceptor rod cell. In particular embodiments, the cell expresses a wild type, functional, and/or biologically-active hMYO7A polypeptide that is encoded by a nucleic acid segment present in a vector system as disclosed herein. In particular embodiments, the hMYO7A polypeptide is encoded by the nucleotide sequence shown in SEQ ID NO: 5 or SEQ ID NO: 7.

In particular embodiments, the cell is a photoreceptor cell. In particular embodiments, the cell is a cone cell; preferably, it is a human cone cell or a human rod cell. Such cells may express one or more nucleotide sequences provided in at least a first AAV-based, dual vector system of the disclosure. In particular embodiments, the cell expresses a wild-type, functional, and/or biologically active hMYO7A polypeptide that is encoded by a nucleic acid segment comprised within one or more of the AAV-based vector systems as disclosed herein. In particular embodiments, the hMYO7A polypeptide is encoded by the nucleotide sequence of SEQ ID NO: 5 or SEQ ID NO: 7.

Accordingly, in certain embodiments, the disclosure provides for methods for transducing or expressing a polynucleotide vector system in one or more photoreceptor cells or one or more RPE cells of a mammal (e.g., a human). In an overall and general sense, such a method includes administering (for example, directly administering subretinally) to one or both eyes of the mammal one or more of the rAAV particles disclosed herein, wherein the polynucleotide further comprises at least a first polynucleotide that comprises a PR- or an RPE-cell-specific promoter operably linked to at least a first heterologous nucleic acid segment that encodes a therapeutic agent (full-length polypeptide), for a time effective to produce the therapeutic agent in the one or more PR cells or RPE cells of the mammal. In certain embodiments, the therapeutic polypeptide is stably expressed in a photoreceptor cell, retinal pigment epithelium cell, retinal ganglion cell, bipolar cell, Muller glial cell or astrocyte cell, or combinations thereof. In certain embodiments, the therapeutic polypeptide is stably expressed in a vestibular hair cell, inner ear hair cell, or outer ear hair cell.

### Methods of Treatment and Transduction

In some aspects, the disclosure provides methods for treating or ameliorating a disease or condition, such as an eye disease, in a human or animal using gene therapy and an AAV-based dual vector system of the disclosure. In particular embodiments, a method of the disclosure comprises administering a vector system of the disclosure that encodes a polypeptide that provides for treatment or amelioration of the disease or condition. In particular embodiments, the vectors of the disclosure are provided in an AAV virus or virion. The vector system can be administered in vivo or ex vivo.

In particular embodiments, a vector system of the disclosure is administered in a recombinant AAV particle by parenteral administration, such as intravitreal, subretinal, intravenous, intramuscular, intraocular, utricle, or intranasal injection. In some embodiments, vector systems are administered to, e.g., hair cells of the ear, by injection into the utricle, which is one of two sac-like otolith organs sensitive to gravity, as described in Lee et al., Hearing Research Vol. 394 (2020) 107882, incorporated by reference herein. Administration to, e.g., hair cells of the ear may be by a round window injection, or during cochlear implant surgery. In particular embodiments, a vector system of the disclosure is administered to the human or animal by intraocular, intravitreal or subretinal injection.

In some embodiments, the recombinant AAV particle of the disclosure is administered via subretinal injection in a titer of about 1×10⁸ vg/ml, 5×10⁸ vg/ml, 8×10⁸ vg/ml, 1×10⁹ vg/ml, 5×10⁹ vg/ml, 1×10¹⁰ vg/ml, 5×10¹⁰ vg/ml, 1×10¹¹ vg/ml, 5×10¹¹ vg/ml, 1×10¹² vg/ml, 2×10¹² vg/ml, 3×10¹² vg/ml, 4×10¹² vg/ml, about 5×10¹² vg/ml, about 1×10¹³ vg/ml, or about 5×10¹³ vg/ml. In particular embodiments, the rAAV particle is administered in a titer of 5.0×10⁸ vg or 8.0×10⁸ vg.

In some embodiments, the subretinal injection is provided in a volume of about 200 μL, about 175 μL, about 160 μL, about 145 μL, about 130 μL, about 115 μL, about 100 μL, about 90 μL, about 80 μL, about 70 μL, about 60 μL, about 55 μL, about 50 μL, about 45 μL, about 35 μL, about 20 μL, about 10 μL, or about 5 μL. In particular embodiments, the injection is provided in a volume of about 50 μL. Dosage regimes and effective amounts to be administered can be determined by ordinarily skilled clinicians. Administration may be in the form of a single dose or multiple doses. General methods for performing gene therapy using polynucleotides, expression constructs, and vectors are known in the art (see, e.g., Gene Therapy: Principles and Applications (1999); and U.S. Pat. Nos. 6,461,606; 6,204,251 and 6,106,826, each of which is specifically incorporated herein in its entirety by express reference thereto).

In particular embodiments, the disease, disorder or condition to be treated is Usher Syndrome. In some embodiments, the disease or disorder to be treated is autosomal recessive isolated deafness (DFNB2). In other embodiments, the disease, disorder or condition such as age-related macular degeneration (AMD), wet AMD, dry AMD, or geographic atrophy. In certain embodiments, the disease or disorder is retinitis pigmentosa or glaucoma.

The disclosed dual vector systems may be introduced into one or more selected mammalian cells using any one or more of the methods that are known to those of ordinary skill in the gene therapy and/or viral arts. Such methods include, without limitation, transfection, microinjection, electroporation, lipofection, cell fusion, and calcium phosphate precipitation, as well as biolistic methods. In particular embodiments, the vectors of the disclosure may be introduced in vivo, including, for example, by lipofection (for example, DNA transfection via liposomes prepared from one or more cationic lipids) (see, e.g., Felgner et al., 1987). Synthetic cationic lipids (LIPOFECTIN®, Invitrogen Corp., La Jolla, CA, USA) may be used to prepare liposomes that will encapsulate the vectors to facilitate their introduction into one or more selected cells. A vector system of the disclosure can also be introduced in vivo as “naked” DNA using methods known to those of ordinary skill in the art.

In an overall and general sense, the disclosed methods include at least the step of administering to one or both eyes of the mammal in need thereof, one or more of the disclosed rAAV particles herein, in an amount and for a time sufficient to treat or ameliorate the one or more symptoms of the disease, the disorder, the dysfunction, the injury, the abnormal condition, or the trauma in the mammal. In some embodiments, the mammal is a human. In some embodiments, the human is a neonate, a newborn, an infant, or a juvenile. In the practice of the present disclosure, it is contemplated that suitable patients will include, for example, humans that have, are suspected of having, are at risk for developing, or have been diagnosed with one or more retinal disorders, diseases, or dystrophies, including, without limitation, retinal disorders, diseases, and dystrophies that are genetically linked, or inheritable.

In some aspects, the present disclosure provides methods of use of the particles, vectors, virions, expression systems, compositions, and host cells described herein in a method for treating or ameliorating the symptoms, or in the preparation of medicaments for, treating or ameliorating the symptoms of various deficiencies in an eye of a mammal, and in particular one or more deficiencies in human photoreceptors or RPE cells. Exemplary diseases and disorders of the eye (e.g., caused by one or more genetic deficiencies in a PR or RPE cell) for treatment or amelioration of symptoms include Retinitis pigmentosa, Leber Congenital Amaurosis (e.g., LCA10), Age Related Macular Degeneration (AMD), wet AMD, dry AMD, uveitis, Best disease, Stargardt disease, Usher Syndrome, Geographic Atrophy, Diabetic Retinopathy, Retinoschisis, Achromatopsia, Choroideremia, Bardet Biedl Syndrome, and glycogen storage diseases (ocular manifestation).

In some embodiments, administration of any of the disclosed vectors, virions, or compositions to a subject in need thereof provides a partial or complete restoration of melanosome migration in retinal pigment epithelium (RPE) cells. In exemplary embodiments, administration of any of the polynucleotide vector systems, virions, or compositions provides a partial or complete restoration of vision loss.

In some aspects, the present disclosure provides methods of use of the particles, vectors, virions, expression systems, compositions, and host cells described herein in a method for treating or ameliorating the symptoms, or in the preparation of medicaments for, treating or ameliorating the symptoms of various deficiencies in an ear of a mammal, and in particular one or more deficiencies in hair cells of the auditory and hair cells of the vestibular systems. In exemplary embodiments, the subject in need thereof suffers from a disease or disorder selected from Usher syndrome or autosomal recessive isolated deafness (DFNB2). In some embodiments, the subject suffers from Usher Syndrome type 1B, 1D, 1F, or 2A.

In some embodiments, the subject suffers from a disease or condition of the eye, and/or a disease or disorder of the ear, selected from Stargardt Disease; LCA10; Retinitis Pigmentosa, Alstrom syndrome; Usher Syndrome type 1B, 1D, 1F, or 2A; Duchenne muscular dystrophy; Cystic fibrosis; Glycogen storage disease III; non-syndromic deafness; Hemophilia A, or a dysferlinopathy.

Such methods may involve intravitreal or subretinal administration to one or both eyes of a subject in need thereof, one or more of the disclosed particles vectors, virions, host cells, or compositions, in an amount and for a time sufficient to treat or ameliorate the symptoms of such a deficiency in the affected mammal. The methods may also encompass prophylactic treatment of animals suspected of having such conditions, or administration of such compositions to those animals at risk for developing such conditions either following diagnosis, or prior to the onset of symptoms.

### Pharmaceutical Compositions and Kits

Pharmaceutical dosage forms suitable for injection or infusion can include sterile aqueous solutions or dispersions or sterile powders comprising the active ingredient, which are adapted for the extemporaneous preparation of sterile injectable or infusible solutions or dispersions, optionally encapsulated in liposomes. The ultimate dosage form should be sterile, fluid and stable under the conditions of manufacture and storage. The liquid carrier or vehicle can be a solvent or liquid dispersion medium comprising, for example, water, ethanol, a polyol (e.g., glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. Optionally, the prevention of the action of microorganisms can be brought about by various other antibacterial and antifungal agents, e.g., parabens, chlorobutanol, phenol, sorbic acid, thimerosal, and the like. In many cases, it will be preferable to include isotonic agents, e.g., sugars, buffers or sodium chloride. Prolonged absorption of the injectable compositions can be brought about by the inclusion of agents that delay absorption, e.g., aluminum monostearate and gelatin.

The disclosure also provides pharmaceutical compositions comprising a vector system of the disclosure in combination with a pharmaceutically acceptable carrier. Pharmaceutical compositions adapted for topical or parenteral administration, comprising an amount of a compound constitute a preferred embodiment of the disclosure. The dose administered to a patient, particularly a human, in the context of the disclosure should be sufficient to achieve a therapeutic response in the patient over a reasonable timeframe, without lethal toxicity, and preferably causing no more than an acceptable level of side effects or morbidity. One skilled in the art will recognize that dosage will depend upon a variety of factors including the condition (health) of the subject, the body weight of the subject, kind of concurrent treatment, if any, frequency of treatment, therapeutic ratio, as well as the severity and stage of the pathological condition.

The disclosure also provides kits comprising a vector system of the disclosure in one or more containers. Kits of the disclosure can optionally include pharmaceutically acceptable carriers and/or diluents. In particular embodiments, a kit of the disclosure includes one or more other components, adjuncts, or adjuvants as described herein. In particular embodiments, a kit of the disclosure includes instructions or packaging materials that describe how to administer a vector system contained within the kit to a selected mammalian recipient.

Containers of the disclosed kits may be of any suitable material, e.g., glass, plastic, metal, etc., and of any suitable size, shape, or configuration. In particular embodiments, a vector system of the disclosure is provided in the kit as a solid. In another embodiment, a vector system of the disclosure is provided in the kit as a liquid or solution. In certain embodiments, the kits may include one or more ampoules or syringes that contain a vector system of the disclosure in a suitable liquid or solution form.

Further contemplated herein are kits containing a pre-mixture of any of the disclosed dual vectors (front half vector and back half vector). These pre-mixtures may be in a single container and/or a single drug product in a suitable liquid or solution form.

The disclosure also provides for the use of the buffers and compositions disclosed herein in the manufacture of a medicament for treating, preventing or ameliorating the symptoms of a disease, disorder, dysfunction, injury or trauma, including, but not limited to, the treatment, prevention, and/or prophylaxis of a disease, disorder or dysfunction, and/or the amelioration of one or more symptoms of such a disease, disorder or dysfunction.

The amount of AAV compositions and time of administration of such compositions will be within the purview of the skilled artisan having benefit of the present teachings. The administration of therapeutically-effective amounts of the disclosed compositions may be achieved by a single administration, such as for example, a single injection of sufficient numbers of infectious particles to provide therapeutic benefit to the patient undergoing such treatment. Alternatively, in some circumstances, it may be desirable to provide multiple, or successive administrations of the AAV vector compositions, either over a relatively short, or over a relatively prolonged period, as may be determined by the medical practitioner overseeing the administration of such compositions.

For example, the number of infectious particles administered to a mammal may be approximately 10⁷, 10⁸, 10⁹, 10¹⁰, 10¹¹, 10¹², 10¹³, or even higher, infectious particles/mL, given either as a single dose (or divided into two or more administrations, etc.) as may be required to achieve therapy of the particular disease or disorder being treated. In fact, in certain embodiments, it may be desirable to administer two or more different rAAV particle-or vector-based compositions, either alone, or in combination with one or more other diagnostic agents, drugs, bioactives, or such like, to achieve the desired effects of a particular regimen or therapy. In most rAAV-vectored, gene therapy-based regimens, the inventors contemplate that lower titers of infectious particles will be required when practicing the disclosed methods of pre-treating and co-administering AAV capsids with HA.

To express a therapeutic agent in accordance with the present disclosure one may prepare a rAAV particle that comprises a therapeutic agent-encoding nucleic acid segment under the control of one or more promoters. To bring a sequence “under the control of” a promoter, one positions the 5′ end of the transcription initiation site of the transcriptional reading frame generally between about 1 and about 50 nucleotides “downstream” of (for example, 3 of) the chosen promoter. The “upstream” promoter stimulates transcription of the DNA and promotes expression of the encoded polypeptide. This is the meaning of “recombinant expression” in this context. In some embodiments, recombinant vector constructs are those that include a capsid-protein modified rAAV vector that contains an RPE cell- or a photoreceptor cell-specific promoter, operably linked to at least one nucleic acid segment encoding one or more diagnostic, and/or therapeutic agents.

When the use of such vectors is contemplated for introduction of one or more exogenous proteins, polypeptides, peptides, ribozymes, and/or antisense oligonucleotides, to a particular cell transfected with the vector, one may employ the rAAV particles disclosed herein to deliver one or more exogenous polynucleotides to a selected host cell, e.g., to one or more selected cells within the mammalian eye.

In some embodiments, the number of viral particles administered to a subject may be on the order ranging from 10⁶ to 10¹⁴ particles/ml or 10³ to 10¹⁵ particles/ml, or any values therebetween for either range, such as for example, about 10⁶, 10⁷, 10⁸, 10⁹, 10¹⁰, 10¹¹, 10¹², 10¹³, or 10¹⁴ particles/ml. In one embodiment, viral particles of higher than 10¹³ particles/ml may be administered. In some embodiments, the number of viral particles administered to a subject may be on the order ranging from 10⁶ to 10¹⁴ vector genomes(vgs)/ml or 10³ to 10¹⁵ vgs/ml, or any values therebetween for either range, such as for example, about 10⁶, 107, 10, 10⁹, 10¹⁰, 10¹¹, 10¹², 10¹³, or 10¹⁴ vgs/ml. In one embodiment, viral particles of higher than 10¹³ vgs/ml are administered. The viral particles can be administered as a single dose, or divided into two or more administrations as may be required to achieve therapy of the particular disease or disorder being treated. In some embodiments, doses of 0.0001 ml to 10 ml, e.g., 0.001 ml, 0.01 ml, 0.1 ml, 1 ml, 2 ml, 5 ml or 10 ml, are delivered to a subject.

In some embodiments, the disclosure provides formulations of one or more viral-based compositions disclosed herein in pharmaceutically acceptable solutions for administration to a cell or an animal, either alone or in combination with one or more other modalities of therapy, and in particular, for therapy of human cells, tissues, and diseases affecting man.

If desired, rAAV particles described herein may be administered in combination with other agents as well, such as, e.g., proteins or polypeptides or various pharmaceutically-active agents, including one or more systemic or topical administrations of therapeutic polypeptides, biologically active fragments, or variants thereof. In fact, there is virtually no limit to other components that may also be included, given that the additional agents do not cause a significant adverse effect upon contact with the target cells or host tissues. The rAAV particles may thus be delivered along with various other agents as required in the particular instance. Such compositions may be purified from host cells or other biological sources, or alternatively may be chemically synthesized as described herein.

Formulation of pharmaceutically-acceptable buffer, excipients and carrier solutions is well known to those of skill in the art, as is the development of suitable dosing and treatment regimens for using the particular compositions described herein in a variety of treatment regimens, including e.g., oral, parenteral, intraocular (e.g., subretinal or intravitreal), intravenous, intranasal, intra-articular, intra-utricle, intracochlear and intramuscular administration and formulation.

Typically, these formulations may contain at least about 0.1% of the therapeutic agent (e.g., rAAV particle) or more, although the percentage of the active ingredient(s) may, of course, be varied and may conveniently be between about 1 or 2% and about 70% or 80% or more of the weight or volume of the total formulation. Naturally, the amount of therapeutic agent(s) in each therapeutically-useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

The term “excipient” refers to a diluent, adjuvant, carrier, or vehicle with which the rAAV particle is administered. Such pharmaceutical excipients can be sterile liquids, such as water and oils, including those of petroleum oil such as mineral oil, vegetable oil such as peanut oil, soybean oil, and sesame oil, animal oil, or oil of synthetic origin. Saline solutions and aqueous dextrose and glycerol solutions can also be employed as liquid carriers. Exemplary excipients and vehicles include, but are not limited to, HA, BSS, artificial CSF, PBS, Ringer's lactate solution, TMN200 solution, polysorbate 20, and poloxamer 100.

The amount of rAAV particle compositions and time of administration of such compositions will be within the purview of the skilled artisan having benefit of the present teachings. It is likely, however, that the administration of therapeutically-effective amounts of the disclosed compositions may be achieved by a single administration, such as for example, a single injection of sufficient numbers of viral particles to provide therapeutic benefit to the patient undergoing such treatment. Alternatively, in some circumstances, it may be desirable to provide multiple, or successive administrations of the compositions, either over a relatively short, or a relatively prolonged period of time, as may be determined by the medical practitioner overseeing the administration of such compositions.

Exemplary compositions may include rAAV particles or nucleic acid vectors either alone, or in combination with one or more additional active ingredients, which may be obtained from natural or recombinant sources or chemically synthesized.

Methods of Manufacturing rAAV Particles

Recombinant adeno-associated virus (rAAV) vectors have been used successfully for in vivo gene transfer in numerous pre-clinical animal models of human disease, and have been used successfully for long-term expression of a wide variety of therapeutic genes (Daya and Berns, 2008; Niemeyer et al., 2009; Owen et al., 2002; Keen-Rhinehart et al., 2005; Scallan et al., 2003; Song et al., 2004). AAV vectors have also generated long-term clinical benefit in humans when targeted to immune-privileged sites, for example, ocular delivery for Leber congenital amaurosis (Bainbridge et al., 2008; Maguire et al., 2008; Cideciyan et al., 2008). A major advantage of this vector is its comparatively low immune profile, eliciting only limited inflammatory responses and, in some cases, even directing immune tolerance to transgene products (LoDuca et al., 2009). Nonetheless, the therapeutic efficiency, when targeted to non-immune privileged organs, has been limited in humans due to antibody and CD8⁺ T cell responses against the viral capsid, while in animal models, adaptive responses to the transgene product have also been reported (Manno et al., 2006; Mingozzi et al., 2007; Muruve et al., 2008; Vandenberghe and Wilson, 2007; Mingozzi and High, 2007). These results suggested that immune responses remain a concern for AAV vector-mediated gene transfer.

Adeno-associated virus (AAV) is considered the optimal vector for ocular gene therapy due to its efficiency, persistence and low immunogenicity (Daya and Berns, 2008). Identifying vectors capable of transducing PRs via the vitreous has historically relied on identifying which serotypes have native tropism for this cell type following local delivery. Several serotypes have been used to successfully target transgene to PRs following subretinal injection (including, e.g., AAV2, AAV5 and AAV8) with all three demonstrating efficacy in experiments performed across multiple mammalian species (e.g., mouse, rat, dog, pig and non-human primate) (Ali et al., 1996; Auricchio et al., 2001; Weber et al., 2003; Yang et al., 2002; Acland et al., 2001; Vandenberghe et al., 2011; Bennett et al., 1999; Allocca et al., 2007; Petersen-Jones et al., 2009; Lotery et al., 2003; Boye et al., 2012; Stieger et al., 2008; Mussolino et al., 2011; Vandenberghe et al., 2011).

Studies comparing their relative efficiency following subretinal delivery in the rodent show that both AAV5 and AAV8 transduce PRs more efficiently than AAV2, with AAV8 being the most efficient (Yang et al., 2002; Allocca et al., 2007; Rabinowitz et al., 2002; Boye et al., 2011; Pang et al., 2011). It was previously shown that AAV2 and AAV8 vectors containing point mutations of surface-exposed tyrosine residues (tyrosine to phenylalanine, Y-F) display increased transgene expression in a variety of retinal cell types relative to unmodified vectors following both subretinal and intravitreal injection (Petrs-Silva et al., 2009; Petrs-Silva et al., 2011). Of the vectors initially tested by those authors, an AAV2 triple mutant (designated “triple Y-F”) exhibited the highest transduction efficiency following intravitreal injection, whereas an AAV2 quadruple mutant (“quad Y-F”) exhibited the novel property of enhanced transduction of outer retina (Petrs-Silva et al., 2011).

Further improvements in transduction efficiency have been achieved via directed mutagenesis of surface-exposed threonine (T) or serine (S) residues to non-native amino acids at one of more of those amino acids. Both Y-F and T-V/T-A mutations have been shown to increase efficiency by decreasing phosphorylation of capsid and subsequent ubiquitination as part of the proteosomal degradation pathway (Zhong et al., 2008; Aslanidi et al., In Press; Gabriel et al., 2013). It has been found that the transduction profile of intravitreally-delivered AAV is heavily dependent upon the injection procedure itself. Due to the small size of the mouse eye, it is not uncommon for trans-scleral, intravitreal injections to result in damage to the retina that might allow delivery of some vector directly to the subretinal space.

Exemplary rAAV nucleic acid vectors useful according to the disclosure include single-stranded (ss) or self-complementary (sc) AAV nucleic acid vectors, such as single-stranded or self-complementary recombinant viral genomes.

Methods of producing rAAV particles and nucleic acid vectors are also known in the art and commercially available (see, e.g., Zolotukhin et al., Production and purification of serotype 1, 2, and 5 recombinant adeno-associated viral vectors, Methods 28 (2002) 158-167; and U.S. Patent Publication Nos. US 2007/0015238 and US 2012/0322861, which are incorporated herein by reference; and plasmids and kits available from ATCC and Cell Biolabs, Inc.). For example, a plasmid containing the nucleic acid vector sequence may be combined with one or more helper plasmids, e.g., that contain a rep gene (e.g., encoding Rep78, Rep68, Rep52 and Rep40) and a cap gene (encoding VP1, VP2, and VP3, including a modified VP3 region as described herein), and transfected into a producer cell line such that the rAAV particle can be packaged and subsequently purified.

In some embodiments, the one or more helper plasmids includes a first helper plasmid comprising a rep gene and a cap gene and a second helper plasmid comprising a Ela gene, a Elb gene, a E4 gene, a E2a gene, and a VA gene. In some embodiments, the rep gene is a rep gene derived from AAV2 and the cap gene is derived from AAV2 and includes modifications to the gene in order to produce a modified capsid protein described herein. Helper plasmids, and methods of making such plasmids, are known in the art and commercially available (see, e.g., pDM, pDG, pDP1rs, pDP2rs, pDP3rs, pDP4rs, pDP5rs, pDP6rs, pDG(R484E/R585E), and pDP8.ape plasmids from PlasmidFactory, Bielefeld, Germany; other products and services available from Vector Biolabs, Philadelphia, PA; Cellbiolabs, San Diego, CA; Agilent Technologies, Santa Clara, CA; and Addgene, Cambridge, MA; pxx6; Grimm et al. (1998), Novel Tools for Production and Purification of Recombinant Adenoassociated Virus Vectors, Human Gene Therapy, Vol. 9, 2745-2760; Kern, A. et al. (2003), Identification of a Heparin-Binding Motif on Adeno-Associated Virus Type 2 Capsids, Journal of Virology, Vol. 77, 11072-11081; Grimm et al. (2003), Helper Virus-Free, Optically Controllable, and Two-Plasmid-Based Production of Adeno-associated Virus Vectors of Serotypes 1 to 6, Molecular Therapy, Vol. 7, 839-850; Kronenberg et al. (2005), A Conformational Change in the Adeno-Associated Virus Type 2 Capsid Leads to the Exposure of Hidden VP1 N Termini, Journal of Virology, Vol. 79, 5296-5303; and Moullier, P. and Snyder, R. O. (2008), International efforts for recombinant adeno-associated viral vector reference standards, Molecular Therapy, Vol. 16, 1185-1188).

An exemplary, non-limiting, rAAV particle production method is described next. One or more helper plasmids are produced or obtained, which comprise rep and cap ORFs for the desired AAV serotype and the adenoviral VA, E2A (DBP), and E4 genes under the transcriptional control of their native promoters. The cap ORF may also comprise one or more modifications to produce a modified capsid protein as described herein. HEK293 cells (available from ATCC®) are transfected via CaPO4-mediated transfection, lipids or polymeric molecules such as Polyethylenimine (PEI) with the helper plasmid(s) and a plasmid containing a nucleic acid vector described herein. The HEK293 cells are then incubated for at least 60 hours to allow for rAAV particle production. Alternatively, in another example Sf9-based producer stable cell lines are infected with a single recombinant baculovirus containing the nucleic acid vector. As a further alternative, in another example HEK293 or BHK cell lines are infected with a HSV containing the nucleic acid vector and optionally one or more helper HSVs containing rep and cap ORFs as described herein and the adenoviral VA, E2A (DBP), and E4 genes under the transcriptional control of their native promoters. The HEK293, BHK, or Sf9 cells are then incubated for at least 60 hours to allow for rAAV particle production. The rAAV particles can then be purified using any method known the art or described herein, e.g., by iodixanol step gradient, CsCl gradient, chromatography, or polyethylene glycol (PEG) precipitation.

### Exemplary Definitions

In accordance with the disclosure, polynucleotides, nucleic acid segments, nucleic acid sequences, and the like, include, but are not limited to, DNAs (including, but not limited to, genomic and/or extragenomic DNAs), genes, peptide nucleic acids (PNAs) RNAs (including, but not limited to, rRNAs, mRNAs, and/or tRNAs), nucleosides, as well as one or more nucleic acid segments obtained from natural sources, chemically synthesized, genetically modified, or otherwise prepared or synthesized in whole or in part by the hand of man.

Unless defined otherwise, all technical and scientific terms used herein have the same meaning as commonly understood by one of ordinary skill in the art to which this invention belongs. Although any methods and compositions similar or equivalent to those described herein can be used in the practice or testing of the disclosure, the preferred methods and compositions are described herein. For purposes of the disclosure, the following terms are defined below: As used herein, the terms “nucleic acid” and “polynucleotide sequence” refer to a deoxyribonucleotide or ribonucleotide polymer in either single- or double-stranded form, and unless otherwise limited, encompass known analogs of natural nucleotides that can function in a similar manner as naturally occurring nucleotides. The polynucleotide sequences include both full-length sequences, as well as shorter sequences derived from the full-length sequences. It is understood that a particular polynucleotide sequence includes the degenerate codons of the native sequence or sequences that may be introduced to provide codon preference in a specific host cell. The polynucleotide sequences falling within the scope of the disclosure further include sequences that specifically hybridize with the sequences coding for a peptide of the disclosure. The polynucleotide includes both the sense and antisense strands, either as individual strands or in the duplex.

Fragments and variants of a polynucleotide of the disclosure can be generated as described herein and tested for the presence of function using standard techniques known in the art. Thus, an ordinarily skilled artisan can readily prepare and test fragments and variants of a polynucleotide or polypeptide of the disclosure and determine whether the fragment or variant retains functional activity that is the same or similar to a full-length or a non-variant polynucleotide or polypeptide, such as a myosin VIIa polynucleotide or polypeptide.

Also within the scope of the disclosure are polynucleotides that have the same, or substantially the same, nucleotide sequence of a polynucleotide exemplary herein, except for the presence of one or more nucleotide substitutions, additions, or deletions within the sequence of the polynucleotide, so long as these variant polynucleotides retain substantially the same relevant functional activity as the polynucleotides exemplary herein (for example, they encode a protein having the same amino acid sequence or the same functional activity as one of the polynucleotides specifically exemplary herein). Thus, the polynucleotides disclosed herein should also be understood to include variants and fragments thereof.

As one of ordinary skill in the molecular biological arts can readily appreciate, there can be a number of variant sequences of a gene or polynucleotide found in nature, in addition to those variants that may be artificially prepared or synthesized by an ordinary-skilled artisan in a laboratory environment. The polynucleotides of the disclosure encompasses those specifically exemplary herein, as well as any natural variants thereof, as well as any variants which can be created artificially, so long as those variants retain the desired biological activity.

Also within the scope of the disclosure are polynucleotides which have the same nucleotide sequences of a polynucleotide exemplary herein except for nucleotide substitutions, additions, or deletions within the sequence of the polynucleotide, as long as these variant polynucleotides retain substantially the same relevant biological activity as the polynucleotides specifically exemplary herein. Thus, the polynucleotides disclosed herein should be understood to include variants and fragments, as discussed above, of the specifically exemplary sequences.

Polynucleotides described herein can also be defined in terms of more particular identity and/or similarity ranges with those exemplary herein. The sequence identity will typically be greater than 60%, preferably greater than 75%, more preferably greater than 80%, even more preferably greater than 90%, and can be greater than 95%. The identity and/or similarity of a sequence can be 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99% or greater as compared to a sequence exemplary herein.

Unless otherwise specified, as used herein percent sequence identity and/or similarity of two sequences can be determined using the algorithm of Karlin and Altschul (1990), modified as in Karlin and Altschul (1993). Such an algorithm is incorporated into the NBLAST and XBLAST programs of Altschul et al. (1990). BLAST searches can be performed with the NBLAST program, score=100, word-length=12, to obtain sequences with the desired percent sequence identity. To obtain gapped alignments for comparison purposes, Gapped BLAST can be used as described (Altschul et al., 1997). When utilizing BLAST and Gapped BLAST programs, the default parameters of the respective programs (NBLAST and XBLAST) can be used in accordance with published methods.

The disclosure also contemplates those polynucleotide molecules having sequences that are sufficiently homologous with the polynucleotide sequences of the disclosure to permit hybridization with that sequence under standard stringent conditions and standard methods (Maniatis et al., 1982). As used herein, “stringent” conditions for hybridization refers to conditions wherein hybridization is typically carried out overnight at 20-25 degrees Celsius below the melting temperature (Tₘ) of the DNA hybrid in 6×SSPE, 5×Denhardt's solution, and 0.1% SDS, containing 0.1 mg/mL of a suitable non-specific denatured DNA.

The term “effective amount,” as used herein, refers to an amount that is capable of treating or ameliorating a disease or condition or otherwise capable of producing an intended therapeutic effect.

The term “operably linked,” as used herein, refers to that the nucleic acid sequences being linked are typically contiguous, or substantially contiguous, and, where necessary to join two protein coding regions, contiguous and in reading frame. However, since enhancers generally function when separated from the promoter by several kilobases and intronic sequences may be of variable lengths, some polynucleotide elements may be operably linked but not contiguous.

The term “promoter,” as used herein, refers to a region or regions of a nucleic acid sequence that regulates transcription. Exemplary promoters provided herein include, but are not limited to, a CMV promoter, an EF-1 alpha promoter, a cone arrestin promoter, a chimeric CMV R actin promoter (CBA), a truncated chimeric CMV R actin (smCBA) promoter, a human myosin 7a gene-derived promoter, a TaC gene-derived promoter, a rhodopsin promoter, a cGMP-phosphodiesterase β-subunit promoter, human or mouse rhodopsin promoter, a hGRK1 promoter, a synapsin promoter, a glial fibrillary acidic protein (GFAP) promoter, a rod specific IRBP promoter, a VMD2 promoter.

The term “regulatory element,” as used herein, refers to a region or regions of a nucleic acid sequence that regulates transcription. Exemplary regulatory elements include, but are not limited to, enhancers, post-transcriptional elements, transcriptional control sequences, and such like.

The term “substantially corresponds to,” “substantially homologous,” or “substantial identity,” as used herein, denote a characteristic of a nucleic acid or an amino acid sequence, wherein a selected nucleic acid or amino acid sequence has at least about 70 or about 75 percent sequence identity as compared to a selected reference nucleic acid or amino acid sequence. More typically, the selected sequence and the reference sequence will have at least about 76, 77, 78, 79, 80, 81, 82, 83, 84 or even 85 percent sequence identity, and more preferably, at least about 86, 87, 88, 89, 90, 91, 92, 93, 94, or 95 percent sequence identity. More preferably still, highly homologous sequences often share greater than at least about 96, 97, 98, or 99 percent sequence identity between the selected sequence and the reference sequence to which it was compared.

The percentage of sequence identity may be calculated over the entire length of the sequences to be compared, or may be calculated by excluding small deletions or additions which total less than about 25 percent or so of the chosen reference sequence. The reference sequence may be a subset of a larger sequence, such as a portion of a gene or flanking sequence, or a repetitive portion of a chromosome. However, in the case of sequence homology of two or more polynucleotide sequences, the reference sequence will typically comprise at least about 18-25 nucleotides, more typically at least about 26 to 35 nucleotides, and even more typically at least about 40, 50, 60, 70, 80, 90, or even 100 or so nucleotides.

When highly-homologous fragments are desired, the extent of percent identity between the two sequences will be at least about 80%, preferably at least about 85%, and more preferably about 90% or 95% or higher, as readily determined by one or more of the sequence comparison algorithms well-known to those of ordinary skill in the art, such as e.g., the FASTA program analysis described by Pearson and Lipman (1988).

The term “subject,” as used herein, describes an organism, including mammals such as primates, to which treatment with the compositions according to the disclosure can be provided. Mammalian species that can benefit from the disclosed methods of treatment include, but are not limited to, humans, non-human primates such as apes; chimpanzees; monkeys, and orangutans, domesticated animals, including dogs and cats, as well as livestock such as horses, cattle, pigs, sheep, and goats, or other mammalian species including, without limitation, mice, rats, guinea pigs, rabbits, hamsters, and the like.

The term “treatment” or any grammatical variation thereof (e.g., treat, treating, and treatment, etc.), as used herein, includes but is not limited to, alleviating a symptom of a disease or condition; and/or reducing, suppressing, inhibiting, lessening, ameliorating or affecting the progression, severity, and/or scope of a disease or condition.

The term “vector,” as used herein, refers to a nucleic acid molecule (typically one containing DNA) that is capable of replication in a suitable host cell, or one to which another nucleic acid segment can be operatively linked so as to facilitate replication of the operably-linked nucleic acid segment. Exemplary vectors include, without limitation, plasmids, cosmids, viruses and the like.

As used herein, the term “variant” refers to a molecule (e.g., a polynucleotide) having characteristics that deviate from what occurs in nature, e.g., a “variant” is at least about 80% identical, at least about 90% identical, at least about 95% identical, at least about 96% identical, at least about 97% identical, at least about 98% identical, at least about 99% identical, at least about 99.5% identical, or at least about 99.9% identical to the wild type polynucleotide. Variants of a protein molecule, e.g. a capsid, may contain modifications to the amino acid sequence (e.g., having 1, 2, 3, 4, 5, 6, 7, 8, 9, 10, 10-15, or 15-20 amino acid substitutions) relative to the wild type protein sequence, which arise from point mutations installed into the nucleic acid sequence encoding the capsid protein. These modifications include chemical modifications as well as truncations.

Variants of a nucleic acid molecule, e.g. a polynucleotide vector system, may contain modifications to the sequence (e.g., having 1, 2, 3, 4, 5, 6, 7, 8, 9, 10, 10-15, or 15-20 nucleotide substitutions) relative to the wild type nucleic acid sequence. These modifications may comprise truncations at a 5′ terminus or a 3′ terminus.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the disclosure.

### Example 1 Shows an Exemplary Overlap Dual Vector System, which has a hMYO7A Coding Overlap

In an exemplary overlap system (FIG. 2), the overlapping DNA sequence shared by both vector A and vector B consists of a 1350-bp coding region for the human MYO7A gene. This is the simplest system of the disclosure, and appears to be highly efficient in terms of full-length gene reconstitution, and MYO7A expression. Advantageously, each vector is of standard AAV packaging size, and as such, each packages DNA with a high degree of efficiency, and is readily adaptable to conventional GMP standards. Such vectors are also readily characterized to permit requisite regulatory approval prior to use in humans.

### Example 2 Shows an Exemplary Hybrid Dual-AAV Vector System, which Utilizes hMYO7A Intron 23 Splicing

In an exemplary system (FIG. 3) the overlapping DNA sequence is composed of the native intron 23 of human MYO7A. Vector A contains the coding sequence corresponding to the amino-terminal portion of the hMYO7A cDNA relative to intron 23 (hMYO7ANT) and the native splice-donor site, followed by a 250 bp fragment of intron 23 of hMYO7A (minus the native acceptor site). Vector B contains the carboxyl-terminal portion of the hMYO7A cDNA relative to intron 23 (hMYO7ACT), and a 250 bp fragment of intron 23 of MYO7A (minus the native splice-donor site), followed by the native splice-acceptor site. Upon co-delivery to suitable mammalian host cells, the DNA of vectors A and B recombine to form a reconstituted full-length gene cassette. The resulting RNA transcript will then ‘splice out’ the native intron. Alternatively, recombination and formation of the gene cassette can occur via the AAV TRs. In this case, the RNA transcript will ‘splice out’ the native intron23-TR-intron23 motif. In both cases, however, the resulting mRNA is that of the reconstituted full-length hMYO7A gene sequence.

### Example 3 Shows In Vitro Performance of an Exemplary Overlap Vector System, which Contains the hMYO7A Coding Overlap

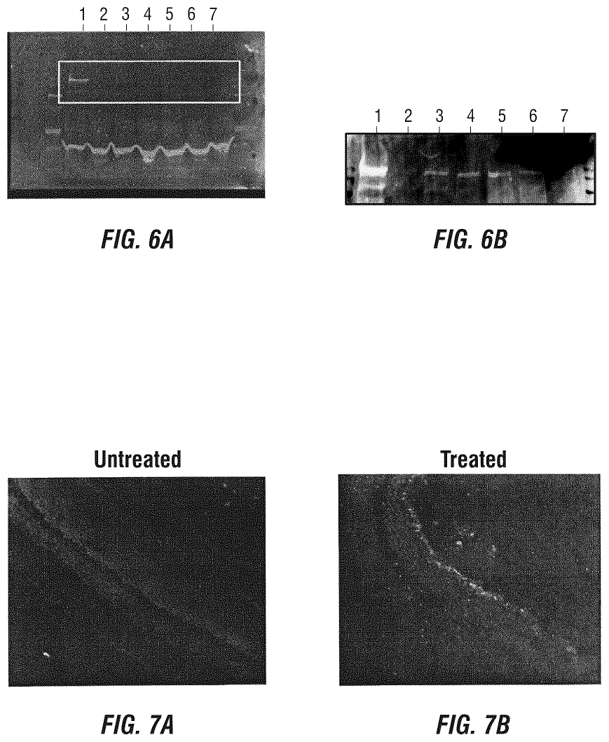

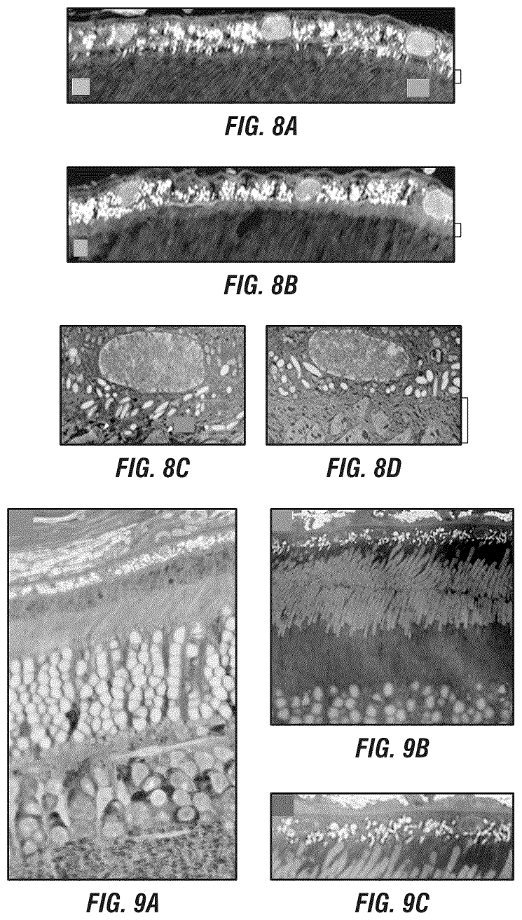

HEK293 cells were infected simultaneously with vector A and vector B of an overlap dual vector system (FIG. 2) at a ratio of 10000:1 vg/cell for each vector. The AAV vectors were packaged in AAV2 virions that contain three Y-F mutations in the capsid protein (see, e.g., Zhong et al., 2008). As a positive control, cells were transfected with plasmid containing full-length hMYO7A under the control of smCBA. Protein was recovered from cells at 3-, 4-, 5-, 6- and 7-days post-infection, and an antibody directed against MYO7A was used to assay for its presence in the infected cells via immunoblotting. The results are shown in FIG. 6A and FIG. 6B. An area of interest from inside FIG. 6A is magnified, and presented at higher contrast in FIG. 6B. Starting at 3-days post-infection, the full-length human MYO7A protein was visible; peak expression of the protein occurred around Day 5.

### Example 4 Shows In Vivo Performance of an Exemplary Overlap Vector System, which Contains the hMYO7A Coding Overlap

Six week old shaker-1 (MYO7A null) mice were sub-retinally co-injected with 1 μL of the same preparations of vector A and vector B used in the above in vitro study. Both vectors were delivered at ˜1×10¹² vg/mL. Four weeks post-injection, retinas from treated and untreated eyes were collected and immunohistochemistry (IHC) was performed using an antibody directed against MYO7A (see FIG. 7A and FIG. 7B). Brighter areas indicated MYO7A-specific staining, while the darker areas correspond to the nuclear-specific, DAPI stain. In the treated eye, MYO7A expression was clearly visible, and it appeared to be restricted to photoreceptors—more precisely to the juncture of the photoreceptor inner and outer segments.

### Example 5 Shows that AAV Dual Vectors Efficiently Deliver Oversized Genes

### Methods and Materials

Animals. Shaker-1 mice carrying the 4626SB allele, an effective null mutation (Liu et al., 1999; Hasson et al., 1997), were used on the C57BL6 genetic background, and maintained and genotyped as described (Liu et al., 1999; Gibbs et al., 2003a). They were maintained on a 12-hr light/12-hr dark cycle, with exposure to 10-50 lux of fluorescent lighting during the light phase, and were treated according to federal and institutional animal care guidelines. Homozygous mutants were distinguished from the heterozygous controls by their hyperactivity, head-tossing and circling behavior (Gibson et al., 1995), and/or by a PCR/restriction digest assay.

Construction of AAV Vectors. Single-vector platform: AAV vector plasmid, containing the truncated chimeric CMV/chicken β-actin promoter (smCBA) (Haire et al., 2006) and MYO7A cDNA was constructed by removing the full MYO7A cDNA from pEGFP-C2 by EagI and SalI digest, and then ligating into pTR-smCBA-GFP that had been digested with NotI and SalI to remove GFP. The MYO7A cDNA (˜6.7 kb) corresponded to isoform 2 of human MYO7A, and was the same as that described previously by Hashimoto et al. (2007), which was based on the sequence published by Chen et al. (1996) (see SEQ ID NO: 8). MYO7A isoform 2 is 114-kb shorter than isoform 1 (Chen et al., 1996; Weil et al., 1996). Both the MYO7A cDNA, and the resulting junctions were fully sequenced prior to packaging. All vectors intended for in vitro analyses were separately packaged in wild type AAV2, or alternatively in the AAV2 (tripleY-F) capsid mutant vector (Petrs-Silva et al., 2011).

As noted above, AAV2-based vectors were chosen for the in vitro experiments due to their increased transduction efficiency relative to other serotypes (Ryals et al., 2011). All vectors were packaged, purified, and titered using standard methods as previously described (Zolotukhin et al., 2002; Jacobson et al., 2006). Human embryonic kidney (HEK293) cells were transfected by the calcium phosphate method with vector plasmid carrying the full-length MYO7A coding sequence of variant 2 (the plasmid used to package fragmented AAV). These transfected cells were then used as a positive control throughout immunoblot analyses to indicate the appropriate size of full-length MYO7A protein. Vector infections were carried out in HEK293 cells with titer-matched AAV vectors. In brief, cells were grown to 60-70% confluency. All vectors were diluted in a balanced salt solution to achieve the desired multiplicity of infection (MOI). If not specifically mentioned, cells were infected at 10,000 genome-containing particles/cell of each vector, resulting in an MOI of 20,000 total for each vector pair. Cells were incubated in medium containing 10% serum for 3 days post-infection at 37 degrees C. under 7% C02, and then analyzed via immunoblot. Titers of 10¹² to 10¹³ particles/mL were obtained for different lots of AAV2-MYO7A and AAV5-MYO7A.

Oligonucleotide Sequences. For in vivo studies, a human influenza hemagglutinin (HA) tag was added to the 3′ termini of the full-length, simple overlap, trans-splicing, and hybrid 3′ vectors by utilizing a unique BamHI site (P19), and replacing the non-tagged 3′-end with an HA-tagged (P20) version. All constructs were sequence verified by Sanger sequencing.

AAV Vector Plasmid Design and Cloning. The full-length coding sequence of MYO7A (human isoform 2; GenBank Accession No. NM_001127180) was cloned into a vector plasmid containing the strong, ubiquitous CMV/chicken β-actin (smCBA) promoter (Haire et al., 2006), a polyadenylation signal, and the AAV2 ITRs.

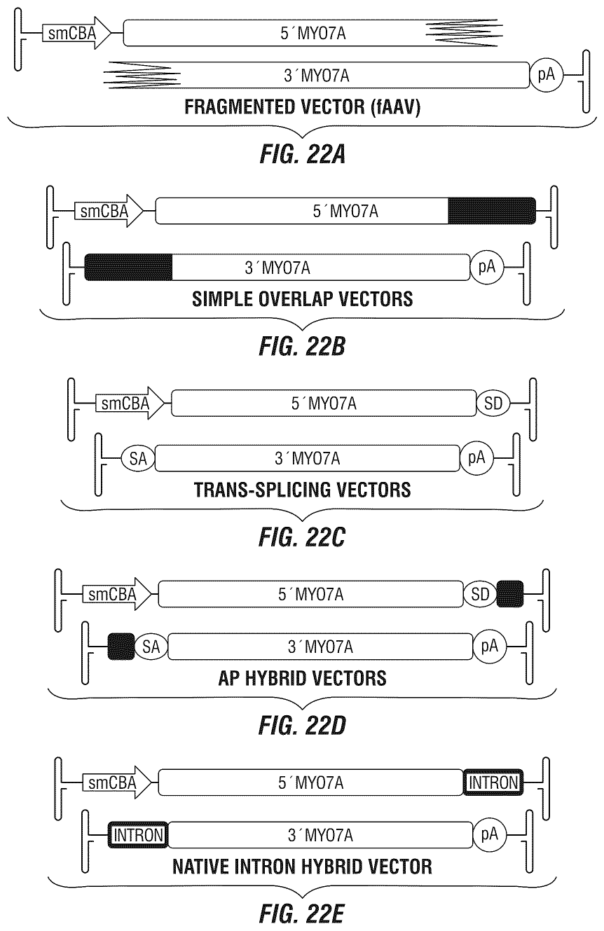

Packaging of this plasmid generated the fAAV vector (FIG. 22A). In all systems, the 5′ vectors shared the smCBA promoter and a 5′ portion of MYO7A, whereas the 3′ vectors contained a 3′-portion of MYO7A, and a bovine growth hormone (bGH) polyadenylation signal. Oligonucleotides used for vector construction are listed in Table 1. The simple overlap contained nucleotides 1 through 3644 of MYO7A cDNA from the ATG in the 5′ vector, and nucleotides 2279 through 6534 in the 3′ vector. The fragments were amplified with oligonucleotides P1 and P3 by polymerase chain reaction (PCR) and cloned into the 5′ vector via NotI and NheI, and the 3′ vector with P3 (AflII) and P4 (KpnI), respectively.

The resulting two vector plasmids share 1365 bp of overlapping MYO7A sequence (FIG. 22B). The trans-splicing and hybrid vectors utilize splice junctions composed of either ideal splice donor and acceptor sites derived from AP coding sequence or native MYO7A splice junctions from exons 23 and 24 (Yan et al., 2002). To create the 5′ trans-splicing vector, the splice-acceptor site was amplified using oligonucleotides P5 and P6 (NheI), and the amplicon was then used in a second reaction with oligonucleotide P7 (NsiI) to add a part of the MYO7A coding sequence for cloning.

The corresponding 3′ vector was similarly created by amplifying the splice-acceptor site with oligonucleotides P8 (AflII) and P9 in a first PCR, and adding part of the 3′ MYO7A coding sequence with oligonucleotide P10 (AgeI) in a second PCR (see FIG. 22C). The AP hybrid vectors were created by adding 270 bp of AP overlap sequence to the respective trans-splicing vectors (Ghosh et al., 2011). The sequence was amplified by PCR and, in so doing, appropriate restriction endonuclease sites were added. For the 5′ vector oligonucleotides P11 (NheI) and P12 (SalI) were used, while oligonucleotides P13 (NotI) and P14 (AflII) were used for the 3′ vector (FIG. 22D).

A fourth vector pair, “native intron hybrid” vector, was also created to exploit the natural sequence in and around intron 23 of MYO7A as a recombination locus, and subsequent splicing signal. The 5′-portion was created by amplifying intron 23 with oligonucleotides P15 and P16 (NheI) first, and then using the resulting amplicon in a second reaction with oligonucleotide P7 (NsiI) to facilitate cloning. The corresponding 3′-vector was constructed by amplifying the intron 23 with oligonucleotides P17 and P18 (AflII), and the resulting amplicon, with oligonucleotide P10 (AgeI) in a second reaction (see FIG. 22E).

TABLE 1
Oligonucleotides used in this study

| | | Restriction | |
|---|---|---|---|
| OliGo | 5′-3′ sequence (restriction sites underlined) | site | (SEQ ID NO:) |
| P1 | GCGGCGGCCGCCACCATGGTGATTCTTCAGCAGGGGGAC | NotI | SEQ ID NO: 9 |
| P2 | GCGGCTAGCGAAGTTCCGCAGGTACTTGAC | NheI | SEQ ID NO: 10 |
| P3 | GCGCTTAAGCAGGTCTAACTTTCTGAAGCTG | AflII | SEQ ID NO: 11 |
| P4 | GCGGGTACCTCACTTGCCGCTCCTGGAGCC | KpnI | SEQ ID NO: 12 |
| P5 | GGCACCTAGTGGCTTTGAGGTAAGTATCAAGGTTACAAGAC | | SEQ ID NO: 13 |
| P6 | GCGGCTAGCTCAGAAACGCAAGAGTCTTC | NheI | SEQ ID NO: 14 |
| P7 | CTTCTTTGTGCGATGCATCAAG | Nsii | SEQ ID NO: 15 |
| P8 | GCGCTTAAGCGACGCATGCTCGCGATAG | AflII | SEQ ID NO: 16 |
| P9 | CGCCCTCGCTCCAGGTCCTGTGGAGAGAAAGGCAAAG | | SEQ ID NO: 17 |
| P10 | GAACCCGAACCGGTCCTTG | AgeI | SEQ ID NO: 18 |
| P11 | GCGGCTAGCCCCCGGGTGCGCGGC | NheI | SEQ ID NO: 19 |
| P12 | GCGGTCGACGAAACGGTCCAGGCTATGTG | SalI | SEQ ID NO: 20 |
| P13 | GCGGCGGCCGCCCCCGGGTGCGCGGCG | NotI | SEQ ID NO: 21 |
| P14 | GCGCTTAAGGAAACGGTCCAGGCTATGTG | AflII | SEQ ID NO: 22 |
| P15 | CAGGCACCTAGTGGCTTTGAGGTACCAGGCTAGGGACAGG | | SEQ ID NO: 23 |
| P16 | GCGGCTAGCCGCCTGAGCCCAGAAGTTC | NheI | SEQ ID NO: 24 |
| P17 | CGCCCTCGCTCCAGGTCCTGAAGGAGACAAGAGGTATG | | SEQ ID NO: 25 |
| P18 | GCGCTTAAGCACCGCTTGTGTTGATCCTC | AflII | SEQ ID NO: 26 |
| P19 | GCCAGGGAAGGATCCCATG | BamHI | SEQ ID NO: 27 |
| P20 | GCGGGTACCTCATGCGTAATCCGGTACATCGTAAGGGTACTTGCCGCTCCT | KpnI | SEQ ID NO: 28 |
| | GGAGCC | | |
| P21 | AGCTTCGTAGAGTTTGTGGAGCGG | | SEQ ID NO: 29 |
| P22 | GAGGGGCAAACAACAGATG | | SEQ ID NO: 30 |

Oligonucleotides were used to make 5′ and 3′ vectors of the dual vector platforms (P1-P20). Oligonucleotides were used to characterize the fidelity of the overlap in simple overlap, trans-splicing and AP hybrid vector platforms (P21-P22). Restriction sites used for cloning are underlined and the introduced hemagglutinin (HA) tag is noted in italics (P19).

Dual Vector Platform. Two separate vector plasmids were constructed: Vector A contains the strong, ubiquitous “smCBA” promoter and MYO7A cDNA encoding the N-terminal portion. Vector B contains MYO7A cDNA encoding the C-terminal portion and a poly-A signal sequence. Each vector plasmid contained both inverted terminal repeats (ITRs). Using PCR with full-length MYO7A cDNA as a template, the MYO7A cDNA was divided roughly in half with amplicons encompassing nucleotide positions 1 through 3644 (Vector A) and 2279 through 6647 (Vector B) relative to ATG start position 1. The resulting two-vector plasmids shared 1365 bp of overlapping MYO7A sequence, and were 5.0- and 4.9-Kb in length, respectively. This was well within the size limitation of standard AAV vectors. Both vector plasmids were sequence verified and separately packaged by standard AAV production methods (Zolotukhin et al., 2002; Jacobson et al., 2006). The titer of the first lot contained 2.5×10¹² particles/mL of each vector, and the second lot contained 4×10¹² particles/mL of each vector.

Reverse Transcription and Characterization of Overlap Region. HEK293 cells were infected with dual vectors, and total RNA was extracted with the RNeasy® kit (Qiagen, Hilden, Germany) according to the manufacturer's recommended protocol. Two micrograms of RNA were then subjected to DNaseI (NEB) digestion for 30 min at 37 degrees Celsius, followed by heat inactivation at 75 degrees Celsius for 10 min. Reverse transcription to cDNA was achieved with the SuperscriptIII® kit (Life Technologies, Grand Island, NY, USA) according to the standard protocol utilizing the oligo dT primer. Two microliters of cDNA was used as template in a PCR (95 degrees Celsius for 3 min. initial denature, 35 cycles of 95 degrees Celsius for 45 sec., 55 degrees Celsius for 45 sec., 72 degrees Celsius for 12 min., and a final 72 degrees Celsius for 15 min.) using oligonucleotide primers P21 and P22 (see Table 1). Annealing sites for these primers are located 5′ and 3′, respectively, of the area of cDNA overlap (in other words, outside the region of overlap) in the simple overlap and hybrid vector pairs. The 3′-primer annealed to sequence that was complimentary to the bGH polyA. Resulting products were digested with either PpuMI or BglII, separated on a 1.5% agarose gel, and subsequently analyzed on a UV screen. Separately, products were digested with KpnI and AgeI, and subsequently cloned into a pUC vector for sequencing of the entire overlap region. M13˜forward and reverse primers that were specific for the vector were used to obtain sense and antisense reads resulting in an 140 bp overlap of the sense and antisense reads. To demonstrate that these methods were capable of detecting aberrant sequence (for example, for quality control), a MYO7A sequence was generated using either an artificial insertion (HindIII fill-in at position 2635) or a point mutation (T->C) at position 2381, and the analyses were repeated.

Viral Delivery in Vitro. HEK293A cells (Invitrogen), grown in DMEM with 10% FBS and 1×NEAA and Pen/Strep (Invitrogen) were plated in 6 well-plates. The next day cells were incubated, at 37 degrees Celsius and 5% C02, with AAV2- and AAV5-MYO7A at an MOI of 10,000 viral particles/cell in 500 μL of complete medium, containing also 40 μM of calpain inhibitor (Roche, Pleasanton, CA, USA). Two hours later complete medium was added. The next day, the medium was changed and cells were incubated for an additional 48 hrs. Alternatively, some cells were transfected with 1 μg of vector pTR-smCBA-MYO7A, complexed with Lipofectamine 2000 (ratio 1:3), according to the manufacturer's instructions (Invitrogen).

Primary mouse RPE cells were derived from P14-P16 MYO7A-null animals and cultured in 24-well dishes, as described (Gibbs et al., 2003a; Gibbs and Williams, 2003b). After 48 hrs. in culture, cells were transduced with viruses. Cells were incubated in 100 μL of complete medium containing 40 μM of calpain inhibitor, and 10,000 viral particles/cell from full-strength AAV stocks. After 2 hrs, 400 μL of complete medium was added to each well, and incubated overnight. The medium was changed the following day, and cells were incubated for an additional 48 hrs.

ARPE19 cells (American Type Culture Collection, Manassas, VA, USA) were cultivated in DMEM/F-12 with 10% FBS and split into 24-well plates with glass coverslips. Cells were grown to confluency and then transduced in the same manner, as were the primary RPE cells.

MYO7A expression analysis by Western blot and Immunofluorescence. HEK293A and primary mouse RPE cells that were transduced with AAV-MYO7A were collected 3 days post-transduction. For western blot analyses, cells were collected and lysed in 20 mM TRIS, pH 7.4, 5 mM MgCl₂, 10 mM NaCl, 1 mM DTT and 1× protease inhibitor cocktail (Sigma-Aldrich Chemical Co., St. Louis, MO, USA). Equivalent amounts of total protein were separated on a 7.5% SDS-PAGE gel. After transfer, blots were blocked with 5% non-fat milk, and probed with mouse anti-MYO7A antibody, generated against residues 927-1203 of human MYO7A (Developmental Studies Hybridoma Bank, Iowa City, IA USA) (Soni et al., 2005), and mouse anti-actin antibody (Sigma-Aldrich) as a loading control.

Immunofluorescence was performed with ARPE19 and mouse RPE primary cells, 3 days after infection. Cells were fixed in 4% formaldehyde, blocked with blocking solution (0.5% BSA/0.05% saponin in PBS), incubated with the mouse anti-MYO7A followed by goat anti-mouse Alexa-568 (Molecular Probes, Carlsbad, CA, USA). Coverslips were mounted with mounting medium containing DAPI (Fluorogel II, Electron Microscopy Sciences, Hatfield, PA, USA) and visualized on a Leica confocal system.

Protein extraction and immunoblotting. Transfected and infected HEK293 cells were harvested and washed twice in PBS and processed as previously reported with minor modifications (Boye et al., 2012). The cells were lysed by 3×30 second pulses of sonication in 200 μL of sucrose buffer (0.23 M sucrose, 2 mM EDTA, 5 mM Tris-HCl, pH 7.5) containing protease inhibitors (Roche, Mannheim, Germany). Unlysed cells and cell debris were removed by centrifugation at 14,000 rpm for 10 min. The protein concentration of the supernatant was measured with BCA (Thermo Fisher Scientific, Rockland, IL, USA). Equal amounts of protein were then loaded on 7.5% sodium dodecyl sulfate polyacrylamide gel electrophoresis gels (BioRad, Hercules, CA, USA) and transferred in CAPS buffer (pH 11) onto PVDF membranes (Millipore, Billerica, MA). Blots were then labeled with antibodies against MYO7A (monoclonal antibody raised against amino acids 11-70 of human MYO7A; Santa Cruz, Dallas, TX, USA; 1:1000) or HA (MMS-101P; Covance, Gaithersburg, MD, USA; 1:500) and R-actin (ab 34731; Abcam, Cambridge, MA, USA; 1:5000). For visualization with the Odyssey system (Li-Cor, Lincoln, NE, USA), an anti-mouse and an anti-rabbit secondary antibody conjugated with CW800 and IR680 dyes (Li-Cor), respectively, were used. Semi-quantitative densitometric measurements were performed with Odyssey acquisition and analysis software (Li-Cor). The dual-color images were separated in their respective channels and converted to gray scale for presentation purposes. Size markers present in one channel of each blot were added to both channels for visualization of protein sizes.

Viral Delivery in vivo. Mice were anesthetized with 2.0-3.0% isoflurane inhalation. The pupils of the animals were dilated with 1% (wt./vol.) atropine sulfate and 2.5% phenylephrine. A local anesthetic (0.5% proparacaine hydrochloride) was also administered. A sclerotomy in the temporal limbus was performed with a 27-Ga needle. A 32-Ga blunt needle, attached to a microsyringe pump (WPI, Sarasota, FL, USA) was inserted and 1 μL of viral solution was injected into the ventral subretinal space of P14-P16 animals. Retinal detachment was visualized under a dissecting microscope, and registered as indication of a positive subretinal injection. One microliter of the following AAV8(Y733F)-based vectors was injected subretinally in one eye of C57BL/6 mice: single fAAV (1×10¹³ vg/mL), front and back half “hybrid” vectors combined equally (each vector=1×10¹³ vg/mL), or front and back half “simple overlap” vectors combined equally (each vector=1×10¹³vg/mL). Subretinal injections were performed as previously described (Timmers et al., 2001). Further analysis was carried out only on animals that received comparable, successful injections (>60% retinal detachment with minimal surgical complications).

Light Microscopy and Immunoelectron Microscopy of Retinas. Eyecups were processed for embedment in either LR White or Epon, and semithin and ultrathin sections were prepared. Semithin sections were stained with toluidine blue and visualized on a Leica confocal system. Ultrathin sections were labeled with purified MYO7A pAb 2.2 (Liu et al., 1997) and monoclonal anti-opsin (1D4, R. Molday), followed by gold-conjugated secondary antibodies (Electron Microscopy Sciences), as described previously (Lopes et al., 2011). Negative control sections processed at the same time included those from MYO7A-null retinas, and, as positive control, WT animals were used.

MYO7A immunogold density was determined on sections of age-matched WT, MYO7A-null retinas and retinas of MYO7A-null animals that had been injected with AAV-MYO7A at P14-16 and dissected three weeks later. For quantification of the immunolabel, all of the gold particles in a complete section of each RPE cell were counted. The area of each cell's profile was determined using ImageJ software. For background labeling, the concentration of label in sections of untreated MYO7A-null animals was measured. Data were expressed with this background labeling subtracted.

The concentration of MYO7A and opsin immunogold labeling in the connecting cilia of photoreceptor cells was determined by counting gold particles along longitudinal profiles of connecting cilia and measuring the length of each profile. Analysis and quantifications were performed in a minimum of three different retinas, from three different animals. Statistical analysis was performed using one-tail Student's t-test.

Six weeks post-injection, C57BL/6 mice were enucleated and their eyes processed and immunostained as previously described (Boye et al., 2011) with minor modifications. Retinas were immunostained with an antibody specific for hemagglutinin (HA) (monoclonal Ab clone 12CA5; Roche), counterstained with DAPI, and imaged with a spinning disk confocal microscope (Nikon Eclipse TE2000 microscope equipped with Perkin Elmer Ultraview Modular Laser System and Hamamatsu O-RCA-R2 camera). Images were obtained sequentially using a 20×(air) objective lens. All settings (exposure, gain, laser power) were identical across images. All image analysis was performed using Volocity 5.5 software (Perkin Elmer, Waltham, MA, USA).

### Results

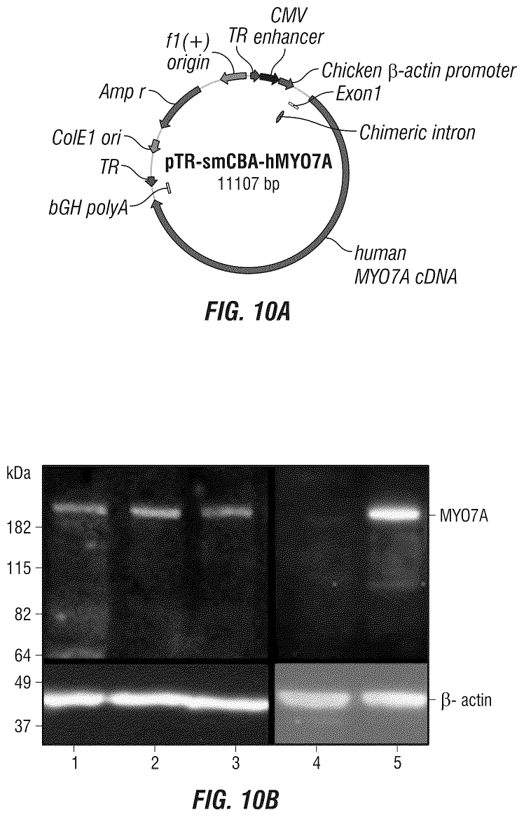

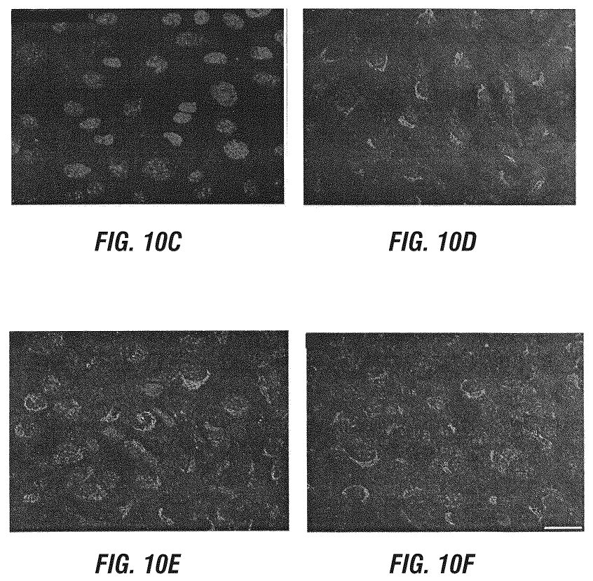

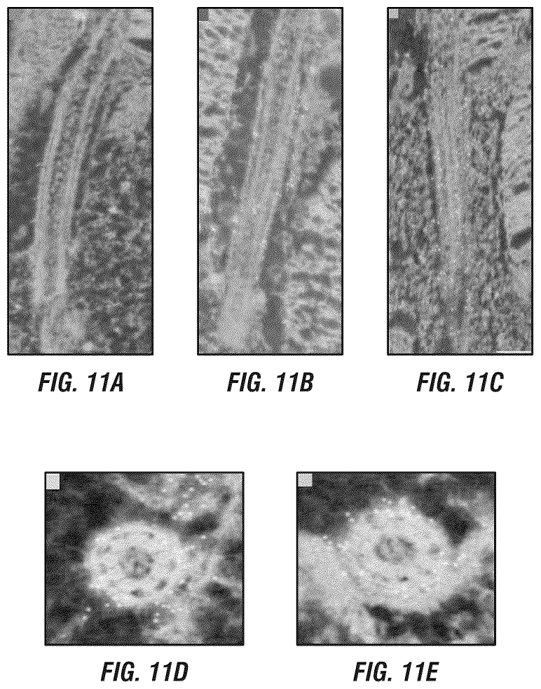

AA V-MYO7A single vector preparations. AAV vector plasmid was engineered to contain a truncated chimeric CMV/chicken β-actin promoter, smCBA (Haire et al., 2006) and the 6.7-kb cDNA encoding the full-length isoform 2 of human MYO7A (NCBI #NM_001127180) (FIG. 10A). The smCBA promoter exhibits the same tropism and activity in mouse retinas as that of the full-length CBA promoter (Haire et al., 2006; Pang et al., 2008). Titers of 10¹² to 10¹³ particles/mL were obtained for different lots of AAV2-MYO7A and AAV5-MYO7A. A concentration of 10¹² particles/mL was regarded as the standard concentration (lx), from which dilutions were made. The experiments were performed with virus obtained from three separate preparations. No differences in expression or phenotype correction, as described below, were observed among the different lots for AAV2-MYO7A or AAV5-MYO7A at a given concentration.

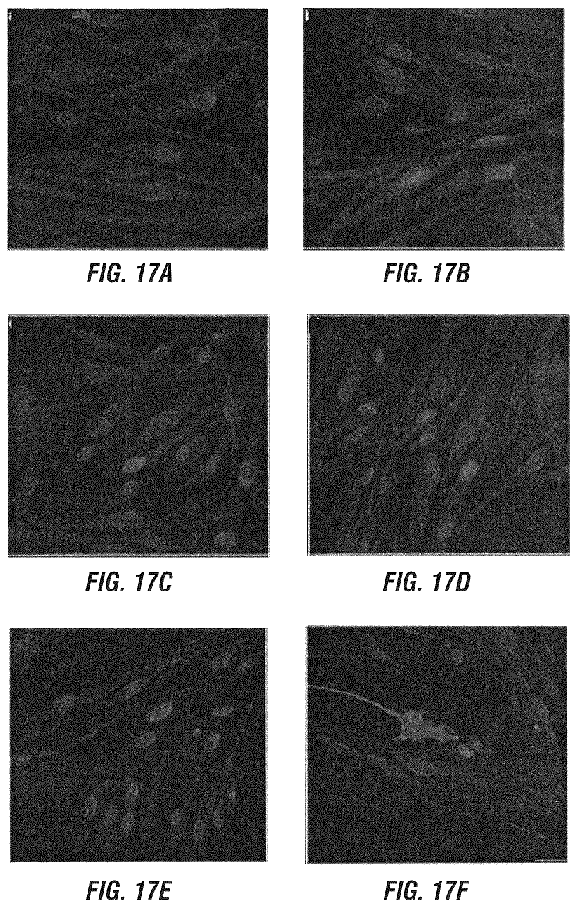

MYO7A Expression in Cell Culture. Transduction of primary cultures of MYO7A-null RPE cells with 1× single AAV2-MYO7A or AAV5-MYO7A resulted in the expression of a polypeptide that, by western blot analysis, had an apparent mass that was comparable to that of WT MYO7A protein, and was present at similar levels to that found in primary cultures of MYO7A^{+/-} RPE cells (FIG. 10B). Likewise, a single band of appropriate size was detected on western blots of HEK293A cells. Immunofluorescence of the primary RPE cells showed that the MYO7A protein, resulting from 1×single AAV-MYO7A treatment of MYO7A-null cells, had a subcellular localization pattern that was comparable to that of endogenous MYO7A in control cells, indicating the generation of appropriately targeted protein (FIGS. 10C-10F). ARPE19 cells were also infected with 1× or diluted (1:100) AAV2-MYO7A or AAV5-MYO7A, and compared with non-treated cells. An increase in MYO7A immunofluorescence was detected in the treated cells, and the intracellular localization of the label was comparable to that in untreated cells (FIGS. 17A-17F).

Localization of MYO7A in Vivo. Most retinal MYO7A is found in the RPE (Hasson et al., 1995), however, the protein is also present in the connecting cilium and pericilium of the photoreceptor cells (Liu et al., 1997; Williams, 2008). A diagram illustrating this distribution and the retinal functions of MYO7A has been published in a recent review (Williams and Lopes, 2011).

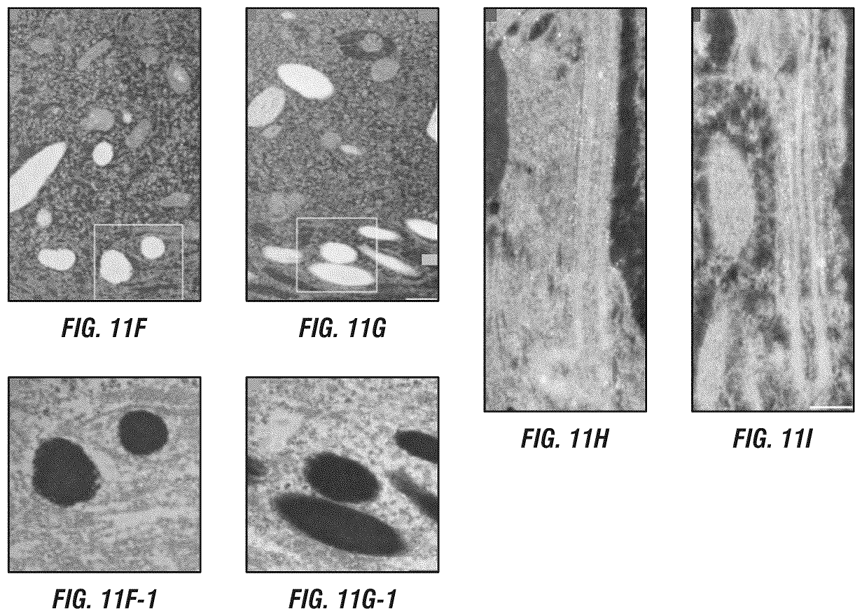

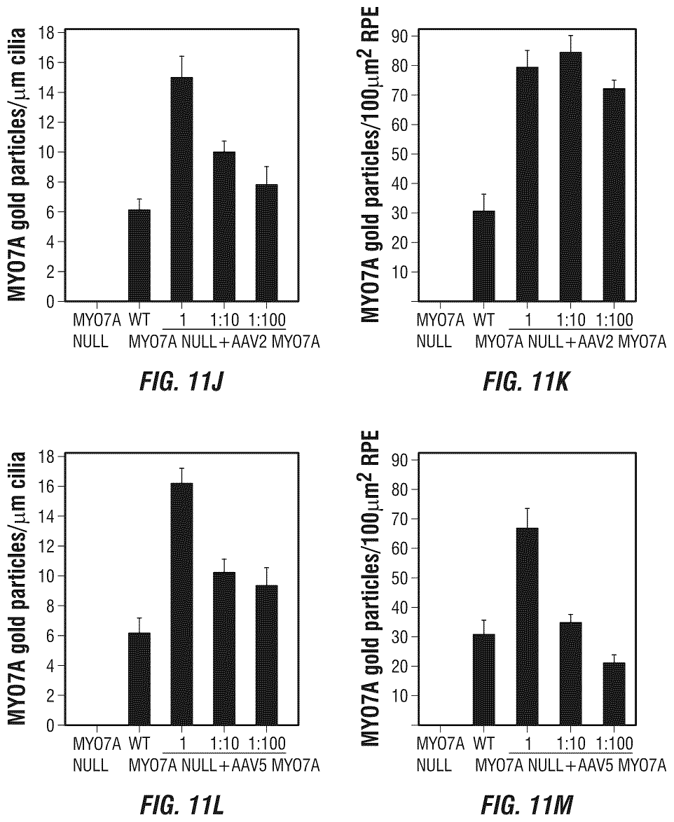

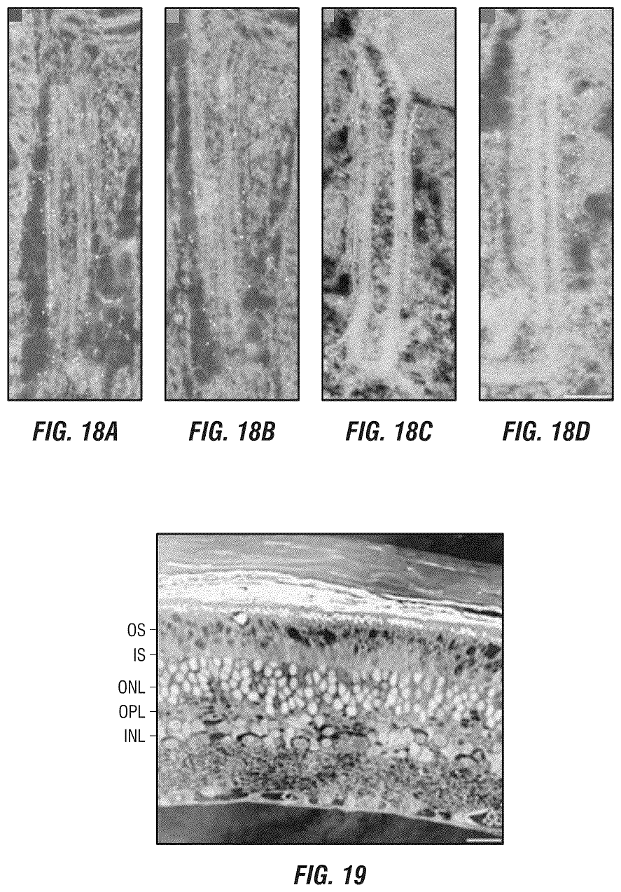

Three weeks following injection of 1×AAV2-MYO7A or AAV5-MYO7A into the subretinal space of MYO7A-null mice, retinal tissue was examined by immunoelectron microscopy to test for MYO7A expression. Immunogold label was evident in the photoreceptor cells, where it was localized in the connecting cilium and pericilium, comparable to that in WT retinas (FIGS. 11A-E). Label was also present throughout the RPE cells, particularly in the apical cell body region (FIG. 11F, FIG. 11F-1, FIG. 11G, and FIG. 11G-1; see FIGS. 18A-18D for controls), as found in WT retinas (Gibbs et al., 2004; Liu et al., 1997).

MYO7A has a similar distribution in both rod and cone photoreceptor cells (Liu et al., 1999). To test whether treatment with AAV-MYO7A also affected cone photoreceptor cells, it was determined whether MYO7A was also present in the ciliary region of cone photoreceptors. Double immunoEM of treated retinas was performed, using a MYO7A antibody together with an antibody specific for rod opsin. Although there are only a small number of cones with aligned connecting cilia found in each ultrathin section, MYO7A immunogold label was evident in the connecting cilium and periciliary region of these cones, which were identified by lack of rod opsin labeling in their outer segments (in contrast to the surrounding rod outer segments) (FIG. 11H and FIG. 11I). Hence, AAV2-MYO7A and AAV5-MYO7A can transduce cone as well as rod photoreceptor cells.

Dose-dependent MYO7A expression in photoreceptor and RPE cells. To determine the levels of MYO7A expression following treatment with different concentrations of AAV2-MYO7A and AAV5-MYO7A (lx, 1:10 or 1:100 dilutions), MYO7A immunogold labeling was quantified in EM images, taken within 1.4 mm of the injection site. Reliable detection of MYO7A in the photoreceptor cells, where its distribution is limited to the connecting cilium and pericilium, requires the higher resolution provided by electron microscopy (Liu et al., 1997). Immunogold particle density was measured in images of the photoreceptor connecting cilium and pericilium, shown in complete longitudinal section (from the basal bodies to the base of the outer segment), and in images showing the RPE cells in apical to basal section. Particle density was expressed as particles per length of cilium for the photoreceptor cells (each connecting cilium is ˜1.2 m long), and as particles per area for the RPE cells (the entire area between the apical and basal surfaces was included). Particle density is dependent on exposure of epitopes on the surface of the section, and, as such, provides a relative linear measure of antigen density under the conditions used here (for example, grids were etched and labeled in an identical manner, and the labeling was not so dense as to be affected by steric hindrance).

Treatment with 1×AAV2-MYO7A or AAV5-MYO7A resulted in 2.5-2.7 times the density of immunolabel in the photoreceptor cilium, compared with that found in WT retinas, while the 1:10 and 1:100 dilutions resulted in a density of immunolabel that was more comparable to WT levels (FIG. 11J, FIG. 11L, and FIG. 19). Quantification of immunogold label in the RPE showed that injection of AAV2-MYO7A resulted in 2.7 times more label than in WT, with the 1:10 and 1:100 dilutions showing no significant difference (FIG. 11K). In contrast, the level of MYO7A immunolabel in the RPE of retinas injected with AAV5-MYO7A varied in relation to virus titer, with the full dose virus effecting 2.2-fold more MYO7A than that found in WT RPE, the 1:10 dilution effecting WT levels, and the 1:100 dilution resulting, on average, ˜60% of WT levels (FIG. 11M).

These counts of labeling density indicate that 1×AAV-MYO7A resulted in more than double the normal level of MYO7A expression in both the photoreceptor and RPE cells. The distribution of MYO7A was not affected by this overexpression in the photoreceptor cells. In the RPE cells, the overall distribution of MYO7A was comparable to WT, with a higher concentration in the apical cell body region. However, with 1×AAV2-MYO7A or 1×AAV5-MYO7A, the proportion of MYO7A that was associated with melanosomes was only 55% of that in WT RPE. This difference is possibly because the proteins that link MYO7A to the melanosomes, MYRIP and RAB27A (Klomp et al., 2007; Lopes et al., 2007), may have remained near WT levels, and thus limited the absolute amount of MYO7A that could associate with the melanosomes.

Despite the overexpression of MYO7A, no pathology was evident in retinas, up to 3 months after injection of 1× (or 1:10) AAV2-MYO7A. However, two out of six retinas injected with 10¹³ particles/mL of AAV5-MYO7A (for example, 10×) showed evidence of photoreceptor cell loss across the retina after 3 weeks (AAV2-MYO7A was not tested at this titer) (FIG. 19).

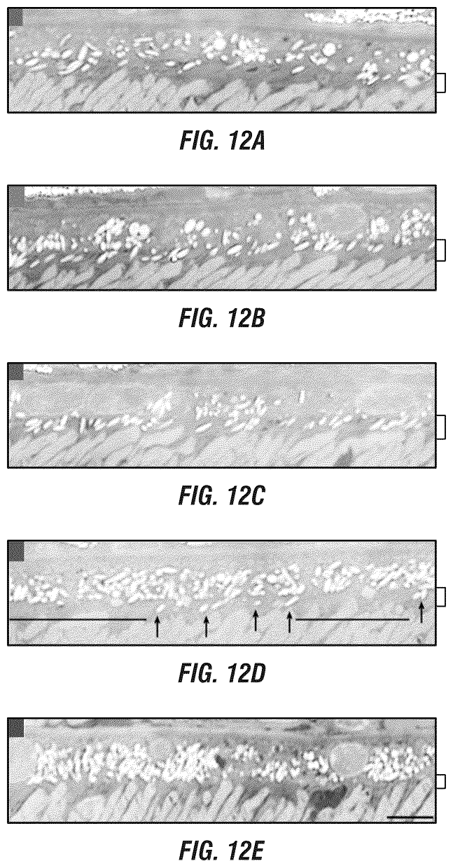

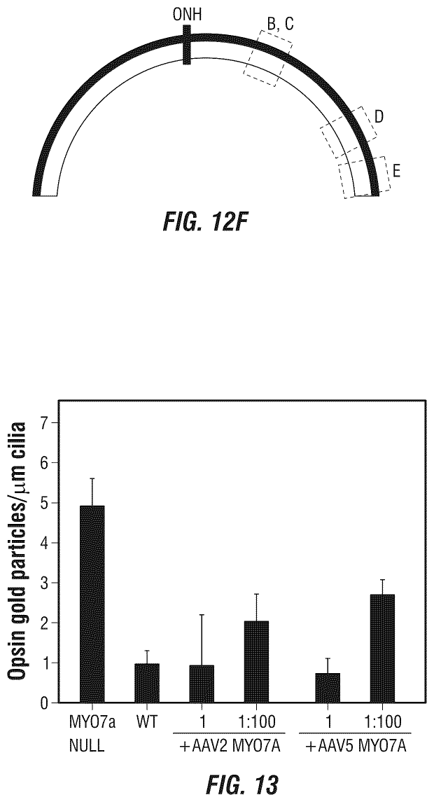

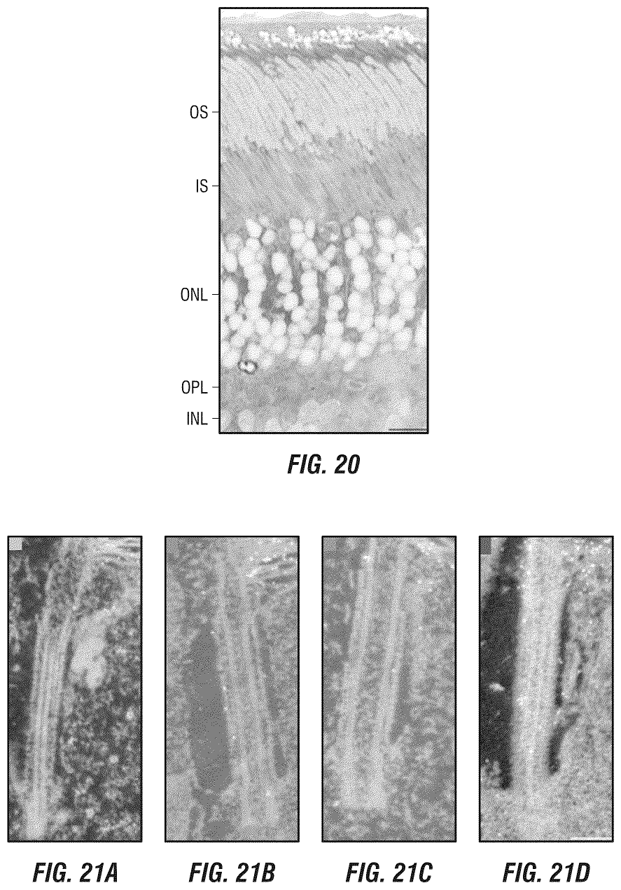

Correction of melanosome localization in the RPE. In MYO7A-mutant mice, melanosomes are absent from the apical processes of the RPE cells (Liu et al., 1998). This mutant phenotype is evident at all neonatal ages, and is due to loss of actin-based transport of the melanosomes by the myosin 7a motor (Gibbs et al., 2004). Three weeks following injection of 1×AAV2-MYO7A or AAV5-MYO7A into the subretinal space of MYO7A-null mice, melanosomes were observed to have a normal distribution in all RPE cells near the site of injection (within 1.4 mm) (n=10 each for AAV2-MYO7A and AAV5-MYO7A) (FIGS. 12A-12C). Well away from the injection site, a mixture of corrected and uncorrected RPE cells was evident, while, at the periphery of the retina, the cells all exhibited the MYO7A-mutant phenotype, indicating lack of correction in this region (FIGS. 12D-12F). The correction of melanosomes was still evident in retinas that were fixed 3 months after injection (FIG. 20). Correction was also observed in all eyes injected with 1:10 dilution AAV2-MYO7A (n=6) or AAV5-MYO7A (n=6), as well as in all eyes injected with 1:100 dilution AAV2-MYO7A (n=6) or AAV5-MYO7A (n=6), although with the 1:100 dilution some of the RPE cells near the site of injection were not corrected.

Correction of opsin distribution. MYO7A-mutant mice have an abnormal accumulation of opsin in the connecting cilia of the photoreceptor cells, a phenotype that is evident by immunoEM with opsin antibodies (Liu et al., 1999). This mutant phenotype suggested that myosin 7a functions in the vectorial delivery of opsin to the outer segment (Liu et al., 1999). Quantification of immunogold opsin labeling in the connecting cilia, demonstrated that this phenotype was corrected with 1×AAV2-MYO7A or AAV5-MYO7A (FIG. 13 and FIGS. 21A-21D). This analysis also showed phenotype correction with 1:100 dilutions, although the data indicated that a full WT phenotype was not achieved (FIG. 13), despite WT levels of MYO7A (FIG. 11J and FIG. 11L), suggesting that some of the MYO7A may not be fully functional.

AAV2-MYO7A dual vector preparations. The preceding results demonstrate that a single AAV vector is capable of delivering functional MYO7A to the RPE and photoreceptor cells in vivo. Because the size of smCBA-MYO7A is ˜2 kb larger than the nominal carrying capacity of an AAV (Grieger and Samulski, 2005), this transduction may involve undefined fragmentation of the smCBA-MYO7A cDNA followed by reassembly of plus and minus cDNA strands after delivery to the cell as shown for other large genes (Dong et al., 2010; Lai et al., 2010; Wu et al., 2010). To evaluate whether two AAV vectors containing defined, overlapping fragments of MYO7A cDNA (1365 bases) were also capable of mediating full-length MYO7A expression, an AAV2-based dual vector system (FIG. 14A-1 and FIG. 14A-2) was developed. Two separate lots of the AAV2-MYO7A (dual vector) were prepared, each containing equal concentrations of AAV2-smCBA-MYO7A(5′-half) and AAV2-MYO7A(3′-half). The titer of the first lot contained 2.5×10¹² particles/mL of each vector, and the second lot contained 4×10¹² particles/mL.

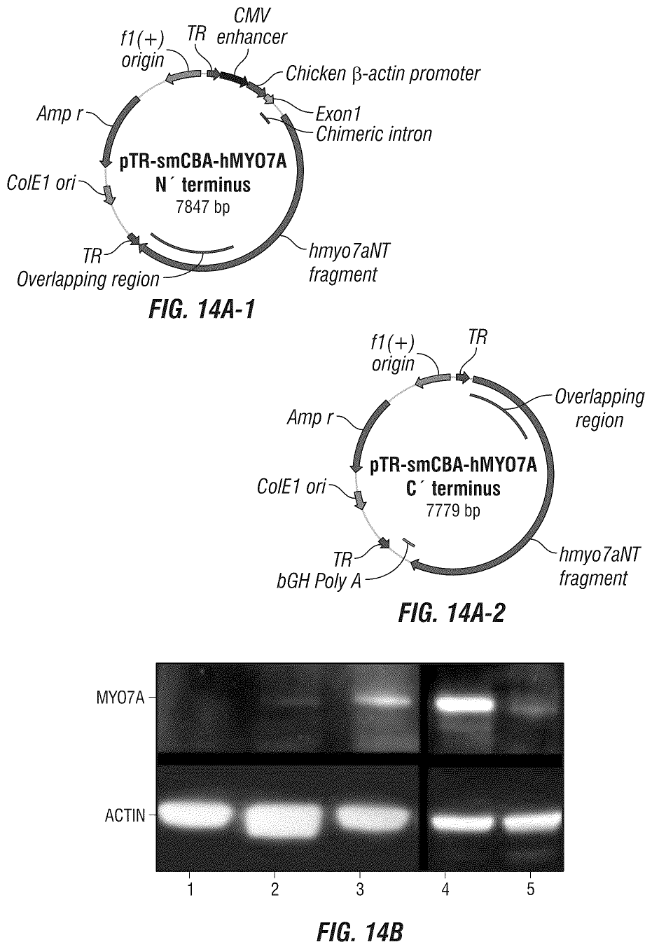

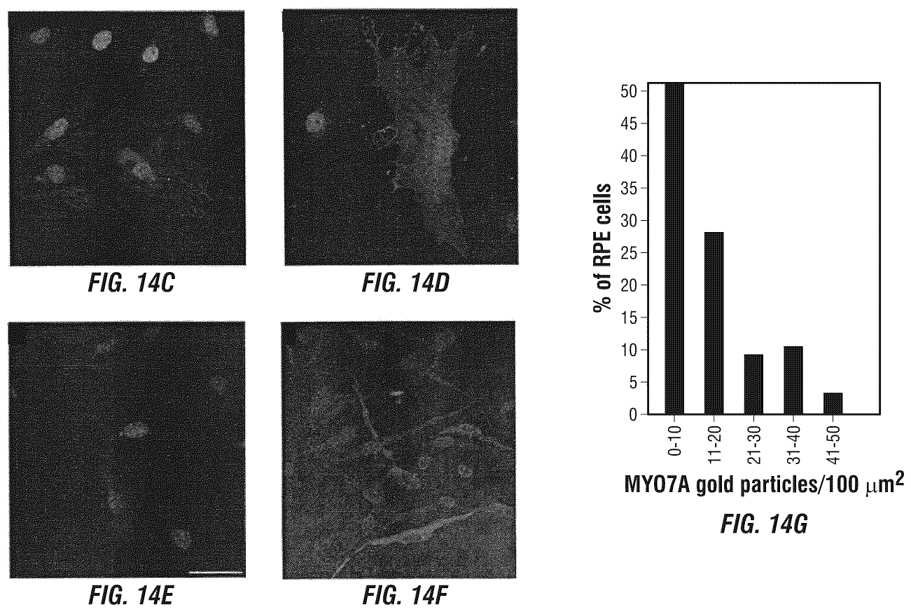

MYO7A expression with AAV2 dual vectors. Western blot analysis of primary cultures of MYO7A-null RPE cells, infected with AAV2-MYO7A (dual vector) of either lot, showed that the cells expressed a MYO7A-immunolabeled polypeptide of comparable mass to that of WT MYO7A (FIG. 14B). However, the expression level of MYO7A in the MYO7A-null RPE cells was significantly less than that found in primary cultures of MYO7A^{+/-} RPE cells (cf. lanes 2 and 3 in FIG. 14B), unlike that found for the single AAV2 or AAV5 vectors (FIG. 10B). Quantitative analysis of western blots showed that MYO7A-null RPE cells, transduced with the single vectors (1×), AAV2-MYO7A or AAV5-MYO7A, or with AAV2-MYO7A (dual vector), expressed MYO7A at levels that were 82%, 111%, and 10%, respectively, of the level of MYO7A in MYO7A^{+/-} RPE cells.

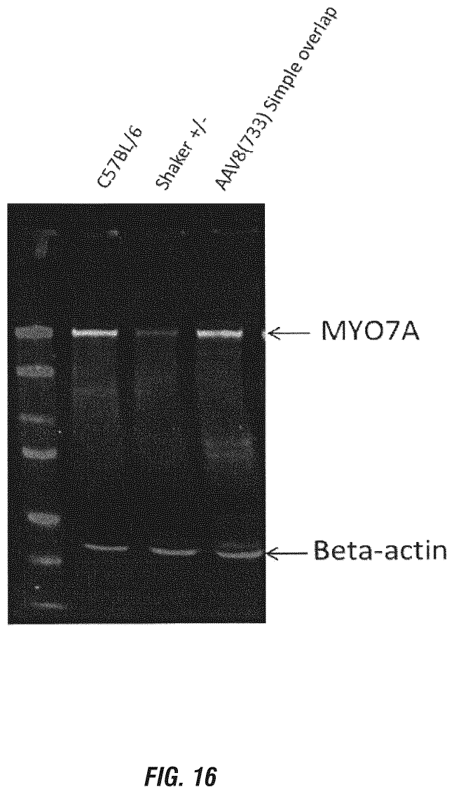

FIG. 16 is a Western blot using the same dual vector system as above except in an AAV8 serotype. FIG. 16 shows expression level of MYO7A using the dual vector system that was nearly equivalent to the wild type MYO7A expression level. While the reasons for the discrepancy are unclear, much better results were obtained by the inventors using the dual vector systems than were obtained by several outside collaborators. Wild-type-like levels of MYO7A expression were observed in shaker-1 retinas following injection with dual-AAV8(Y733F) vectors. Thus, very good expression of MYO7A with the dual AAV platform has been achieved.

Immunofluorescence of primary MYO7A-null RPE cells, infected with AAV2-MYO7A (dual vector), showed that a few cells scattered throughout the culture exhibited very high levels of MYO7A, but all other cells contained insignificant levels (FIGS. 14C-14E). The cells overexpressing MYO7A typically had altered morphology, suggesting that the high levels of MYO7A may be toxic. Similarly, immunofluorescence of ARPE19 cells, infected with AAV2-MYO7A (dual vector), resulted in a minority of cells that were labeled intensely with MYO7A antibody, with most of the cells appearing to express only endogenous levels of MYO7A (FIG. 14F and FIG. 17F).

Immunolabeling of retinas, prepared 3 weeks after subretinal injection with AAV2-MYO7A (dual vector) of either lot, also showed only a few RPE cells and photoreceptor cells with clear MYO7A expression, although significant overexpression was not evident in this in vivo experiment. Immunogold particle counts from images of ultrathin sections were used to quantify the level of MYO7A expression in MYO7A-null retinas that were treated with the second lot of AAV2-MYO7A (dual vector). Within 1.4 mm of the injection site, MYO7A immunolabeling of the connecting cilium and pericilium of the photoreceptor cells was a mean of 48% of that in WT retinas: 2.8 particles/μm (n=3 retinas) compared with 6.5 particles/μm for WT (n=3 retinas). The mean label density in apical-basal sections of the RPE was 35% of that in WT retinas: 11 particles/100 μm² compared with 31 particles/100 μm² for WT. However, it was clear that these lower means were achieved by some cells expressing near normal amounts of MYO7A and the majority expressing very little; over half the cells had fewer than 10 particles/100 μm² (FIG. 14G).

Correction of MYO7A-mutant phenotypes with AAV2 dual vectors. Eyes were analyzed for correction of melanosome localization and ciliary opsin distribution within 1.4 mm of the injection site. With either lot of AAV2-MYO7A (dual vector), some RPE cells (29% for lot 1 treatment [n=6 retinas], 35% for lot 2 treatment [n=9 retinas]) were observed to have a normal apical melanosome distribution, but most of the cells in this region retained the MYO7A-mutant phenotype, resulting in a mosaic effect (FIG. 15A) that contained a much lower proportion of corrected cells than that observed with a 1:100 dilution of either of the single vectors. The only correction observed in 3 eyes injected with a 1:10 dilution of AAV2-MYO7A (dual vector) (first lot), was in 18% of the RPE cells in one of the retinas. With full-strength of AAV2-MYO7A (dual vector) (second lot), opsin immunogold density averaged 3.2±0.4 particles/μm of cilium length, which was reduced from untreated retinas (4.2±0.8 particles/μm; p=0.003), but still greater than WT levels (1.1±0.2 particles/μm), suggesting that most cells were not corrected.

Using immunoelectron microscopy, a correlation between phenotype correction and the expression level of MYO7A was identified (determined by the mean concentration of immunogold particles in an apical-basal section of each RPE cell) (FIGS. 15B-15E). From the eyes injected with AAV2-MYO7A (dual vector) (second lot), it was shown that the corrected RPE cells contained a mean of 108% of the WT level of MYO7A (the minimum level was 82%). RPE cells that were not corrected contained a mean of 26% of the WT level of MYO7A (the maximum level was 92%). While these data showed that higher expression of MYO7A is correlated with phenotype correction (FIG. 15F), it also indicated that some of the labeled MYO7A protein was not functional, given that melanosomes are localized normally in mice that are heterozygous for the MYO7A-null allele and have only ˜50% of the WT level of MYO7A.

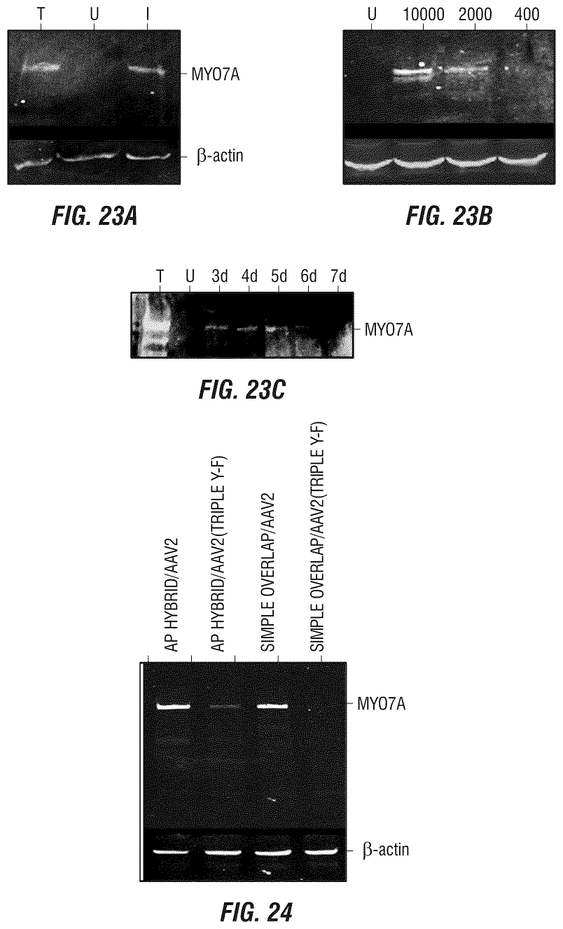

Expression of MYO7A with simple overlap vectors. AAV2-based simple overlap vectors were evaluated in vitro at a variety of MOIs to evaluate how the concentration of vector pairs related to MYO7A expression. How levels of MYO7A changed over time was also evaluated in infected cells. HEK293 cells were infected with simple overlap vector pairs packaged in AAV2(tripleY-F) vector (FIG. 23A). A preliminary co-infection with AAV2(tripleY-F) simple overlap vectors (MOI of 10,000 for each vector) indicated that MYO7A is expressed, and that migration of the protein on gel is identical to a full-length transfection control (FIG. 23A). Coinfection at MOIs of 400, 2000, and 10,000 of each vector shows that the efficiency of the simple overlap system is proportional to the amount of 5′ and 3′ vectors used (FIG. 23B). MYO7A expression increased as a function of incubation time up to 5 days post-injection in HEK293 cells (FIG. 23C). The visible expression decline was because of a reduction of viable cells in the culture vessel at the later times.

Comparison of fAAV-MYO7A to dual-AAV-MYO7A expression and evaluation of AA V serotype efficiency. Previously, it was shown that fragmented AAV encoding MYO7A was able to ameliorate the retinal phenotype of the shaker1 mouse (Colella et al., 2013; Lopes et al., 2013; Trapani et al., 2013). To provide a basis for comparison dual-AAV-vector expression was evaluated relative to fAAV in vitro. After infection in HEK293 cells, all dual vector systems expressed MYO7A more efficiently than fAAV (FIG. 24). The AP hybrid platform showed the strongest expression, followed by the simple overlap system.

Other studies have shown, in the context of a conventionally sized DNA payload, that the transduction efficiency and kinetics of AAV2(tripleY-F) vectors are increased relative to standard AAV2 both in vitro and in vivo (Li et al., 2010; Markusic et al., 2010; Ryals et al., 2011). The efficiency of AAV2 versus AAV2(tripleY-F) dual vectors was directly compared in HEK293 cells. Surprisingly, standard AAV2-mediated MYO7A expression was higher than that seen with titer-matched AAV2(tripleY-F) (FIG. 24). Identical results were obtained when comparing different AAV2 and AAV2(tripleY-F) dual vector preparation packaged with identical vector plasmid.

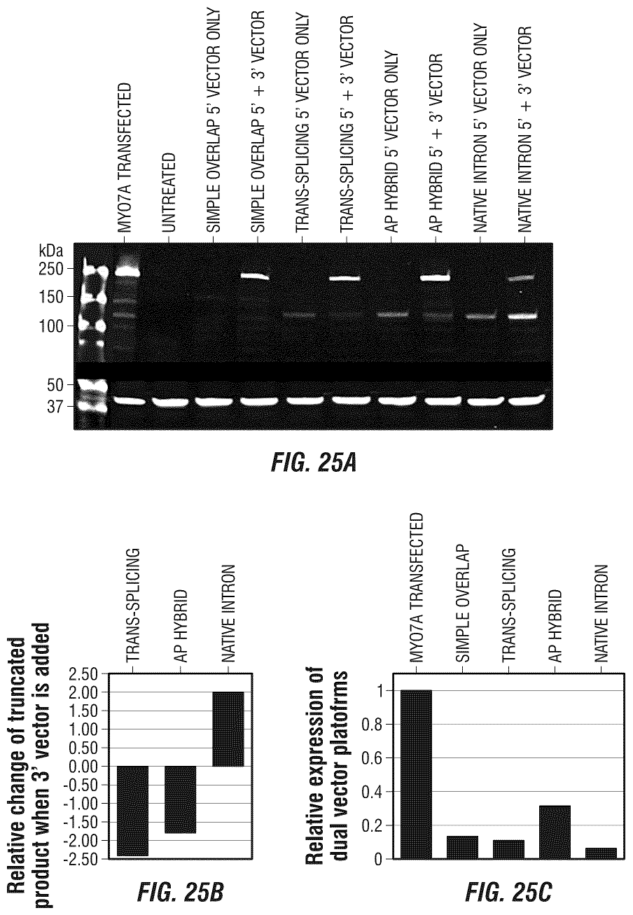

Comparison of relative efficiencies and specificity of full-length MYO7A expression. To quantitatively evaluate the relative expression efficiencies of the dual vector platforms and to assess specificity of full-length protein, HEK293 cells were infected with either the 5′ and 3′ AAV2-based vector pairs combined or the corresponding 5′ vector alone. An additional hybrid vector pair was included that incorporated native MYO7A intronic sequence (intron 23) that served as overlapping sequence and provided appropriate splicing signals. All 5′ vectors produced low amounts of a defined, less than full-length peptide detectable on Western blot with the exception of the simple overlap vector (FIG. 25A). However, the trans-splicing and the AP hybrid platforms revealed a distinct decrease of this undesired product when the 3′ vector was added to the sample (FIG. 25A). The native intron hybrid platform also showed this secondary band on Western blots, again suggestive of a truncated protein originating from the 5′ vector alone. In contrast to all other platforms tested, this band intensity increased with the addition of the 3′ vector. Each platform's relative ability to promote reconstitution was compared by quantifying the amount of 5′ vector-mediated truncated protein product in the presence or absence of the respective 3′ vector (FIG. 25B). Full-length MYO7A expression on Western blot was then quantified relative to transfection control (FIG. 25C). AP hybrid-mediated MYO7A was the strongest followed by simple overlap, trans-splicing, and native intron hybrid (FIG. 25C).

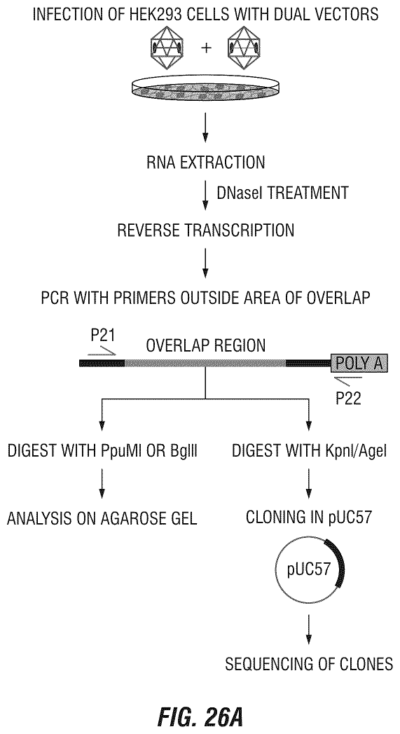

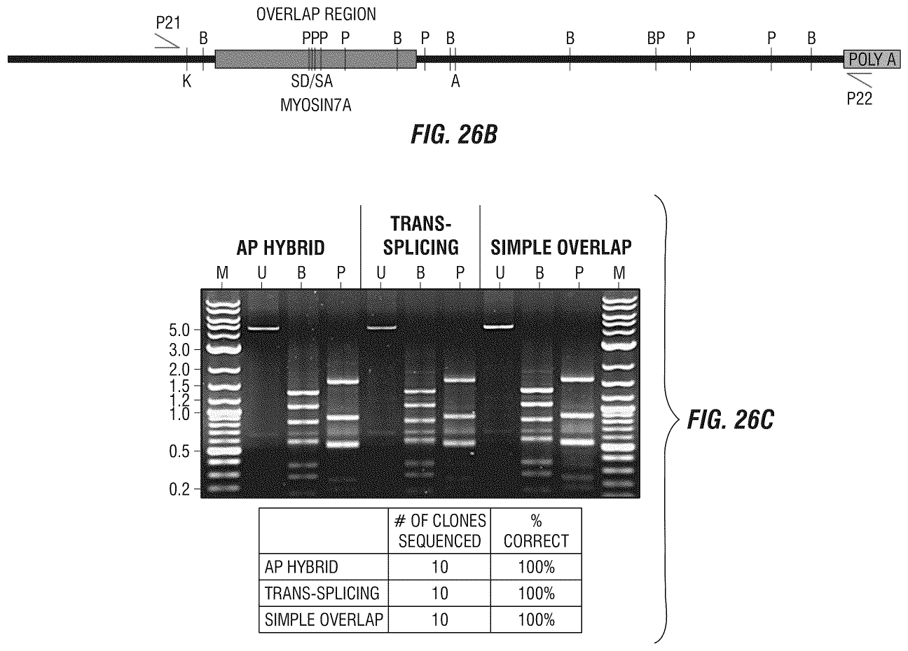

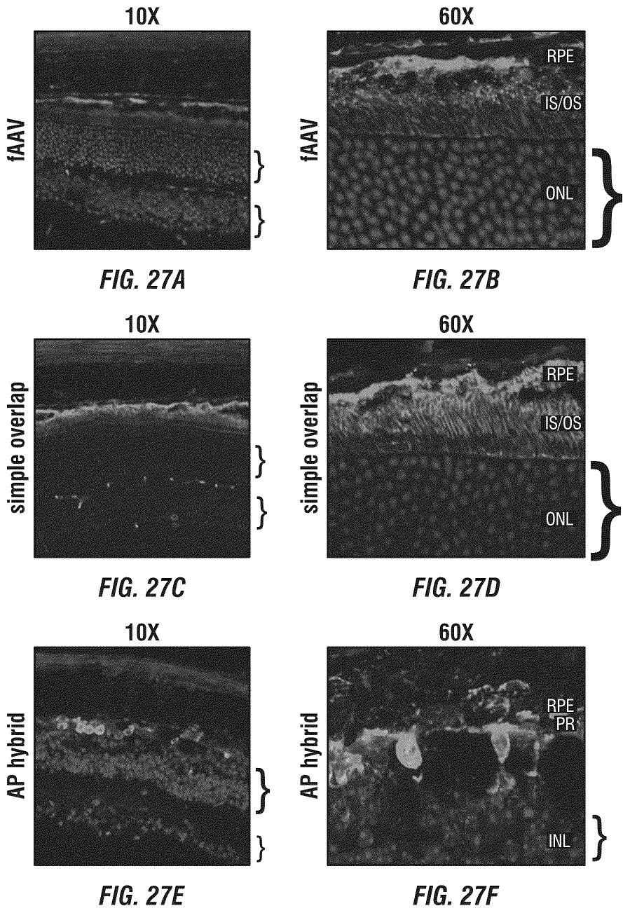

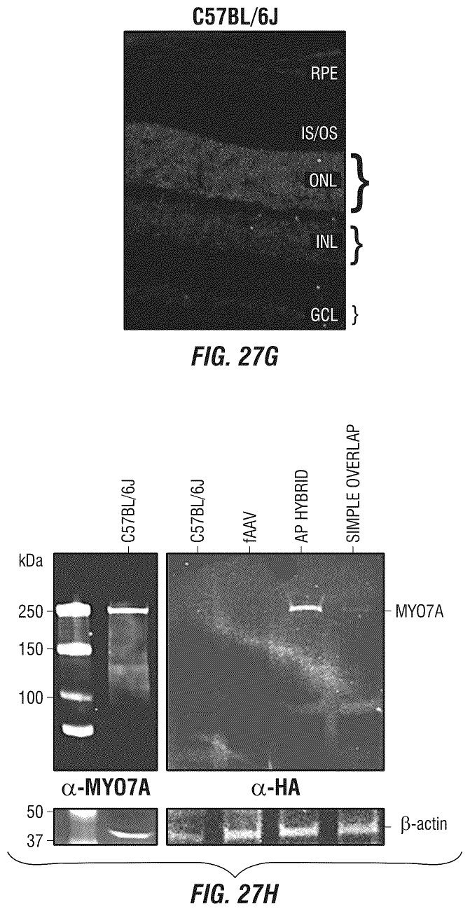

Characterization of the overlap/splice region of the expressed MYO7A. To characterize the fidelity of the mRNA arising from dual vectors, HEK293 cells were infected with dual vectors and RNA extracted, reverse transcribed, and subjected to PCR utilizing primers binding upstream of the overlap region and in the bGH polyA signal region producing a 4.5 kb PCR fragment (FIG. 26A). An identically treated sample not containing reverse transcriptase was used as control for chromosomal DNA contamination. Plasmid containing the full-length MYO7A coding sequence was used as positive control for PCR. A preliminary screen of AAV-mediated MYO7A mRNA was performed by analyzing the pattern of fragment migration on agarose gel following restriction endonuclease digests with PpuMI and BglII (FIG. 26A). Identical banding patterns, consistent with the predicted pattern (PpuMI: 1591, 876, 556, 548, 541, 238, 168, 42, and 36 bp; BglII: 1335, 1074, 827, 583, 360, 272, and 146 bp), were observed following digests of amplicons from each dual vector platform tested, indicating that no gross alterations (deletions/insertions) occurred as a consequence of either homologous recombination of vector pairs and/or RNA splicing (FIG. 26B). To further characterize the fidelity of the overlap region, a fragment containing the complete overlap area (1829 bp) was restricted and cloned into pUC57 (FIG. 26A). Sequencing results of 10 clones picked at random per vector platform revealed that the overlap region was 100% identical to the consensus/predicted MYO7A sequence (FIG. 26C). This indicated that, in the context of the simple overlap platform, homologous recombination was accurate. Additionally, in the context of trans-splicing vectors, accurate splicing occurred. Finally, for the AP hybrid vectors, a combination of accurate homologous recombination and/or splicing took place. To determine whether this protocol was capable of detecting aberrant sequence in reconstituted MYO7A, a sequence that contained either an insertion of a HindIII recognition site (TAGC) at position 2635 or a point mutation (T-C) at position 2381 was also generated.

MYO7A expression mediated by dual vectors in mouse retina. To investigate the expression of MYO7A from the two best performing dual vector platforms in vivo, C57BL/6J mice were subretinally injected with 1×10¹⁰ vector genomes per eye of simple overlap and AP hybrid systems packaged in AAV8(733) and analyzed 4 weeks later by Western blot and immunohistochemistry. AAV8(733)-fAAV-MYO7A vector was also injected to provide a basis for comparison. To distinguish between endogenous MYO7A and exogenous expression mediated by vectors, sequence coding for an HA tag was added to the C prime terminus of the MYO7A cDNA in all constructs. Resulting retinas were immunostained for HA to reveal that fAAV vector along with both dual vector platforms mediated expression of MYO7A in photoreceptors and RPE. A recent report concluded that simple overlap vectors were more efficient for gene transfer to the RPE than photoreceptors (Trapani et al., 2013). Simple overlap-mediated MYO7A expression was observed in both RPE and photoreceptors. In contrast to previous results showing “spotty” MYO7A expression mediated by AAV2-based simple overlap vectors (Lopes et al., 2013), it was found, when packaged in AAV8(733), that simple overlap vectors mediated MYO7A expression in the majority of RPE and photoreceptor cells. Photoreceptor degeneration/outer nuclear layer thinning was apparent in eyes injected with the AP hybrid vector system. Despite the observed degeneration, AP hybrid-mediated MYO7A was clearly detected in residual PR cell bodies and RPE and was sufficient to be detected by immunoblot. By Western blot analysis using HA antibody, simple overlap-mediated MYO7A was present in just detectable amounts. In contrast, fAAV-mediated protein levels were insufficient to be detected in this assay. Using an antibody against MYO7A, immunoblot of WT mouse retina revealed that both endogenous MYO7A and dual vector-mediated, HA-tagged MYO7A migrated similarly.

### Discussion

In this example, it was shown that dual AAV vectors with defined genetic payloads can be used to deliver a large transgene in vitro and in vivo. The initial experiments using the simplest of all dual vector platforms revealed that efficiency of AAV2-based simple overlap vectors is proportional to the amount of 5′ and 3′ vectors used and that MYO7A expression mediated by this system increased as a function of incubation time in HEK293 cells. Next, three distinct dual vector platforms were evaluated and compared to single, fragmented fAAV vector in vitro. All dual vectors analyzed drove higher levels of MYO7A expression than fAAV. Of all platforms tested, a hybrid vector system containing overlapping, recombinogenic sequence and splice donor/acceptor sites from the AP gene (AP hybrid) was the most efficient.

Regarding the specificity with which the dual vector platforms express the correct-sized gene product, it was noted in vitro that trans-splicing and hybrid dual vector platforms generated an additional band of lower molecular weight as detected by immunoblot (monoclonal antibody used was raised against the amino terminus MYO7A). The expression of this truncated protein product was much more pronounced for infections with 5′ vectors alone.

After entry into the host cell, the virus capsid is removed and the single-stranded DNA payload is released. The ITRs carried by the single strand serve as primer for DNA polymerases to produce a double strand. The resulting circular intermediates consist mainly of monomers that, over time, convert into multimeric concatemers through intermolecular recombination (Duan et al., 1998; Yang et al., 1999). The dual vector systems of this disclosure utilize this strategy to achieve full-length protein expression. A limiting factor lies in the fact that the highly recombinogenic ITRs flanking the expression cassettes are identical in nature leading to a random recombination and consequently a random orientation of the vector parts relative to each other. This random recombination inevitably results in reduced efficiency because only concatemers that have the two vector parts in 5′ to 3′ orientation are able to express the full-length protein. This concatemerization over time is consistent with the observation that the amount of single-vector product is reduced in favor of the full-length protein when both 5′ and 3′ vectors are combined. Interestingly, the simple overlap system does not generate truncated product, even when only the 5′ vector is used for infections. In contrast to the trans-splicing and hybrid vectors, there is virtually no intervening sequence between the end of the MYO7A coding sequence and the right-hand ITR.

Notably, in this disclosure, it was found that the sequence in the overlap region of all dual vectors tested in vitro was 100% identical to the consensus/predicted MYO7A sequence. This indicates that homologous recombination and/or splicing was accurate in each dual vector platform.

Similar to the in vitro results, the highest levels of MYO7A expression was found in retinas of mice subretinally injected with AAV8-based AP hybrid vectors (as assessed by probing for HA on Western blot). Notably, no truncated proteins were evident in retinas expressing either simple overlap or AP-hybrid mediated MYO7A. The reason for this observed difference remains to be elucidated but may involve differences in the DNA repair machinery that mediate recombination in actively dividing cells versus post-mitotic photoreceptors/RPE (Hirsch et al., 2013). Dual vector-mediated MYO7A-HA expression was observed in the photoreceptors and RPE of WT mice, locations where MYO7A is thought to have a functional role (Williams and Lopes, 2011). In eyes injected with AP hybrid vectors, marked thinning of the outer nuclear layer was observed. It has previously been shown that vector-mediated overexpression of MYO7A leads to retinal toxicity (Hashimoto et al. 2007). Taken together with the high efficiency of transduction observed in vitro for the AP hybrid platform, the most likely explanation for the observed pathology is excessive production of MYO7A. Despite the marked degeneration, significant amounts of AP hybrid-mediated, full-length MYO7A-HA were detected on Western blot. As high concentrations of vectors were used in these experiments, a simple solution to circumvent cytotoxicity could be to reduce vector genomes injected or replace the strong, ubiquitous smCBA promoter with an endogenous or homologous promoter, and/or a promoter with attenuated strength; or reduce expression of undesired products, like the observed protein expressed from the 5′ vectors alone in vitro. However, it was noted that only full-size MYO7A-HA was apparent on Western blot of the AP hybrid-treated retina.

With the goal of developing an AAV-based treatment for USH1B, animal models of this disease have provided an abundance of useful information. Similar to previous observations that fAAV-MYO7A and simple-overlap, dual vectors were capable of restoring melanosome migration and opsin localization in the shaker1 mouse (Lopes et al., 2013), a recent study by an independent lab confirmed the usefulness of the vectors disclosed herein, when it was reported that they were capable of restoring the ultrastructural retinal phenotypes in the animal model. Notably, shaker1 mice lack retinal degeneration, and the severe functional abnormalities seen in USH1B patients (Liu et al., 1997). This fact renders in vivo analysis of therapeutic outcomes in the shaker retina problematic. Alternative animal models for evaluating a treatment for this devastating disease may be useful in adaptation of the present methods to human clinical use.

The results presented here also demonstrated that MYO7A can be efficiently expressed using dual-AAV-vector systems. The platforms containing overlapping elements, namely, the simple overlap system, and the AP hybrid system were both highly efficient. AP hybrid vectors showed the strongest expression of all systems tested, with little observable truncated protein in vitro and none observed in vivo. Simple overlap vectors showed good expression and were the most specific (no truncated protein products were observed) even when the 5′-only vector was used to infect cells. AAV has emerged as the preferred clinical vector and it efficiently transduces both photoreceptors and RPE. Because it has now been demonstrated that MYO7A sequence fidelity is preserved following recombination and/or splicing of dual-AAV-vector platforms and because only full-length MYO7A was detectable in mouse retinas injected with dual vectors, the dual-AAV-vector strategy presented herein represents a valid option for the treatment of retinal disorders associated with mutations in large genes such as USHIB.

### Nucleotide Sequences of the Vectors Used in Examples 1-5

SEQ ID NO: 1 is the nucleotide sequence of the first generation front-half vector of the
overlap system (i.e., AAV-smCBA-hMYO7A-NTlong; “hMyo7a coding overlap vector A”);
CTCCCTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCC
ATCACTAGGGGTTCTCAGATCTGGCGCGCCCAATTCGGTACCCTAGTTATTAATAGTAATCA
ATTACGGGGTCATTAGTTCATAGCCCATATATGGAGTTCCGCGTTACATAACTTACGGTAAA
TGGCCCGCCTGGCTGACCGCCCAACGACCCCCGCCCATTGACGTCAATAATGACGTATGTTC
CCATAGTAACGCCAATAGGGACTTTCCATTGACGTCAATGGGTGGACTATTTACGGTAAAC
TGCCCACTTGGCAGTACATCAAGTGTATCATATGCCAAGTACGCCCCCTATTGACGTCAATG
ACGGTAAATGGCCCGCCTGGCATTATGCCCAGTACATGACCTTATGGGACTTTCCTACTTGG
CAGTACATCTACGTATTAGTCATCGCTATTACCATGGTCGAGGTGAGCCCCACGTTCTGCTT
CACTCTCCCCATCTCCCCCCCCTCCCCACCCCCAATTTTGTATTTATTTATTTTTTAATTATTT
TGTGCAGCGATGGGGGCGGGGGGGGGGGGGGGGCGCGCGCCAGGCGGGGCGGGGCGGGG
CGAGGGGCGGGGCGGGGCGAGGCGGAGAGGTGCGGCGGCAGCCAATCAGAGCGGCGCGC
TCCGAAAGTTTCCTTTTATGGCGAGGCGGCGGCGGCGGCGGCCCTATAAAAAGCGAAGCGC
GCGGCGGGCGGGAGTCGCTGCGCGCTGCCTTCGCCCCGTGCCCCGCTCCGCCGCCGCCTCG
CGCCGCCCGCCCCGGCTCTGACTGACCGCGTTACTCCCACAGGTGAGCGGGCGGGACGGCC
CTTCTCCTCCGGGCTGTAATTAGCGCTTGGTTTAATGACGGCTTGTTTCTTTTCTGTGGCTGC
GTGAAAGCCTTGAGGGGCTCCGGGAGCTAGAGCCTCTGCTAACCATGTTCATGCCTTCTTCT
TTTTCCTACAGCTCCTGGGCAACGTGCTGGTTATTGTGCTGTCTCATCATTTTGGCAAAGAA
TTCTAGCGGCCGCCACCATGGTGATTCTTCAGCAGGGGGACCATGTGTGGATGGACCTGAG
ATTGGGGCAGGAGTTCGACGTGCCCATCGGGGCGGTGGTGAAGCTCTGCGACTCTGGGCAG
GTCCAGGTGGTGGATGATGAAGACAATGAACACTGGATCTCTCCGCAGAACGCAACGCACA
TCAAGCCTATGCACCCCACGTCGGTCCACGGCGTGGAGGACATGATCCGCCTGGGGGACCT
CAACGAGGCGGGCATCTTGCGCAACCTGCTTATCCGCTACCGGGACCACCTCATCTACACG
TATACGGGCTCCATCCTGGTGGCTGTGAACCCCTACCAGCTGCTCTCCATCTACTCGCCAGA
GCACATCCGCCAGTATACCAACAAGAAGATTGGGGAGATGCCCCCCCACATCTTTGCCATT
GCTGACAACTGCTACTTCAACATGAAACGCAACAGCCGAGACCAGTGCTGCATCATCAGTG
GGGAATCTGGGGCCGGGAAGACGGAGAGCACAAAGCTGATCCTGCAGTTCCTGGCAGCCA
TCAGTGGGCAGCACTCGTGGATTGAGCAGCAGGTCTTGGAGGCCACCCCCATTCTGGAAGC
ATTTGGGAATGCCAAGACCATCCGCAATGACAACTCAAGCCGTTTCGGAAAGTACATCGAC
ATCCACTTCAACAAGCGGGGCGCCATCGAGGGCGCGAAGATTGAGCAGTACCTGCTGGAA
AAGTCACGTGTCTGTCGCCAGGCCCTGGATGAAAGGAACTACCACGTGTTCTACTGCATGC
TGGAGGGTATGAGTGAGGATCAGAAGAAGAAGCTGGGCTTGGGCCAGGCCTCTGACTACA
ACTACTTGGCCATGGGTAACTGCATAACCTGTGAGGGCCGGGTGGACAGCCAGGAGTACGC
CAACATCCGCTCCGCCATGAAGGTGCTCATGTTCACTGACACCGAGAACTGGGAGATCTCG
AAGCTCCTGGCTGCCATCCTGCACCTGGGCAACCTGCAGTATGAGGCACGCACATTTGAAA
ACCTGGATGCCTGTGAGGTTCTCTTCTCCCCATCGCTGGCCACAGCTGCATCCCTGCTTGAG
GTGAACCCCCCAGACCTGATGAGCTGCCTGACTAGCCGCACCCTCATCACCCGCGGGGAGA
CGGTGTCCACCCCACTGAGCAGGGAACAGGCACTGGACGTGCGCGACGCCTTCGTAAAGGG
GATCTACGGGCGGCTGTTCGTGTGGATTGTGGACAAGATCAACGCAGCAATTTACAAGCCT
CCCTCCCAGGATGTGAAGAACTCTCGCAGGTCCATCGGCCTCCTGGACATCTTTGGGTTTGA
GAACTTTGCTGTGAACAGCTTTGAGCAGCTCTGCATCAACTTCGCCAATGAGCACCTGCAGC
AGTTCTTTGTGCGGCACGTGTTCAAGCTGGAGCAGGAGGAATATGACCTGGAGAGCATTGA
CTGGCTGCACATCGAGTTCACTGACAACCAGGATGCCCTGGACATGATTGCCAACAAGCCC
ATGAACATCATCTCCCTCATCGATGAGGAGAGCAAGTTCCCCAAGGGCACAGACACCACCA
TGTTACACAAGCTGAACTCCCAGCACAAGCTCAACGCCAACTACATCCCCCCCAAGAACAA
CCATGAGACCCAGTTTGGCATCAACCATTTTGCAGGCATCGTCTACTATGAGACCCAAGGCT
TCCTGGAGAAGAACCGAGACACCCTGCATGGGGACATTATCCAGCTGGTCCACTCCTCCAG
GAACAAGTTCATCAAGCAGATCTTCCAGGCCGATGTCGCCATGGGCGCCGAGACCAGGAAG
CGCTCGCCCACACTTAGCAGCCAGTTCAAGCGGTCACTGGAGCTGCTGATGCGCACGCTGG
GTGCCTGCCAGCCCTTCTTTGTGCGATGCATCAAGCCCAATGAGTTCAAGAAGCCCATGCTG
TTCGACCGGCACCTGTGCGTGCGCCAGCTGCGGTACTCAGGAATGATGGAGACCATCCGAA
TCCGCCGAGCTGGCTACCCCATCCGCTACAGCTTCGTAGAGTTTGTGGAGCGGTACCGTGTG
CTGCTGCCAGGTGTGAAGCCGGCCTACAAGCAGGGCGACCTCCGCGGGACTTGCCAGCGCA
TGGCTGAGGCTGTGCTGGGCACCCACGATGACTGGCAGATAGGCAAAACCAAGATCTTTCT
GAAGGACCACCATGACATGCTGCTGGAAGTGGAGCGGGACAAAGCCATCACCGACAGAGT
CATCCTCCTTCAGAAAGTCATCCGGGGATTCAAAGACAGGTCTAACTTTCTGAAGCTGAAG
AACGCTGCCACACTGATCCAGAGGCACTGGCGGGGTCACAACTGTAGGAAGAACTACGGG
CTGATGCGTCTGGGCTTCCTGCGGCTGCAGGCCCTGCACCGCTCCCGGAAGCTGCACCAGC
AGTACCGCCTGGCCCGCCAGCGCATCATCCAGTTCCAGGCCCGCTGCCGCGCCTATCTGGTG
CGCAAGGCCTTCCGCCACCGCCTCTGGGCTGTGCTCACCGTGCAGGCCTATGCCCGGGGCA
TGATCGCCCGCAGGCTGCACCAACGCCTCAGGGCTGAGTATCTGTGGCGCCTCGAGGCTGA
GAAAATGCGGCTGGCGGAGGAAGAGAAGCTTCGGAAGGAGATGAGCGCCAAGAAGGCCA
AGGAGGAGGCCGAGCGCAAGCATCAGGAGCGCCTGGCCCAGCTGGCTCGTGAGGACGCTG
AGCGGGAGCTGAAGGAGAAGGAGGCCGCTCGGCGGAAGAAGGAGCTCCTGGAGCAGATG
GAAAGGGCCCGCCATGAGCCTGTCAATCACTCAGACATGGTGGACAAGATGTTTGGCTTCC
TGGGGACTTCAGGTGGCCTGCCAGGCCAGGAGGGCCAGGCACCTAGTGGCTTTGAGGACCT
GGAGCGAGGGCGGAGGGAGATGGTGGAGGAGGACCTGGATGCAGCCCTGCCCCTGCCTGA
CGAGGATGAGGAGGACCTCTCTGAGTATAAATTTGCCAAGTTCGCGGCCACCTACTTCCAG
GGGACAACCACGCACTCCTACACCCGGCGGCCACTCAAACAGCCACTGCTCTACCATGACG
ACGAGGGTGACCAGCTGGCAGCCCTGGCGGTCTGGATCACCATCCTCCGCTTCATGGGGGA
CCTCCCTGAGCCCAAGTACCACACAGCCATGAGTGATGGCAGTGAGAAGATCCCTGTGATG
ACCAAGATTTATGAGACCCTGGGCAAGAAGACGTACAAGAGGGAGCTGCAGGCCCTGCAG
GGCGAGGGCGAGGCCCAGCTCCCCGAGGGCCAGAAGAAGAGCAGTGTGAGGCACAAGCTG
GTGCATTTGACTCTGAAAAAGAAGTCCAAGCTCACAGAGGAGGTGACCAAGAGGCTGCAT
GACGGGGAGTCCACAGTGCAGGGCAACAGCATGCTGGAGGACCGGCCCACCTCCAACCTG
GAGAAGCTGCACTTCATCATCGGCAATGGCATCCTGCGGCCAGCACTCCGGGACGAGATCT
ACTGCCAGATCAGCAAGCAGCTGACCCACAACCCCTCCAAGAGCAGCTATGCCCGGGGCTG
GATTCTCGTGTCTCTCTGCGTGGGCTGTTTCGCCCCCTCCGAGAAGTTTGTCAAGTACCTGC
GGAACTTCGCTAGCGGGCACTAGTCCGTCGACTGTTAATTAAGCATGCTGGGGAGAGATCT
AGGAAACCCCTAGTGATGGAGTTGGCCACTCCCTCTCTGCGCGCTCGCTCGCTCACTGAGGC
CGGGCGACCAAAGGTCGCCCGACGCCCGGGCTTTGCCCGGGCGGCCTCAGTGAGCGAGCG
AGCGCGCAGAGAGGGAG
SEQ ID NO: 2 is the nucleotide sequence of the first generation back-half vector of the
overlap system (i.e., AAV-hMYO7A-CTlong.HA; “hMyo7a coding overlap vector B”);
CTCCCTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCC
ATCACTAGGGGTTCAGATCTGGCGCGCCCAATTGGCTTCGAATTCTAGCGGCCGCTGCTTAA
GCAGGTCTAACTTTCTGAAGCTGAAGAACGCTGCCACACTGATCCAGAGGCACTGGCGGGG
TCACAACTGTAGGAAGAACTACGGGCTGATGCGTCTGGGCTTCCTGCGGCTGCAGGCCCTG
CACCGCTCCCGGAAGCTGCACCAGCAGTACCGCCTGGCCCGCCAGCGCATCATCCAGTTCC
AGGCCCGCTGCCGCGCCTATCTGGTGCGCAAGGCCTTCCGCCACCGCCTCTGGGCTGTGCTC
ACCGTGCAGGCCTATGCCCGGGGCATGATCGCCCGCAGGCTGCACCAACGCCTCAGGGCTG
AGTATCTGTGGCGCCTCGAGGCTGAGAAAATGCGGCTGGCGGAGGAAGAGAAGCTTCGGA
AGGAGATGAGCGCCAAGAAGGCCAAGGAGGAGGCCGAGCGCAAGCATCAGGAGCGCCTG
GCCCAGCTGGCTCGTGAGGACGCTGAGCGGGAGCTGAAGGAGAAGGAGGCCGCTCGGCGG
AAGAAGGAGCTCCTGGAGCAGATGGAAAGGGCCCGCCATGAGCCTGTCAATCACTCAGAC
ATGGTGGACAAGATGTTTGGCTTCCTGGGGACTTCAGGTGGCCTGCCAGGCCAGGAGGGCC
AGGCACCTAGTGGCTTTGAGGACCTGGAGCGAGGGCGGAGGGAGATGGTGGAGGAGGACC
TGGATGCAGCCCTGCCCCTGCCTGACGAGGATGAGGAGGACCTCTCTGAGTATAAATTTGC
CAAGTTCGCGGCCACCTACTTCCAGGGGACAACCACGCACTCCTACACCCGGCGGCCACTC
AAACAGCCACTGCTCTACCATGACGACGAGGGTGACCAGCTGGCAGCCCTGGCGGTCTGGA
TCACCATCCTCCGCTTCATGGGGGACCTCCCTGAGCCCAAGTACCACACAGCCATGAGTGA
TGGCAGTGAGAAGATCCCTGTGATGACCAAGATTTATGAGACCCTGGGCAAGAAGACGTAC
AAGAGGGAGCTGCAGGCCCTGCAGGGCGAGGGCGAGGCCCAGCTCCCCGAGGGCCAGAAG
AAGAGCAGTGTGAGGCACAAGCTGGTGCATTTGACTCTGAAAAAGAAGTCCAAGCTCACA
GAGGAGGTGACCAAGAGGCTGCATGACGGGGAGTCCACAGTGCAGGGCAACAGCATGCTG
GAGGACCGGCCCACCTCCAACCTGGAGAAGCTGCACTTCATCATCGGCAATGGCATCCTGC
GGCCAGCACTCCGGGACGAGATCTACTGCCAGATCAGCAAGCAGCTGACCCACAACCCCTC
CAAGAGCAGCTATGCCCGGGGCTGGATTCTCGTGTCTCTCTGCGTGGGCTGTTTCGCCCCCT
CCGAGAAGTTTGTCAAGTACCTGCGGAACTTCATCCACGGGGGCCCGCCCGGCTACGCCCC
GTACTGTGAGGAGCGCCTGAGAAGGACCTTTGTCAATGGGACACGGACACAGCCGCCCAGC
TGGCTGGAGCTGCAGGCCACCAAGTCCAAGAAGCCAATCATGTTGCCCGTGACATTCATGG
ATGGGACCACCAAGACCCTGCTGACGGACTCGGCAACCACGGCCAAGGAGCTCTGCAACG
CGCTGGCCGACAAGATCTCTCTCAAGGACCGGTTCGGGTTCTCCCTCTACATTGCCCTGTTT
GACAAGGTGTCCTCCCTGGGCAGCGGCAGTGACCACGTCATGGACGCCATCTCCCAGTGCG
AGCAGTACGCCAAGGAGCAGGGCGCCCAGGAGCGCAACGCCCCCTGGAGGCTCTTCTTCCG
CAAAGAGGTCTTCACGCCCTGGCACAGCCCCTCCGAGGACAACGTGGCCACCAACCTCATC
TACCAGCAGGTGGTGCGAGGAGTCAAGTTTGGGGAGTACAGGTGTGAGAAGGAGGACGAC
CTGGCTGAGCTGGCCTCCCAGCAGTACTTTGTAGACTATGGCTCTGAGATGATCCTGGAGCG
CCTCCTGAACCTCGTGCCCACCTACATCCCCGACCGCGAGATCACGCCCCTGAAGACGCTG
GAGAAGTGGGCCCAGCTGGCCATCGCCGCCCACAAGAAGGGGATTTATGCCCAGAGGAGA
ACTGATGCCCAGAAGGTCAAAGAGGATGTGGTCAGTTATGCCCGCTTCAAGTGGCCCTTGC
TCTTCTCCAGGTTTTATGAAGCCTACAAATTCTCAGGCCCCAGTCTCCCCAAGAACGACGTC
ATCGTGGCCGTCAACTGGACGGGTGTGTACTTTGTGGATGAGCAGGAGCAGGTACTTCTGG
AGCTGTCCTTCCCAGAGATCATGGCCGTGTCCAGCAGCAGGGGAGCGAAAACGACGGCCCC
CAGCTTCACGCTGGCCACCATCAAGGGGGACGAATACACCTTCACCTCCAGCAATGCTGAG
GACATTCGTGACCTGGTGGTCACCTTCCTAGAGGGGCTCCGGAAGAGATCTAAGTATGTTG
TGGCCCTGCAGGATAACCCCAACCCCGCAGGCGAGGAGTCAGGCTTCCTCAGCTTTGCCAA
GGGAGACCTCATCATCCTGGACCATGACACGGGCGAGCAGGTCATGAACTCGGGCTGGGCC
AACGGCATCAATGAGAGGACCAAGCAGCGTGGGGACTTCCCCACCGACAGTGTGTACGTCA
TGCCCACTGTCACCATGCCACCGCGGGAGATTGTGGCCCTGGTCACCATGACTCCCGATCA
GAGGCAGGACGTTGTCCGGCTCTTGCAGCTGCGAACGGCGGAGCCCGAGGTGCGTGCCAAG
CCCTACACGCTGGAGGAGTTTTCCTATGACTACTTCAGGCCCCCACCCAAGCACACGCTGA
GCCGTGTCATGGTGTCCAAGGCCCGAGGCAAGGACCGGCTGTGGAGCCACACGCGGGAAC
CGCTCAAGCAGGCGCTGCTCAAGAAGCTCCTGGGCAGTGAGGAGCTCTCGCAGGAGGCCTG
CCTGGCCTTCATTGCTGTGCTCAAGTACATGGGCGACTACCCGTCCAAGAGGACACGCTCC
GTCAACGAGCTCACCGACCAGATCTTTGAGGGTCCCCTGAAAGCCGAGCCCCTGAAGGACG
AGGCATATGTGCAGATCCTGAAGCAGCTGACCGACAACCACATCAGGTACAGCGAGGAGC
GGGGTTGGGAGCTGCTCTGGCTGTGCACGGGCCTTTTCCCACCCAGCAACATCCTCCTGCCC
CACGTGCAGCGCTTCCTGCAGTCCCGAAAGCACTGCCCACTCGCCATCGACTGCCTGCAAC
GGCTCCAGAAAGCCCTGAGAAACGGGTCCCGGAAGTACCCTCCGCACCTGGTGGAGGTGG
AGGCCATCCAGCACAAGACCACCCAGATTTTCCACAAAGTCTACTTCCCTGATGACACTGA
CGAGGCCTTCGAAGTGGAGTCCAGCACCAAGGCCAAGGACTTCTGCCAGAACATCGCCACC
AGGCTGCTCCTCAAGTCCTCAGAGGGATTCAGCCTCTTTGTCAAAATTGCAGACAAGGTCAT
CAGCGTTCCTGAGAATGACTTCTTCTTTGACTTTGTTCGACACTTGACAGACTGGATAAAGA
AAGCTCGGCCCATCAAGGACGGAATTGTGCCCTCACTCACCTACCAGGTGTTCTTCATGAA
GAAGCTGTGGACCACCACGGTGCCAGGGAAGGATCCCATGGCCGATTCCATCTTCCACTAT
TACCAGGAGTTGCCCAAGTATCTCCGAGGCTACCACAAGTGCACGCGGGAGGAGGTGCTGC
AGCTGGGGGCGCTGATCTACAGGGTCAAGTTCGAGGAGGACAAGTCCTACTTCCCCAGCAT
CCCCAAGCTGCTGCGGGAGCTGGTGCCCCAGGACCTTATCCGGCAGGTCTCACCTGATGAC
TGGAAGCGGTCCATCGTCGCCTACTTCAACAAGCACGCAGGGAAGTCCAAGGAGGAGGCC
AAGCTGGCCTTCCTGAAGCTCATCTTCAAGTGGCCCACCTTTGGCTCAGCCTTCTTCGAGGT
GAAGCAAACTACGGAGCCAAACTTCCCTGAGATCCTCCTAATTGCCATCAACAAGTATGGG
GTCAGCCTCATCGATCCCAAAACGAAGGATATCCTCACCACTCATCCCTTCACCAAGATCTC
CAACTGGAGCAGCGGCAACACCTACTTCCACATCACCATTGGGAACTTGGTGCGCGGGAGC
AAACTGCTCTGCGAGACGTCACTGGGCTACAAGATGGATGACCTCCTGACTTCCTACATTA
GCCAGATGCTCACAGCCATGAGCAAACAGCGGGGCTCCAGGAGCGGCAAGTACCCTTACG
ATGTACCGGATTACGCATGAGGTACCAAGGGCGAATTCTGCAGTCGACTAGAGCTCGCTGA
TCAGCCTCGACTGTGCCTTCTAGTTGCCAGCCATCTGTTGTTTGCCCCTCCCCCGTGCCTTCC
TTGACCCTGGAAGGTGCCACTCCCACTGTCCTTTCCTAATAAAATGAGGAAATTGCATCGCA
TTGTCTGAGTAGGTGTCATTCTATTCTGGGGGGTGGGGTGGGGCAGGACAGCAAGGGGGAG
GATTGGGAAGACAATAGCAGGCATGCTGGGGAGAGATCTGAGGACTAGTCCGTCGACTGTT
AATTAAGCATGCTGGGGAGAGATCTAACCCCTAGTGATGGAGTTGGCCACTCCCTCTCTGC
GCGCTCGCTCGCTCACTGAGGCCGGGCGACCAAAGGTCGCCCGACGCCCGGGCTTTGCCCG
GGCGGCCTCAGTGAGCGAGCGAGCGCGCAGAGAGGGAG
SEQ ID NO: 3 is the nucleotide sequence of the front-half vector of the exon 23/24 hybrid
vector system (i.e., AAV-smCBA-hMYO7ANT-APSD-ApHead);
CTCCCTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCC
ATCACTAGGGGTTCTCAGATCTGGCGCGCCCAATTCGGTACCCTAGTTATTAATAGTAATCA
ATTACGGGGTCATTAGTTCATAGCCCATATATGGAGTTCCGCGTTACATAACTTACGGTAAA
TGGCCCGCCTGGCTGACCGCCCAACGACCCCCGCCCATTGACGTCAATAATGACGTATGTTC
CCATAGTAACGCCAATAGGGACTTTCCATTGACGTCAATGGGTGGACTATTTACGGTAAAC
TGCCCACTTGGCAGTACATCAAGTGTATCATATGCCAAGTACGCCCCCTATTGACGTCAATG
ACGGTAAATGGCCCGCCTGGCATTATGCCCAGTACATGACCTTATGGGACTTTCCTACTTGG
CAGTACATCTACGTATTAGTCATCGCTATTACCATGGTCGAGGTGAGCCCCACGTTCTGCTT
CACTCTCCCCATCTCCCCCCCCTCCCCACCCCCAATTTTGTATTTATTTATTTTTTAATTATTT
TGTGCAGCGATGGGGGCGGGGGGGGGGGGGGGGCGCGCGCCAGGCGGGGCGGGGCGGGG
CGAGGGGCGGGGCGGGGCGAGGCGGAGAGGTGCGGCGGCAGCCAATCAGAGCGGCGCGC
TCCGAAAGTTTCCTTTTATGGCGAGGCGGCGGCGGCGGCGGCCCTATAAAAAGCGAAGCGC
GCGGCGGGCGGGAGTCGCTGCGCGCTGCCTTCGCCCCGTGCCCCGCTCCGCCGCCGCCTCG
CGCCGCCCGCCCCGGCTCTGACTGACCGCGTTACTCCCACAGGTGAGCGGGCGGGACGGCC
CTTCTCCTCCGGGCTGTAATTAGCGCTTGGTTTAATGACGGCTTGTTTCTTTTCTGTGGCTGC
GTGAAAGCCTTGAGGGGCTCCGGGAGCTAGAGCCTCTGCTAACCATGTTCATGCCTTCTTCT
TTTTCCTACAGCTCCTGGGCAACGTGCTGGTTATTGTGCTGTCTCATCATTTTGGCAAAGAA
TTCTAGCGGCCGCCACCATGGTGATTCTTCAGCAGGGGGACCATGTGTGGATGGACCTGAG
ATTGGGGCAGGAGTTCGACGTGCCCATCGGGGCGGTGGTGAAGCTCTGCGACTCTGGGCAG
GTCCAGGTGGTGGATGATGAAGACAATGAACACTGGATCTCTCCGCAGAACGCAACGCACA
TCAAGCCTATGCACCCCACGTCGGTCCACGGCGTGGAGGACATGATCCGCCTGGGGGACCT
CAACGAGGCGGGCATCTTGCGCAACCTGCTTATCCGCTACCGGGACCACCTCATCTACACG
TATACGGGCTCCATCCTGGTGGCTGTGAACCCCTACCAGCTGCTCTCCATCTACTCGCCAGA
GCACATCCGCCAGTATACCAACAAGAAGATTGGGGAGATGCCCCCCCACATCTTTGCCATT
GCTGACAACTGCTACTTCAACATGAAACGCAACAGCCGAGACCAGTGCTGCATCATCAGTG
GGGAATCTGGGGCCGGGAAGACGGAGAGCACAAAGCTGATCCTGCAGTTCCTGGCAGCCA
TCAGTGGGCAGCACTCGTGGATTGAGCAGCAGGTCTTGGAGGCCACCCCCATTCTGGAAGC
ATTTGGGAATGCCAAGACCATCCGCAATGACAACTCAAGCCGTTTCGGAAAGTACATCGAC
ATCCACTTCAACAAGCGGGGCGCCATCGAGGGCGCGAAGATTGAGCAGTACCTGCTGGAA
AAGTCACGTGTCTGTCGCCAGGCCCTGGATGAAAGGAACTACCACGTGTTCTACTGCATGC
TGGAGGGTATGAGTGAGGATCAGAAGAAGAAGCTGGGCTTGGGCCAGGCCTCTGACTACA
ACTACTTGGCCATGGGTAACTGCATAACCTGTGAGGGCCGGGTGGACAGCCAGGAGTACGC
CAACATCCGCTCCGCCATGAAGGTGCTCATGTTCACTGACACCGAGAACTGGGAGATCTCG
AAGCTCCTGGCTGCCATCCTGCACCTGGGCAACCTGCAGTATGAGGCACGCACATTTGAAA
ACCTGGATGCCTGTGAGGTTCTCTTCTCCCCATCGCTGGCCACAGCTGCATCCCTGCTTGAG
GTGAACCCCCCAGACCTGATGAGCTGCCTGACTAGCCGCACCCTCATCACCCGCGGGGAGA
CGGTGTCCACCCCACTGAGCAGGGAACAGGCACTGGACGTGCGCGACGCCTTCGTAAAGGG
GATCTACGGGCGGCTGTTCGTGTGGATTGTGGACAAGATCAACGCAGCAATTTACAAGCCT
CCCTCCCAGGATGTGAAGAACTCTCGCAGGTCCATCGGCCTCCTGGACATCTTTGGGTTTGA
GAACTTTGCTGTGAACAGCTTTGAGCAGCTCTGCATCAACTTCGCCAATGAGCACCTGCAGC
AGTTCTTTGTGCGGCACGTGTTCAAGCTGGAGCAGGAGGAATATGACCTGGAGAGCATTGA
CTGGCTGCACATCGAGTTCACTGACAACCAGGATGCCCTGGACATGATTGCCAACAAGCCC
ATGAACATCATCTCCCTCATCGATGAGGAGAGCAAGTTCCCCAAGGGCACAGACACCACCA
TGTTACACAAGCTGAACTCCCAGCACAAGCTCAACGCCAACTACATCCCCCCCAAGAACAA
CCATGAGACCCAGTTTGGCATCAACCATTTTGCAGGCATCGTCTACTATGAGACCCAAGGCT
TCCTGGAGAAGAACCGAGACACCCTGCATGGGGACATTATCCAGCTGGTCCACTCCTCCAG
GAACAAGTTCATCAAGCAGATCTTCCAGGCCGATGTCGCCATGGGCGCCGAGACCAGGAAG
CGCTCGCCCACACTTAGCAGCCAGTTCAAGCGGTCACTGGAGCTGCTGATGCGCACGCTGG
GTGCCTGCCAGCCCTTCTTTGTGCGATGCATCAAGCCCAATGAGTTCAAGAAGCCCATGCTG
TTCGACCGGCACCTGTGCGTGCGCCAGCTGCGGTACTCAGGAATGATGGAGACCATCCGAA
TCCGCCGAGCTGGCTACCCCATCCGCTACAGCTTCGTAGAGTTTGTGGAGCGGTACCGTGTG
CTGCTGCCAGGTGTGAAGCCGGCCTACAAGCAGGGCGACCTCCGCGGGACTTGCCAGCGCA
TGGCTGAGGCTGTGCTGGGCACCCACGATGACTGGCAGATAGGCAAAACCAAGATCTTTCT
GAAGGACCACCATGACATGCTGCTGGAAGTGGAGCGGGACAAAGCCATCACCGACAGAGT
CATCCTCCTTCAGAAAGTCATCCGGGGATTCAAAGACAGGTCTAACTTTCTGAAGCTGAAG
AACGCTGCCACACTGATCCAGAGGCACTGGCGGGGTCACAACTGTAGGAAGAACTACGGG
CTGATGCGTCTGGGCTTCCTGCGGCTGCAGGCCCTGCACCGCTCCCGGAAGCTGCACCAGC
AGTACCGCCTGGCCCGCCAGCGCATCATCCAGTTCCAGGCCCGCTGCCGCGCCTATCTGGTG
CGCAAGGCCTTCCGCCACCGCCTCTGGGCTGTGCTCACCGTGCAGGCCTATGCCCGGGGCA
TGATCGCCCGCAGGCTGCACCAACGCCTCAGGGCTGAGTATCTGTGGCGCCTCGAGGCTGA
GAAAATGCGGCTGGCGGAGGAAGAGAAGCTTCGGAAGGAGATGAGCGCCAAGAAGGCCA
AGGAGGAGGCCGAGCGCAAGCATCAGGAGCGCCTGGCCCAGCTGGCTCGTGAGGACGCTG
AGCGGGAGCTGAAGGAGAAGGAGGCCGCTCGGCGGAAGAAGGAGCTCCTGGAGCAGATG
GAAAGGGCCCGCCATGAGCCTGTCAATCACTCAGACATGGTGGACAAGATGTTTGGCTTCC
TGGGGACTTCAGGTGGCCTGCCAGGCCAGGAGGGCCAGGCACCTAGTGGCTTTGAGGTAAG
TATCAAGGTTACAAGACAGGTTTAAGGAGACCAATAGAAACTGGGCTTGTCGAGACAGAG
AAGACTCTTGCGTTTCTGAGCTAGCCCCCGGGTGCGCGGCGTCGGTGGTGCCGGCGGGGGG
CGCCAGGTCGCAGGCGGTGTAGGGCTCCAGGCAGGCGGCGAAGGCCATGACGTGCGCTAT
GAAGGTCTGCTCCTGCACGCCGTGAACCAGGTGCGCCTGCGGGCCGCGCGCGAACACCGCC
ACGTCCTCGCCTGCGTGGGTCTCTTCGTCCAGGGGCACTGCTGACTGCTGCCGATACTCGGG
GCTCCCGCTCTCGCTCTCGGTAACATCCGGCCGGGCGCCGTCCTTGAGCACATAGCCTGGAC
CGTTTCGTCGACTGTTAATTAAGCATGCTGGGGAGAGATCTAGGAACCCCTAGTGATGGAG
TTGGCCACTCCCTCTCTGCGCGCTCGCTCGCTCACTGAGGCCGGGCGACCAAAGGTCGCCCG
ACGCCCGGGCTTTGCCCGGGCGGCCTCAGTGAGCGAGCGAGCGCGCAGAGAGGGAG
SEQ ID NO: 67 (only the N-myosin7A portion of SEQ ID NO: 3 (AAV-smCBA-
hMYO7ANT-APSD-ApHead))
ATGGTGATTCTTCAGCAGGGGGACCATGTGTGGATGGACCTGAGATTGGGGCAGGAGTTCG
ACGTGCCCATCGGGGCGGTGGTGAAGCTCTGCGACTCTGGGCAGGTCCAGGTGGTGGATGA
TGAAGACAATGAACACTGGATCTCTCCGCAGAACGCAACGCACATCAAGCCTATGCACCCC
ACGTCGGTCCACGGCGTGGAGGACATGATCCGCCTGGGGGACCTCAACGAGGCGGGCATCT
TGCGCAACCTGCTTATCCGCTACCGGGACCACCTCATCTACACGTATACGGGCTCCATCCTG
GTGGCTGTGAACCCCTACCAGCTGCTCTCCATCTACTCGCCAGAGCACATCCGCCAGTATAC
CAACAAGAAGATTGGGGAGATGCCCCCCCACATCTTTGCCATTGCTGACAACTGCTACTTC
AACATGAAACGCAACAGCCGAGACCAGTGCTGCATCATCAGTGGGGAATCTGGGGCCGGG
AAGACGGAGAGCACAAAGCTGATCCTGCAGTTCCTGGCAGCCATCAGTGGGCAGCACTCGT
GGATTGAGCAGCAGGTCTTGGAGGCCACCCCCATTCTGGAAGCATTTGGGAATGCCAAGAC
CATCCGCAATGACAACTCAAGCCGTTTCGGAAAGTACATCGACATCCACTTCAACAAGCGG
GGCGCCATCGAGGGCGCGAAGATTGAGCAGTACCTGCTGGAAAAGTCACGTGTCTGTCGCC
AGGCCCTGGATGAAAGGAACTACCACGTGTTCTACTGCATGCTGGAGGGTATGAGTGAGGA
TCAGAAGAAGAAGCTGGGCTTGGGCCAGGCCTCTGACTACAACTACTTGGCCATGGGTAAC
TGCATAACCTGTGAGGGCCGGGTGGACAGCCAGGAGTACGCCAACATCCGCTCCGCCATGA
AGGTGCTCATGTTCACTGACACCGAGAACTGGGAGATCTCGAAGCTCCTGGCTGCCATCCT
GCACCTGGGCAACCTGCAGTATGAGGCACGCACATTTGAAAACCTGGATGCCTGTGAGGTT
CTCTTCTCCCCATCGCTGGCCACAGCTGCATCCCTGCTTGAGGTGAACCCCCCAGACCTGAT
GAGCTGCCTGACTAGCCGCACCCTCATCACCCGCGGGGAGACGGTGTCCACCCCACTGAGC
AGGGAACAGGCACTGGACGTGCGCGACGCCTTCGTAAAGGGGATCTACGGGCGGCTGTTCG
TGTGGATTGTGGACAAGATCAACGCAGCAATTTACAAGCCTCCCTCCCAGGATGTGAAGAA
CTCTCGCAGGTCCATCGGCCTCCTGGACATCTTTGGGTTTGAGAACTTTGCTGTGAACAGCT
TTGAGCAGCTCTGCATCAACTTCGCCAATGAGCACCTGCAGCAGTTCTTTGTGCGGCACGTG
TTCAAGCTGGAGCAGGAGGAATATGACCTGGAGAGCATTGACTGGCTGCACATCGAGTTCA
CTGACAACCAGGATGCCCTGGACATGATTGCCAACAAGCCCATGAACATCATCTCCCTCAT
CGATGAGGAGAGCAAGTTCCCCAAGGGCACAGACACCACCATGTTACACAAGCTGAACTCC
CAGCACAAGCTCAACGCCAACTACATCCCCCCCAAGAACAACCATGAGACCCAGTTTGGCA
TCAACCATTTTGCAGGCATCGTCTACTATGAGACCCAAGGCTTCCTGGAGAAGAACCGAGA
CACCCTGCATGGGGACATTATCCAGCTGGTCCACTCCTCCAGGAACAAGTTCATCAAGCAG
ATCTTCCAGGCCGATGTCGCCATGGGCGCCGAGACCAGGAAGCGCTCGCCCACACTTAGCA
GCCAGTTCAAGCGGTCACTGGAGCTGCTGATGCGCACGCTGGGTGCCTGCCAGCCCTTCTTT
GTGCGATGCATCAAGCCCAATGAGTTCAAGAAGCCCATGCTGTTCGACCGGCACCTGTGCG
TGCGCCAGCTGCGGTACTCAGGAATGATGGAGACCATCCGAATCCGCCGAGCTGGCTACCC
CATCCGCTACAGCTTCGTAGAGTTTGTGGAGCGGTACCGTGTGCTGCTGCCAGGTGTGAAG
CCGGCCTACAAGCAGGGCGACCTCCGCGGGACTTGCCAGCGCATGGCTGAGGCTGTGCTGG
GCACCCACGATGACTGGCAGATAGGCAAAACCAAGATCTTTCTGAAGGACCACCATGACAT
GCTGCTGGAAGTGGAGCGGGACAAAGCCATCACCGACAGAGTCATCCTCCTTCAGAAAGTC
ATCCGGGGATTCAAAGACAGGTCTAACTTTCTGAAGCTGAAGAACGCTGCCACACTGATCC
AGAGGCACTGGCGGGGTCACAACTGTAGGAAGAACTACGGGCTGATGCGTCTGGGCTTCCT
GCGGCTGCAGGCCCTGCACCGCTCCCGGAAGCTGCACCAGCAGTACCGCCTGGCCCGCCAG
CGCATCATCCAGTTCCAGGCCCGCTGCCGCGCCTATCTGGTGCGCAAGGCCTTCCGCCACCG
CCTCTGGGCTGTGCTCACCGTGCAGGCCTATGCCCGGGGCATGATCGCCCGCAGGCTGCAC
CAACGCCTCAGGGCTGAGTATCTGTGGCGCCTCGAGGCTGAGAAAATGCGGCTGGCGGAGG
AAGAGAAGCTTCGGAAGGAGATGAGCGCCAAGAAGGCCAAGGAGGAGGCCGAGCGCAAG
CATCAGGAGCGCCTGGCCCAGCTGGCTCGTGAGGACGCTGAGCGGGAGCTGAAGGAGAAG
GAGGCCGCTCGGCGGAAGAAGGAGCTCCTGGAGCAGATGGAAAGGGCCCGCCATGAGCCT
GTCAATCACTCAGACATGGTGGACAAGATGTTTGGCTTCCTGGGGACTTCAGGTGGCCTGC
CAGGCCAGGAGGGCCAGGCACCTAGTGGCTTTGAG
SEQ ID NO: 68 (only the N-myosin7A portion encoded by SEQ ID NO: 3 (AAV-
smCBA-hMYO7ANT-APSD-APhead))
MVILQQGDHVWMDLRLGQEFDVPIGAVVKLCDSGQVQVVDDEDNEHWISPQNATHIKPMHPT
SVHGVEDMIRLGDLNEAGILRNLLIRYRDHLIYTYTGSILVAVNPYQLLSIYSPEHIRQYTNKKIG
EMPPHIFAIADNCYFNMKRNSRDQCCIISGESGAGKTESTKLILQFLAAISGQHSWIEQQVLEATP
ILEAFGNAKTIRNDNSSRFGKYIDIHFNKRGAIEGAKIEQYLLEKSRVCRQALDERNYHVFYCML
EGMSEDQKKKLGLGQASDYNYLAMGNCITCEGRVDSQEYANIRSAMKVLMFTDTENWEISKL
LAAILHLGNLQYEARTFENLDACEVLFSPSLATAASLLEVNPPDLMSCLTSRTLITRGETVSTPLS
REQALDVRDAFVKGIYGRLFVWIVDKINAAIYKPPSQDVKNSRRSIGLLDIFGFENFAVNSFEQL
CINFANEHLQQFFVRHVFKLEQEEYDLESIDWLHIEFTDNQDALDMIANKPMNIISLIDEESKFPK
GTDTTMLHKLNSQHKLNANYIPPKNNHETQFGINHFAGIVYYETQGFLEKNRDTLHGDIIQLVH
SSRNKFIKQIFQADVAMGAETRKRSPTLSSQFKRSLELLMRTLGACQPFFVRCIKPNEFKKPMLF
DRHLCVRQLRYSGMMETIRIRRAGYPIRYSFVEFVERYRVLLPGVKPAYKQGDLRGTCQRMAE
AVLGTHDDWQIGKTKIFLKDHHDMLLEVERDKAITDRVILLQKVIRGFKDRSNFLKLKNAATLI
QRHWRGHNCRKNYGLMRLGFLRLQALHRSRKLHQQYRLARQRIIQFQARCRAYLVRKAFRHR
LWAVLTVQAYARGMIARRLHQRLRAEYLWRLEAEKMRLAEEEKLRKEMSAKKAKEEAERKH
QERLAQLAREDAERELKEKEAARRKKELLEQMERARHEPVNHSDMVDKMFGFLGTSGGLPGQ
EGQAPSGFE
SEQ ID NO: 69 (Alkaline phosphatase head sequence (APhead)(e.g., AAV-
smCBA-hMYO7A-NT-APSD-APhead, AAV-smCBA-hMYO7A-NT-Ex21-APSD-APhead,
AAV-smCBA-hMYO7A-NT-Ex21-APSD-APhead CMv1, AAV-APhead-APSA-
hMYO7ACTex22.HA-MIN, AAV-APhead-APSA-hMYO7ACTex22-MIN))
CCCCGGGTGCGCGGCGTCGGTGGTGCCGGCGGGGGGCGCCAGGTCGCAGGCGGTGTAGGG
CTCCAGGCAGGCGGCGAAGGCCATGACGTGCGCTATGAAGGTCTGCTCCTGCACGCCGTGA
ACCAGGTGCGCCTGCGGGCCGCGCGCGAACACCGCCACGTCCTCGCCTGCGTGGGTCTCTT
CGTCCAGGGGCACTGCTGACTGCTGCCGATACTCGGGGCTCCCGCTCTCGCTCTCGGTAACA
TCCGGCCGGGCGCCGTCCTTGAGCACATAGCCTGGACCGTTTC
SEQ ID NO: 70 Alkaline phosphatase head sequence (APhead)(e.g., AAV-
smCB A-hMYO7A-NT-ex21-APSD-APhead-CMv2, AAV-smCBA-hMYO7A-NT-ex21-APSD-
APhead-CMv3, AAV-APhead-APSA-hMYO7ACT-ex22-APSD-CMv2.HA, AAV-APhead-
APSA-hMYO7ACT-ex22-APSD-CMv2.HA-MIN, AAV-APhead-APSA-hMYO7ACT-ex22-
APSD-CMv2-MIN)
CCCCGGGTGCGCGGCGTCGGTGGTGCCGGCGGGGGGCGCCAGGTCGCAGGCGGTGTAGGG
CTCCAGGCAGGCGGCGAAGGCCATGACGTGCGCTATGAAGGTCTGCTCCTGCACGCCGTGA
ACCAGGTGCGCCTGCGGGCCGCGCGCGAACACCGCCACGTCCTCGCCTGCGTGGGTCTCTT
CGTCCAGGGGCACTGCGCACTGCTGCCGATACTCGGGGCTCCCGCTCTCGCTCTCGGTAACA
TCCGGCCGGGCGCCGTCCTTGAGCACATAGCCTGGACCGTTTC
SEQ ID NO: 4 is the nucleotide sequence of the back-half vector of the exon 23/24 hybrid
vector system (i.e., AAV-APhead-APSA-hMYO7ACT.HA);
CTCCCTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCC
ATCACTAGGGGTTCTCAGATCTGGCGCGCCCAATTGGCTTCGAATTCTAGCGGCCGCCCCCG
GGTGCGCGGCGTCGGTGGTGCCGGCGGGGGGCGCCAGGTCGCAGGCGGTGTAGGGCTCCA
GGCAGGCGGCGAAGGCCATGACGTGCGCTATGAAGGTCTGCTCCTGCACGCCGTGAACCAG
GTGCGCCTGCGGGCCGCGCGCGAACACCGCCACGTCCTCGCCTGCGTGGGTCTCTTCGTCCA
GGGGCACTGCTGACTGCTGCCGATACTCGGGGCTCCCGCTCTCGCTCTCGGTAACATCCGGC
CGGGCGCCGTCCTTGAGCACATAGCCTGGACCGTTTCCTTAAGCGACGCATGCTCGCGATA
GGCACCTATTGGTCTTACTGACATCCACTTTGCCTTTCTCTCCACAGGACCTGGAGCGAGGG
CGGAGGGAGATGGTGGAGGAGGACCTGGATGCAGCCCTGCCCCTGCCTGACGAGGATGAG
GAGGACCTCTCTGAGTATAAATTTGCCAAGTTCGCGGCCACCTACTTCCAGGGGACAACCA
CGCACTCCTACACCCGGCGGCCACTCAAACAGCCACTGCTCTACCATGACGACGAGGGTGA
CCAGCTGGCAGCCCTGGCGGTCTGGATCACCATCCTCCGCTTCATGGGGGACCTCCCTGAGC
CCAAGTACCACACAGCCATGAGTGATGGCAGTGAGAAGATCCCTGTGATGACCAAGATTTA
TGAGACCCTGGGCAAGAAGACGTACAAGAGGGAGCTGCAGGCCCTGCAGGGCGAGGGCGA
GGCCCAGCTCCCCGAGGGCCAGAAGAAGAGCAGTGTGAGGCACAAGCTGGTGCATTTGAC
TCTGAAAAAGAAGTCCAAGCTCACAGAGGAGGTGACCAAGAGGCTGCATGACGGGGAGTC
CACAGTGCAGGGCAACAGCATGCTGGAGGACCGGCCCACCTCCAACCTGGAGAAGCTGCA
CTTCATCATCGGCAATGGCATCCTGCGGCCAGCACTCCGGGACGAGATCTACTGCCAGATC
AGCAAGCAGCTGACCCACAACCCCTCCAAGAGCAGCTATGCCCGGGGCTGGATTCTCGTGT
CTCTCTGCGTGGGCTGTTTCGCCCCCTCCGAGAAGTTTGTCAAGTACCTGCGGAACTTCATC
CACGGGGGCCCGCCCGGCTACGCCCCGTACTGTGAGGAGCGCCTGAGAAGGACCTTTGTCA
ATGGGACACGGACACAGCCGCCCAGCTGGCTGGAGCTGCAGGCCACCAAGTCCAAGAAGC
CAATCATGTTGCCCGTGACATTCATGGATGGGACCACCAAGACCCTGCTGACGGACTCGGC
AACCACGGCCAAGGAGCTCTGCAACGCGCTGGCCGACAAGATCTCTCTCAAGGACCGGTTC
GGGTTCTCCCTCTACATTGCCCTGTTTGACAAGGTGTCCTCCCTGGGCAGCGGCAGTGACCA
CGTCATGGACGCCATCTCCCAGTGCGAGCAGTACGCCAAGGAGCAGGGCGCCCAGGAGCG
CAACGCCCCCTGGAGGCTCTTCTTCCGCAAAGAGGTCTTCACGCCCTGGCACAGCCCCTCCG
AGGACAACGTGGCCACCAACCTCATCTACCAGCAGGTGGTGCGAGGAGTCAAGTTTGGGGA
GTACAGGTGTGAGAAGGAGGACGACCTGGCTGAGCTGGCCTCCCAGCAGTACTTTGTAGAC
TATGGCTCTGAGATGATCCTGGAGCGCCTCCTGAACCTCGTGCCCACCTACATCCCCGACCG
CGAGATCACGCCCCTGAAGACGCTGGAGAAGTGGGCCCAGCTGGCCATCGCCGCCCACAA
GAAGGGGATTTATGCCCAGAGGAGAACTGATGCCCAGAAGGTCAAAGAGGATGTGGTCAG
TTATGCCCGCTTCAAGTGGCCCTTGCTCTTCTCCAGGTTTTATGAAGCCTACAAATTCTCAG
GCCCCAGTCTCCCCAAGAACGACGTCATCGTGGCCGTCAACTGGACGGGTGTGTACTTTGT
GGATGAGCAGGAGCAGGTACTTCTGGAGCTGTCCTTCCCAGAGATCATGGCCGTGTCCAGC
AGCAGGGGAGCGAAAACGACGGCCCCCAGCTTCACGCTGGCCACCATCAAGGGGGACGAA
TACACCTTCACCTCCAGCAATGCTGAGGACATTCGTGACCTGGTGGTCACCTTCCTAGAGGG
GCTCCGGAAGAGATCTAAGTATGTTGTGGCCCTGCAGGATAACCCCAACCCCGCAGGCGAG
GAGTCAGGCTTCCTCAGCTTTGCCAAGGGAGACCTCATCATCCTGGACCATGACACGGGCG
AGCAGGTCATGAACTCGGGCTGGGCCAACGGCATCAATGAGAGGACCAAGCAGCGTGGGG
ACTTCCCCACCGACAGTGTGTACGTCATGCCCACTGTCACCATGCCACCGCGGGAGATTGTG
GCCCTGGTCACCATGACTCCCGATCAGAGGCAGGACGTTGTCCGGCTCTTGCAGCTGCGAA
CGGCGGAGCCCGAGGTGCGTGCCAAGCCCTACACGCTGGAGGAGTTTTCCTATGACTACTT
CAGGCCCCCACCCAAGCACACGCTGAGCCGTGTCATGGTGTCCAAGGCCCGAGGCAAGGAC
CGGCTGTGGAGCCACACGCGGGAACCGCTCAAGCAGGCGCTGCTCAAGAAGCTCCTGGGC
AGTGAGGAGCTCTCGCAGGAGGCCTGCCTGGCCTTCATTGCTGTGCTCAAGTACATGGGCG
ACTACCCGTCCAAGAGGACACGCTCCGTCAACGAGCTCACCGACCAGATCTTTGAGGGTCC
CCTGAAAGCCGAGCCCCTGAAGGACGAGGCATATGTGCAGATCCTGAAGCAGCTGACCGA
CAACCACATCAGGTACAGCGAGGAGCGGGGTTGGGAGCTGCTCTGGCTGTGCACGGGCCTT
TTCCCACCCAGCAACATCCTCCTGCCCCACGTGCAGCGCTTCCTGCAGTCCCGAAAGCACTG
CCCACTCGCCATCGACTGCCTGCAACGGCTCCAGAAAGCCCTGAGAAACGGGTCCCGGAAG
TACCCTCCGCACCTGGTGGAGGTGGAGGCCATCCAGCACAAGACCACCCAGATTTTCCACA
AAGTCTACTTCCCTGATGACACTGACGAGGCCTTCGAAGTGGAGTCCAGCACCAAGGCCAA
GGACTTCTGCCAGAACATCGCCACCAGGCTGCTCCTCAAGTCCTCAGAGGGATTCAGCCTCT
TTGTCAAAATTGCAGACAAGGTCATCAGCGTTCCTGAGAATGACTTCTTCTTTGACTTTGTT
CGACACTTGACAGACTGGATAAAGAAAGCTCGGCCCATCAAGGACGGAATTGTGCCCTCAC
TCACCTACCAGGTGTTCTTCATGAAGAAGCTGTGGACCACCACGGTGCCAGGGAAGGATCC
CATGGCCGATTCCATCTTCCACTATTACCAGGAGTTGCCCAAGTATCTCCGAGGCTACCACA
AGTGCACGCGGGAGGAGGTGCTGCAGCTGGGGGCGCTGATCTACAGGGTCAAGTTCGAGG
AGGACAAGTCCTACTTCCCCAGCATCCCCAAGCTGCTGCGGGAGCTGGTGCCCCAGGACCT
TATCCGGCAGGTCTCACCTGATGACTGGAAGCGGTCCATCGTCGCCTACTTCAACAAGCAC
GCAGGGAAGTCCAAGGAGGAGGCCAAGCTGGCCTTCCTGAAGCTCATCTTCAAGTGGCCCA
CCTTTGGCTCAGCCTTCTTCGAGGTGAAGCAAACTACGGAGCCAAACTTCCCTGAGATCCTC
CTAATTGCCATCAACAAGTATGGGGTCAGCCTCATCGATCCCAAAACGAAGGATATCCTCA
CCACTCATCCCTTCACCAAGATCTCCAACTGGAGCAGCGGCAACACCTACTTCCACATCACC
ATTGGGAACTTGGTGCGCGGGAGCAAACTGCTCTGCGAGACGTCACTGGGCTACAAGATGG
ATGACCTCCTGACTTCCTACATTAGCCAGATGCTCACAGCCATGAGCAAACAGCGGGGCTC
CAGGAGCGGCAAGTACCCTTACGATGTACCGGATTACGCATGAGGTACCAAGGGCGAATTC
TGCAGTCGACTAGAGCTCGCTGATCAGCCTCGACTGTGCCTTCTAGTTGCCAGCCATCTGTT
GTTTGCCCCTCCCCCGTGCCTTCCTTGACCCTGGAAGGTGCCACTCCCACTGTCCTTTCCTAA
TAAAATGAGGAAATTGCATCGCATTGTCTGAGTAGGTGTCATTCTATTCTGGGGGGTGGGG
TGGGGCAGGACAGCAAGGGGGAGGATTGGGAAGACAATAGCAGGCATGCTGGGGAGAGA
TCTGAGGACTAGTCCGTCGACTGTTAATTAAGCATGCTGGGGAGAGATCTAGGAAACCCCT
AGTGATGGAGTTGGCCACTCCCTCTCTGCGCGCTCGCTCGCTCACTGAGGCCGGGCGACCA
AAGGTCGCCCGACGCCCGGGCTTTGCCCGGGCGGCCTCAGTGAGCGAGCGAGCGCGCAGA
GAGGGAG
SEQ ID NO: 71 myosin7A (e.g., AAV-APhead-APSA-hMYO7ACT.HA)
GACCTGGAGCGAGGGCGGAGGGAGATGGTGGAGGAGGACCTGGATGCAGCCCTGCCCCTG
CCTGACGAGGATGAGGAGGACCTCTCTGAGTATAAATTTGCCAAGTTCGCGGCCACCTACT
TCCAGGGGACAACCACGCACTCCTACACCCGGCGGCCACTCAAACAGCCACTGCTCTACCA
TGACGACGAGGGTGACCAGCTGGCAGCCCTGGCGGTCTGGATCACCATCCTCCGCTTCATG
GGGGACCTCCCTGAGCCCAAGTACCACACAGCCATGAGTGATGGCAGTGAGAAGATCCCTG
TGATGACCAAGATTTATGAGACCCTGGGCAAGAAGACGTACAAGAGGGAGCTGCAGGCCC
TGCAGGGCGAGGGCGAGGCCCAGCTCCCCGAGGGCCAGAAGAAGAGCAGTGTGAGGCACA
AGCTGGTGCATTTGACTCTGAAAAAGAAGTCCAAGCTCACAGAGGAGGTGACCAAGAGGC
TGCATGACGGGGAGTCCACAGTGCAGGGCAACAGCATGCTGGAGGACCGGCCCACCTCCA
ACCTGGAGAAGCTGCACTTCATCATCGGCAATGGCATCCTGCGGCCAGCACTCCGGGACGA
GATCTACTGCCAGATCAGCAAGCAGCTGACCCACAACCCCTCCAAGAGCAGCTATGCCCGG
GGCTGGATTCTCGTGTCTCTCTGCGTGGGCTGTTTCGCCCCCTCCGAGAAGTTTGTCAAGTA
CCTGCGGAACTTCATCCACGGGGGCCCGCCCGGCTACGCCCCGTACTGTGAGGAGCGCCTG
AGAAGGACCTTTGTCAATGGGACACGGACACAGCCGCCCAGCTGGCTGGAGCTGCAGGCC
ACCAAGTCCAAGAAGCCAATCATGTTGCCCGTGACATTCATGGATGGGACCACCAAGACCC
TGCTGACGGACTCGGCAACCACGGCCAAGGAGCTCTGCAACGCGCTGGCCGACAAGATCTC
TCTCAAGGACCGGTTCGGGTTCTCCCTCTACATTGCCCTGTTTGACAAGGTGTCCTCCCTGG
GCAGCGGCAGTGACCACGTCATGGACGCCATCTCCCAGTGCGAGCAGTACGCCAAGGAGC
AGGGCGCCCAGGAGCGCAACGCCCCCTGGAGGCTCTTCTTCCGCAAAGAGGTCTTCACGCC
CTGGCACAGCCCCTCCGAGGACAACGTGGCCACCAACCTCATCTACCAGCAGGTGGTGCGA
GGAGTCAAGTTTGGGGAGTACAGGTGTGAGAAGGAGGACGACCTGGCTGAGCTGGCCTCC
CAGCAGTACTTTGTAGACTATGGCTCTGAGATGATCCTGGAGCGCCTCCTGAACCTCGTGCC
CACCTACATCCCCGACCGCGAGATCACGCCCCTGAAGACGCTGGAGAAGTGGGCCCAGCTG
GCCATCGCCGCCCACAAGAAGGGGATTTATGCCCAGAGGAGAACTGATGCCCAGAAGGTC
AAAGAGGATGTGGTCAGTTATGCCCGCTTCAAGTGGCCCTTGCTCTTCTCCAGGTTTTATGA
AGCCTACAAATTCTCAGGCCCCAGTCTCCCCAAGAACGACGTCATCGTGGCCGTCAACTGG
ACGGGTGTGTACTTTGTGGATGAGCAGGAGCAGGTACTTCTGGAGCTGTCCTTCCCAGAGA
TCATGGCCGTGTCCAGCAGCAGGGGAGCGAAAACGACGGCCCCCAGCTTCACGCTGGCCAC
CATCAAGGGGGACGAATACACCTTCACCTCCAGCAATGCTGAGGACATTCGTGACCTGGTG
GTCACCTTCCTAGAGGGGCTCCGGAAGAGATCTAAGTATGTTGTGGCCCTGCAGGATAACC
CCAACCCCGCAGGCGAGGAGTCAGGCTTCCTCAGCTTTGCCAAGGGAGACCTCATCATCCT
GGACCATGACACGGGCGAGCAGGTCATGAACTCGGGCTGGGCCAACGGCATCAATGAGAG
GACCAAGCAGCGTGGGGACTTCCCCACCGACAGTGTGTACGTCATGCCCACTGTCACCATG
CCACCGCGGGAGATTGTGGCCCTGGTCACCATGACTCCCGATCAGAGGCAGGACGTTGTCC
GGCTCTTGCAGCTGCGAACGGCGGAGCCCGAGGTGCGTGCCAAGCCCTACACGCTGGAGGA
GTTTTCCTATGACTACTTCAGGCCCCCACCCAAGCACACGCTGAGCCGTGTCATGGTGTCCA
AGGCCCGAGGCAAGGACCGGCTGTGGAGCCACACGCGGGAACCGCTCAAGCAGGCGCTGC
TCAAGAAGCTCCTGGGCAGTGAGGAGCTCTCGCAGGAGGCCTGCCTGGCCTTCATTGCTGT
GCTCAAGTACATGGGCGACTACCCGTCCAAGAGGACACGCTCCGTCAACGAGCTCACCGAC
CAGATCTTTGAGGGTCCCCTGAAAGCCGAGCCCCTGAAGGACGAGGCATATGTGCAGATCC
TGAAGCAGCTGACCGACAACCACATCAGGTACAGCGAGGAGCGGGGTTGGGAGCTGCTCT
GGCTGTGCACGGGCCTTTTCCCACCCAGCAACATCCTCCTGCCCCACGTGCAGCGCTTCCTG
CAGTCCCGAAAGCACTGCCCACTCGCCATCGACTGCCTGCAACGGCTCCAGAAAGCCCTGA
GAAACGGGTCCCGGAAGTACCCTCCGCACCTGGTGGAGGTGGAGGCCATCCAGCACAAGA
CCACCCAGATTTTCCACAAAGTCTACTTCCCTGATGACACTGACGAGGCCTTCGAAGTGGA
GTCCAGCACCAAGGCCAAGGACTTCTGCCAGAACATCGCCACCAGGCTGCTCCTCAAGTCC
TCAGAGGGATTCAGCCTCTTTGTCAAAATTGCAGACAAGGTCATCAGCGTTCCTGAGAATG
ACTTCTTCTTTGACTTTGTTCGACACTTGACAGACTGGATAAAGAAAGCTCGGCCCATCAAG
GACGGAATTGTGCCCTCACTCACCTACCAGGTGTTCTTCATGAAGAAGCTGTGGACCACCA
CGGTGCCAGGGAAGGATCCCATGGCCGATTCCATCTTCCACTATTACCAGGAGTTGCCCAA
GTATCTCCGAGGCTACCACAAGTGCACGCGGGAGGAGGTGCTGCAGCTGGGGGCGCTGATC
TACAGGGTCAAGTTCGAGGAGGACAAGTCCTACTTCCCCAGCATCCCCAAGCTGCTGCGGG
AGCTGGTGCCCCAGGACCTTATCCGGCAGGTCTCACCTGATGACTGGAAGCGGTCCATCGT
CGCCTACTTCAACAAGCACGCAGGGAAGTCCAAGGAGGAGGCCAAGCTGGCCTTCCTGAA
GCTCATCTTCAAGTGGCCCACCTTTGGCTCAGCCTTCTTCGAGGTGAAGCAAACTACGGAGC
CAAACTTCCCTGAGATCCTCCTAATTGCCATCAACAAGTATGGGGTCAGCCTCATCGATCCC
AAAACGAAGGATATCCTCACCACTCATCCCTTCACCAAGATCTCCAACTGGAGCAGCGGCA
ACACCTACTTCCACATCACCATTGGGAACTTGGTGCGCGGGAGCAAACTGCTCTGCGAGAC
GTCACTGGGCTACAAGATGGATGACCTCCTGACTTCCTACATTAGCCAGATGCTCACAGCC
ATGAGCAAACAGCGGGGCTCCAGGAGCGGCAAG
SEQ ID NO: 72 (hemagglutinin (HA) tag (AAV-APhead-APSA-hMYO7ACT.HA,
AAV-hMYO7A-CTlong.HA)
TACCCTTACGATGTACCGGATTACGCATGA

### Example 6 Shows Second Generation Hybrid Vectors that Minimize Expression of Truncated MYO7A Protein

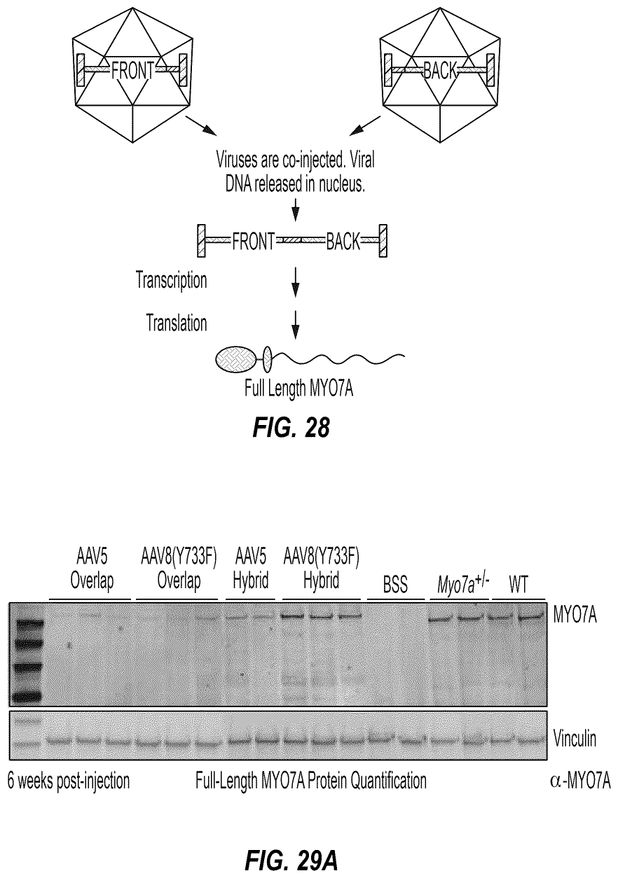

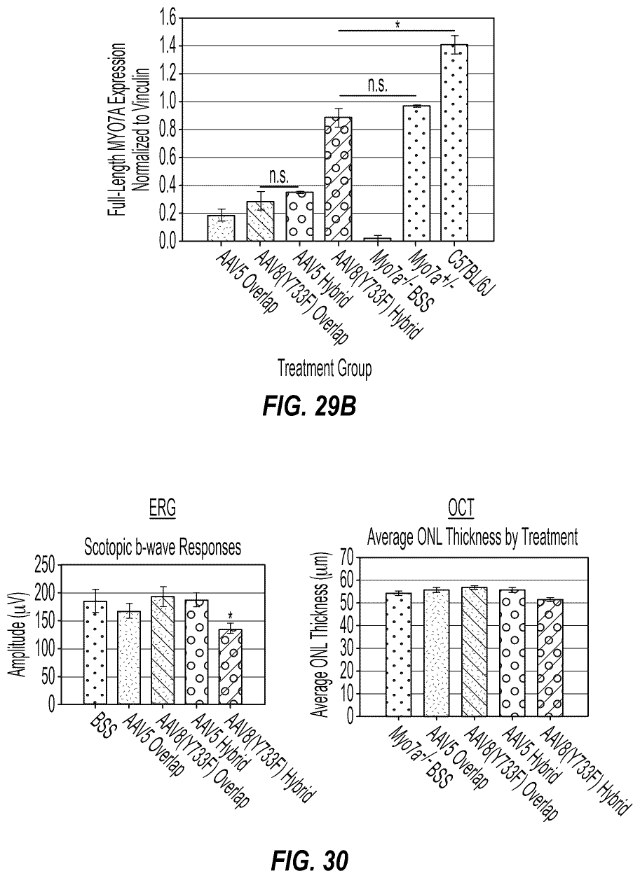

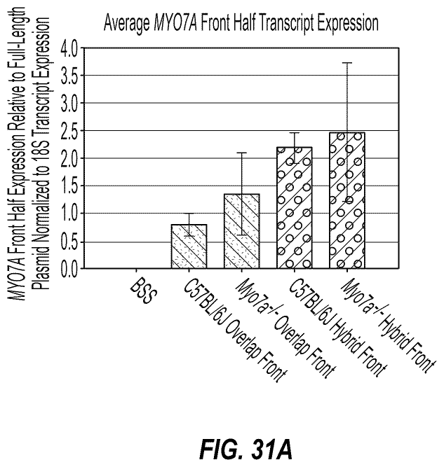

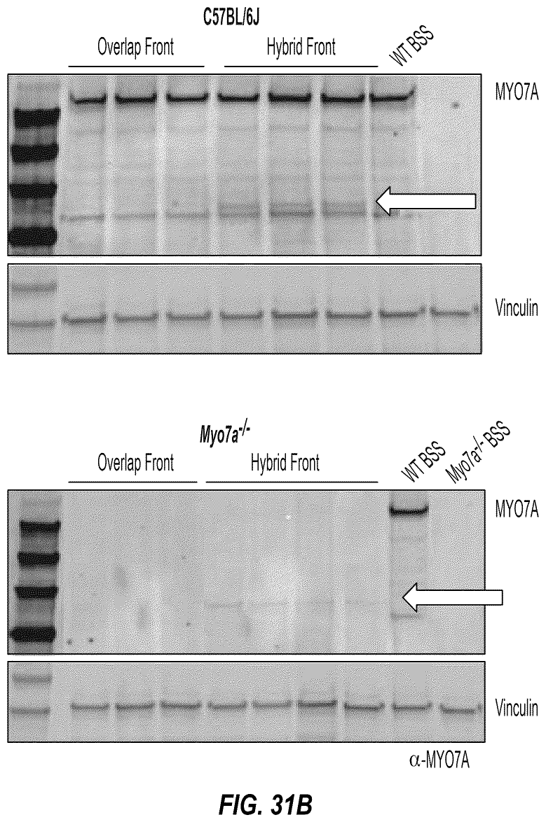

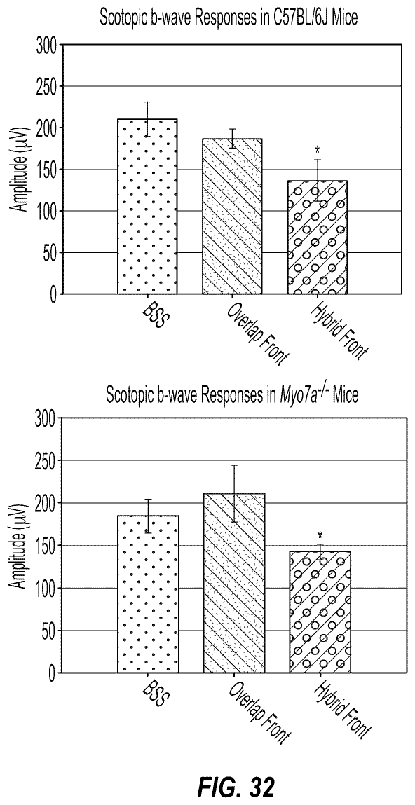

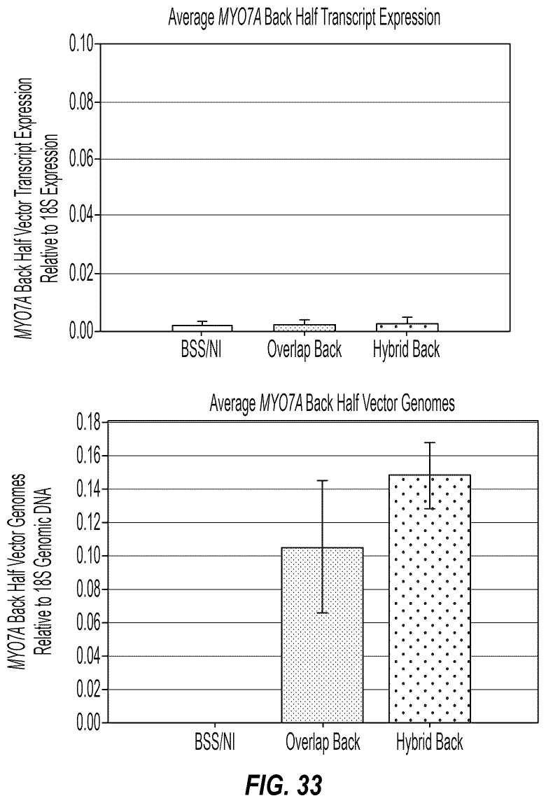

The hybrid and simple overlap front half vectors as described in Examples 1-5 contained a MYO7A cDNA sequence that encoded a portion of the MYO7A protein tail domain. The tail domain of MYO7A is known for its ability to bind other cellular proteins. In Examples 1-5 of the present application, it is shown that the original hybrid front half vector was capable of encoding a MYO7A protein (FIGS. 28-30) (Dyka, et al. 2014). However, some loss of retinal structure/function was observed following injection of original hybrid front half vectors into mouse retina. This loss of retinal structure/function was hypothesized to be a result of the gain of function, which is exerted by the truncated MYO7A protein containing a partial tail domain. The truncated MYO7A protein was shown to be produced from only the front-half vectors (FIGS. 31-32). As a control, the expression levels of any truncated protein encoded by the back-half vectors was also measured. These results demonstrate that back-half vectors do not produce a truncated product and do not lead to a loss of retinal structure/function (FIG. 33).

To eliminate this gain of function toxicity associated with the truncated MYO7A protein fragment, improved, second generation hybrid and simple overlap vectors were developed with the goal of eliminating the formation of the truncated protein from the front-half vectors. Though the previously developed simple overlap vector did not produce observable quantities of truncated MYO7A protein, and did not exhibit observable loss of structure/function following its injection in mice, a second generation simple overlap vector was nevertheless developed in conjunction with the second generation hybrid vector. This was done as a precaution to hedge against the possibility that the original simple overlap front-half vector may express truncated MYO7A at levels unable to be detected via immunoblot and tolerability studies in mouse.

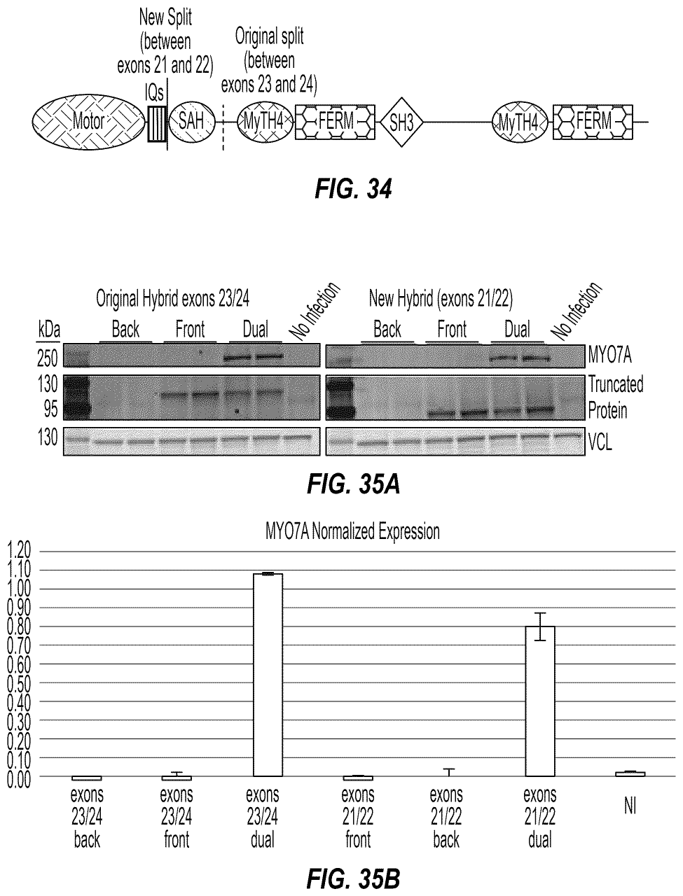

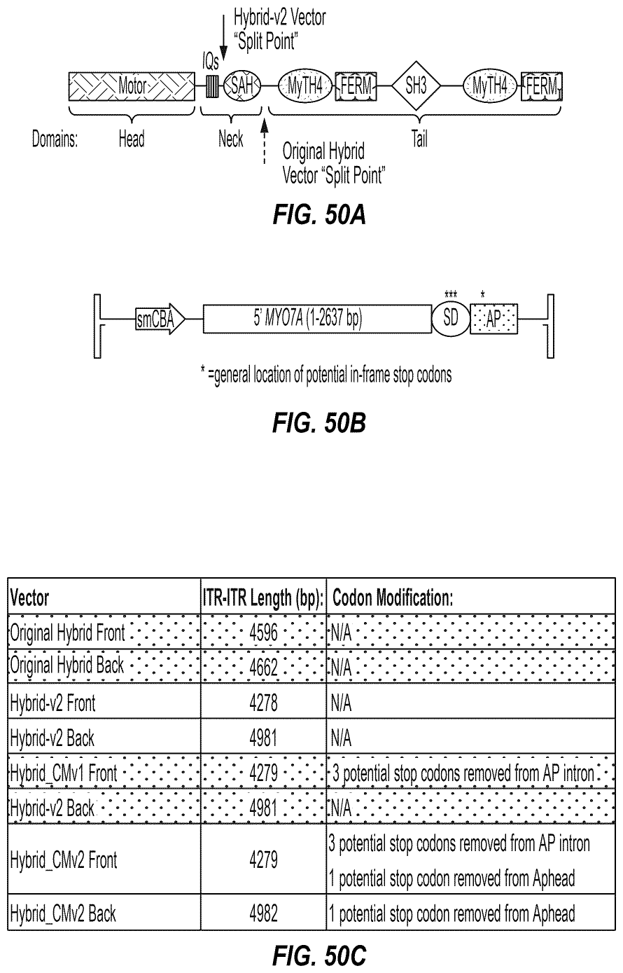

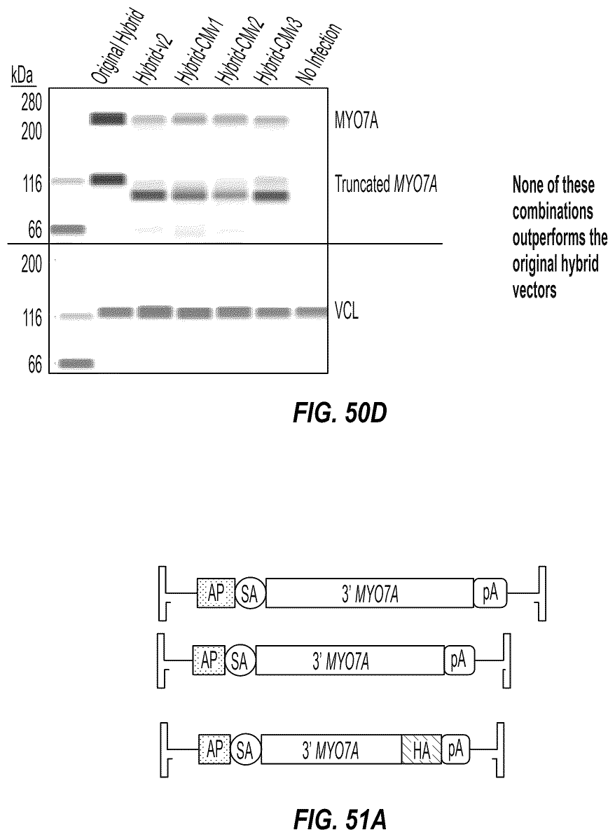

In both the hybrid and simple overlap platforms, sequence corresponding to the tail domain from the front-half vectors was moved to the back-half vectors. The MYO7A protein is an actin-based molecular motor, wherein the N-terminal (head) contains an actin-binding site and an ATP-binding site. The 5IQ (neck) is stabilized by calmodulin, and there is a single a-helix (SAH) that acts as a lever. The C-terminal (tail) domain of the MYO7A protein determines the functional specificity. Notably, in the hybrid vector, the ‘split point’ was moved from exon 23/24 in the original to exon 21/22 in the second generation (FIGS. 34 and 50A), such that the second generation vector systems have the split point moved from one side of the single alpha-helix (SAH) to the other. This new split between exons 21 and 22 is positioned between the 5IQ (neck) and SAH.

In the simple overlap vector, the amount of overlap sequence was reduced such that no sequence corresponding to the tail domain remained in the front vector (this new vector construct will be hereinafter referred to as the “second generation overlap”). Thus, the second generation overlap vector would contain a shorter segment of overlapping sequence such that the overlap ended at the split point between exons 21 and 22. The second generation hybrid and second generation overlap vectors described herein ensure that no portion of tail domain is encoded by either the hybrid front-, or simple overlap front-half vector. The vector systems were altered in this way to reduce production of truncated MYO7A protein.

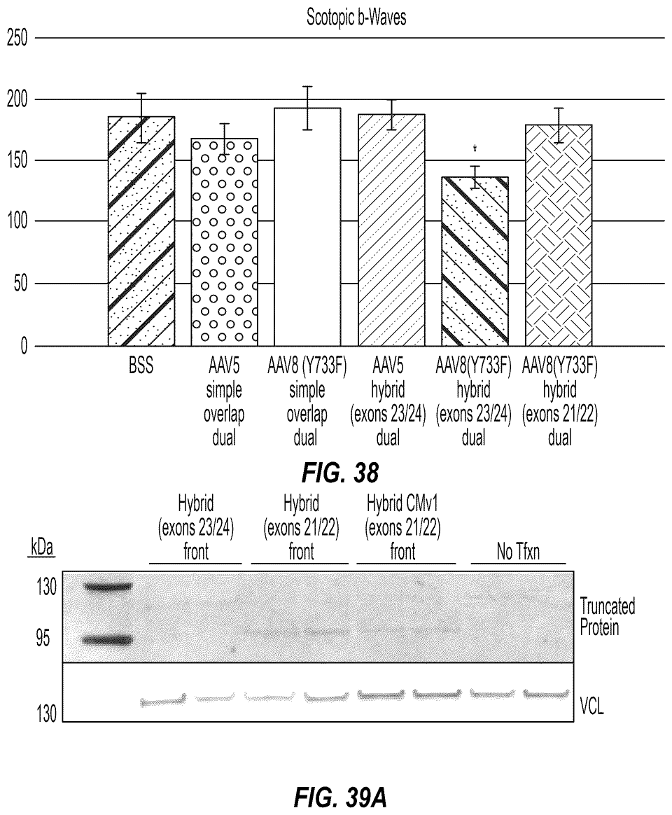

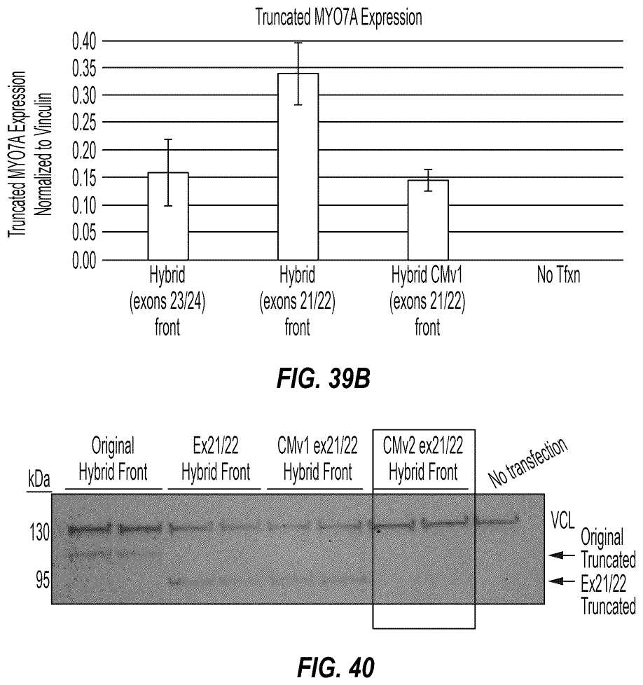

When the second generation hybrid vectors containing the exon 21/22 split point were administered in vitro to HEK293 cells, a truncated MYO7A protein of smaller size, corresponding to the change in vector sequence was observed (FIGS. 35 and 39). When the second generation hybrid vectors containing the exon 21/22 split point were administered in vivo via subretinal injection in Myo7a^{-/-} mice, the smaller sized MYO7A protein was also observed (FIGS. 36 and 38). The second generation hybrid vectors containing the exon 21/22 split point express equivalent amounts of full-length MYO7A compared to original hybrid vectors containing the exon 23/24 split point (see, e.g., FIGS. 35 and 36), but do not produce the MYO7A tail protein fragment observed in original hybrid vectors (FIG. 38). In sum, it is demonstrated herein that the second generation hybrid and simple overlap vectors that do not express a tail domain sequence in the front half vectors result in a MYO7A protein product that is better tolerated in retina.

The front half AAV-MYO7A vectors (both hybrid and simple overlap) contain promoters and inverted terminal repeats (ITRs). It is possible that the ITRs provide a polyadenylation signal that, together with the promoter, leads to the production and maturation of messenger RNA for translation. In the context of the hybrid vectors, it is also possible that the alkaline phosphatase (AP) splice donor and APhead ‘intron’ sequence are also facilitating maturation of the mRNA by providing a splicing signal. While the exact mechanism promoting mRNA maturation is unclear, it is observable that hybrid front half vectors do produce truncated protein (FIGS. 31-33).

Though it is demonstrated herein that the second generation hybrid vectors result in diminished production of undesired products, additional improvements were made to the vectors to reduce the production of truncated MYO7A, thereby further increasing safety and efficiency. In the hybrid vectors, which contain the second generation exon 21/22 split point as described above, potential in-frame stop codons located downstream of the MYO7A sequence were removed from the front hybrid vector plasmids (FIG. 42). The single nucleotide substitutions removing these potential stop codons are designed to take advantage of the ‘non-stop’ decay mechanism of the cell, thereby eliminating the spurious RNA before it can be translated. In-frame stop codons were removed in two stages. First, three potential stop codons within the AP splice donor sequence were removed from the second generation hybrid vector to create a further improved vector still containing the exon 21/22 split point (“CMv1 hybrid”; SEQ ID NOs: 33 and 32; FIG. 42).

An additional potential in-frame stop codon was located within the shared recombinogenic AP region. Thus, a separate, further improved vector was generated wherein there were functional improvements made in both the front-half and back-half second generation hybrid vectors containing the exon 21/22 split point (“CMv2 hybrid”; SEQ ID NOs: 34 and 35). The CMv2 hybrid front half vector has the three potential stop codons located in the AP splice donor sequence, as well as the one potential stop codon located in the APhead recombinogenic sequence, removed. The CMv2 hybrid back half vector has an identical change made in the APhead recombinogenic sequence so as to match the front half vector.

Upon making the modifications that result in the CMv2 hybrid back-half vector as described herein, the CMv2 hybrid back-half vector becomes close to exceeding, but does not actually exceed, the packaging limit of an AAV vector construct. To mitigate the possibility that the back-half vector is too large, a construct was designed that modifies the CMv2 hybrid back-half vector to remove any extraneous sequence existing between elements in the construct, as well as the HA sequence. Once modified, the resulting back-half vector is designated as the CMv2.1 hybrid back-half vector (SEQ ID NO: 44).

The CMv1 and CMv2 hybrid vectors were tested in vitro, by transfecting these plasmids into HEK293 cells. The first generation hybrid front half vector (“original”) and the second generation hybrid front half vector (“ex21/22”) were also used for comparison (FIG. 40). It was found that the CMv1 hybrid front half vector still produced truncated MYO7A. However, the CMv2 hybrid front half vector did not produce any truncated protein. Vinculin (VCL) was used as a loading control in all samples.

Corresponding changes were made in the simple overlap vector, despite the absence of truncated protein encoded by this vector (FIG. 43). Because the putative stop codons that were removed in the CMv1 and CMv2 hybrid vectors do not exist in the front-half of the simple overlap vectors, slightly different modifications were made to the simple overlap vectors. In the CMv1 overlap vector, the simple overlap vector has been modified in the 3′ untranslated region, between the MYO7A partial coding sequence and the 3′ AAV ITR, to remove potential in-frame stop codons (“CMv1 overlap”; SEQ ID NOs: 36 and 38; FIG. 43).

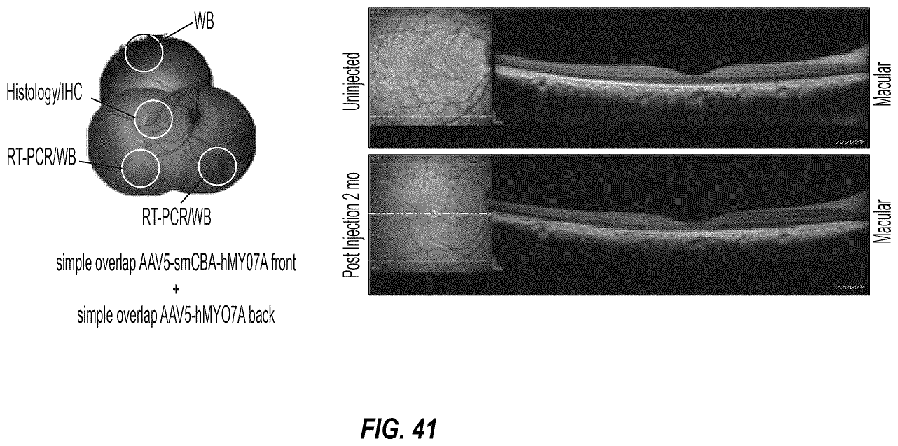

As shown in FIG. 41, AAV-mediated MYO7A transcript is expressed in macaque retina, and tolerability of dual AAV5-MYO7A vectors in subretinally injected macaque retinas is shown. Macaque retinas were evaluated 2 months after injection.

These data show great promise for application of the technology to human patients.

### Example 7 Provides Third Generation Hybrid Vectors

### Third Generation Overlap Vectors

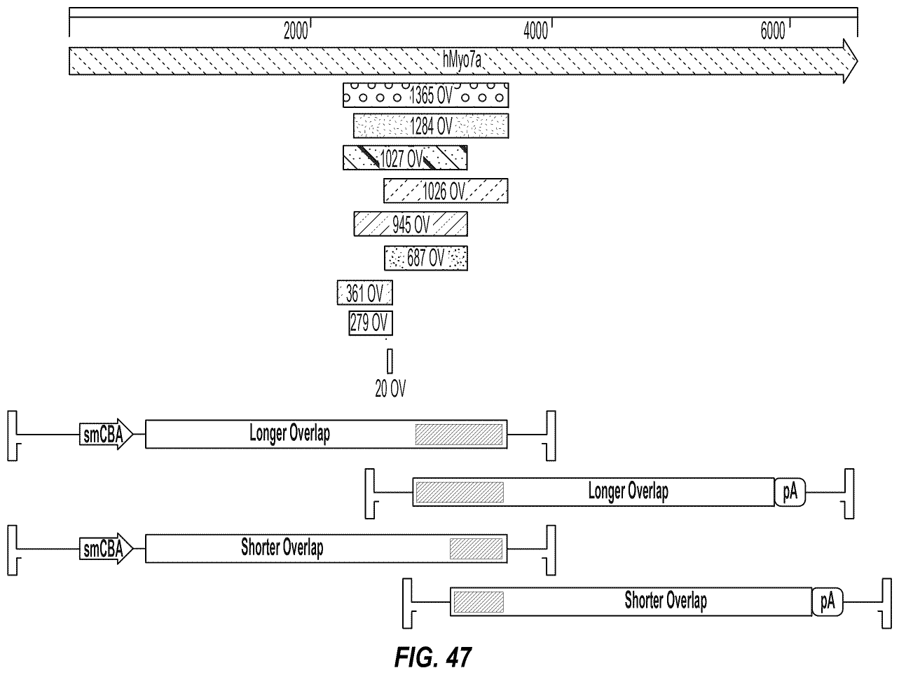

An improved third generation (V3) overlap vector pair was generated by altering the overlapping regions of the MYO7A coding sequence. This V3 overlap pair consists of a front half vector (“AAV-smCBA-hMYO7A-NTlong-v3”) comprising the nucleotide sequence of SEQ ID NO: 50, and a back half vector (“AAV-smCBA-hMYO7A-CTlong-v3.HA”) comprising the nucleotide sequence of SEQ ID NO: 51. This V3 overlap pair contains an N-terminal myosin 7A coding sequence comprising SEQ ID NO: 66 and a C-terminal myosin 7A coding sequence comprising SEQ ID NO: 80. These vectors contain shortened overlapping region length (see FIGS. 46 and 47), such as overlapping region lengths of 945 bp and 687 bp. Reduction of the length of the overlapping region has the effect of reducing the size of the vector genome overall.

The V3 Overlap dual vector system was shown to produce increased levels of full-length MYO7A protein as compared to the original vectors, as quantified by the Protein Simple Jess system, as shown in FIGS. 48A and 49A. Overlap vectors containing 687 bp and 945 bp of overlapping MYO7A sequence provided optimal expression levels.

As shown in FIG. 49B, the V3 overlap vectors do not produce an appreciable amount of undesired truncated MYO7A fragment when their overlapping region length remains above 361 bp. Thus, reducing overlap length to a certain point, and therefore ensuring neither vector genome is pushing the packaging capacity of AAV capsid (4.7 to 4.9 kb), leads to increased expression of full length MYO7A. If overlap length is too small (<361 bp), full length MYO7A expression is reduced, and truncated protein may appear.

By virtue of the shorter length of their overlapping regions, these improved, third generation overlap vectors contain shorter ITR to ITR (ITR-ITR) lengths (see FIG. 48B). In particular embodiments, the length between the inverted terminal repeats at each end of the first AAV vector polynucleotide is about 4615 nucleotides (nt) or fewer. The ITR-ITR length in the improved first vector polynucleotide is 4615 nt. In particular embodiments, the length between the inverted terminal repeats at each end of the second AAV vector polynucleotide is about 4800 nt or fewer. The ITR-ITR length in the improved second vector polynucleotide is about 4560 nt. hMYO7A overlapping regions, e.g., SEQ ID NOs: 39 and 53-59, may be used as the polynucleotide sequence that overlaps in additional overlap dual vectors expressing large genes (other than MYO7A). In particular, disclosed herein are overlap dual vectors that express a large gene other than MYO7A and that comprises a nucleotide sequence having at least 80%, 85%, 90%, 95%, 98%, or 99% identity to any one of SEQ ID NOs: 39 and 52-59. In particular embodiments, these overlap dual vectors comprises the nucleotide sequence of any one of SEQ ID NOs: 39 and 53-59, e.g., SEQ ID NO: 56 or 57, and express a large gene selected from ABCA4, CEP290, EYS, RP1, ALMS1, CDH23, PCDH15, USH1C, USH1G, USH2A, DNFB31, DMD, CFTR, GDE, DYSF, F8, and DFNB2. In some embodiments, these overlap vectors contain two overlapping sequences disclosed herein, e.g., the mutually exclusive sequences SEQ ID NOs: 39 and 56, or the mutually exclusive sequences SEQ ID NOs: 39 and 57. rAAV virions containing V3 overlap vector pairs containing 687 bp and 945 bp of overlapping region length are packaged and administered to retinal cells. rAAV virions containing V3 overlap vector pairs containing 687 bp and 945 bp of overlapping region length are packaged and administered to auditory hair cells.

Overlap vectors containing the following pairs of myosin7A-encoding nucleotide sequences are evaluated in their abilities to produce full-length MYO7A polypeptide, in vitro or in vivo. In vitro evaluation may be performed using Protein Simple Jess Western blotting.
the first AAV vector polynucleotide comprises the nucleotide sequence of SEQ ID NO: 63, and the second AAV vector polynucleotide comprises the nucleotide sequence of SEQ ID NO: 83;
the first AAV vector polynucleotide comprises the nucleotide sequence of SEQ ID NO: 63, and the second AAV vector polynucleotide comprises the nucleotide sequence of SEQ ID NO: 90;
the first AAV vector polynucleotide comprises the nucleotide sequence of SEQ ID NO: 101, and the second AAV vector polynucleotide comprises the nucleotide sequence of SEQ ID NO: 83;
the first AAV vector polynucleotide comprises the nucleotide sequence of SEQ ID NO: 101, and the second AAV vector polynucleotide comprises the nucleotide sequence of SEQ ID NO: 90;
the first AAV vector polynucleotide comprises the nucleotide sequence of SEQ ID NO: 66, and the second AAV vector polynucleotide comprises the nucleotide sequence of SEQ ID NO: 83; and
the first AAV vector polynucleotide comprises the nucleotide sequence of SEQ ID NO: 66, and the second AAV vector polynucleotide comprises the nucleotide sequence of SEQ ID NO: 90.

Accordingly, in some embodiments, provided herein are polynucleotide vector systems in which the first AAV vector polynucleotide comprises a nucleotide sequence that is at least about 80%, at least 85%, at least 90%, at least 95%, at least 98%, or at least 99% identical to the SEQ ID NO: 63, and the second AAV vector polynucleotide comprises a nucleotide sequence that is at least about 80%, at least 85%, at least 90%, at least 95%, at least 98%, or at least 99% identical to nucleotide sequence of SEQ ID NO: 83. In some embodiments, the second AAV vector polynucleotide comprises a nucleotide sequence that is at least about 80%, at least 85%, at least 90%, at least 95%, at least 98%, or at least 99% identical to SEQ ID NO: 90.

In some embodiments, the first AAV vector polynucleotide comprises a nucleotide sequence that is at least about 80%, at least 85%, at least 90%, at least 95%, at least 98%, or at least 99% identical to SEQ ID NO: 101. In some embodiments, the first AAV vector polynucleotide comprises a nucleotide sequence that is at least about 80%, at least 85%, at least 90%, at least 95%, at least 98%, or at least 99% identical to SEQ ID NO: 66. In some embodiments, provided herein are polynucleotide vector systems in which the first AAV vector polynucleotide comprises a nucleotide sequence that is at least about 80%, at least 85%, at least 90%, at least 95%, at least 98%, or at least 99% identical to the SEQ ID NO: 66, and the second AAV vector polynucleotide comprises a nucleotide sequence that is at least about 80%, at least 85%, at least 90%, at least 95%, at least 98%, or at least 99% identical to nucleotide sequence of SEQ ID NO: 90.

The following overlap vectors are evaluated in their abilities to produce full-length MYO7A polypeptide, in vitro or in vivo. In vitro evaluation may be performed using Protein Simple Jess Western blotting.
the first AAV vector comprises the nucleotide sequence of SEQ ID NO: 50, and the second AAV vector comprises the nucleotide sequence of SEQ ID NO: 51;
the first AAV vector comprises the nucleotide sequence of SEQ ID NO: 50, and the second AAV vector comprises the nucleotide sequence of SEQ ID NO: 38;
the first AAV vector comprises the nucleotide sequence of SEQ ID NO: 1, and the second AAV vector comprises the nucleotide sequence of SEQ ID NO: 38;
the first AAV vector comprises the nucleotide sequence of SEQ ID NO: 50, and the second AAV vector comprises the nucleotide sequence of SEQ ID NO: 2;
the first AAV vector comprises the nucleotide sequence of SEQ ID NO: 1, and the second AAV vector comprises the nucleotide sequence of SEQ ID NO: 51;
the first AAV vector comprises the nucleotide sequence of SEQ ID NO: 36, and the second AAV vector comprises the nucleotide sequence of SEQ ID NO: 2;
the first AAV vector comprises the nucleotide sequence of SEQ ID NO: 36, and the second AAV vector comprises the nucleotide sequence of SEQ ID NO: 38;
the first AAV vector comprises the nucleotide sequence of SEQ ID NO: 36, and the second AAV vector comprises the nucleotide sequence of SEQ ID NO: 51;
the first AAV vector comprises the nucleotide sequence of SEQ ID NO: 37, and the second AAV vector comprises the nucleotide sequence of SEQ ID NO: 38; and
the first AAV vector comprises the nucleotide sequence of SEQ ID NO: 37, and the second AAV vector comprises the nucleotide sequence of SEQ ID NO: 51.

Accordingly, in some embodiments, provided herein are polynucleotide vector systems in which the first AAV vector polynucleotide comprises a nucleotide sequence that is at least about 80%, at least 85%, at least 90%, at least 95%, at least 98%, or at least 99% identical to the SEQ ID NO: 50, and the second AAV vector polynucleotide comprises a nucleotide sequence that is at least about 80%, at least 85%, at least 90%, at least 95%, at least 98%, or at least 99% identical to nucleotide sequence of SEQ ID NO: 51. In some embodiments, the second AAV vector polynucleotide comprises a nucleotide sequence that is at least about 80%, at least 85%, at least 90%, at least 95%, at least 98%, or at least 99% identical to SEQ ID NO: 38. In some embodiments, the second AAV vector polynucleotide comprises a nucleotide sequence that is at least about 80%, at least 85%, at least 90%, at least 95%, at least 98%, or at least 99% identical to SEQ ID NO: 2.

In some embodiments, the first AAV vector polynucleotide comprises a nucleotide sequence that is at least about 80%, at least 85%, at least 90%, at least 95%, at least 98%, or at least 99% identical to SEQ ID NO: 1. In some embodiments, the first AAV vector polynucleotide comprises a nucleotide sequence that is at least about 80%, at least 85%, at least 90%, at least 95%, at least 98%, or at least 99% identical to SEQ ID NO: 36. In some embodiments, the first AAV vector polynucleotide comprises a nucleotide sequence that is at least about 80%, at least 85%, at least 90%, at least 95%, at least 98%, or at least 99% identical to SEQ ID NO: 37. In some embodiments, provided herein are polynucleotide vector systems in which the first AAV vector polynucleotide comprises a nucleotide sequence that is at least about 80%, at least 85%, at least 90%, at least 95%, at least 98%, or at least 99% identical to the SEQ ID NO: 37, and the second AAV vector polynucleotide comprises a nucleotide sequence that is at least about 80%, at least 85%, at least 90%, at least 95%, at least 98%, or at least 99% identical to nucleotide sequence of SEQ ID NO: 38 or SEQ ID NO: 51.

### Third Generation Hybrid Vectors

An improved third generation (CMv3) hybrid system, or vector pair, was generated by making substitutions into three putative stop codons in the 3′ untranslated region (UTR) of the front-half vector of the CMv2 hybrid system. As such, the CMv3 Hybrid Front Half vector contains substitutions in (i.e., removal of) one in-frame stop codon in the APhead sequence, three in-frame stop codons in the AP intron sequence, and three in-frame stop codons in the 3′ UTR sequence. In exemplary embodiments, the CMv3 hybrid system is a CMv3 MIN system as residual, unseeded legacy sequences (such as restriction enzyme sites) have been removed.

Exemplary CMv3 hybrid systems consist of i) the front half vector (“AAV-smCBA-hMYO7A-NT-Ex21-APSD-APhead-CMv3”) comprising the nucleotide sequence of SEQ ID NO: 46, and ii) a CMv2 back half vector (“AAV-APhead-APSA-ex22hMYO7A-CT.HA-CMv2)”) comprising the nucleotide sequence of SEQ ID NO: 35, a CMv2.1 back half vector (“AAV-APhead-APSA-hMYO7ACTex22-CMv2.1”) comprising the nucleotide sequence of SEQ ID NO: 44, or a minimized version of either vector, e.g. a CMv2 (or V2-)Back MIN comprising the nucleotide sequence of SEQ ID NO: 49.

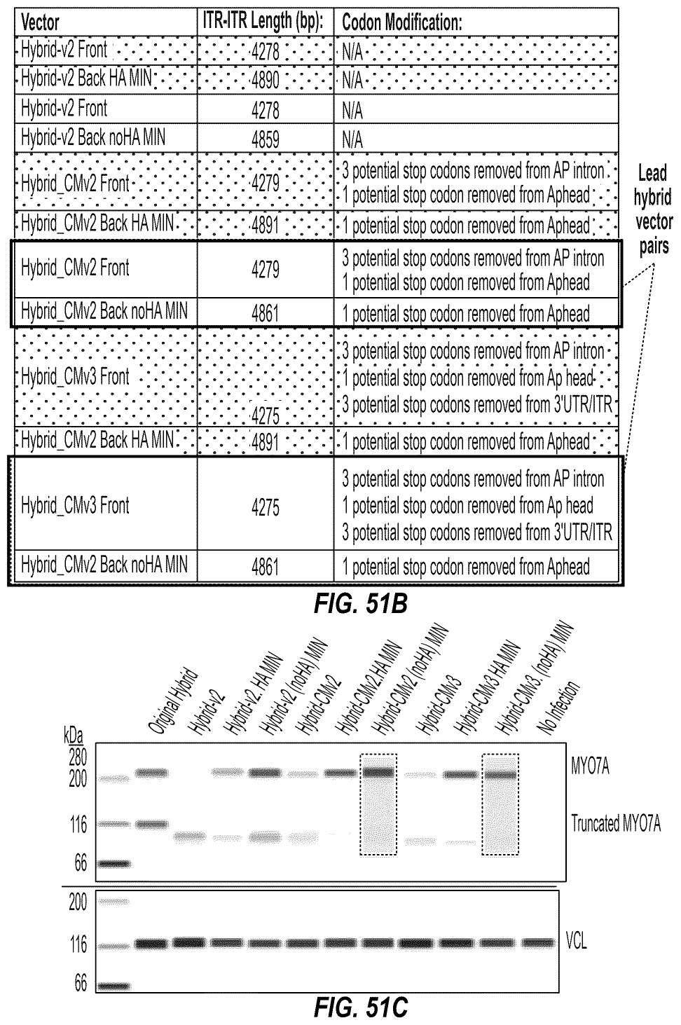

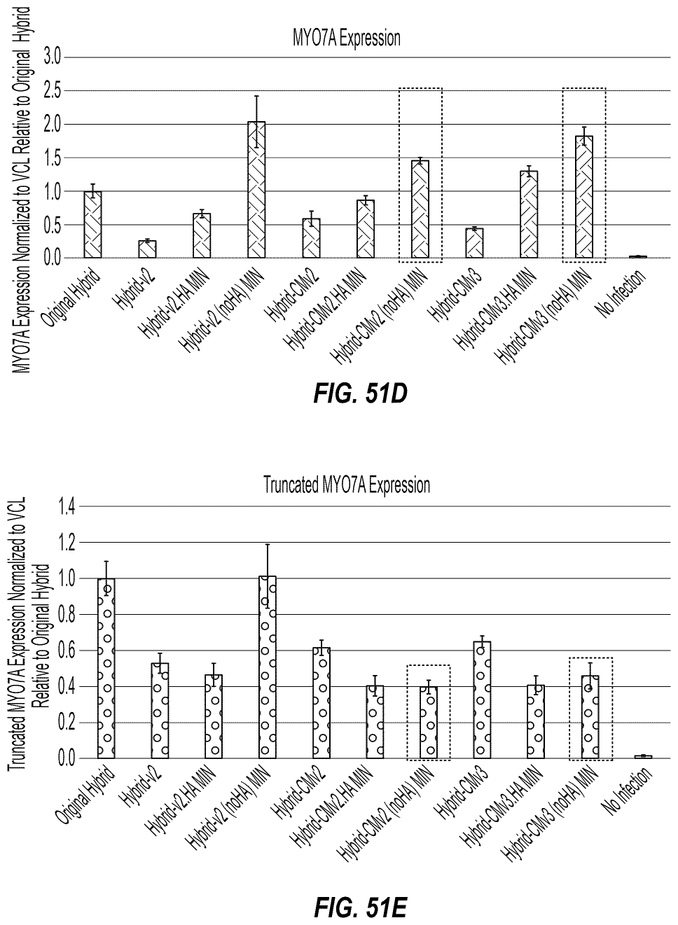

To generate the Hybrid-CMv3 back MIN and Hybrid-CMv2 back MIN vectors, an ‘unneeded legacy’ sequence was removed from the back half vector to ensure the vector size did not exceed the packaging capacity of an AAV capsid. By virtue of the removal of unseeded legacy sequences from the back, these improved, third generation hybrid vectors contain shorter ITR to ITR (ITR-ITR) lengths (see FIGS. 50C and 51B). In particular embodiments, the length between the inverted terminal repeats at each end of a first AAV vector polynucleotide of the hybrid vectors is about 4279 nucleotides (nt) or fewer. This has the effect of minimizing the length of the back half vector, which improved packaging efficiency. The Hybrid-V2 back MIN is 122 bp smaller (4981 vs. 4859 bp) than the associated original vector, and the Hybrid_CMv2 back MIN is 121 bp smaller (4982 vs. 4861 bp) than the associated original vector. For the Hybrid-V2 back MIN HA and Hybrid-CMV2 back MIN HA vectors, a hemagglutinin (HA) tag was added to the minimized back half vectors (Hybrid-V2 back MIN and Hybrid-CMV2 back MIN) to allow for detection in normal monkeys.

The CMv3 Hybrid vector system was shown to produce comparably low levels of the truncated MYO7A fragment as compared to Hybrid CMv1 and Hybrid CMv2 vectors, as shown in FIG. 50D. Further, as shown in FIGS. 51C and 51D, the pairing the hybrid CMv3 front half vector with a CMv3 MIN back half vector produced full-length MYO7A at levels equal to or above that seen with the original (1^{st} generation) hybrid vectors. This pair of vectors produced comparably low levels of truncated fragment, as shown in FIG. 51E.

The inventors have also discovered that wherein a hMYO7A sequence may be used as the intronic sequence mediating recombination in the cell following administration in hybrid dual vectors expressing large genes (other than MYO7A). Such hybrid vectors are generated to contain one or more overlapping regions identified through improvement of the MYO7A overlap vectors provided herein. (Such vectors may or do not contain an APhead sequence and/or an AP intronic sequence, to allow for the insertion of one of these overlapping regions.) In particular, disclosed herein are hybrid dual vectors that contain a sequence between the first intron and second intron of the first and second AAV vector polynucleotides, respectively, that comprises a nucleotide sequence having at least 80%, 85%, 90%, 95%, 98%, or 99% identity to any one of SEQ ID NOs: 39 and 52-59. In particular embodiments, hybrid dual vectors contain a sequence between the first intron and second intron of the first and second AAV vector polynucleotides, respectively, that comprises the nucleotide sequence of any one of SEQ ID NOs: 39 and 53-59, e.g., SEQ ID NO: 56 or 57. Additional large genes that may be delivered with these hybrid vectors include ABCA4, CEP290, EYS, RP1, ALMS1, CDH23, PCDH15, USH1C, USH1G, USH2A, DNFB31, DMD, CFTR, GDE, DYSF, F8, and DFNB2. In some embodiments, these hybrid dual vectors contain an intron sequence containing two overlapping sequences disclosed herein, e.g., the mutually exclusive sequences SEQ ID NOs: 39 and 56, or the mutually exclusive sequences SEQ ID NOs: 39 and 57. rAAV virions containing CMv3 hybrid vector pairs comprising the SEQ ID NO: 46 front-half vector and SEQ ID NO: 35 back-half vector are packaged and administered to retinal cells. rAAV virions containing CMv3 hybrid vector pairs comprising the SEQ ID NO: 46 front-half vector and SEQ ID NO: 35 back-half vector are packaged and administered to auditory hair cells.

Hybrid vectors containing the following pair of myosin7A-encoding nucleotide sequences are evaluated in their abilities to produce full-length MYO7A polypeptide, in vitro or in vivo:
the first AAV vector polynucleotide comprises the nucleotide sequence of SEQ ID NO: 73, and the second AAV vector polynucleotide comprises the nucleotide sequence of SEQ ID NO: 75.

Accordingly, in some embodiments, provided herein are polynucleotide vector systems in which the first AAV vector polynucleotide comprises a nucleotide sequence that is at least about 80%, at least 85%, at least 90%, at least 95%, at least 98%, or at least 99% identical to the SEQ ID NO: 73, and the second AAV vector polynucleotide comprises a nucleotide sequence that is at least about 80%, at least 85%, at least 90%, at least 95%, at least 98%, or at least 99% identical to nucleotide sequence of SEQ ID NO: 75.

Hybrid vector systems comprising the following pairs of vector sequences are evaluated in their abilities to produce full-length MYO7A polypeptide, in vitro or in vivo.
the first AAV vector comprises the nucleotide sequence of SEQ ID NO: 31, and the second AAV vector comprises the nucleotide sequence of SEQ ID NO: 32;
the first AAV vector comprises the nucleotide sequence of SEQ ID NO: 46, and the second AAV vector comprises the nucleotide sequence of SEQ ID NO: 35;
the first AAV vector comprises the nucleotide sequence of SEQ ID NO: 46, and the second AAV vector comprises the nucleotide sequence of SEQ ID NO: 49;
the first AAV vector comprises the nucleotide sequence of SEQ ID NO: 34, and the second AAV vector comprises the nucleotide sequence of SEQ ID NO: 47;
the first AAV vector comprises the nucleotide sequence of SEQ ID NO: 31, and the second AAV vector comprises the nucleotide sequence of SEQ ID NO: 48;
the first AAV vector comprises the nucleotide sequence of SEQ ID NO: 31, and the second AAV vector comprises the nucleotide sequence of SEQ ID NO: 49;
the first AAV vector comprises the nucleotide sequence of SEQ ID NO: 33, and the second AAV vector comprises the nucleotide sequence of SEQ ID NO: 32;
the first AAV vector comprises the nucleotide sequence of SEQ ID NO: 34, and the second AAV vector comprises the nucleotide sequence of SEQ ID NO: 35; and
the first AAV vector comprises the nucleotide sequence of SEQ ID NO: 34, and the second AAV vector comprises the nucleotide sequence of SEQ ID NO: 44.

Accordingly, in some embodiments, provided herein are polynucleotide vector systems in which the first AAV vector polynucleotide comprises a nucleotide sequence that is at least about 80%, at least 85%, at least 90%, at least 95%, at least 98%, or at least 99% identical to the SEQ ID NO: 31, and the second AAV vector polynucleotide comprises a nucleotide sequence that is at least about 80%, at least 85%, at least 90%, at least 95%, at least 98%, or at least 99% identical to nucleotide sequence of SEQ ID NO: 32. In some embodiments, the second AAV vector polynucleotide comprises a nucleotide sequence that is at least about 80%, at least 85%, at least 90%, at least 95%, at least 98%, or at least 99% identical to SEQ ID NO: 48 or SEQ ID NO: 49. In some embodiments, the first AAV vector polynucleotide comprises a nucleotide sequence that is at least about 80%, at least 85%, at least 90%, at least 95%, at least 98%, or at least 99% identical to SEQ ID NO: 33.

In some embodiments, provided herein are polynucleotide vector systems in which the first AAV vector polynucleotide comprises a nucleotide sequence that is at least about 80%, at least 85%, at least 90%, at least 95%, at least 98%, or at least 99% identical to the SEQ ID NO: 46, and the second AAV vector polynucleotide comprises a nucleotide sequence that is at least about 80%, at least 85%, at least 90%, at least 95%, at least 98%, or at least 99% identical to nucleotide sequence of SEQ ID NO: 35. In some embodiments, the second AAV vector polynucleotide comprises a nucleotide sequence that is at least about 80%, at least 85%, at least 90%, at least 95%, at least 98%, or at least 99% identical to SEQ ID NO: 49. In some embodiments, the first AAV vector polynucleotide comprises a nucleotide sequence that is at least about 80%, at least 85%, at least 90%, at least 95%, at least 98%, or at least 99% identical to SEQ ID NO: 34.

In some embodiments, provided herein are polynucleotide vector systems in which the first AAV vector polynucleotide comprises a nucleotide sequence that is at least about 80%, at least 85%, at least 90%, at least 95%, at least 98%, or at least 99% identical to the SEQ ID NO: 34, and the second AAV vector polynucleotide comprises a nucleotide sequence that is at least about 80%, at least 85%, at least 90%, at least 95%, at least 98%, or at least 99% identical to nucleotide sequence of SEQ ID NO: 47. In some embodiments, the second AAV vector polynucleotide comprises a nucleotide sequence that is at least about 80%, at least 85%, at least 90%, at least 95%, at least 98%, or at least 99% identical to SEQ ID NO: 35 or 44.

The polynucleotide vector systems of the disclosure may comprise a nucleotide sequence that is at least 80%, 85%, 90%, 92.5%, 95%, 96%, 97%, 98%, 99%, or 99.5% identical to any of the following sequences (e.g., any of SEQ ID NOs: 31-38, 44, and 46-51). In some embodiments, the vectors comprise a sequence comprising any one of SEQ ID NOs: 31-38, 44, and 46-51. In some embodiments, the vector systems of the disclosure comprise a nucleotide sequence that contains that differs from any of the sequences of SEQ ID NOs: 31-38, 44, and 46-51 by 1, 2, 3, 4, 5, 5-10, 10-15, 15-20, 20-25, 25-30, 30-35, 35-40, 40-45, 45-50, or more than 50 nucleotides. The disclosed vectors may differ in any of the following vector sequences in the presence, or absence, of a tag such as an HA tag. The disclosed vectors may contain stretches of 5-10, 10-15, 15-20, 20-25, 25-35, 35-45, 45-60, 60-75 or more than 75 consecutive nucleotides in common with any of SEQ ID NOs: 31-38, 44, and 46-51.

The myosin7a-encoding sequences of any of the polynucleotide vectors provided herein may comprise a nucleotide sequence that is at least 80%, 85%, 90%, 92.5%, 95%, 96%, 97%, 98%, 99%, or 99.5% identical to any of the hMyosin7a-encoding sequences in the N-terminal (front half, “—NT”) or C-terminal (back half, “—CT”) vectors that follow, e.g., SEQ ID NOs: 63, 66, 73, 75, 77, 80, and 90. The disclosed N-terminal and C-terminal myosin7a-encoding sequences may contain stretches of 5-10, 10-15, 15-20, 20-25, 25-35, 35-45, 45-60, 60-75 or more than 75 consecutive nucleotides in common with any of SEQ ID NOs: 63, 66, 73, 75, 77, 80, and 90.

The polynucleotide vectors and myosin7a-encoding sequences provided herein may comprise a nucleotide sequence that differs from any of the following sequences (e.g., any of the vectors of SEQ ID NOs: 31-38, 44, and 46-51; or any of the hMyosin7a-encoding sequences in the N-terminal (front half, “—NT”) or C-terminal (back half, “—CT”) vectors set forth as SEQ ID NOs: 63, 66, 73, 75, 77, 80, and 90) by 1, 2, 3, 4, 5, 6, 7, 8, 9, 10, 10-15, 15-20, 20-25, or more than 25 nucleotides. The polynucleotide vectors provided herein may comprise a nucleotide sequence that differs from any of the following sequences by 1, 2, 3, 4, 5, 6, 7, 8, 9, 10, or more than 10 nucleotides at or near the 5′ terminus of the vector; and may differ from any of these sequences by 1, 2, 3, 4, 5, 6, 7, 8, 9, 10, or more than 10 nucleotides at or near the 3′ terminus. The vectors provided herein may comprise truncations by 1, 2, 3, 4, 5, 6, 7, 8, 9, or 10 nucleotides at the 5′ or 3′ terminus.

### Nucleotide Sequences of the Vectors Provided in Examples 6 and 7

SEQ ID NO: 31 is the nucleotide sequence of the second generation hybrid
front-half vector (i.e., AAV-smCBA-hMYO7A-NT-Ex21-APSD-APhead):
CTCCCTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCC
ATCACTAGGGGTTCTCAGATCTGGCGCGCCCAATTCGGTACCCTAGTTATTAATAGTAATCA
ATTACGGGGTCATTAGTTCATAGCCCATATATGGAGTTCCGCGTTACATAACTTACGGTAAA
TGGCCCGCCTGGCTGACCGCCCAACGACCCCCGCCCATTGACGTCAATAATGACGTATGTTC
CCATAGTAACGCCAATAGGGACTTTCCATTGACGTCAATGGGTGGACTATTTACGGTAAAC
TGCCCACTTGGCAGTACATCAAGTGTATCATATGCCAAGTACGCCCCCTATTGACGTCAATG
ACGGTAAATGGCCCGCCTGGCATTATGCCCAGTACATGACCTTATGGGACTTTCCTACTTGG
CAGTACATCTACGTATTAGTCATCGCTATTACCATGGTCGAGGTGAGCCCCACGTTCTGCTT
CACTCTCCCCATCTCCCCCCCCTCCCCACCCCCAATTTTGTATTTATTTATTTTTTAATTATTT
TGTGCAGCGATGGGGGCGGGGGGGGGGGGGGGGCGCGCGCCAGGCGGGGCGGGGCGGGG
CGAGGGGCGGGGCGGGGCGAGGCGGAGAGGTGCGGCGGCAGCCAATCAGAGCGGCGCGC
TCCGAAAGTTTCCTTTTATGGCGAGGCGGCGGCGGCGGCGGCCCTATAAAAAGCGAAGCGC
GCGGCGGGCGGGAGTCGCTGCGCGCTGCCTTCGCCCCGTGCCCCGCTCCGCCGCCGCCTCG
CGCCGCCCGCCCCGGCTCTGACTGACCGCGTTACTCCCACAGGTGAGCGGGCGGGACGGCC
CTTCTCCTCCGGGCTGTAATTAGCGCTTGGTTTAATGACGGCTTGTTTCTTTTCTGTGGCTGC
GTGAAAGCCTTGAGGGGCTCCGGGAGCTAGAGCCTCTGCTAACCATGTTCATGCCTTCTTCT
TTTTCCTACAGCTCCTGGGCAACGTGCTGGTTATTGTGCTGTCTCATCATTTTGGCAAAGAA
TTCTAGCGGCCGCCACCATGGTGATTCTTCAGCAGGGGGACCATGTGTGGATGGACCTGAG
ATTGGGGCAGGAGTTCGACGTGCCCATCGGGGCGGTGGTGAAGCTCTGCGACTCTGGGCAG
GTCCAGGTGGTGGATGATGAAGACAATGAACACTGGATCTCTCCGCAGAACGCAACGCACA
TCAAGCCTATGCACCCCACGTCGGTCCACGGCGTGGAGGACATGATCCGCCTGGGGGACCT
CAACGAGGCGGGCATCTTGCGCAACCTGCTTATCCGCTACCGGGACCACCTCATCTACACG
TATACGGGCTCCATCCTGGTGGCTGTGAACCCCTACCAGCTGCTCTCCATCTACTCGCCAGA
GCACATCCGCCAGTATACCAACAAGAAGATTGGGGAGATGCCCCCCCACATCTTTGCCATT
GCTGACAACTGCTACTTCAACATGAAACGCAACAGCCGAGACCAGTGCTGCATCATCAGTG
GGGAATCTGGGGCCGGGAAGACGGAGAGCACAAAGCTGATCCTGCAGTTCCTGGCAGCCA
TCAGTGGGCAGCACTCGTGGATTGAGCAGCAGGTCTTGGAGGCCACCCCCATTCTGGAAGC
ATTTGGGAATGCCAAGACCATCCGCAATGACAACTCAAGCCGTTTCGGAAAGTACATCGAC
ATCCACTTCAACAAGCGGGGCGCCATCGAGGGCGCGAAGATTGAGCAGTACCTGCTGGAA
AAGTCACGTGTCTGTCGCCAGGCCCTGGATGAAAGGAACTACCACGTGTTCTACTGCATGC
TGGAGGGTATGAGTGAGGATCAGAAGAAGAAGCTGGGCTTGGGCCAGGCCTCTGACTACA
ACTACTTGGCCATGGGTAACTGCATAACCTGTGAGGGCCGGGTGGACAGCCAGGAGTACGC
CAACATCCGCTCCGCCATGAAGGTGCTCATGTTCACTGACACCGAGAACTGGGAGATCTCG
AAGCTCCTGGCTGCCATCCTGCACCTGGGCAACCTGCAGTATGAGGCACGCACATTTGAAA
ACCTGGATGCCTGTGAGGTTCTCTTCTCCCCATCGCTGGCCACAGCTGCATCCCTGCTTGAG
GTGAACCCCCCAGACCTGATGAGCTGCCTGACTAGCCGCACCCTCATCACCCGCGGGGAGA
CGGTGTCCACCCCACTGAGCAGGGAACAGGCACTGGACGTGCGCGACGCCTTCGTAAAGGG
GATCTACGGGCGGCTGTTCGTGTGGATTGTGGACAAGATCAACGCAGCAATTTACAAGCCT
CCCTCCCAGGATGTGAAGAACTCTCGCAGGTCCATCGGCCTCCTGGACATCTTTGGGTTTGA
GAACTTTGCTGTGAACAGCTTTGAGCAGCTCTGCATCAACTTCGCCAATGAGCACCTGCAGC
AGTTCTTTGTGCGGCACGTGTTCAAGCTGGAGCAGGAGGAATATGACCTGGAGAGCATTGA
CTGGCTGCACATCGAGTTCACTGACAACCAGGATGCCCTGGACATGATTGCCAACAAGCCC
ATGAACATCATCTCCCTCATCGATGAGGAGAGCAAGTTCCCCAAGGGCACAGACACCACCA
TGTTACACAAGCTGAACTCCCAGCACAAGCTCAACGCCAACTACATCCCCCCCAAGAACAA
CCATGAGACCCAGTTTGGCATCAACCATTTTGCAGGCATCGTCTACTATGAGACCCAAGGCT
TCCTGGAGAAGAACCGAGACACCCTGCATGGGGACATTATCCAGCTGGTCCACTCCTCCAG
GAACAAGTTCATCAAGCAGATCTTCCAGGCCGATGTCGCCATGGGCGCCGAGACCAGGAAG
CGCTCGCCCACACTTAGCAGCCAGTTCAAGCGGTCACTGGAGCTGCTGATGCGCACGCTGG
GTGCCTGCCAGCCCTTCTTTGTGCGATGCATCAAGCCCAATGAGTTCAAGAAGCCCATGCTG
TTCGACCGGCACCTGTGCGTGCGCCAGCTGCGGTACTCAGGAATGATGGAGACCATCCGAA
TCCGCCGAGCTGGCTACCCCATCCGCTACAGCTTCGTAGAGTTTGTGGAGCGGTACCGTGTG
CTGCTGCCAGGTGTGAAGCCGGCCTACAAGCAGGGCGACCTCCGCGGGACTTGCCAGCGCA
TGGCTGAGGCTGTGCTGGGCACCCACGATGACTGGCAGATAGGCAAAACCAAGATCTTTCT
GAAGGACCACCATGACATGCTGCTGGAAGTGGAGCGGGACAAAGCCATCACCGACAGAGT
CATCCTCCTTCAGAAAGTCATCCGGGGATTCAAAGACAGGTCTAACTTTCTGAAGCTGAAG
AACGCTGCCACACTGATCCAGAGGCACTGGCGGGGTCACAACTGTAGGAAGAACTACGGG
CTGATGCGTCTGGGCTTCCTGCGGCTGCAGGCCCTGCACCGCTCCCGGAAGCTGCACCAGC
AGTACCGCCTGGCCCGCCAGCGCATCATCCAGTTCCAGGCCCGCTGCCGCGCCTATCTGGTG
CGCAAGGCCTTCCGCCACCGCCTCTGGGCTGTGCTCACCGTGCAGGCCTATGCCCGGGGCA
TGATCGCCCGCAGGCTGCACCAACGCCTCAGGGCTGAGGTAAGTATCAAGGTTACAAGACA
GGTTTAAGGAGACCAATAGAAACTGGGCTTGTCGAGACAGAGAAGACTCTTGCGTTTCTGA
GCTAGCCCCCGGGTGCGCGGCGTCGGTGGTGCCGGCGGGGGGCGCCAGGTCGCAGGCGGT
GTAGGGCTCCAGGCAGGCGGCGAAGGCCATGACGTGCGCTATGAAGGTCTGCTCCTGCACG
CCGTGAACCAGGTGCGCCTGCGGGCCGCGCGCGAACACCGCCACGTCCTCGCCTGCGTGGG
TCTCTTCGTCCAGGGGCACTGCTGACTGCTGCCGATACTCGGGGCTCCCGCTCTCGCTCTCG
GTAACATCCGGCCGGGCGCCGTCCTTGAGCACATAGCCTGGACCGTTTCGTCGACTGTTAAT
TAAGCATGCTGGGGAGAGATCTAGGAAACCCCTAGTGATGGAGTTGGCCACTCCCTCTCTG
CGCGCTCGCTCGCTCACTGAGGCCGGGCGACCAAAGGTCGCCCGACGCCCGGGCTTTGCCC
GGGCGGCCTCAGTGAGCGAGCGAGCGCGCAGAGAGGGAG
SEQ ID NO: 73 (hMYO7A-NT (AAV-smCBA-hMYO7A-NT-Ex21-APSD-APhead),
(AAV-smCBA-hMYO7A-NT-Ex21-APSD-APhead CMv1), (AAV-smCBA-hMYO7A-NT-
Ex21-APSD-APhead CMv2), and (AAV-smCBA-hMY07A- NT-Ex21-APSD-APhead-CMv3))
ATGGTGATTCTTCAGCAGGGGGACCATGTGTGGATGGACCTGAGATTGGGGCAGGAGTTCG
ACGTGCCCATCGGGGCGGTGGTGAAGCTCTGCGACTCTGGGCAGGTCCAGGTGGTGGATGA
TGAAGACAATGAACACTGGATCTCTCCGCAGAACGCAACGCACATCAAGCCTATGCACCCC
ACGTCGGTCCACGGCGTGGAGGACATGATCCGCCTGGGGGACCTCAACGAGGCGGGCATCT
TGCGCAACCTGCTTATCCGCTACCGGGACCACCTCATCTACACGTATACGGGCTCCATCCTG
GTGGCTGTGAACCCCTACCAGCTGCTCTCCATCTACTCGCCAGAGCACATCCGCCAGTATAC
CAACAAGAAGATTGGGGAGATGCCCCCCCACATCTTTGCCATTGCTGACAACTGCTACTTC
AACATGAAACGCAACAGCCGAGACCAGTGCTGCATCATCAGTGGGGAATCTGGGGCCGGG
AAGACGGAGAGCACAAAGCTGATCCTGCAGTTCCTGGCAGCCATCAGTGGGCAGCACTCGT
GGATTGAGCAGCAGGTCTTGGAGGCCACCCCCATTCTGGAAGCATTTGGGAATGCCAAGAC
CATCCGCAATGACAACTCAAGCCGTTTCGGAAAGTACATCGACATCCACTTCAACAAGCGG
GGCGCCATCGAGGGCGCGAAGATTGAGCAGTACCTGCTGGAAAAGTCACGTGTCTGTCGCC
AGGCCCTGGATGAAAGGAACTACCACGTGTTCTACTGCATGCTGGAGGGTATGAGTGAGGA
TCAGAAGAAGAAGCTGGGCTTGGGCCAGGCCTCTGACTACAACTACTTGGCCATGGGTAAC
TGCATAACCTGTGAGGGCCGGGTGGACAGCCAGGAGTACGCCAACATCCGCTCCGCCATGA
AGGTGCTCATGTTCACTGACACCGAGAACTGGGAGATCTCGAAGCTCCTGGCTGCCATCCT
GCACCTGGGCAACCTGCAGTATGAGGCACGCACATTTGAAAACCTGGATGCCTGTGAGGTT
CTCTTCTCCCCATCGCTGGCCACAGCTGCATCCCTGCTTGAGGTGAACCCCCCAGACCTGAT
GAGCTGCCTGACTAGCCGCACCCTCATCACCCGCGGGGAGACGGTGTCCACCCCACTGAGC
AGGGAACAGGCACTGGACGTGCGCGACGCCTTCGTAAAGGGGATCTACGGGCGGCTGTTCG
TGTGGATTGTGGACAAGATCAACGCAGCAATTTACAAGCCTCCCTCCCAGGATGTGAAGAA
CTCTCGCAGGTCCATCGGCCTCCTGGACATCTTTGGGTTTGAGAACTTTGCTGTGAACAGCT
TTGAGCAGCTCTGCATCAACTTCGCCAATGAGCACCTGCAGCAGTTCTTTGTGCGGCACGTG
TTCAAGCTGGAGCAGGAGGAATATGACCTGGAGAGCATTGACTGGCTGCACATCGAGTTCA
CTGACAACCAGGATGCCCTGGACATGATTGCCAACAAGCCCATGAACATCATCTCCCTCAT
CGATGAGGAGAGCAAGTTCCCCAAGGGCACAGACACCACCATGTTACACAAGCTGAACTCC
CAGCACAAGCTCAACGCCAACTACATCCCCCCCAAGAACAACCATGAGACCCAGTTTGGCA
TCAACCATTTTGCAGGCATCGTCTACTATGAGACCCAAGGCTTCCTGGAGAAGAACCGAGA
CACCCTGCATGGGGACATTATCCAGCTGGTCCACTCCTCCAGGAACAAGTTCATCAAGCAG
ATCTTCCAGGCCGATGTCGCCATGGGCGCCGAGACCAGGAAGCGCTCGCCCACACTTAGCA
GCCAGTTCAAGCGGTCACTGGAGCTGCTGATGCGCACGCTGGGTGCCTGCCAGCCCTTCTTT
GTGCGATGCATCAAGCCCAATGAGTTCAAGAAGCCCATGCTGTTCGACCGGCACCTGTGCG
TGCGCCAGCTGCGGTACTCAGGAATGATGGAGACCATCCGAATCCGCCGAGCTGGCTACCC
CATCCGCTACAGCTTCGTAGAGTTTGTGGAGCGGTACCGTGTGCTGCTGCCAGGTGTGAAG
CCGGCCTACAAGCAGGGCGACCTCCGCGGGACTTGCCAGCGCATGGCTGAGGCTGTGCTGG
GCACCCACGATGACTGGCAGATAGGCAAAACCAAGATCTTTCTGAAGGACCACCATGACAT
GCTGCTGGAAGTGGAGCGGGACAAAGCCATCACCGACAGAGTCATCCTCCTTCAGAAAGTC
ATCCGGGGATTCAAAGACAGGTCTAACTTTCTGAAGCTGAAGAACGCTGCCACACTGATCC
AGAGGCACTGGCGGGGTCACAACTGTAGGAAGAACTACGGGCTGATGCGTCTGGGCTTCCT
GCGGCTGCAGGCCCTGCACCGCTCCCGGAAGCTGCACCAGCAGTACCGCCTGGCCCGCCAG
CGCATCATCCAGTTCCAGGCCCGCTGCCGCGCCTATCTGGTGCGCAAGGCCTTCCGCCACCG
CCTCTGGGCTGTGCTCACCGTGCAGGCCTATGCCCGGGGCATGATCGCCCGCAGGCTGCAC
CAACGCCTCAGGGCTGAG
SEQ ID NO: 74 (hMYO7A-NT (AAV-smCBA-hMYO7A-NT-Ex21-APSD-APhead),
(AAV-smCBA-hMYO7A-NT-Ex21-APSD-APhead CMvl), (AAV-smCBA-hMY07A-NT-
Ex21-APSD-APhead-CMv3))
MVILQQGDHVWMDLRLGQEFDVPIGAVVKLCDSGQVQVVDDEDNEHWISPQNATHIKPMHPT
SVHGVEDMIRLGDLNEAGILRNLLIRYRDHLIYTYTGSILVAVNPYQLLSIYSPEHIRQYTNKKIG
EMPPHIFAIADNCYFNMKRNSRDQCCIISGESGAGKTESTKLILQFLAAISGQHSWIEQQVLEATP
ILEAFGNAKTIRNDNSSRFGKYIDIHFNKRGAIEGAKIEQYLLEKSRVCRQALDERNYHVFYCML
EGMSEDQKKKLGLGQASDYNYLAMGNCITCEGRVDSQEYANIRSAMKVLMFTDTENWEISKL
LAAILHLGNLQYEARTFENLDACEVLFSPSLATAASLLEVNPPDLMSCLTSRTLITRGETVSTPLS
REQALDVRDAFVKGIYGRLFVWIVDKINAAIYKPPSQDVKNSRRSIGLLDIFGFENFAVNSFEQL
CINFANEHLQQFFVRHVFKLEQEEYDLESIDWLHIEFTDNQDALDMIANKPMNIISLIDEESKFPK
GTDTTMLHKLNSQHKLNANYIPPKNNHETQFGINHFAGIVYYETQGFLEKNRDTLHGDIIQLVH
SSRNKFIKQIFQADVAMGAETRKRSPTLSSQFKRSLELLMRTLGACQPFFVRCIKPNEFKKPMLF
DRHLCVRQLRYSGMMETIRIRRAGYPIRYSFVEFVERYRVLLPGVKPAYKQGDLRGTCQRMAE
AVLGTHDDWQIGKTKIFLKDHHDMLLEVERDKAITDRVILLQKVIRGFKDRSNFLKLKNAATLI
QRHWRGHNCRKNYGLMRLGFLRLQALHRSRKLHQQYRLARQRIIQFQARCRAYLVRKAFRHR
LWAVLTVQAYARGMIARRLHQRLRAE
SEQ ID NO: 32 is the nucleotide sequence of the second generation
hybrid back-half vector (i.e., AAV-APhead-APSA-ex22hMYO7A-CT.HA (with
hemagglutinin (HA) tag)):
CTCCCTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCC
ATCACTAGGGGTTCTCAGATCTGGCGCGCCCAATTGGCTTCGAATTCTAGCGGCCGCCCCCG
GGTGCGCGGCGTCGGTGGTGCCGGCGGGGGGCGCCAGGTCGCAGGCGGTGTAGGGCTCCA
GGCAGGCGGCGAAGGCCATGACGTGCGCTATGAAGGTCTGCTCCTGCACGCCGTGAACCAG
GTGCGCCTGCGGGCCGCGCGCGAACACCGCCACGTCCTCGCCTGCGTGGGTCTCTTCGTCCA
GGGGCACTGCTGACTGCTGCCGATACTCGGGGCTCCCGCTCTCGCTCTCGGTAACATCCGGC
CGGGCGCCGTCCTTGAGCACATAGCCTGGACCGTTTCCTTAAGCGACGCATGCTCGCGATA
GGCACCTATTGGTCTTACTGACATCCACTTTGCCTTTCTCTCCACAGTATCTGTGGCGCCTCG
AGGCTGAGAAAATGCGGCTGGCGGAGGAAGAGAAGCTTCGGAAGGAGATGAGCGCCAAG
AAGGCCAAGGAGGAGGCCGAGCGCAAGCATCAGGAGCGCCTGGCCCAGCTGGCTCGTGAG
GACGCTGAGCGGGAGCTGAAGGAGAAGGAGGCCGCTCGGCGGAAGAAGGAGCTCCTGGAG
CAGATGGAAAGGGCCCGCCATGAGCCTGTCAATCACTCAGACATGGTGGACAAGATGTTTG
GCTTCCTGGGGACTTCAGGTGGCCTGCCAGGCCAGGAGGGCCAGGCACCTAGTGGCTTTGA
GGACCTGGAGCGAGGGCGGAGGGAGATGGTGGAGGAGGACCTGGATGCAGCCCTGCCCCT
GCCTGACGAGGATGAGGAGGACCTCTCTGAGTATAAATTTGCCAAGTTCGCGGCCACCTAC
TTCCAGGGGACAACCACGCACTCCTACACCCGGCGGCCACTCAAACAGCCACTGCTCTACC
ATGACGACGAGGGTGACCAGCTGGCAGCCCTGGCGGTCTGGATCACCATCCTCCGCTTCAT
GGGGGACCTCCCTGAGCCCAAGTACCACACAGCCATGAGTGATGGCAGTGAGAAGATCCCT
GTGATGACCAAGATTTATGAGACCCTGGGCAAGAAGACGTACAAGAGGGAGCTGCAGGCC
CTGCAGGGCGAGGGCGAGGCCCAGCTCCCCGAGGGCCAGAAGAAGAGCAGTGTGAGGCAC
AAGCTGGTGCATTTGACTCTGAAAAAGAAGTCCAAGCTCACAGAGGAGGTGACCAAGAGG
CTGCATGACGGGGAGTCCACAGTGCAGGGCAACAGCATGCTGGAGGACCGGCCCACCTCC
AACCTGGAGAAGCTGCACTTCATCATCGGCAATGGCATCCTGCGGCCAGCACTCCGGGACG
AGATCTACTGCCAGATCAGCAAGCAGCTGACCCACAACCCCTCCAAGAGCAGCTATGCCCG
GGGCTGGATTCTCGTGTCTCTCTGCGTGGGCTGTTTCGCCCCCTCCGAGAAGTTTGTCAAGT
ACCTGCGGAACTTCATCCACGGGGGCCCGCCCGGCTACGCCCCGTACTGTGAGGAGCGCCT
GAGAAGGACCTTTGTCAATGGGACACGGACACAGCCGCCCAGCTGGCTGGAGCTGCAGGC
CACCAAGTCCAAGAAGCCAATCATGTTGCCCGTGACATTCATGGATGGGACCACCAAGACC
CTGCTGACGGACTCGGCAACCACGGCCAAGGAGCTCTGCAACGCGCTGGCCGACAAGATCT
CTCTCAAGGACCGGTTCGGGTTCTCCCTCTACATTGCCCTGTTTGACAAGGTGTCCTCCCTG
GGCAGCGGCAGTGACCACGTCATGGACGCCATCTCCCAGTGCGAGCAGTACGCCAAGGAG
CAGGGCGCCCAGGAGCGCAACGCCCCCTGGAGGCTCTTCTTCCGCAAAGAGGTCTTCACGC
CCTGGCACAGCCCCTCCGAGGACAACGTGGCCACCAACCTCATCTACCAGCAGGTGGTGCG
AGGAGTCAAGTTTGGGGAGTACAGGTGTGAGAAGGAGGACGACCTGGCTGAGCTGGCCTC
CCAGCAGTACTTTGTAGACTATGGCTCTGAGATGATCCTGGAGCGCCTCCTGAACCTCGTGC
CCACCTACATCCCCGACCGCGAGATCACGCCCCTGAAGACGCTGGAGAAGTGGGCCCAGCT
GGCCATCGCCGCCCACAAGAAGGGGATTTATGCCCAGAGGAGAACTGATGCCCAGAAGGT
CAAAGAGGATGTGGTCAGTTATGCCCGCTTCAAGTGGCCCTTGCTCTTCTCCAGGTTTTATG
AAGCCTACAAATTCTCAGGCCCCAGTCTCCCCAAGAACGACGTCATCGTGGCCGTCAACTG
GACGGGTGTGTACTTTGTGGATGAGCAGGAGCAGGTACTTCTGGAGCTGTCCTTCCCAGAG
ATCATGGCCGTGTCCAGCAGCAGGGGAGCGAAAACGACGGCCCCCAGCTTCACGCTGGCCA
CCATCAAGGGGGACGAATACACCTTCACCTCCAGCAATGCTGAGGACATTCGTGACCTGGT
GGTCACCTTCCTAGAGGGGCTCCGGAAGAGATCTAAGTATGTTGTGGCCCTGCAGGATAAC
CCCAACCCCGCAGGCGAGGAGTCAGGCTTCCTCAGCTTTGCCAAGGGAGACCTCATCATCC
TGGACCATGACACGGGCGAGCAGGTCATGAACTCGGGCTGGGCCAACGGCATCAATGAGA
GGACCAAGCAGCGTGGGGACTTCCCCACCGACAGTGTGTACGTCATGCCCACTGTCACCAT
GCCACCGCGGGAGATTGTGGCCCTGGTCACCATGACTCCCGATCAGAGGCAGGACGTTGTC
CGGCTCTTGCAGCTGCGAACGGCGGAGCCCGAGGTGCGTGCCAAGCCCTACACGCTGGAGG
AGTTTTCCTATGACTACTTCAGGCCCCCACCCAAGCACACGCTGAGCCGTGTCATGGTGTCC
AAGGCCCGAGGCAAGGACCGGCTGTGGAGCCACACGCGGGAACCGCTCAAGCAGGCGCTG
CTCAAGAAGCTCCTGGGCAGTGAGGAGCTCTCGCAGGAGGCCTGCCTGGCCTTCATTGCTG
TGCTCAAGTACATGGGCGACTACCCGTCCAAGAGGACACGCTCCGTCAACGAGCTCACCGA
CCAGATCTTTGAGGGTCCCCTGAAAGCCGAGCCCCTGAAGGACGAGGCATATGTGCAGATC
CTGAAGCAGCTGACCGACAACCACATCAGGTACAGCGAGGAGCGGGGTTGGGAGCTGCTC
TGGCTGTGCACGGGCCTTTTCCCACCCAGCAACATCCTCCTGCCCCACGTGCAGCGCTTCCT
GCAGTCCCGAAAGCACTGCCCACTCGCCATCGACTGCCTGCAACGGCTCCAGAAAGCCCTG
AGAAACGGGTCCCGGAAGTACCCTCCGCACCTGGTGGAGGTGGAGGCCATCCAGCACAAG
ACCACCCAGATTTTCCACAAAGTCTACTTCCCTGATGACACTGACGAGGCCTTCGAAGTGG
AGTCCAGCACCAAGGCCAAGGACTTCTGCCAGAACATCGCCACCAGGCTGCTCCTCAAGTC
CTCAGAGGGATTCAGCCTCTTTGTCAAAATTGCAGACAAGGTCATCAGCGTTCCTGAGAAT
GACTTCTTCTTTGACTTTGTTCGACACTTGACAGACTGGATAAAGAAAGCTCGGCCCATCAA
GGACGGAATTGTGCCCTCACTCACCTACCAGGTGTTCTTCATGAAGAAGCTGTGGACCACC
ACGGTGCCAGGGAAGGATCCCATGGCCGATTCCATCTTCCACTATTACCAGGAGTTGCCCA
AGTATCTCCGAGGCTACCACAAGTGCACGCGGGAGGAGGTGCTGCAGCTGGGGGCGCTGAT
CTACAGGGTCAAGTTCGAGGAGGACAAGTCCTACTTCCCCAGCATCCCCAAGCTGCTGCGG
GAGCTGGTGCCCCAGGACCTTATCCGGCAGGTCTCACCTGATGACTGGAAGCGGTCCATCG
TCGCCTACTTCAACAAGCACGCAGGGAAGTCCAAGGAGGAGGCCAAGCTGGCCTTCCTGAA
GCTCATCTTCAAGTGGCCCACCTTTGGCTCAGCCTTCTTCGAGGTGAAGCAAACTACGGAGC
CAAACTTCCCTGAGATCCTCCTAATTGCCATCAACAAGTATGGGGTCAGCCTCATCGATCCC
AAAACGAAGGATATCCTCACCACTCATCCCTTCACCAAGATCTCCAACTGGAGCAGCGGCA
ACACCTACTTCCACATCACCATTGGGAACTTGGTGCGCGGGAGCAAACTGCTCTGCGAGAC
GTCACTGGGCTACAAGATGGATGACCTCCTGACTTCCTACATTAGCCAGATGCTCACAGCC
ATGAGCAAACAGCGGGGCTCCAGGAGCGGCAAGTACCCTTACGATGTACCGGATTACGCAT
GAGGTACCAAGGGCGAATTCTGCAGTCGACTAGAGCTCGCTGATCAGCCTCGACTGTGCCT
TCTAGTTGCCAGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAGTAGGTGTCA
TTCTATTCTGGGGGGTGGGGTGGGGCAGGACAGCAAGGGGGAGGATTGGGAAGACAATAG
CAGGCATGCTGGGGAGAGATCTGGAGGACTAGTCCGTCGACTGTTAATTAAGCATGCTGGG
GAGAGATCTAGGAAACCCCTAGTGATGGAGTTGGCCACTCCCTCTCTGCGCGCTCGCTCGCT
CACTGAGGCCGGGCGACCAAAGGTCGCCCGACGCCCGGGCTTTGCCCGGGCGGCCTCAGTG
AGCGAGCGAGCGCGCAGAGAGGGAG
SEQ ID NO: 75, ex22hMYO7A (e.g., AAV-APhead-APSA-ex22hMYO7A-CT.HA,
AAV-APhead-APSA-ex22hMYO7A -CT.HA-CMv2, AAV-APhead-APSA-hMYO7ACTex22-
CMv2.1.HA, AAV-APhead-APSA-hMYO7ACTex22-CMv2, AAV-APhead-APSA-
hMYO7ACTex 22.HA-MIN, and AAV-APhead-APSA-hMYO7ACTex 22-MIN))
TATCTGTGGCGCCTCGAGGCTGAGAAAATGCGGCTGGCGGAGGAAGAGAAGCTTCGGAAG
GAGATGAGCGCCAAGAAGGCCAAGGAGGAGGCCGAGCGCAAGCATCAGGAGCGCCTGGCC
CAGCTGGCTCGTGAGGACGCTGAGCGGGAGCTGAAGGAGAAGGAGGCCGCTCGGCGGAAG
AAGGAGCTCCTGGAGCAGATGGAAAGGGCCCGCCATGAGCCTGTCAATCACTCAGACATGG
TGGACAAGATGTTTGGCTTCCTGGGGACTTCAGGTGGCCTGCCAGGCCAGGAGGGCCAGGC
ACCTAGTGGCTTTGAGGACCTGGAGCGAGGGCGGAGGGAGATGGTGGAGGAGGACCTGGA
TGCAGCCCTGCCCCTGCCTGACGAGGATGAGGAGGACCTCTCTGAGTATAAATTTGCCAAGT
TCGCGGCCACCTACTTCCAGGGGACAACCACGCACTCCTACACCCGGCGGCCACTCAAACA
GCCACTGCTCTACCATGACGACGAGGGTGACCAGCTGGCAGCCCTGGCGGTCTGGATCACC
ATCCTCCGCTTCATGGGGGACCTCCCTGAGCCCAAGTACCACACAGCCATGAGTGATGGCA
GTGAGAAGATCCCTGTGATGACCAAGATTTATGAGACCCTGGGCAAGAAGACGTACAAGAG
GGAGCTGCAGGCCCTGCAGGGCGAGGGCGAGGCCCAGCTCCCCGAGGGCCAGAAGAAGAG
CAGTGTGAGGCACAAGCTGGTGCATTTGACTCTGAAAAAGAAGTCCAAGCTCACAGAGGAG
GTGACCAAGAGGCTGCATGACGGGGAGTCCACAGTGCAGGGCAACAGCATGCTGGAGGAC
CGGCCCACCTCCAACCTGGAGAAGCTGCACTTCATCATCGGCAATGGCATCCTGCGGCCAG
CACTCCGGGACGAGATCTACTGCCAGATCAGCAAGCAGCTGACCCACAACCCCTCCAAGAG
CAGCTATGCCCGGGGCTGGATTCTCGTGTCTCTCTGCGTGGGCTGTTTCGCCCCCTCCGAGA
AGTTTGTCAAGTACCTGCGGAACTTCATCCACGGGGGCCCGCCCGGCTACGCCCCGTACTGT
GAGGAGCGCCTGAGAAGGACCTTTGTCAATGGGACACGGACACAGCCGCCCAGCTGGCTGG
AGCTGCAGGCCACCAAGTCCAAGAAGCCAATCATGTTGCCCGTGACATTCATGGATGGGAC
CACCAAGACCCTGCTGACGGACTCGGCAACCACGGCCAAGGAGCTCTGCAACGCGCTGGCC
GACAAGATCTCTCTCAAGGACCGGTTCGGGTTCTCCCTCTACATTGCCCTGTTTGACAAGGT
GTCCTCCCTGGGCAGCGGCAGTGACCACGTCATGGACGCCATCTCCCAGTGCGAGCAGTAC
GCCAAGGAGCAGGGCGCCCAGGAGCGCAACGCCCCCTGGAGGCTCTTCTTCCGCAAAGAGG
TCTTCACGCCCTGGCACAGCCCCTCCGAGGACAACGTGGCCACCAACCTCATCTACCAGCAG
GTGGTGCGAGGAGTCAAGTTTGGGGAGTACAGGTGTGAGAAGGAGGACGACCTGGCTGAG
CTGGCCTCCCAGCAGTACTTTGTAGACTATGGCTCTGAGATGATCCTGGAGCGCCTCCTGAA
CCTCGTGCCCACCTACATCCCCGACCGCGAGATCACGCCCCTGAAGACGCTGGAGAAGTGG
GCCCAGCTGGCCATCGCCGCCCACAAGAAGGGGATTTATGCCCAGAGGAGAACTGATGCCC
AGAAGGTCAAAGAGGATGTGGTCAGTTATGCCCGCTTCAAGTGGCCCTTGCTCTTCTCCAGG
TTTTATGAAGCCTACAAATTCTCAGGCCCCAGTCTCCCCAAGAACGACGTCATCGTGGCCGT
CAACTGGACGGGTGTGTACTTTGTGGATGAGCAGGAGCAGGTACTTCTGGAGCTGTCCTTCC
CAGAGATCATGGCCGTGTCCAGCAGCAGGGGAGCGAAAACGACGGCCCCCAGCTTCACGCT
GGCCACCATCAAGGGGGACGAATACACCTTCACCTCCAGCAATGCTGAGGACATTCGTGAC
CTGGTGGTCACCTTCCTAGAGGGGCTCCGGAAGAGATCTAAGTATGTTGTGGCCCTGCAGG
ATAACCCCAACCCCGCAGGCGAGGAGTCAGGCTTCCTCAGCTTTGCCAAGGGAGACCTCAT
CATCCTGGACCATGACACGGGCGAGCAGGTCATGAACTCGGGCTGGGCCAACGGCATCAAT
GAGAGGACCAAGCAGCGTGGGGACTTCCCCACCGACAGTGTGTACGTCATGCCCACTGTCA
CCATGCCACCGCGGGAGATTGTGGCCCTGGTCACCATGACTCCCGATCAGAGGCAGGACGT
TGTCCGGCTCTTGCAGCTGCGAACGGCGGAGCCCGAGGTGCGTGCCAAGCCCTACACGCTG
GAGGAGTTTTCCTATGACTACTTCAGGCCCCCACCCAAGCACACGCTGAGCCGTGTCATGGT
GTCCAAGGCCCGAGGCAAGGACCGGCTGTGGAGCCACACGCGGGAACCGCTCAAGCAGGC
GCTGCTCAAGAAGCTCCTGGGCAGTGAGGAGCTCTCGCAGGAGGCCTGCCTGGCCTTCATT
GCTGTGCTCAAGTACATGGGCGACTACCCGTCCAAGAGGACACGCTCCGTCAACGAGCTCA
CCGACCAGATCTTTGAGGGTCCCCTGAAAGCCGAGCCCCTGAAGGACGAGGCATATGTGCA
GATCCTGAAGCAGCTGACCGACAACCACATCAGGTACAGCGAGGAGCGGGGTTGGGAGCT
GCTCTGGCTGTGCACGGGCCTTTTCCCACCCAGCAACATCCTCCTGCCCCACGTGCAGCGCT
TCCTGCAGTCCCGAAAGCACTGCCCACTCGCCATCGACTGCCTGCAACGGCTCCAGAAAGC
CCTGAGAAACGGGTCCCGGAAGTACCCTCCGCACCTGGTGGAGGTGGAGGCCATCCAGCAC
AAGACCACCCAGATTTTCCACAAAGTCTACTTCCCTGATGACACTGACGAGGCCTTCGAAGT
GGAGTCCAGCACCAAGGCCAAGGACTTCTGCCAGAACATCGCCACCAGGCTGCTCCTCAAG
TCCTCAGAGGGATTCAGCCTCTTTGTCAAAATTGCAGACAAGGTCATCAGCGTTCCTGAGAA
TGACTTCTTCTTTGACTTTGTTCGACACTTGACAGACTGGATAAAGAAAGCTCGGCCCATCA
AGGACGGAATTGTGCCCTCACTCACCTACCAGGTGTTCTTCATGAAGAAGCTGTGGACCACC
ACGGTGCCAGGGAAGGATCCCATGGCCGATTCCATCTTCCACTATTACCAGGAGTTGCCCAA
GTATCTCCGAGGCTACCACAAGTGCACGCGGGAGGAGGTGCTGCAGCTGGGGGCGCTGATC
TACAGGGTCAAGTTCGAGGAGGACAAGTCCTACTTCCCCAGCATCCCCAAGCTGCTGCGGG
AGCTGGTGCCCCAGGACCTTATCCGGCAGGTCTCACCTGATGACTGGAAGCGGTCCATCGTC
GCCTACTTCAACAAGCACGCAGGGAAGTCCAAGGAGGAGGCCAAGCTGGCCTTCCTGAAGC
TCATCTTCAAGTGGCCCACCTTTGGCTCAGCCTTCTTCGAGGTGAAGCAAACTACGGAGCCA
AACTTCCCTGAGATCCTCCTAATTGCCATCAACAAGTATGGGGTCAGCCTCATCGATCCCAA
AACGAAGGATATCCTCACCACTCATCCCTTCACCAAGATCTCCAACTGGAGCAGCGGCAAC
ACCTACTTCCACATCACCATTGGGAACTTGGTGCGCGGGAGCAAACTGCTCTGCGAGACGTC
ACTGGGCTACAAGATGGATGACCTCCTGACTTCCTACATTAGCCAGATGCTCACAGCCATGA
GCAAACAGCGGGGCTCCAGGAGCGGCAAG
SEQ ID NO: 76, ex22hMYO7A (e.g., AAV-APhead-APSA-ex22hMYO7A-CT.HA,
AAV-APhead-APSA-ex22hMYO7A -CT.HA-CMv2, and AAV-APhead-APSA-
hMYO7ACTex22-CMv2.1 .HA))
YLWRLEAEKMRLAEEEKLRKEMSAKKAKEEAERKHQERLAQLAREDAERELKEKEAARRKKE
LLEQMERARHEPVNHSDMVDKMFGFLGTSGGLPGQEGQAPSGFEDLERGRREMVEEDLDAAL
PLPDEDEEDLSEYKFAKFAATYFQGTTTHSYTRRPLKQPLLYHDDEGDQLAALAVWITILRFMG
DLPEPKYHTAMSDGSEKIPVMTKIYETLGKKTYKRELQALQGEGEAQLPEGQKKSSVRHKLVH
LTLKKKSKLTEEVTKRLHDGESTVQGNSMLEDRPTSNLEKLHFIIGNGILRPALRDEIYCQISKQL
THNPSKSSYARGWILVSLCVGCFAPSEKFVKYLRNFIHGGPPGYAPYCEERLRRTFVNGTRTQPP
SWLELQATKSKKPIMLPVTFMDGTTKTLLTDSATTAKELCNALADKISLKDRFGFSLYIALFDK
VSSLGSGSDHVMDAISQCEQYAKEQGAQERNAPWRLFFRKEVFTPWHSPSEDNVATNLIYQQV
VRGVKFGEYRCEKEDDLAELASQQYFVDYGSEMILERLLNLVPTYIPDREITPLKTLEKWAQLAI
AAHKKGIYAQRRTDAQKVKEDVVSYARFKWPLLFSRFYEAYKFSGPSLPKNDVIVAVNWTGV
YFVDEQEQVLLELSFPEIMAVSSSRGAKTTAPSFTLATIKGDEYTFTSSNAEDIRDLVVTFLEGLR
KRSKYVVALQDNPNPAGEESGFLSFAKGDLIILDHDTGEQVMNSGWANGINERTKQRGDFPTD
SVYVMPTVTMPPREIVALVTMTPDQRQDVVRLLQLRTAEPEVRAKPYTLEEFSYDYFRPPPKHT
LSRVMVSKARGKDRLWSHTREPLKQALLKKLLGSEELSQEACLAFIAVLKYMGDYPSKRTRSV
NELTDQIFEGPLKAEPLKDEAYVQILKQLTDNHIRYSEERGWELLWLCTGLFPPSNILLPHVQRF
LQSRKHCPLAIDCLQRLQKALRNGSRKYPPHLVEVEAIQHKTTQIFHKVYFPDDTDEAFEVESST
KAKDFCQNIATRLLLKSSEGFSLFVKIADKVISVPENDFFFDFVRHLTDWIKKARPIKDGIVPSLT
YQVFFMKKLWTTTVPGKDPMADSIFHYYQELPKYLRGYHKCTREEVLQLGALIYRVKFEEDKS
YFPSIPKLLRELVPQDLIRQVSPDDWKRSIVAYFNKHAGKSKEEAKLAFLKLIFKWPTFGSAFFE
VKQTTEPNFPEILLIAINKYGVSLIDPKTKDILTTHPFTKISNWSSGNTYFHITIGNLVRGSKLLCET
SLGYKMDDLLTSYISQMLTAMSKQRGSRSGK
SEQ ID NO: 33 is the nucleotide sequence of the CMv1 hybrid front-
half vector (i.e., AAV-smCBA-hMYO7A-NT-Ex21-APSD-APhead CMv1):
CTCCCTCTCTGCGCGCTCGCTCGCTCACTGAGGCCGCCCGGGCAAAGCCCGGGCGTCGGGCG
ACCTTTGGTCGCCCGGCCTCAGTGAGCGAGCGAGCGCGCAGAGAGGGAGTGGCCAACTCCA
TCACTAGGGGTTCTCAGATCTGGCGCGCCCAATTCGGTACCCTAGTTATTAATAGTAATCAA
TTACGGGGTCATTAGTTCATAGCCCATATATGGAGTTCCGCGTTACATAACTTACGGTAAAT
GGCCCGCCTGGCTGACCGCCCAACGACCCCCGCCCATTGACGTCAATAATGACGTATGTTCC
CATAGTAACGCCAATAGGGACTTTCCATTGACGTCAATGGGTGGACTATTTACGGTAAACTG
CCCACTTGGCAGTACATCAAGTGTATCATATGCCAAGTACGCCCCCTATTGACGTCAATGAC
GGTAAATGGCCCGCCTGGCATTATGCCCAGTACATGACCTTATGGGACTTTCCTACTTGGCA
GTACATCTACGTATTAGTCATCGCTATTACCATGGTCGAGGTGAGCCCCACGTTCTGCTTCA
CTCTCCCCATCTCCCCCCCCTCCCCACCCCCAATTTTGTATTTATTTATTTTTTAATTATTTTG
TGCAGCGATGGGGGCGGGGGGGGGGGGGGGGCGCGCGCCAGGCGGGGCGGGGCGGGGCG
AGGGGCGGGGCGGGGCGAGGCGGAGAGGTGCGGCGGCAGCCAATCAGAGCGGCGCGCTCC
GAAAGTTTCCTTTTATGGCGAGGCGGCGGCGGCGGCGGCCCTATAAAAAGCGAAGCGCGCG
GCGGGCGGGAGTCGCTGCGCGCTGCCTTCGCCCCGTGCCCCGCTCCGCCGCCGCCTCGCGCC
GCCCGCCCCGGCTCTGACTGACCGCGTTACTCCCACAGGTGAGCGGGCGGGACGGCCCTTCT
CCTCCGGGCTGTAATTAGCGCTTGGTTTAATGACGGCTTGTTTCTTTTCTGTGGCTGCGTGAA
AGCCTTGAGGGGCTCCGGGAGCTAGAGCCTCTGCTAACCATGTTCATGCCTTCTTCTTTTTCC
TACAGCTCCTGGGCAACGTGCTGGTTATTGTGCTGTCTCATCATTTTGGCAAAGAATTCTAG
CGGCCGCCACCATGGTGATTCTTCAGCAGGGGGACCATGTGTGGATGGACCTGAGATTGGG
GCAGGAGTTCGACGTGCCCATCGGGGCGGTGGTGAAGCTCTGCGACTCTGGGCAGGTCCAG
GTGGTGGATGATGAAGACAATGAACACTGGATCTCTCCGCAGAACGCAACGCACATCAAGC
CTATGCACCCCACGTCGGTCCACGGCGTGGAGGACATGATCCGCCTGGGGGACCTCAACGA
GGCGGGCATCTTGCGCAACCTGCTTATCCGCTACCGGGACCACCTCATCTACACGTATACGG
GCTCCATCCTGGTGGCTGTGAACCCCTACCAGCTGCTCTCCATCTACTCGCCAGAGCACATC
CGCCAGTATACCAACAAGAAGATTGGGGAGATGCCCCCCCACATCTTTGCCATTGCTGACA
ACTGCTACTTCAACATGAAACGCAACAGCCGAGACCAGTGCTGCATCATCAGTGGGGAATC
TGGGGCCGGGAAGACGGAGAGCACAAAGCTGATCCTGCAGTTCCTGGCAGCCATCAGTGGG
CAGCACTCGTGGATTGAGCAGCAGGTCTTGGAGGCCACCCCCATTCTGGAAGCATTTGGGA
ATGCCAAGACCATCCGCAATGACAACTCAAGCCGTTTCGGAAAGTACATCGACATCCACTT
CAACAAGCGGGGCGCCATCGAGGGCGCGAAGATTGAGCAGTACCTGCTGGAAAAGTCACG
TGTCTGTCGCCAGGCCCTGGATGAAAGGAACTACCACGTGTTCTACTGCATGCTGGAGGGTA
TGAGTGAGGATCAGAAGAAGAAGCTGGGCTTGGGCCAGGCCTCTGACTACAACTACTTGGC
CATGGGTAACTGCATAACCTGTGAGGGCCGGGTGGACAGCCAGGAGTACGCCAACATCCGC
TCCGCCATGAAGGTGCTCATGTTCACTGACACCGAGAACTGGGAGATCTCGAAGCTCCTGG
CTGCCATCCTGCACCTGGGCAACCTGCAGTATGAGGCACGCACATTTGAAAACCTGGATGC
CTGTGAGGTTCTCTTCTCCCCATCGCTGGCCACAGCTGCATCCCTGCTTGAGGTGAACCCCC
CAGACCTGATGAGCTGCCTGACTAGCCGCACCCTCATCACCCGCGGGGAGACGGTGTCCAC
CCCACTGAGCAGGGAACAGGCACTGGACGTGCGCGACGCCTTCGTAAAGGGGATCTACGGG
CGGCTGTTCGTGTGGATTGTGGACAAGATCAACGCAGCAATTTACAAGCCTCCCTCCCAGGA
TGTGAAGAACTCTCGCAGGTCCATCGGCCTCCTGGACATCTTTGGGTTTGAGAACTTTGCTG
TGAACAGCTTTGAGCAGCTCTGCATCAACTTCGCCAATGAGCACCTGCAGCAGTTCTTTGTG
CGGCACGTGTTCAAGCTGGAGCAGGAGGAATATGACCTGGAGAGCATTGACTGGCTGCACA
TCGAGTTCACTGACAACCAGGATGCCCTGGACATGATTGCCAACAAGCCCATGAACATCAT
CTCCCTCATCGATGAGGAGAGCAAGTTCCCCAAGGGCACAGACACCACCATGTTACACAAG
CTGAACTCCCAGCACAAGCTCAACGCCAACTACATCCCCCCCAAGAACAACCATGAGACCC
AGTTTGGCATCAACCATTTTGCAGGCATCGTCTACTATGAGACCCAAGGCTTCCTGGAGAAG
AACCGAGACACCCTGCATGGGGACATTATCCAGCTGGTCCACTCCTCCAGGAACAAGTTCA
TCAAGCAGATCTTCCAGGCCGATGTCGCCATGGGCGCCGAGACCAGGAAGCGCTCGCCCAC
ACTTAGCAGCCAGTTCAAGCGGTCACTGGAGCTGCTGATGCGCACGCTGGGTGCCTGCCAG
CCCTTCTTTGTGCGATGCATCAAGCCCAATGAGTTCAAGAAGCCCATGCTGTTCGACCGGCA
CCTGTGCGTGCGCCAGCTGCGGTACTCAGGAATGATGGAGACCATCCGAATCCGCCGAGCT
GGCTACCCCATCCGCTACAGCTTCGTAGAGTTTGTGGAGCGGTACCGTGTGCTGCTGCCAGG
TGTGAAGCCGGCCTACAAGCAGGGCGACCTCCGCGGGACTTGCCAGCGCATGGCTGAGGCT
GTGCTGGGCACCCACGATGACTGGCAGATAGGCAAAACCAAGATCTTTCTGAAGGACCACC
ATGACATGCTGCTGGAAGTGGAGCGGGACAAAGCCATCACCGACAGAGTCATCCTCCTTCA
GAAAGTCATCCGGGGATTCAAAGACAGGTCTAACTTTCTGAAGCTGAAGAACGCTGCCACA
CTGATCCAGAGGCACTGGCGGGGTCACAACTGTAGGAAGAACTACGGGCTGATGCGTCTGG
GCTTCCTGCGGCTGCAGGCCCTGCACCGCTCCCGGAAGCTGCACCAGCAGTACCGCCTGGCC
CGCCAGCGCATCATCCAGTTCCAGGCCCGCTGCCGCGCCTATCTGGTGCGCAAGGCCTTCCG
CCACCGCCTCTGGGCTGTGCTCACCGTGCAGGCCTATGCCCGGGGCATGATCGCCCGCAGGC
TGCACCAACGCCTCAGGGCTGAGGTAAGTATCAAGGTTACAAGACAGGTTAACGGAGACCA
ATTGAAACTGGGCTTGTCGAGACAGAGAAGACTCTTGCGTTTCAGCGCTAGCCCCCGGGTG
CGCGGCGTCGGTGGTGCCGGCGGGGGGCGCCAGGTCGCAGGCGGTGTAGGGCTCCAGGCA
GGCGGCGAAGGCCATGACGTGCGCTATGAAGGTCTGCTCCTGCACGCCGTGAACCAGGTGC
GCCTGCGGGCCGCGCGCGAACACCGCCACGTCCTCGCCTGCGTGGGTCTCTTCGTCCAGGGG
CACTGCTGACTGCTGCCGATACTCGGGGCTCCCGCTCTCGCTCTCGGTAACATCCGGCCGGG
CGCCGTCCTTGAGCACATAGCCTGGACCGTTTCGTCGACTGTTAATTAAGCATGCTGGGGAG
AGATCTGAGGAAACCCCTAGTGATGGAGTTGGCCACTCCCTCTCTGCGCGCTCGCTCGCTCA
CTGAGGCCGGGCGACCAAAGGTCGCCCGACGCCCGGGCTTTGCCCGGGCGGCCTCAGTGAG
CGAGCGAGCGCGCAGAGAGGGAG
SEQ ID NO: 34 is the nucleotide sequence of the CMv2 hybrid front-half
vector (i.e., AAV-smCBA-hMYO7A-NT-Ex21-APSD-APhead CMv2):
CTCCCTCTCTGCGCGCTCGCTCGCTCACTGAGGCCGCCCGGGCAAAGCCCGGGCGTCGGGCG
ACCTTTGGTCGCCCGGCCTCAGTGAGCGAGCGAGCGCGCAGAGAGGGAGTGGCCAACTCCA
TCACTAGGGGTTCTCAGATCTGGCGCGCCCAATTCGGTACCCTAGTTATTAATAGTAATCAA
TTACGGGGTCATTAGTTCATAGCCCATATATGGAGTTCCGCGTTACATAACTTACGGTAAAT
GGCCCGCCTGGCTGACCGCCCAACGACCCCCGCCCATTGACGTCAATAATGACGTATGTTCC
CATAGTAACGCCAATAGGGACTTTCCATTGACGTCAATGGGTGGACTATTTACGGTAAACTG
CCCACTTGGCAGTACATCAAGTGTATCATATGCCAAGTACGCCCCCTATTGACGTCAATGAC
GGTAAATGGCCCGCCTGGCATTATGCCCAGTACATGACCTTATGGGACTTTCCTACTTGGCA
GTACATCTACGTATTAGTCATCGCTATTACCATGGTCGAGGTGAGCCCCACGTTCTGCTTCA
CTCTCCCCATCTCCCCCCCCTCCCCACCCCCAATTTTGTATTTATTTATTTTTTAATTATTTTG
TGCAGCGATGGGGGCGGGGGGGGGGGGGGGGCGCGCGCCAGGCGGGGCGGGGCGGGGCG
AGGGGCGGGGCGGGGCGAGGCGGAGAGGTGCGGCGGCAGCCAATCAGAGCGGCGCGCTCC
GAAAGTTTCCTTTTATGGCGAGGCGGCGGCGGCGGCGGCCCTATAAAAAGCGAAGCGCGCG
GCGGGCGGGAGTCGCTGCGCGCTGCCTTCGCCCCGTGCCCCGCTCCGCCGCCGCCTCGCGCC
GCCCGCCCCGGCTCTGACTGACCGCGTTACTCCCACAGGTGAGCGGGCGGGACGGCCCTTCT
CCTCCGGGCTGTAATTAGCGCTTGGTTTAATGACGGCTTGTTTCTTTTCTGTGGCTGCGTGAA
AGCCTTGAGGGGCTCCGGGAGCTAGAGCCTCTGCTAACCATGTTCATGCCTTCTTCTTTTTCC
TACAGCTCCTGGGCAACGTGCTGGTTATTGTGCTGTCTCATCATTTTGGCAAAGAATTCTAG
CGGCCGCCACCATGGTGATTCTTCAGCAGGGGGACCATGTGTGGATGGACCTGAGATTGGG
GCAGGAGTTCGACGTGCCCATCGGGGCGGTGGTGAAGCTCTGCGACTCTGGGCAGGTCCAG
GTGGTGGATGATGAAGACAATGAACACTGGATCTCTCCGCAGAACGCAACGCACATCAAGC
CTATGCACCCCACGTCGGTCCACGGCGTGGAGGACATGATCCGCCTGGGGGACCTCAACGA
GGCGGGCATCTTGCGCAACCTGCTTATCCGCTACCGGGACCACCTCATCTACACGTATACGG
GCTCCATCCTGGTGGCTGTGAACCCCTACCAGCTGCTCTCCATCTACTCGCCAGAGCACATC
CGCCAGTATACCAACAAGAAGATTGGGGAGATGCCCCCCCACATCTTTGCCATTGCTGACA
ACTGCTACTTCAACATGAAACGCAACAGCCGAGACCAGTGCTGCATCATCAGTGGGGAATC
TGGGGCCGGGAAGACGGAGAGCACAAAGCTGATCCTGCAGTTCCTGGCAGCCATCAGTGGG
CAGCACTCGTGGATTGAGCAGCAGGTCTTGGAGGCCACCCCCATTCTGGAAGCATTTGGGA
ATGCCAAGACCATCCGCAATGACAACTCAAGCCGTTTCGGAAAGTACATCGACATCCACTT
CAACAAGCGGGGCGCCATCGAGGGCGCGAAGATTGAGCAGTACCTGCTGGAAAAGTCACG
TGTCTGTCGCCAGGCCCTGGATGAAAGGAACTACCACGTGTTCTACTGCATGCTGGAGGGTA
TGAGTGAGGATCAGAAGAAGAAGCTGGGCTTGGGCCAGGCCTCTGACTACAACTACTTGGC
CATGGGTAACTGCATAACCTGTGAGGGCCGGGTGGACAGCCAGGAGTACGCCAACATCCGC
TCCGCCATGAAGGTGCTCATGTTCACTGACACCGAGAACTGGGAGATCTCGAAGCTCCTGG
CTGCCATCCTGCACCTGGGCAACCTGCAGTATGAGGCACGCACATTTGAAAACCTGGATGC
CTGTGAGGTTCTCTTCTCCCCATCGCTGGCCACAGCTGCATCCCTGCTTGAGGTGAACCCCC
CAGACCTGATGAGCTGCCTGACTAGCCGCACCCTCATCACCCGCGGGGAGACGGTGTCCAC
CCCACTGAGCAGGGAACAGGCACTGGACGTGCGCGACGCCTTCGTAAAGGGGATCTACGGG
CGGCTGTTCGTGTGGATTGTGGACAAGATCAACGCAGCAATTTACAAGCCTCCCTCCCAGGA
TGTGAAGAACTCTCGCAGGTCCATCGGCCTCCTGGACATCTTTGGGTTTGAGAACTTTGCTG
TGAACAGCTTTGAGCAGCTCTGCATCAACTTCGCCAATGAGCACCTGCAGCAGTTCTTTGTG
CGGCACGTGTTCAAGCTGGAGCAGGAGGAATATGACCTGGAGAGCATTGACTGGCTGCACA
TCGAGTTCACTGACAACCAGGATGCCCTGGACATGATTGCCAACAAGCCCATGAACATCAT
CTCCCTCATCGATGAGGAGAGCAAGTTCCCCAAGGGCACAGACACCACCATGTTACACAAG
CTGAACTCCCAGCACAAGCTCAACGCCAACTACATCCCCCCCAAGAACAACCATGAGACCC
AGTTTGGCATCAACCATTTTGCAGGCATCGTCTACTATGAGACCCAAGGCTTCCTGGAGAAG
AACCGAGACACCCTGCATGGGGACATTATCCAGCTGGTCCACTCCTCCAGGAACAAGTTCA
TCAAGCAGATCTTCCAGGCCGATGTCGCCATGGGCGCCGAGACCAGGAAGCGCTCGCCCAC
ACTTAGCAGCCAGTTCAAGCGGTCACTGGAGCTGCTGATGCGCACGCTGGGTGCCTGCCAG
CCCTTCTTTGTGCGATGCATCAAGCCCAATGAGTTCAAGAAGCCCATGCTGTTCGACCGGCA
CCTGTGCGTGCGCCAGCTGCGGTACTCAGGAATGATGGAGACCATCCGAATCCGCCGAGCT
GGCTACCCCATCCGCTACAGCTTCGTAGAGTTTGTGGAGCGGTACCGTGTGCTGCTGCCAGG
TGTGAAGCCGGCCTACAAGCAGGGCGACCTCCGCGGGACTTGCCAGCGCATGGCTGAGGCT
GTGCTGGGCACCCACGATGACTGGCAGATAGGCAAAACCAAGATCTTTCTGAAGGACCACC
ATGACATGCTGCTGGAAGTGGAGCGGGACAAAGCCATCACCGACAGAGTCATCCTCCTTCA
GAAAGTCATCCGGGGATTCAAAGACAGGTCTAACTTTCTGAAGCTGAAGAACGCTGCCACA
CTGATCCAGAGGCACTGGCGGGGTCACAACTGTAGGAAGAACTACGGGCTGATGCGTCTGG
GCTTCCTGCGGCTGCAGGCCCTGCACCGCTCCCGGAAGCTGCACCAGCAGTACCGCCTGGCC
CGCCAGCGCATCATCCAGTTCCAGGCCCGCTGCCGCGCCTATCTGGTGCGCAAGGCCTTCCG
CCACCGCCTCTGGGCTGTGCTCACCGTGCAGGCCTATGCCCGGGGCATGATCGCCCGCAGGC
TGCACCAACGCCTCAGGGCTGAGGTAAGTATCAAGGTTACAAGACAGGTTAACGGAGACCA
ATTGAAACTGGGCTTGTCGAGACAGAGAAGACTCTTGCGTTTCAGCGCTAGCCCCCGGGTG
CGCGGCGTCGGTGGTGCCGGCGGGGGGCGCCAGGTCGCAGGCGGTGTAGGGCTCCAGGCA
GGCGGCGAAGGCCATGACGTGCGCTATGAAGGTCTGCTCCTGCACGCCGTGAACCAGGTGC
GCCTGCGGGCCGCGCGCGAACACCGCCACGTCCTCGCCTGCGTGGGTCTCTTCGTCCAGGGG
CACTGCGCACTGCTGCCGATACTCGGGGCTCCCGCTCTCGCTCTCGGTAACATCCGGCCGGG
CGCCGTCCTTGAGCACATAGCCTGGACCGTTTCGTCGACTGTTAATTAAGCATGCTGGGGAG
AGATCTGTAACCCCTAGTGATGGAGTTGGCCACTCCCTCTCTGCGCGCTCGCTCGCTCACTG
AGGCCGGGCGACCAAAGGTCGCCCGACGCCCGGGCTTTGCCCGGGCGGCCTCAGTGAGCGA
GCGAGCGCGCAGAGAGGGAG
SEQ ID NO: 35 is the nucleotide sequence of the CMv2 hybrid back-half
vector (i.e., AAV-APhead-APSA-ex22hMYO7A-CT.HA-CMv2):
CTCCCTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCC
ATCACTAGGGGTTCAGATCTGGCGCGCCCAATTGGCTTCGAATTCTAGCGGCCGCCCCCGG
GTGCGCGGCGTCGGTGGTGCCGGCGGGGGGCGCCAGGTCGCAGGCGGTGTAGGGCTCCAG
GCAGGCGGCGAAGGCCATGACGTGCGCTATGAAGGTCTGCTCCTGCACGCCGTGAACCAGG
TGCGCCTGCGGGCCGCGCGCGAACACCGCCACGTCCTCGCCTGCGTGGGTCTCTTCGTCCAG
GGGCACTGCGCACTGCTGCCGATACTCGGGGCTCCCGCTCTCGCTCTCGGTAACATCCGGCC
GGGCGCCGTCCTTGAGCACATAGCCTGGACCGTTTCTCTTAAGCGACGCATGCTCGCGATA
GGCACCTATTGGTCTTACTGACATCCACTTTGCCTTTCTCTCCACAGTATCTGTGGCGCCTCG
AGGCTGAGAAAATGCGGCTGGCGGAGGAAGAGAAGCTTCGGAAGGAGATGAGCGCCAAG
AAGGCCAAGGAGGAGGCCGAGCGCAAGCATCAGGAGCGCCTGGCCCAGCTGGCTCGTGAG
GACGCTGAGCGGGAGCTGAAGGAGAAGGAGGCCGCTCGGCGGAAGAAGGAGCTCCTGGAG
CAGATGGAAAGGGCCCGCCATGAGCCTGTCAATCACTCAGACATGGTGGACAAGATGTTTG
GCTTCCTGGGGACTTCAGGTGGCCTGCCAGGCCAGGAGGGCCAGGCACCTAGTGGCTTTGA
GGACCTGGAGCGAGGGCGGAGGGAGATGGTGGAGGAGGACCTGGATGCAGCCCTGCCCCT
GCCTGACGAGGATGAGGAGGACCTCTCTGAGTATAAATTTGCCAAGTTCGCGGCCACCTAC
TTCCAGGGGACAACCACGCACTCCTACACCCGGCGGCCACTCAAACAGCCACTGCTCTACC
ATGACGACGAGGGTGACCAGCTGGCAGCCCTGGCGGTCTGGATCACCATCCTCCGCTTCAT
GGGGGACCTCCCTGAGCCCAAGTACCACACAGCCATGAGTGATGGCAGTGAGAAGATCCCT
GTGATGACCAAGATTTATGAGACCCTGGGCAAGAAGACGTACAAGAGGGAGCTGCAGGCC
CTGCAGGGCGAGGGCGAGGCCCAGCTCCCCGAGGGCCAGAAGAAGAGCAGTGTGAGGCAC
AAGCTGGTGCATTTGACTCTGAAAAAGAAGTCCAAGCTCACAGAGGAGGTGACCAAGAGG
CTGCATGACGGGGAGTCCACAGTGCAGGGCAACAGCATGCTGGAGGACCGGCCCACCTCC
AACCTGGAGAAGCTGCACTTCATCATCGGCAATGGCATCCTGCGGCCAGCACTCCGGGACG
AGATCTACTGCCAGATCAGCAAGCAGCTGACCCACAACCCCTCCAAGAGCAGCTATGCCCG
GGGCTGGATTCTCGTGTCTCTCTGCGTGGGCTGTTTCGCCCCCTCCGAGAAGTTTGTCAAGT
ACCTGCGGAACTTCATCCACGGGGGCCCGCCCGGCTACGCCCCGTACTGTGAGGAGCGCCT
GAGAAGGACCTTTGTCAATGGGACACGGACACAGCCGCCCAGCTGGCTGGAGCTGCAGGC
CACCAAGTCCAAGAAGCCAATCATGTTGCCCGTGACATTCATGGATGGGACCACCAAGACC
CTGCTGACGGACTCGGCAACCACGGCCAAGGAGCTCTGCAACGCGCTGGCCGACAAGATCT
CTCTCAAGGACCGGTTCGGGTTCTCCCTCTACATTGCCCTGTTTGACAAGGTGTCCTCCCTG
GGCAGCGGCAGTGACCACGTCATGGACGCCATCTCCCAGTGCGAGCAGTACGCCAAGGAG
CAGGGCGCCCAGGAGCGCAACGCCCCCTGGAGGCTCTTCTTCCGCAAAGAGGTCTTCACGC
CCTGGCACAGCCCCTCCGAGGACAACGTGGCCACCAACCTCATCTACCAGCAGGTGGTGCG
AGGAGTCAAGTTTGGGGAGTACAGGTGTGAGAAGGAGGACGACCTGGCTGAGCTGGCCTC
CCAGCAGTACTTTGTAGACTATGGCTCTGAGATGATCCTGGAGCGCCTCCTGAACCTCGTGC
CCACCTACATCCCCGACCGCGAGATCACGCCCCTGAAGACGCTGGAGAAGTGGGCCCAGCT
GGCCATCGCCGCCCACAAGAAGGGGATTTATGCCCAGAGGAGAACTGATGCCCAGAAGGT
CAAAGAGGATGTGGTCAGTTATGCCCGCTTCAAGTGGCCCTTGCTCTTCTCCAGGTTTTATG
AAGCCTACAAATTCTCAGGCCCCAGTCTCCCCAAGAACGACGTCATCGTGGCCGTCAACTG
GACGGGTGTGTACTTTGTGGATGAGCAGGAGCAGGTACTTCTGGAGCTGTCCTTCCCAGAG
ATCATGGCCGTGTCCAGCAGCAGGGGAGCGAAAACGACGGCCCCCAGCTTCACGCTGGCCA
CCATCAAGGGGGACGAATACACCTTCACCTCCAGCAATGCTGAGGACATTCGTGACCTGGT
GGTCACCTTCCTAGAGGGGCTCCGGAAGAGATCTAAGTATGTTGTGGCCCTGCAGGATAAC
CCCAACCCCGCAGGCGAGGAGTCAGGCTTCCTCAGCTTTGCCAAGGGAGACCTCATCATCC
TGGACCATGACACGGGCGAGCAGGTCATGAACTCGGGCTGGGCCAACGGCATCAATGAGA
GGACCAAGCAGCGTGGGGACTTCCCCACCGACAGTGTGTACGTCATGCCCACTGTCACCAT
GCCACCGCGGGAGATTGTGGCCCTGGTCACCATGACTCCCGATCAGAGGCAGGACGTTGTC
CGGCTCTTGCAGCTGCGAACGGCGGAGCCCGAGGTGCGTGCCAAGCCCTACACGCTGGAGG
AGTTTTCCTATGACTACTTCAGGCCCCCACCCAAGCACACGCTGAGCCGTGTCATGGTGTCC
AAGGCCCGAGGCAAGGACCGGCTGTGGAGCCACACGCGGGAACCGCTCAAGCAGGCGCTG
CTCAAGAAGCTCCTGGGCAGTGAGGAGCTCTCGCAGGAGGCCTGCCTGGCCTTCATTGCTG
TGCTCAAGTACATGGGCGACTACCCGTCCAAGAGGACACGCTCCGTCAACGAGCTCACCGA
CCAGATCTTTGAGGGTCCCCTGAAAGCCGAGCCCCTGAAGGACGAGGCATATGTGCAGATC
CTGAAGCAGCTGACCGACAACCACATCAGGTACAGCGAGGAGCGGGGTTGGGAGCTGCTC
TGGCTGTGCACGGGCCTTTTCCCACCCAGCAACATCCTCCTGCCCCACGTGCAGCGCTTCCT
GCAGTCCCGAAAGCACTGCCCACTCGCCATCGACTGCCTGCAACGGCTCCAGAAAGCCCTG
AGAAACGGGTCCCGGAAGTACCCTCCGCACCTGGTGGAGGTGGAGGCCATCCAGCACAAG
ACCACCCAGATTTTCCACAAAGTCTACTTCCCTGATGACACTGACGAGGCCTTCGAAGTGG
AGTCCAGCACCAAGGCCAAGGACTTCTGCCAGAACATCGCCACCAGGCTGCTCCTCAAGTC
CTCAGAGGGATTCAGCCTCTTTGTCAAAATTGCAGACAAGGTCATCAGCGTTCCTGAGAAT
GACTTCTTCTTTGACTTTGTTCGACACTTGACAGACTGGATAAAGAAAGCTCGGCCCATCAA
GGACGGAATTGTGCCCTCACTCACCTACCAGGTGTTCTTCATGAAGAAGCTGTGGACCACC
ACGGTGCCAGGGAAGGATCCCATGGCCGATTCCATCTTCCACTATTACCAGGAGTTGCCCA
AGTATCTCCGAGGCTACCACAAGTGCACGCGGGAGGAGGTGCTGCAGCTGGGGGCGCTGAT
CTACAGGGTCAAGTTCGAGGAGGACAAGTCCTACTTCCCCAGCATCCCCAAGCTGCTGCGG
GAGCTGGTGCCCCAGGACCTTATCCGGCAGGTCTCACCTGATGACTGGAAGCGGTCCATCG
TCGCCTACTTCAACAAGCACGCAGGGAAGTCCAAGGAGGAGGCCAAGCTGGCCTTCCTGAA
GCTCATCTTCAAGTGGCCCACCTTTGGCTCAGCCTTCTTCGAGGTGAAGCAAACTACGGAGC
CAAACTTCCCTGAGATCCTCCTAATTGCCATCAACAAGTATGGGGTCAGCCTCATCGATCCC
AAAACGAAGGATATCCTCACCACTCATCCCTTCACCAAGATCTCCAACTGGAGCAGCGGCA
ACACCTACTTCCACATCACCATTGGGAACTTGGTGCGCGGGAGCAAACTGCTCTGCGAGAC
GTCACTGGGCTACAAGATGGATGACCTCCTGACTTCCTACATTAGCCAGATGCTCACAGCC
ATGAGCAAACAGCGGGGCTCCAGGAGCGGCAAGTACCCTTACGATGTACCGGATTACGCAT
GAGGTACCAAGGGCGAATTCTGCAGTCGACTAGAGCTCGCTGATCAGCCTCGACTGTGCCT
TCTAGTTGCCAGCCATCTGTTGTTTGCCCCTCCCCCGTGCCTTCCTTGACCCTGGAAGGTGCC
ACTCCCACTGTCCTTTCCTAATAAAATGAGGAAATTGCATCGCATTGTCTGAGTAGGTGTCA
TTCTATTCTGGGGGGTGGGGTGGGGCAGGACAGCAAGGGGGAGGATTGGGAAGACAATAG
CAGGCATGCTGGGGAGAGATCTGGAGGACTAGTCCGTCGACTGTTAATTAAGCATGCTGGG
GAGAGATCTAGGAAACCCCTAGTGATGGAGTTGGCCACTCCCTCTCTGCGCGCTCGCTCGCT
CACTGAGGCCGGGCGACCAAAGGTCGCCCGACGCCCGGGCTTTGCCCGGGCGGCCTCAGTG
AGCGAGCGAGCGCGCAGAGAGGGAG
SEQ ID NO: 36 is the nucleotide sequence of the CMvl overlap front-half
vector (i.e., AAV-smCBA-hMYO7A-noDimNT-CMvl):
CTCCCTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCC
ATCACTAGGGGTTCTCAGATCTGGCGCGCCCAATTCGGTACCCTAGTTATTAATAGTAATCA
ATTACGGGGTCATTAGTTCATAGCCCATATATGGAGTTCCGCGTTACATAACTTACGGTAAA
TGGCCCGCCTGGCTGACCGCCCAACGACCCCCGCCCATTGACGTCAATAATGACGTATGTTC
CCATAGTAACGCCAATAGGGACTTTCCATTGACGTCAATGGGTGGACTATTTACGGTAAAC
TGCCCACTTGGCAGTACATCAAGTGTATCATATGCCAAGTACGCCCCCTATTGACGTCAATG
ACGGTAAATGGCCCGCCTGGCATTATGCCCAGTACATGACCTTATGGGACTTTCCTACTTGG
CAGTACATCTACGTATTAGTCATCGCTATTACCATGGTCGAGGTGAGCCCCACGTTCTGCTT
CACTCTCCCCATCTCCCCCCCCTCCCCACCCCCAATTTTGTATTTATTTATTTTTTAATTATTT
TGTGCAGCGATGGGGGCGGGGGGGGGGGGGGGGCGCGCGCCAGGCGGGGCGGGGCGGGG
CGAGGGGCGGGGCGGGGCGAGGCGGAGAGGTGCGGCGGCAGCCAATCAGAGCGGCGCGC
TCCGAAAGTTTCCTTTTATGGCGAGGCGGCGGCGGCGGCGGCCCTATAAAAAGCGAAGCGC
GCGGCGGGCGGGAGTCGCTGCGCGCTGCCTTCGCCCCGTGCCCCGCTCCGCCGCCGCCTCG
CGCCGCCCGCCCCGGCTCTGACTGACCGCGTTACTCCCACAGGTGAGCGGGCGGGACGGCC
CTTCTCCTCCGGGCTGTAATTAGCGCTTGGTTTAATGACGGCTTGTTTCTTTTCTGTGGCTGC
GTGAAAGCCTTGAGGGGCTCCGGGAGCTAGAGCCTCTGCTAACCATGTTCATGCCTTCTTCT
TTTTCCTACAGCTCCTGGGCAACGTGCTGGTTATTGTGCTGTCTCATCATTTTGGCAAAGAA
TTCTAGCGGCCGCCACCATGGTGATTCTTCAGCAGGGGGACCATGTGTGGATGGACCTGAG
ATTGGGGCAGGAGTTCGACGTGCCCATCGGGGCGGTGGTGAAGCTCTGCGACTCTGGGCAG
GTCCAGGTGGTGGATGATGAAGACAATGAACACTGGATCTCTCCGCAGAACGCAACGCACA
TCAAGCCTATGCACCCCACGTCGGTCCACGGCGTGGAGGACATGATCCGCCTGGGGGACCT
CAACGAGGCGGGCATCTTGCGCAACCTGCTTATCCGCTACCGGGACCACCTCATCTACACG
TATACGGGCTCCATCCTGGTGGCTGTGAACCCCTACCAGCTGCTCTCCATCTACTCGCCAGA
GCACATCCGCCAGTATACCAACAAGAAGATTGGGGAGATGCCCCCCCACATCTTTGCCATT
GCTGACAACTGCTACTTCAACATGAAACGCAACAGCCGAGACCAGTGCTGCATCATCAGTG
GGGAATCTGGGGCCGGGAAGACGGAGAGCACAAAGCTGATCCTGCAGTTCCTGGCAGCCA
TCAGTGGGCAGCACTCGTGGATTGAGCAGCAGGTCTTGGAGGCCACCCCCATTCTGGAAGC
ATTTGGGAATGCCAAGACCATCCGCAATGACAACTCAAGCCGTTTCGGAAAGTACATCGAC
ATCCACTTCAACAAGCGGGGCGCCATCGAGGGCGCGAAGATTGAGCAGTACCTGCTGGAA
AAGTCACGTGTCTGTCGCCAGGCCCTGGATGAAAGGAACTACCACGTGTTCTACTGCATGC
TGGAGGGTATGAGTGAGGATCAGAAGAAGAAGCTGGGCTTGGGCCAGGCCTCTGACTACA
ACTACTTGGCCATGGGTAACTGCATAACCTGTGAGGGCCGGGTGGACAGCCAGGAGTACGC
CAACATCCGCTCCGCCATGAAGGTGCTCATGTTCACTGACACCGAGAACTGGGAGATCTCG
AAGCTCCTGGCTGCCATCCTGCACCTGGGCAACCTGCAGTATGAGGCACGCACATTTGAAA
ACCTGGATGCCTGTGAGGTTCTCTTCTCCCCATCGCTGGCCACAGCTGCATCCCTGCTTGAG
GTGAACCCCCCAGACCTGATGAGCTGCCTGACTAGCCGCACCCTCATCACCCGCGGGGAGA
CGGTGTCCACCCCACTGAGCAGGGAACAGGCACTGGACGTGCGCGACGCCTTCGTAAAGGG
GATCTACGGGCGGCTGTTCGTGTGGATTGTGGACAAGATCAACGCAGCAATTTACAAGCCT
CCCTCCCAGGATGTGAAGAACTCTCGCAGGTCCATCGGCCTCCTGGACATCTTTGGGTTTGA
GAACTTTGCTGTGAACAGCTTTGAGCAGCTCTGCATCAACTTCGCCAATGAGCACCTGCAGC
AGTTCTTTGTGCGGCACGTGTTCAAGCTGGAGCAGGAGGAATATGACCTGGAGAGCATTGA
CTGGCTGCACATCGAGTTCACTGACAACCAGGATGCCCTGGACATGATTGCCAACAAGCCC
ATGAACATCATCTCCCTCATCGATGAGGAGAGCAAGTTCCCCAAGGGCACAGACACCACCA
TGTTACACAAGCTGAACTCCCAGCACAAGCTCAACGCCAACTACATCCCCCCCAAGAACAA
CCATGAGACCCAGTTTGGCATCAACCATTTTGCAGGCATCGTCTACTATGAGACCCAAGGCT
TCCTGGAGAAGAACCGAGACACCCTGCATGGGGACATTATCCAGCTGGTCCACTCCTCCAG
GAACAAGTTCATCAAGCAGATCTTCCAGGCCGATGTCGCCATGGGCGCCGAGACCAGGAAG
CGCTCGCCCACACTTAGCAGCCAGTTCAAGCGGTCACTGGAGCTGCTGATGCGCACGCTGG
GTGCCTGCCAGCCCTTCTTTGTGCGATGCATCAAGCCCAATGAGTTCAAGAAGCCCATGCTG
TTCGACCGGCACCTGTGCGTGCGCCAGCTGCGGTACTCAGGAATGATGGAGACCATCCGAA
TCCGCCGAGCTGGCTACCCCATCCGCTACAGCTTCGTAGAGTTTGTGGAGCGGTACCGTGTG
CTGCTGCCAGGTGTGAAGCCGGCCTACAAGCAGGGCGACCTCCGCGGGACTTGCCAGCGCA
TGGCTGAGGCTGTGCTGGGCACCCACGATGACTGGCAGATAGGCAAAACCAAGATCTTTCT
GAAGGACCACCATGACATGCTGCTGGAAGTGGAGCGGGACAAAGCCATCACCGACAGAGT
CATCCTCCTTCAGAAAGTCATCCGGGGATTCAAAGACAGGTCTAACTTTCTGAAGCTGAAG
AACGCTGCCACACTGATCCAGAGGCACTGGCGGGGTCACAACTGTAGGAAGAACTACGGG
CTGATGCGTCTGGGCTTCCTGCGGCTGCAGGCCCTGCACCGCTCCCGGAAGCTGCACCAGC
AGTACCGCCTGGCCCGCCAGCGCATCATCCAGTTCCAGGCCCGCTGCCGCGCCTATCTGGTG
CGCAAGGCCTTCCGCCACCGCCTCTGGGCTGTGCTCACCGTGCAGGCCTATGCCCGGGGCA
TGATCGCCCGCAGGCTGCACCAACGCCTCAGGGCTGAGTATCTGTGGCGCCTCGAGGCTGA
GAAAATGCGGCTGGCGGAGGAAGAGAAGCTTAGAGGATCCTCCCGTCGACTGTTTAAGCAT
GCTGGGGAGAGATCTGAGGAAACCCCTAGTGATGGAGTTGGCCACTCCCTCTCTGCGCGCT
CGCTCGCTCACTGAGGCCGGGCGACCAAAGGTCGCCCGACGCCCGGGCTTTGCCCGGGCGG
CCTCAGTGAGCGAGCGAGCGCGCAGAGAGGGAG
SEQ ID NO: 62 (peptide encoded by the N-myosin7A portion of SEQ ID NO: 36
(AAV-smCBA-hMYO7A-noDimNT-CMv1))
MVILQQGDHVWMDLRLGQEFDVPIGAVVKLCDSGQVQVVDDEDNEHWISPQNATHIKPMHPT
SVHGVEDMIRLGDLNEAGILRNLLIRYRDHLIYTYTGSILVAVNPYQLLSIYSPEHIRQYTNKKIG
EMPPHIFAIADNCYFNMKRNSRDQCCIISGESGAGKTESTKLILQFLAAISGQHSWIEQQVLEATP
ILEAFGNAKTIRNDNSSRFGKYIDIHFNKRGAIEGAKIEQYLLEKSRVCRQALDERNYHVFYCML
EGMSEDQKKKLGLGQASDYNYLAMGNCITCEGRVDSQEYANIRSAMKVLMFTDTENWEISKL
LAAILHLGNLQYEARTFENLDACEVLFSPSLATAASLLEVNPPDLMSCLTSRTLITRGETVSTPLS
REQALDVRDAFVKGIYGRLFVWIVDKINAAIYKPPSQDVKNSRRSIGLLDIFGFENFAVNSFEQL
CINFANEHLQQFFVRHVFKLEQEEYDLESIDWLHIEFTDNQDALDMIANKPMNIISLIDEESKFPK
GTDTTMLHKLNSQHKLNANYIPPKNNHETQFGINHFAGIVYYETQGFLEKNRDTLHGDIIQLVH
SSRNKFIKQIFQADVAMGAETRKRSPTLSSQFKRSLELLMRTLGACQPFFVRCIKPNEFKKPMLF
DRHLCVRQLRYSGMMETIRIRRAGYPIRYSFVEFVERYRVLLPGVKPAYKQGDLRGTCQRMAE
AVLGTHDDWQIGKTKIFLKDHHDMLLEVERDKAITDRVILLQKVIRGFKDRSNFLKLKNAATLI
QRHWRGHNCRKNYGLMRLGFLRLQALHRSRKLHQQYRLARQRIIQFQARCRAYLVRKAFRHR
LWAVLTVQAYARGMIARRLHQRLRAEYLWRLEAEKMRLAEEEKL
SEQ ID NO: 63 (only the N-myosin7A portion of SEQ ID NO: 36 (AAV-smCBA-
hMYO7A-noDimNT-CMv1))
ATGGTGATTCTTCAGCAGGGGGACCATGTGTGGATGGACCTGAGATTGGGGCAGGAGTTCG
ACGTGCCCATCGGGGCGGTGGTGAAGCTCTGCGACTCTGGGCAGGTCCAGGTGGTGGATGA
TGAAGACAATGAACACTGGATCTCTCCGCAGAACGCAACGCACATCAAGCCTATGCACCCC
ACGTCGGTCCACGGCGTGGAGGACATGATCCGCCTGGGGGACCTCAACGAGGCGGGCATCT
TGCGCAACCTGCTTATCCGCTACCGGGACCACCTCATCTACACGTATACGGGCTCCATCCTG
GTGGCTGTGAACCCCTACCAGCTGCTCTCCATCTACTCGCCAGAGCACATCCGCCAGTATAC
CAACAAGAAGATTGGGGAGATGCCCCCCCACATCTTTGCCATTGCTGACAACTGCTACTTC
AACATGAAACGCAACAGCCGAGACCAGTGCTGCATCATCAGTGGGGAATCTGGGGCCGGG
AAGACGGAGAGCACAAAGCTGATCCTGCAGTTCCTGGCAGCCATCAGTGGGCAGCACTCGT
GGATTGAGCAGCAGGTCTTGGAGGCCACCCCCATTCTGGAAGCATTTGGGAATGCCAAGAC
CATCCGCAATGACAACTCAAGCCGTTTCGGAAAGTACATCGACATCCACTTCAACAAGCGG
GGCGCCATCGAGGGCGCGAAGATTGAGCAGTACCTGCTGGAAAAGTCACGTGTCTGTCGCC
AGGCCCTGGATGAAAGGAACTACCACGTGTTCTACTGCATGCTGGAGGGTATGAGTGAGGA
TCAGAAGAAGAAGCTGGGCTTGGGCCAGGCCTCTGACTACAACTACTTGGCCATGGGTAAC
TGCATAACCTGTGAGGGCCGGGTGGACAGCCAGGAGTACGCCAACATCCGCTCCGCCATGA
AGGTGCTCATGTTCACTGACACCGAGAACTGGGAGATCTCGAAGCTCCTGGCTGCCATCCT
GCACCTGGGCAACCTGCAGTATGAGGCACGCACATTTGAAAACCTGGATGCCTGTGAGGTT
CTCTTCTCCCCATCGCTGGCCACAGCTGCATCCCTGCTTGAGGTGAACCCCCCAGACCTGAT
GAGCTGCCTGACTAGCCGCACCCTCATCACCCGCGGGGAGACGGTGTCCACCCCACTGAGC
AGGGAACAGGCACTGGACGTGCGCGACGCCTTCGTAAAGGGGATCTACGGGCGGCTGTTCG
TGTGGATTGTGGACAAGATCAACGCAGCAATTTACAAGCCTCCCTCCCAGGATGTGAAGAA
CTCTCGCAGGTCCATCGGCCTCCTGGACATCTTTGGGTTTGAGAACTTTGCTGTGAACAGCT
TTGAGCAGCTCTGCATCAACTTCGCCAATGAGCACCTGCAGCAGTTCTTTGTGCGGCACGTG
TTCAAGCTGGAGCAGGAGGAATATGACCTGGAGAGCATTGACTGGCTGCACATCGAGTTCA
CTGACAACCAGGATGCCCTGGACATGATTGCCAACAAGCCCATGAACATCATCTCCCTCAT
CGATGAGGAGAGCAAGTTCCCCAAGGGCACAGACACCACCATGTTACACAAGCTGAACTCC
CAGCACAAGCTCAACGCCAACTACATCCCCCCCAAGAACAACCATGAGACCCAGTTTGGCA
TCAACCATTTTGCAGGCATCGTCTACTATGAGACCCAAGGCTTCCTGGAGAAGAACCGAGA
CACCCTGCATGGGGACATTATCCAGCTGGTCCACTCCTCCAGGAACAAGTTCATCAAGCAG
ATCTTCCAGGCCGATGTCGCCATGGGCGCCGAGACCAGGAAGCGCTCGCCCACACTTAGCA
GCCAGTTCAAGCGGTCACTGGAGCTGCTGATGCGCACGCTGGGTGCCTGCCAGCCCTTCTTT
GTGCGATGCATCAAGCCCAATGAGTTCAAGAAGCCCATGCTGTTCGACCGGCACCTGTGCG
TGCGCCAGCTGCGGTACTCAGGAATGATGGAGACCATCCGAATCCGCCGAGCTGGCTACCC
CATCCGCTACAGCTTCGTAGAGTTTGTGGAGCGGTACCGTGTGCTGCTGCCAGGTGTGAAG
CCGGCCTACAAGCAGGGCGACCTCCGCGGGACTTGCCAGCGCATGGCTGAGGCTGTGCTGG
GCACCCACGATGACTGGCAGATAGGCAAAACCAAGATCTTTCTGAAGGACCACCATGACAT
GCTGCTGGAAGTGGAGCGGGACAAAGCCATCACCGACAGAGTCATCCTCCTTCAGAAAGTC
ATCCGGGGATTCAAAGACAGGTCTAACTTTCTGAAGCTGAAGAACGCTGCCACACTGATCC
AGAGGCACTGGCGGGGTCACAACTGTAGGAAGAACTACGGGCTGATGCGTCTGGGCTTCCT
GCGGCTGCAGGCCCTGCACCGCTCCCGGAAGCTGCACCAGCAGTACCGCCTGGCCCGCCAG
CGCATCATCCAGTTCCAGGCCCGCTGCCGCGCCTATCTGGTGCGCAAGGCCTTCCGCCACCG
CCTCTGGGCTGTGCTCACCGTGCAGGCCTATGCCCGGGGCATGATCGCCCGCAGGCTGCAC
CAACGCCTCAGGGCTGAGTATCTGTGGCGCCTCGAGGCTGAGAAAATGCGGCTGGCGGAGG
AAGAGAAGCTT
SEQ ID NO: 37 is the nucleotide sequence of the second generation
overlap front-half vector (i.e., AAV-smCBA-hMYO7A-noDIM-NTlong):
CTCCCTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCC
ATCACTAGGGGTTCTCAGATCTGGCGCGCCCAATTCGGTACCCTAGTTATTAATAGTAATCA
ATTACGGGGTCATTAGTTCATAGCCCATATATGGAGTTCCGCGTTACATAACTTACGGTAAA
TGGCCCGCCTGGCTGACCGCCCAACGACCCCCGCCCATTGACGTCAATAATGACGTATGTTC
CCATAGTAACGCCAATAGGGACTTTCCATTGACGTCAATGGGTGGACTATTTACGGTAAAC
TGCCCACTTGGCAGTACATCAAGTGTATCATATGCCAAGTACGCCCCCTATTGACGTCAATG
ACGGTAAATGGCCCGCCTGGCATTATGCCCAGTACATGACCTTATGGGACTTTCCTACTTGG
CAGTACATCTACGTATTAGTCATCGCTATTACCATGGTCGAGGTGAGCCCCACGTTCTGCTT
CACTCTCCCCATCTCCCCCCCCTCCCCACCCCCAATTTTGTATTTATTTATTTTTTAATTATTT
TGTGCAGCGATGGGGGCGGGGGGGGGGGGGGGGCGCGCGCCAGGCGGGGCGGGGCGGGG
CGAGGGGCGGGGCGGGGCGAGGCGGAGAGGTGCGGCGGCAGCCAATCAGAGCGGCGCGC
TCCGAAAGTTTCCTTTTATGGCGAGGCGGCGGCGGCGGCGGCCCTATAAAAAGCGAAGCGC
GCGGCGGGCGGGAGTCGCTGCGCGCTGCCTTCGCCCCGTGCCCCGCTCCGCCGCCGCCTCG
CGCCGCCCGCCCCGGCTCTGACTGACCGCGTTACTCCCACAGGTGAGCGGGCGGGACGGCC
CTTCTCCTCCGGGCTGTAATTAGCGCTTGGTTTAATGACGGCTTGTTTCTTTTCTGTGGCTGC
GTGAAAGCCTTGAGGGGCTCCGGGAGCTAGAGCCTCTGCTAACCATGTTCATGCCTTCTTCT
TTTTCCTACAGCTCCTGGGCAACGTGCTGGTTATTGTGCTGTCTCATCATTTTGGCAAAGAA
TTCTAGCGGCCGCCACCATGGTGATTCTTCAGCAGGGGGACCATGTGTGGATGGACCTGAG
ATTGGGGCAGGAGTTCGACGTGCCCATCGGGGCGGTGGTGAAGCTCTGCGACTCTGGGCAG
GTCCAGGTGGTGGATGATGAAGACAATGAACACTGGATCTCTCCGCAGAACGCAACGCACA
TCAAGCCTATGCACCCCACGTCGGTCCACGGCGTGGAGGACATGATCCGCCTGGGGGACCT
CAACGAGGCGGGCATCTTGCGCAACCTGCTTATCCGCTACCGGGACCACCTCATCTACACG
TATACGGGCTCCATCCTGGTGGCTGTGAACCCCTACCAGCTGCTCTCCATCTACTCGCCAGA
GCACATCCGCCAGTATACCAACAAGAAGATTGGGGAGATGCCCCCCCACATCTTTGCCATT
GCTGACAACTGCTACTTCAACATGAAACGCAACAGCCGAGACCAGTGCTGCATCATCAGTG
GGGAATCTGGGGCCGGGAAGACGGAGAGCACAAAGCTGATCCTGCAGTTCCTGGCAGCCA
TCAGTGGGCAGCACTCGTGGATTGAGCAGCAGGTCTTGGAGGCCACCCCCATTCTGGAAGC
ATTTGGGAATGCCAAGACCATCCGCAATGACAACTCAAGCCGTTTCGGAAAGTACATCGAC
ATCCACTTCAACAAGCGGGGCGCCATCGAGGGCGCGAAGATTGAGCAGTACCTGCTGGAA
AAGTCACGTGTCTGTCGCCAGGCCCTGGATGAAAGGAACTACCACGTGTTCTACTGCATGC
TGGAGGGTATGAGTGAGGATCAGAAGAAGAAGCTGGGCTTGGGCCAGGCCTCTGACTACA
ACTACTTGGCCATGGGTAACTGCATAACCTGTGAGGGCCGGGTGGACAGCCAGGAGTACGC
CAACATCCGCTCCGCCATGAAGGTGCTCATGTTCACTGACACCGAGAACTGGGAGATCTCG
AAGCTCCTGGCTGCCATCCTGCACCTGGGCAACCTGCAGTATGAGGCACGCACATTTGAAA
ACCTGGATGCCTGTGAGGTTCTCTTCTCCCCATCGCTGGCCACAGCTGCATCCCTGCTTGAG
GTGAACCCCCCAGACCTGATGAGCTGCCTGACTAGCCGCACCCTCATCACCCGCGGGGAGA
CGGTGTCCACCCCACTGAGCAGGGAACAGGCACTGGACGTGCGCGACGCCTTCGTAAAGGG
GATCTACGGGCGGCTGTTCGTGTGGATTGTGGACAAGATCAACGCAGCAATTTACAAGCCT
CCCTCCCAGGATGTGAAGAACTCTCGCAGGTCCATCGGCCTCCTGGACATCTTTGGGTTTGA
GAACTTTGCTGTGAACAGCTTTGAGCAGCTCTGCATCAACTTCGCCAATGAGCACCTGCAGC
AGTTCTTTGTGCGGCACGTGTTCAAGCTGGAGCAGGAGGAATATGACCTGGAGAGCATTGA
CTGGCTGCACATCGAGTTCACTGACAACCAGGATGCCCTGGACATGATTGCCAACAAGCCC
ATGAACATCATCTCCCTCATCGATGAGGAGAGCAAGTTCCCCAAGGGCACAGACACCACCA
TGTTACACAAGCTGAACTCCCAGCACAAGCTCAACGCCAACTACATCCCCCCCAAGAACAA
CCATGAGACCCAGTTTGGCATCAACCATTTTGCAGGCATCGTCTACTATGAGACCCAAGGCT
TCCTGGAGAAGAACCGAGACACCCTGCATGGGGACATTATCCAGCTGGTCCACTCCTCCAG
GAACAAGTTCATCAAGCAGATCTTCCAGGCCGATGTCGCCATGGGCGCCGAGACCAGGAAG
CGCTCGCCCACACTTAGCAGCCAGTTCAAGCGGTCACTGGAGCTGCTGATGCGCACGCTGG
GTGCCTGCCAGCCCTTCTTTGTGCGATGCATCAAGCCCAATGAGTTCAAGAAGCCCATGCTG
TTCGACCGGCACCTGTGCGTGCGCCAGCTGCGGTACTCAGGAATGATGGAGACCATCCGAA
TCCGCCGAGCTGGCTACCCCATCCGCTACAGCTTCGTAGAGTTTGTGGAGCGGTACCGTGTG
CTGCTGCCAGGTGTGAAGCCGGCCTACAAGCAGGGCGACCTCCGCGGGACTTGCCAGCGCA
TGGCTGAGGCTGTGCTGGGCACCCACGATGACTGGCAGATAGGCAAAACCAAGATCTTTCT
GAAGGACCACCATGACATGCTGCTGGAAGTGGAGCGGGACAAAGCCATCACCGACAGAGT
CATCCTCCTTCAGAAAGTCATCCGGGGATTCAAAGACAGGTCTAACTTTCTGAAGCTGAAG
AACGCTGCCACACTGATCCAGAGGCACTGGCGGGGTCACAACTGTAGGAAGAACTACGGG
CTGATGCGTCTGGGCTTCCTGCGGCTGCAGGCCCTGCACCGCTCCCGGAAGCTGCACCAGC
AGTACCGCCTGGCCCGCCAGCGCATCATCCAGTTCCAGGCCCGCTGCCGCGCCTATCTGGTG
CGCAAGGCCTTCCGCCACCGCCTCTGGGCTGTGCTCACCGTGCAGGCCTATGCCCGGGGCA
TGATCGCCCGCAGGCTGCACCAACGCCTCAGGGCTGAGTATCTGTGGCGCCTCGAGGCTGA
GAAAATGCGGCTGGCGGAGGAAGAGAAGCTTTGAAAGTGACATTAGGCTCCCGTCGACTGT
TAATTAAGCATGCTGGGGAGAGATCTGAGGAAACCCCTAGTGATGGAGTTGGCCACTCCCT
CTCTGCGCGCTCGCTCGCTCACTGAGGCCGGGCGACCAAAGGTCGCCCGACGCCCGGGCTT
TGCCCGGGCGGCCTCAGTGAGCGAGCGAGCGCGCAGAGAGGGAG
SEQ ID NO: 90 (only the N-myosin7A portion of SEQ ID NO: 37 (AAV-
smCBA-hMYO7A-noDIM-NTlong))
ATGGTGATTCTTCAGCAGGGGGACCATGTGTGGATGGACCTGAGATTGGGGCAGGAGTTCG
ACGTGCCCATCGGGGCGGTGGTGAAGCTCTGCGACTCTGGGCAGGTCCAGGTGGTGGATGA
TGAAGACAATGAACACTGGATCTCTCCGCAGAACGCAACGCACATCAAGCCTATGCACCCC
ACGTCGGTCCACGGCGTGGAGGACATGATCCGCCTGGGGGACCTCAACGAGGCGGGCATCT
TGCGCAACCTGCTTATCCGCTACCGGGACCACCTCATCTACACGTATACGGGCTCCATCCTG
GTGGCTGTGAACCCCTACCAGCTGCTCTCCATCTACTCGCCAGAGCACATCCGCCAGTATAC
CAACAAGAAGATTGGGGAGATGCCCCCCCACATCTTTGCCATTGCTGACAACTGCTACTTC
AACATGAAACGCAACAGCCGAGACCAGTGCTGCATCATCAGTGGGGAATCTGGGGCCGGG
AAGACGGAGAGCACAAAGCTGATCCTGCAGTTCCTGGCAGCCATCAGTGGGCAGCACTCGT
GGATTGAGCAGCAGGTCTTGGAGGCCACCCCCATTCTGGAAGCATTTGGGAATGCCAAGAC
CATCCGCAATGACAACTCAAGCCGTTTCGGAAAGTACATCGACATCCACTTCAACAAGCGG
GGCGCCATCGAGGGCGCGAAGATTGAGCAGTACCTGCTGGAAAAGTCACGTGTCTGTCGCC
AGGCCCTGGATGAAAGGAACTACCACGTGTTCTACTGCATGCTGGAGGGTATGAGTGAGGA
TCAGAAGAAGAAGCTGGGCTTGGGCCAGGCCTCTGACTACAACTACTTGGCCATGGGTAAC
TGCATAACCTGTGAGGGCCGGGTGGACAGCCAGGAGTACGCCAACATCCGCTCCGCCATGA
AGGTGCTCATGTTCACTGACACCGAGAACTGGGAGATCTCGAAGCTCCTGGCTGCCATCCT
GCACCTGGGCAACCTGCAGTATGAGGCACGCACATTTGAAAACCTGGATGCCTGTGAGGTT
CTCTTCTCCCCATCGCTGGCCACAGCTGCATCCCTGCTTGAGGTGAACCCCCCAGACCTGAT
GAGCTGCCTGACTAGCCGCACCCTCATCACCCGCGGGGAGACGGTGTCCACCCCACTGAGC
AGGGAACAGGCACTGGACGTGCGCGACGCCTTCGTAAAGGGGATCTACGGGCGGCTGTTCG
TGTGGATTGTGGACAAGATCAACGCAGCAATTTACAAGCCTCCCTCCCAGGATGTGAAGAA
CTCTCGCAGGTCCATCGGCCTCCTGGACATCTTTGGGTTTGAGAACTTTGCTGTGAACAGCT
TTGAGCAGCTCTGCATCAACTTCGCCAATGAGCACCTGCAGCAGTTCTTTGTGCGGCACGTG
TTCAAGCTGGAGCAGGAGGAATATGACCTGGAGAGCATTGACTGGCTGCACATCGAGTTCA
CTGACAACCAGGATGCCCTGGACATGATTGCCAACAAGCCCATGAACATCATCTCCCTCAT
CGATGAGGAGAGCAAGTTCCCCAAGGGCACAGACACCACCATGTTACACAAGCTGAACTCC
CAGCACAAGCTCAACGCCAACTACATCCCCCCCAAGAACAACCATGAGACCCAGTTTGGCA
TCAACCATTTTGCAGGCATCGTCTACTATGAGACCCAAGGCTTCCTGGAGAAGAACCGAGA
CACCCTGCATGGGGACATTATCCAGCTGGTCCACTCCTCCAGGAACAAGTTCATCAAGCAG
ATCTTCCAGGCCGATGTCGCCATGGGCGCCGAGACCAGGAAGCGCTCGCCCACACTTAGCA
GCCAGTTCAAGCGGTCACTGGAGCTGCTGATGCGCACGCTGGGTGCCTGCCAGCCCTTCTTT
GTGCGATGCATCAAGCCCAATGAGTTCAAGAAGCCCATGCTGTTCGACCGGCACCTGTGCG
TGCGCCAGCTGCGGTACTCAGGAATGATGGAGACCATCCGAATCCGCCGAGCTGGCTACCC
CATCCGCTACAGCTTCGTAGAGTTTGTGGAGCGGTACCGTGTGCTGCTGCCAGGTGTGAAG
CCGGCCTACAAGCAGGGCGACCTCCGCGGGACTTGCCAGCGCATGGCTGAGGCTGTGCTGG
GCACCCACGATGACTGGCAGATAGGCAAAACCAAGATCTTTCTGAAGGACCACCATGACAT
GCTGCTGGAAGTGGAGCGGGACAAAGCCATCACCGACAGAGTCATCCTCCTTCAGAAAGTC
ATCCGGGGATTCAAAGACAGGTCTAACTTTCTGAAGCTGAAGAACGCTGCCACACTGATCC
AGAGGCACTGGCGGGGTCACAACTGTAGGAAGAACTACGGGCTGATGCGTCTGGGCTTCCT
GCGGCTGCAGGCCCTGCACCGCTCCCGGAAGCTGCACCAGCAGTACCGCCTGGCCCGCCAG
CGCATCATCCAGTTCCAGGCCCGCTGCCGCGCCTATCTGGTGCGCAAGGCCTTCCGCCACCG
CCTCTGGGCTGTGCTCACCGTGCAGGCCTATGCCCGGGGCATGATCGCCCGCAGGCTGCAC
CAACGCCTCAGGGCTGAGTATCTGTGGCGCCTCGAGGCTGAGAAAATGCGGCTGGCGGAGG
AAGAGAAGCTT
SEQ ID NO: 91 (only the N-myosin7A portion of SEQ ID NO: 37 (AAV-
smCBA-hMYO7A-noDIM-NTlong))
MVILQQGDHVWMDLRLGQEFDVPIGAVVKLCDSGQVQVVDDEDNEHWISPQNATHIKPMHPT
SVHGVEDMIRLGDLNEAGILRNLLIRYRDHLIYTYTGSILVAVNPYQLLSIYSPEHIRQYTNKKIG
EMPPHIFAIADNCYFNMKRNSRDQCCIISGESGAGKTESTKLILQFLAAISGQHSWIEQQVLEATP
ILEAFGNAKTIRNDNSSRFGKYIDIHFNKRGAIEGAKIEQYLLEKSRVCRQALDERNYHVFYCML
EGMSEDQKKKLGLGQASDYNYLAMGNCITCEGRVDSQEYANIRSAMKVLMFTDTENWEISKL
LAAILHLGNLQYEARTFENLDACEVLFSPSLATAASLLEVNPPDLMSCLTSRTLITRGETVSTPLS
REQALDVRDAFVKGIYGRLFVWIVDKINAAIYKPPSQDVKNSRRSIGLLDIFGFENFAVNSFEQL
CINFANEHLQQFFVRHVFKLEQEEYDLESIDWLHIEFTDNQDALDMIANKPMNIISLIDEESKFPK
GTDTTMLHKLNSQHKLNANYIPPKNNHETQFGINHFAGIVYYETQGFLEKNRDTLHGDIIQLVH
SSRNKFIKQIFQADVAMGAETRKRSPTLSSQFKRSLELLMRTLGACQPFFVRCIKPNEFKKPMLF
DRHLCVRQLRYSGMMETIRIRRAGYPIRYSFVEFVERYRVLLPGVKPAYKQGDLRGTCQRMAE
AVLGTHDDWQIGKTKIFLKDHHDMLLEVERDKAITDRVILLQKVIRGFKDRSNFLKLKNAATLI
QRHWRGHNCRKNYGLMRLGFLRLQALHRSRKLHQQYRLARQRIIQFQARCRAYLVRKAFRHR
LWAVLTVQAYARGMIARRLHQRLRAEYLWRLEAEKMRLAEEEKL
SEQ ID NO: 38 is the nucleotide sequence of the second generation
overlap back-half vector (i.e., AAV-hMYO7A-CTlong-v2.HA).
CTCCCTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCC
ATCACTAGGGGTTCAGATCTGGCGCGCCGGATCCGGGCTGATGCGTCTGGGCTTCCTGCGG
CTGCAGGCCCTGCACCGCTCCCGGAAGCTGCACCAGCAGTACCGCCTGGCCCGCCAGCGCA
TCATCCAGTTCCAGGCCCGCTGCCGCGCCTATCTGGTGCGCAAGGCCTTCCGCCACCGCCTC
TGGGCTGTGCTCACCGTGCAGGCCTATGCCCGGGGCATGATCGCCCGCAGGCTGCACCAAC
GCCTCAGGGCTGAGTATCTGTGGCGCCTCGAGGCTGAGAAAATGCGGCTGGCGGAGGAAG
AGAAGCTTCGGAAGGAGATGAGCGCCAAGAAGGCCAAGGAGGAGGCCGAGCGCAAGCAT
CAGGAGCGCCTGGCCCAGCTGGCTCGTGAGGACGCTGAGCGGGAGCTGAAGGAGAAGGAG
GCCGCTCGGCGGAAGAAGGAGCTCCTGGAGCAGATGGAAAGGGCCCGCCATGAGCCTGTC
AATCACTCAGACATGGTGGACAAGATGTTTGGCTTCCTGGGGACTTCAGGTGGCCTGCCAG
GCCAGGAGGGCCAGGCACCTAGTGGCTTTGAGGACCTGGAGCGAGGGCGGAGGGAGATGG
TGGAGGAGGACCTGGATGCAGCCCTGCCCCTGCCTGACGAGGATGAGGAGGACCTCTCTGA
GTATAAATTTGCCAAGTTCGCGGCCACCTACTTCCAGGGGACAACCACGCACTCCTACACC
CGGCGGCCACTCAAACAGCCACTGCTCTACCATGACGACGAGGGTGACCAGCTGGCAGCCC
TGGCGGTCTGGATCACCATCCTCCGCTTCATGGGGGACCTCCCTGAGCCCAAGTACCACAC
AGCCATGAGTGATGGCAGTGAGAAGATCCCTGTGATGACCAAGATTTATGAGACCCTGGGC
AAGAAGACGTACAAGAGGGAGCTGCAGGCCCTGCAGGGCGAGGGCGAGGCCCAGCTCCCC
GAGGGCCAGAAGAAGAGCAGTGTGAGGCACAAGCTGGTGCATTTGACTCTGAAAAAGAAG
TCCAAGCTCACAGAGGAGGTGACCAAGAGGCTGCATGACGGGGAGTCCACAGTGCAGGGC
AACAGCATGCTGGAGGACCGGCCCACCTCCAACCTGGAGAAGCTGCACTTCATCATCGGCA
ATGGCATCCTGCGGCCAGCACTCCGGGACGAGATCTACTGCCAGATCAGCAAGCAGCTGAC
CCACAACCCCTCCAAGAGCAGCTATGCCCGGGGCTGGATTCTCGTGTCTCTCTGCGTGGGCT
GTTTCGCCCCCTCCGAGAAGTTTGTCAAGTACCTGCGGAACTTCATCCACGGGGGCCCGCCC
GGCTACGCCCCGTACTGTGAGGAGCGCCTGAGAAGGACCTTTGTCAATGGGACACGGACAC
AGCCGCCCAGCTGGCTGGAGCTGCAGGCCACCAAGTCCAAGAAGCCAATCATGTTGCCCGT
GACATTCATGGATGGGACCACCAAGACCCTGCTGACGGACTCGGCAACCACGGCCAAGGA
GCTCTGCAACGCGCTGGCCGACAAGATCTCTCTCAAGGACCGGTTCGGGTTCTCCCTCTACA
TTGCCCTGTTTGACAAGGTGTCCTCCCTGGGCAGCGGCAGTGACCACGTCATGGACGCCATC
TCCCAGTGCGAGCAGTACGCCAAGGAGCAGGGCGCCCAGGAGCGCAACGCCCCCTGGAGG
CTCTTCTTCCGCAAAGAGGTCTTCACGCCCTGGCACAGCCCCTCCGAGGACAACGTGGCCA
CCAACCTCATCTACCAGCAGGTGGTGCGAGGAGTCAAGTTTGGGGAGTACAGGTGTGAGAA
GGAGGACGACCTGGCTGAGCTGGCCTCCCAGCAGTACTTTGTAGACTATGGCTCTGAGATG
ATCCTGGAGCGCCTCCTGAACCTCGTGCCCACCTACATCCCCGACCGCGAGATCACGCCCCT
GAAGACGCTGGAGAAGTGGGCCCAGCTGGCCATCGCCGCCCACAAGAAGGGGATTTATGC
CCAGAGGAGAACTGATGCCCAGAAGGTCAAAGAGGATGTGGTCAGTTATGCCCGCTTCAAG
TGGCCCTTGCTCTTCTCCAGGTTTTATGAAGCCTACAAATTCTCAGGCCCCAGTCTCCCCAA
GAACGACGTCATCGTGGCCGTCAACTGGACGGGTGTGTACTTTGTGGATGAGCAGGAGCAG
GTACTTCTGGAGCTGTCCTTCCCAGAGATCATGGCCGTGTCCAGCAGCAGGGGAGCGAAAA
CGACGGCCCCCAGCTTCACGCTGGCCACCATCAAGGGGGACGAATACACCTTCACCTCCAG
CAATGCTGAGGACATTCGTGACCTGGTGGTCACCTTCCTAGAGGGGCTCCGGAAGAGATCT
AAGTATGTTGTGGCCCTGCAGGATAACCCCAACCCCGCAGGCGAGGAGTCAGGCTTCCTCA
GCTTTGCCAAGGGAGACCTCATCATCCTGGACCATGACACGGGCGAGCAGGTCATGAACTC
GGGCTGGGCCAACGGCATCAATGAGAGGACCAAGCAGCGTGGGGACTTCCCCACCGACAG
TGTGTACGTCATGCCCACTGTCACCATGCCACCGCGGGAGATTGTGGCCCTGGTCACCATGA
CTCCCGATCAGAGGCAGGACGTTGTCCGGCTCTTGCAGCTGCGAACGGCGGAGCCCGAGGT
GCGTGCCAAGCCCTACACGCTGGAGGAGTTTTCCTATGACTACTTCAGGCCCCCACCCAAG
CACACGCTGAGCCGTGTCATGGTGTCCAAGGCCCGAGGCAAGGACCGGCTGTGGAGCCACA
CGCGGGAACCGCTCAAGCAGGCGCTGCTCAAGAAGCTCCTGGGCAGTGAGGAGCTCTCGCA
GGAGGCCTGCCTGGCCTTCATTGCTGTGCTCAAGTACATGGGCGACTACCCGTCCAAGAGG
ACACGCTCCGTCAACGAGCTCACCGACCAGATCTTTGAGGGTCCCCTGAAAGCCGAGCCCC
TGAAGGACGAGGCATATGTGCAGATCCTGAAGCAGCTGACCGACAACCACATCAGGTACA
GCGAGGAGCGGGGTTGGGAGCTGCTCTGGCTGTGCACGGGCCTTTTCCCACCCAGCAACAT
CCTCCTGCCCCACGTGCAGCGCTTCCTGCAGTCCCGAAAGCACTGCCCACTCGCCATCGACT
GCCTGCAACGGCTCCAGAAAGCCCTGAGAAACGGGTCCCGGAAGTACCCTCCGCACCTGGT
GGAGGTGGAGGCCATCCAGCACAAGACCACCCAGATTTTCCACAAAGTCTACTTCCCTGAT
GACACTGACGAGGCCTTCGAAGTGGAGTCCAGCACCAAGGCCAAGGACTTCTGCCAGAAC
ATCGCCACCAGGCTGCTCCTCAAGTCCTCAGAGGGATTCAGCCTCTTTGTCAAAATTGCAGA
CAAGGTCATCAGCGTTCCTGAGAATGACTTCTTCTTTGACTTTGTTCGACACTTGACAGACT
GGATAAAGAAAGCTCGGCCCATCAAGGACGGAATTGTGCCCTCACTCACCTACCAGGTGTT
CTTCATGAAGAAGCTGTGGACCACCACGGTGCCAGGGAAGGATCCCATGGCCGATTCCATC
TTCCACTATTACCAGGAGTTGCCCAAGTATCTCCGAGGCTACCACAAGTGCACGCGGGAGG
AGGTGCTGCAGCTGGGGGCGCTGATCTACAGGGTCAAGTTCGAGGAGGACAAGTCCTACTT
CCCCAGCATCCCCAAGCTGCTGCGGGAGCTGGTGCCCCAGGACCTTATCCGGCAGGTCTCA
CCTGATGACTGGAAGCGGTCCATCGTCGCCTACTTCAACAAGCACGCAGGGAAGTCCAAGG
AGGAGGCCAAGCTGGCCTTCCTGAAGCTCATCTTCAAGTGGCCCACCTTTGGCTCAGCCTTC
TTCGAGGTGAAGCAAACTACGGAGCCAAACTTCCCTGAGATCCTCCTAATTGCCATCAACA
AGTATGGGGTCAGCCTCATCGATCCCAAAACGAAGGATATCCTCACCACTCATCCCTTCACC
AAGATCTCCAACTGGAGCAGCGGCAACACCTACTTCCACATCACCATTGGGAACTTGGTGC
GCGGGAGCAAACTGCTCTGCGAGACGTCACTGGGCTACAAGATGGATGACCTCCTGACTTC
CTACATTAGCCAGATGCTCACAGCCATGAGCAAACAGCGGGGCTCCAGGAGCGGCAAGTA
CCCTTACGATGTACCGGATTACGCATGAGGTACCAAGGGCGAATTCTGCAGTCGACTAGAG
CTCGCTGATCAGCCTCGACTGTGCCTTCTAGTTGCCAGCCATCTGTTGTTTGCCCCTCCCCCG
TGCCTTCCTTGACCCTGGAAGGTGCCACTCCCACTGTCCTTTCCTAATAAAATGAGGAAATT
GCATCGCATTGTCTGAGTAGGTGTCATTCTATTCTGGGGGGTGGGGTGGGGCAGGACAGCA
AGGGGGAGGATTGGGAAGACAATAGCAGGCATGCTGGGGAGAGATCTGAGGATCCTTAAT
TAAGCATGCTGGGGAGAGATCTGAACCCCTAGTGATGGAGTTGGCCACTCCCTCTCTGCGC
GCTCGCTCGCTCACTGAGGCCGGGCGACCAAAGGTCGCCCGACGCCCGGGCTTTGCCCGGG
CGGCCTCAGTGAGCGAGCGAGCGCGCAGAGAGGGAG
SEQ ID NO: 77 (C-term myosin7A (e.g., AAV-hMYO7A-CTlong-v2.HA))
GGGCTGATGCGTCTGGGCTTCCTGCGGCTGCAGGCCCTGCACCGCTCCCGGAAGCTGCACC
AGCAGTACCGCCTGGCCCGCCAGCGCATCATCCAGTTCCAGGCCCGCTGCCGCGCCTATCT
GGTGCGCAAGGCCTTCCGCCACCGCCTCTGGGCTGTGCTCACCGTGCAGGCCTATGCCCGG
GGCATGATCGCCCGCAGGCTGCACCAACGCCTCAGGGCTGAGTATCTGTGGCGCCTCGAGG
CTGAGAAAATGCGGCTGGCGGAGGAAGAGAAGCTTCGGAAGGAGATGAGCGCCAAGAAG
GCCAAGGAGGAGGCCGAGCGCAAGCATCAGGAGCGCCTGGCCCAGCTGGCTCGTGAGGAC
GCTGAGCGGGAGCTGAAGGAGAAGGAGGCCGCTCGGCGGAAGAAGGAGCTCCTGGAGCAG
ATGGAAAGGGCCCGCCATGAGCCTGTCAATCACTCAGACATGGTGGACAAGATGTTTGGCT
TCCTGGGGACTTCAGGTGGCCTGCCAGGCCAGGAGGGCCAGGCACCTAGTGGCTTTGAGGA
CCTGGAGCGAGGGCGGAGGGAGATGGTGGAGGAGGACCTGGATGCAGCCCTGCCCCTGCC
TGACGAGGATGAGGAGGACCTCTCTGAGTATAAATTTGCCAAGTTCGCGGCCACCTACTTC
CAGGGGACAACCACGCACTCCTACACCCGGCGGCCACTCAAACAGCCACTGCTCTACCATG
ACGACGAGGGTGACCAGCTGGCAGCCCTGGCGGTCTGGATCACCATCCTCCGCTTCATGGG
GGACCTCCCTGAGCCCAAGTACCACACAGCCATGAGTGATGGCAGTGAGAAGATCCCTGTG
ATGACCAAGATTTATGAGACCCTGGGCAAGAAGACGTACAAGAGGGAGCTGCAGGCCCTG
CAGGGCGAGGGCGAGGCCCAGCTCCCCGAGGGCCAGAAGAAGAGCAGTGTGAGGCACAAG
CTGGTGCATTTGACTCTGAAAAAGAAGTCCAAGCTCACAGAGGAGGTGACCAAGAGGCTGC
ATGACGGGGAGTCCACAGTGCAGGGCAACAGCATGCTGGAGGACCGGCCCACCTCCAACC
TGGAGAAGCTGCACTTCATCATCGGCAATGGCATCCTGCGGCCAGCACTCCGGGACGAGAT
CTACTGCCAGATCAGCAAGCAGCTGACCCACAACCCCTCCAAGAGCAGCTATGCCCGGGGC
TGGATTCTCGTGTCTCTCTGCGTGGGCTGTTTCGCCCCCTCCGAGAAGTTTGTCAAGTACCT
GCGGAACTTCATCCACGGGGGCCCGCCCGGCTACGCCCCGTACTGTGAGGAGCGCCTGAGA
AGGACCTTTGTCAATGGGACACGGACACAGCCGCCCAGCTGGCTGGAGCTGCAGGCCACCA
AGTCCAAGAAGCCAATCATGTTGCCCGTGACATTCATGGATGGGACCACCAAGACCCTGCT
GACGGACTCGGCAACCACGGCCAAGGAGCTCTGCAACGCGCTGGCCGACAAGATCTCTCTC
AAGGACCGGTTCGGGTTCTCCCTCTACATTGCCCTGTTTGACAAGGTGTCCTCCCTGGGCAG
CGGCAGTGACCACGTCATGGACGCCATCTCCCAGTGCGAGCAGTACGCCAAGGAGCAGGG
CGCCCAGGAGCGCAACGCCCCCTGGAGGCTCTTCTTCCGCAAAGAGGTCTTCACGCCCTGG
CACAGCCCCTCCGAGGACAACGTGGCCACCAACCTCATCTACCAGCAGGTGGTGCGAGGAG
TCAAGTTTGGGGAGTACAGGTGTGAGAAGGAGGACGACCTGGCTGAGCTGGCCTCCCAGCA
GTACTTTGTAGACTATGGCTCTGAGATGATCCTGGAGCGCCTCCTGAACCTCGTGCCCACCT
ACATCCCCGACCGCGAGATCACGCCCCTGAAGACGCTGGAGAAGTGGGCCCAGCTGGCCAT
CGCCGCCCACAAGAAGGGGATTTATGCCCAGAGGAGAACTGATGCCCAGAAGGTCAAAGA
GGATGTGGTCAGTTATGCCCGCTTCAAGTGGCCCTTGCTCTTCTCCAGGTTTTATGAAGCCT
ACAAATTCTCAGGCCCCAGTCTCCCCAAGAACGACGTCATCGTGGCCGTCAACTGGACGGG
TGTGTACTTTGTGGATGAGCAGGAGCAGGTACTTCTGGAGCTGTCCTTCCCAGAGATCATGG
CCGTGTCCAGCAGCAGGGGAGCGAAAACGACGGCCCCCAGCTTCACGCTGGCCACCATCAA
GGGGGACGAATACACCTTCACCTCCAGCAATGCTGAGGACATTCGTGACCTGGTGGTCACC
TTCCTAGAGGGGCTCCGGAAGAGATCTAAGTATGTTGTGGCCCTGCAGGATAACCCCAACC
CCGCAGGCGAGGAGTCAGGCTTCCTCAGCTTTGCCAAGGGAGACCTCATCATCCTGGACCA
TGACACGGGCGAGCAGGTCATGAACTCGGGCTGGGCCAACGGCATCAATGAGAGGACCAA
GCAGCGTGGGGACTTCCCCACCGACAGTGTGTACGTCATGCCCACTGTCACCATGCCACCG
CGGGAGATTGTGGCCCTGGTCACCATGACTCCCGATCAGAGGCAGGACGTTGTCCGGCTCT
TGCAGCTGCGAACGGCGGAGCCCGAGGTGCGTGCCAAGCCCTACACGCTGGAGGAGTTTTC
CTATGACTACTTCAGGCCCCCACCCAAGCACACGCTGAGCCGTGTCATGGTGTCCAAGGCC
CGAGGCAAGGACCGGCTGTGGAGCCACACGCGGGAACCGCTCAAGCAGGCGCTGCTCAAG
AAGCTCCTGGGCAGTGAGGAGCTCTCGCAGGAGGCCTGCCTGGCCTTCATTGCTGTGCTCA
AGTACATGGGCGACTACCCGTCCAAGAGGACACGCTCCGTCAACGAGCTCACCGACCAGAT
CTTTGAGGGTCCCCTGAAAGCCGAGCCCCTGAAGGACGAGGCATATGTGCAGATCCTGAAG
CAGCTGACCGACAACCACATCAGGTACAGCGAGGAGCGGGGTTGGGAGCTGCTCTGGCTGT
GCACGGGCCTTTTCCCACCCAGCAACATCCTCCTGCCCCACGTGCAGCGCTTCCTGCAGTCC
CGAAAGCACTGCCCACTCGCCATCGACTGCCTGCAACGGCTCCAGAAAGCCCTGAGAAACG
GGTCCCGGAAGTACCCTCCGCACCTGGTGGAGGTGGAGGCCATCCAGCACAAGACCACCCA
GATTTTCCACAAAGTCTACTTCCCTGATGACACTGACGAGGCCTTCGAAGTGGAGTCCAGC
ACCAAGGCCAAGGACTTCTGCCAGAACATCGCCACCAGGCTGCTCCTCAAGTCCTCAGAGG
GATTCAGCCTCTTTGTCAAAATTGCAGACAAGGTCATCAGCGTTCCTGAGAATGACTTCTTC
TTTGACTTTGTTCGACACTTGACAGACTGGATAAAGAAAGCTCGGCCCATCAAGGACGGAA
TTGTGCCCTCACTCACCTACCAGGTGTTCTTCATGAAGAAGCTGTGGACCACCACGGTGCCA
GGGAAGGATCCCATGGCCGATTCCATCTTCCACTATTACCAGGAGTTGCCCAAGTATCTCCG
AGGCTACCACAAGTGCACGCGGGAGGAGGTGCTGCAGCTGGGGGCGCTGATCTACAGGGT
CAAGTTCGAGGAGGACAAGTCCTACTTCCCCAGCATCCCCAAGCTGCTGCGGGAGCTGGTG
CCCCAGGACCTTATCCGGCAGGTCTCACCTGATGACTGGAAGCGGTCCATCGTCGCCTACTT
CAACAAGCACGCAGGGAAGTCCAAGGAGGAGGCCAAGCTGGCCTTCCTGAAGCTCATCTTC
AAGTGGCCCACCTTTGGCTCAGCCTTCTTCGAGGTGAAGCAAACTACGGAGCCAAACTTCC
CTGAGATCCTCCTAATTGCCATCAACAAGTATGGGGTCAGCCTCATCGATCCCAAAACGAA
GGATATCCTCACCACTCATCCCTTCACCAAGATCTCCAACTGGAGCAGCGGCAACACCTACT
TCCACATCACCATTGGGAACTTGGTGCGCGGGAGCAAACTGCTCTGCGAGACGTCACTGGG
CTACAAGATGGATGACCTCCTGACTTCCTACATTAGCCAGATGCTCACAGCCATGAGCAAA
CAGCGGGGCTCCAGGAGCGGCAAG
SEQ ID NO: 78 (N-term myosin7A (e.g., AAV-hMYO7A-CTlong.HA)
GLMRLGFLRLQALHRSRKLHQQYRLARQRIIQFQARCRAYLVRKAFRHRLWAVLTVQAYARG
MIARRLHQRLRAEYLWRLEAEKMRLAEEEKLRKEMSAKKAKEEAERKHQERLAQLAREDAER
ELKEKEAARRKKELLEQMERARHEPVNHSDMVDKMFGFLGTSGGLPGQEGQAPSGFEDLERG
RREMVEEDLDAALPLPDEDEEDLSEYKFAKFAATYFQGTTTHSYTRRPLKQPLLYHDDEGDQL
AALAVWITILRFMGDLPEPKYHTAMSDGSEKIPVMTKIYETLGKKTYKRELQALQGEGEAQLPE
GQKKSSVRHKLVHLTLKKKSKLTEEVTKRLHDGESTVQGNSMLEDRPTSNLEKLHFIIGNGILRP
ALRDEIYCQISKQLTHNPSKSSYARGWILVSLCVGCFAPSEKFVKYLRNFIHGGPPGYAPYCEER
LRRTFVNGTRTQPPSWLELQATKSKKPIMLPVTFMDGTTKTLLTDSATTAKELCNALADKISLK
DRFGFSLYIALFDKVSSLGSGSDHVMDAISQCEQYAKEQGAQERNAPWRLFFRKEVFTPWHSPS
EDNVATNLIYQQVVRGVKFGEYRCEKEDDLAELASQQYFVDYGSEMILERLLNLVPTYIPDREI
TPLKTLEKWAQLAIAAHKKGIYAQRRTDAQKVKEDVVSYARFKWPLLFSRFYEAYKFSGPSLP
KNDVIVAVNWTGVYFVDEQEQVLLELSFPEIMAVSSSRGAKTTAPSFTLATIKGDEYTFTSSNAE
DIRDLVVTFLEGLRKRSKYVVALQDNPNPAGEESGFLSFAKGDLIILDHDTGEQVMNSGWANGI
NERTKQRGDFPTDSVYVMPTVTMPPREIVALVTMTPDQRQDVVRLLQLRTAEPEVRAKPYTLE
EFSYDYFRPPPKHTLSRVMVSKARGKDRLWSHTREPLKQALLKKLLGSEELSQEACLAFIAVLK
YMGDYPSKRTRSVNELTDQIFEGPLKAEPLKDEAYVQILKQLTDNHIRYSEERGWELLWLCTGL
FPPSNILLPHVQRFLQSRKHCPLAIDCLQRLQKALRNGSRKYPPHLVEVEAIQHKTTQIFHKVYFP
DDTDEAFEVESSTKAKDFCQNIATRLLLKSSEGFSLFVKIADKVISVPENDFFFDFVRHLTDWIKK
ARPIKDGIVPSLTYQVFFMKKLWTTTVPGKDPMADSIFHYYQELPKYLRGYHKCTREEVLQLG
ALIYRVKFEEDKSYFPSIPKLLRELVPQDLIRQVSPDDWKRSIVAYFNKHAGKSKEEAKLAFLKLI
FKWPTFGSAFFEVKQTTEPNFPEILLIAINKYGVSLIDPKTKDILTTHPFTKISNWSSGNTYFHITIG
NLVRGSKLLCETSLGYKMDDLLTSYISQMLTAMSKQRGSRSGK

In some embodiments, the vectors provided herein comprise a truncated chimeric CBA promoter. The vectors provided herein may comprise a promoter having a nucleotide sequence that differs from the smCBA promoter set forth as SEQ ID NO: 64 by 1, 2, or 3 nucleotides. In some embodiments, the vectors provided herein comprise a promoter that is not an smCBA promoter. In some embodiments, the vectors provided herein comprise a promoter selected from selected from the group consisting of a CMV promoter, an EF-1 alpha promoter, a cone arrestin promoter, a human myosin 7a gene-derived promoter, a TaC gene-derived promoter, a rhodopsin promoter, a cGMP-phosphodiesterase β-subunit promoter, human or mouse rhodopsin promoter, a hGRK1 promoter, a synapsin promoter, a glial fibrillary acidic protein (GFAP) promoter, a rod specific IRBP promoter, a VMD2 promoter, and combinations thereof. In some embodiments, the promoter is a rhodopsin promoter. In some embodiments, the promoter is a CMV promoter. In some embodiments, the promoter is not a CMV promoter.

In some embodiments, the promoter is a tissue-specific promoter. In some embodiments, the promoter mediates expression in ocular tissue. In some embodiments, the promoter does not mediate expression in ocular tissue. In some embodiments, the promoter mediates expression in hair cells of the auditory system and/or the vestibular system.

SEQ ID NO: 64 smCBA promoter
AATTCGGTACCCTAGTTATTAATAGTAATCAATTACGGGGTCATTAGTTCATAGCCCATATA
TGGAGTTCCGCGTTACATAACTTACGGTAAATGGCCCGCCTGGCTGACCGCCCAACGACCC
CCGCCCATTGACGTCAATAATGACGTATGTTCCCATAGTAACGCCAATAGGGACTTTCCATT
GACGTCAATGGGTGGACTATTTACGGTAAACTGCCCACTTGGCAGTACATCAAGTGTATCA
TATGCCAAGTACGCCCCCTATTGACGTCAATGACGGTAAATGGCCCGCCTGGCATTATGCCC
AGTACATGACCTTATGGGACTTTCCTACTTGGCAGTACATCTACGTATTAGTCATCGCTATT
ACCATGGTCGAGGTGAGCCCCACGTTCTGCTTCACTCTCCCCATCTCCCCCCCCTCCCCACC
CCCAATTTTGTATTTATTTATTTTTTAATTATTTTGTGCAGCGATGGGGGCGGGGGGGGGGG
GGGGGCGCGCGCCAGGCGGGGCGGGGCGGGGCGAGGGGCGGGGCGGGGCGAGGCGGAGA
GGTGCGGCGGCAGCCAATCAGAGCGGCGCGCTCCGAAAGTTTCCTTTTATGGCGAGGCGGC
GGCGGCGGCGGCCCTATAAAAAGCGAAGCGCGCGGCGGGCGGGAGTCGCTGCGCGCTGCC
TTCGCCCCGTGCCCCGCTCCGCCGCCGCCTCGCGCCGCCCGCCCCGGCTCTGACTGACCGCG
TTACTCCCACAGGTGAGCGGGCGGGACGGCCCTTCTCCTCCGGGCTGTAATTAGCGCTTGGT
TTAATGACGGCTTGTTTCTTTTCTGTGGCTGCGTGAAAGCCTTGAGGGGCTCCGGGAGCTAG
AGCCTCTGCTAACCATGTTCATGCCTTCTTCTTTTTCCTACAGCTCCTGGGCAACGTGCTGGT
TATTGTGCTGTCTCATCATTTTGGCAAAG
SEQ ID NO: 39 is the nucleotide sequence of second generation
overlapping portion of the overlap vector system:
CAGGTCTAACTTTCTGAAGCTGAAGAACGCTGCCACACTGATCCAGAGGCACTGGC
GGGGTCACAACTGTAGGAAGAACTACGGGCTGATGCGTCTGGGCTTCCTGCGGCTG
CAGGCCCTGCACCGCTCCCGGAAGCTGCACCAGCAGTACCGCCTGGCCCGCCAGCG
CATCATCCAGTTCCAGGCCCGCTGCCGCGCCTATCTGGTGCGCAAGGCCTTCCGCCA
CCGCCTCTGGGCTGTGCTCACCGTGCAGGCCTATGCCCGGGGCATGATCGCCCGCA
GGCTGCACCAACGCCTCAGGGCTGAGTATCTGTGGCGCCTCGAGGCTGAGAAAATG
CGGCTGGCGGAGGAAGAGAAGCTT
SEQ ID NO: 79 (associated protein sequence from SEQ ID NO: 39)
RSNFLKLKNAATLIQRHWRGHNCRKNYGLMRLGFLRLQALHRSRKLHQQYRLARQRII
QFQARCRAYLVRKAFRHRLWAVLTVQAYARGMIARRLHQRLRAEYLWRLEAEKMRL
AEEEKL
SEQ ID NO: 40 is the relevant fragment of SEQ ID NO: 31 where
potential in-frame stop codons are located in the second
generation hybrid front half AP splice donor region (see,
e.g. FIG. 42).
CAACGCCTCAGGGCTGAGGTAAGTATCAAGGTTACAAGACAGGTTTAAGGAGACCA
ATAGAAACTGGGCTTGTCGAGACAGAGAAGACTCTTGCGTTTCTGAGCTAGCCCC
SEQ ID NO: 41 is the relevant fragment of SEQ ID NO: 33 where
potential in-frame stop codons are removed in the CMv1 hybrid
front half AP splice donor region (see, e.g., FIG. 42).
CAACGCCTCAGGGCTGAGGTAAGTATCAAGGTTACAAGACAGGTTAACGGAGACCA
ATTGAAACTGGGCTTGTCGAGACAGAGAAGACTCTTGCGTTTCAGCGCTAGCCCC
SEQ ID NO: 42 is the forward primer sequence of Gibson Primer
Set 1 (reverse primer sequence is the reverse compliment of
the forward primer (not shown))(see, e.g., FIG. 42).
CCGCAGGCTGCACCAACGCCTCAGGGCTGAGGTAAGTATCAAGGTTACAAGACAG
GTTAACGGAGACCAATTGAAACT
SEQ ID NO: 43 is the forward primer sequence of Gibson Primer
Set 2 (reverse primer sequence is the reverse compliment of
the forward primer (not shown))(see, e.g., FIG. 42).
AACGGAGACCAATTGAAACTGGGCTTGTCGAGACAGAGAAGACTCTTGCGTTTCAG
CGCTAGCCCCCGGGTGCGCGGCG
SEQ ID NO: 44 is the polynucleotide sequence of the CMv2.1
hybrid back half vector
(i.e., AAV-APhead-APSA-hMYO7ACTex22-CMv2.1).
CTCCCTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCC
ATCACTAGGGGTTCTCAGATCTGGCGCGCCCCCCGGGTGCGCGGCGTCGGTGGTGCCGGCG
GGGGGCGCCAGGTCGCAGGCGGTGTAGGGCTCCAGGCAGGCGGCGAAGGCCATGACGTGC
GCTATGAAGGTCTGCTCCTGCACGCCGTGAACCAGGTGCGCCTGCGGGCCGCGCGCGAACA
CCGCCACGTCCTCGCCTGCGTGGGTCTCTTCGTCCAGGGGCACTGCGCACTGCTGCCGATAC
TCGGGGCTCCCGCTCTCGCTCTCGGTAACATCCGGCCGGGCGCCGTCCTTGAGCACATAGCC
TGGACCGTTTCTCTTAAGCGACGCATGCTCGCGATAGGCACCTATTGGTCTTACTGACATCC
ACTTTGCCTTTCTCTCCACAGTATCTGTGGCGCCTCGAGGCTGAGAAAATGCGGCTGGCGGA
GGAAGAGAAGCTTCGGAAGGAGATGAGCGCCAAGAAGGCCAAGGAGGAGGCCGAGCGCA
AGCATCAGGAGCGCCTGGCCCAGCTGGCTCGTGAGGACGCTGAGCGGGAGCTGAAGGAGA
AGGAGGCCGCTCGGCGGAAGAAGGAGCTCCTGGAGCAGATGGAAAGGGCCCGCCATGAGC
CTGTCAATCACTCAGACATGGTGGACAAGATGTTTGGCTTCCTGGGGACTTCAGGTGGCCTG
CCAGGCCAGGAGGGCCAGGCACCTAGTGGCTTTGAGGACCTGGAGCGAGGGCGGAGGGAG
ATGGTGGAGGAGGACCTGGATGCAGCCCTGCCCCTGCCTGACGAGGATGAGGAGGACCTCT
CTGAGTATAAATTTGCCAAGTTCGCGGCCACCTACTTCCAGGGGACAACCACGCACTCCTA
CACCCGGCGGCCACTCAAACAGCCACTGCTCTACCATGACGACGAGGGTGACCAGCTGGCA
GCCCTGGCGGTCTGGATCACCATCCTCCGCTTCATGGGGGACCTCCCTGAGCCCAAGTACCA
CACAGCCATGAGTGATGGCAGTGAGAAGATCCCTGTGATGACCAAGATTTATGAGACCCTG
GGCAAGAAGACGTACAAGAGGGAGCTGCAGGCCCTGCAGGGCGAGGGCGAGGCCCAGCTC
CCCGAGGGCCAGAAGAAGAGCAGTGTGAGGCACAAGCTGGTGCATTTGACTCTGAAAAAG
AAGTCCAAGCTCACAGAGGAGGTGACCAAGAGGCTGCATGACGGGGAGTCCACAGTGCAG
GGCAACAGCATGCTGGAGGACCGGCCCACCTCCAACCTGGAGAAGCTGCACTTCATCATCG
GCAATGGCATCCTGCGGCCAGCACTCCGGGACGAGATCTACTGCCAGATCAGCAAGCAGCT
GACCCACAACCCCTCCAAGAGCAGCTATGCCCGGGGCTGGATTCTCGTGTCTCTCTGCGTGG
GCTGTTTCGCCCCCTCCGAGAAGTTTGTCAAGTACCTGCGGAACTTCATCCACGGGGGCCCG
CCCGGCTACGCCCCGTACTGTGAGGAGCGCCTGAGAAGGACCTTTGTCAATGGGACACGGA
CACAGCCGCCCAGCTGGCTGGAGCTGCAGGCCACCAAGTCCAAGAAGCCAATCATGTTGCC
CGTGACATTCATGGATGGGACCACCAAGACCCTGCTGACGGACTCGGCAACCACGGCCAAG
GAGCTCTGCAACGCGCTGGCCGACAAGATCTCTCTCAAGGACCGGTTCGGGTTCTCCCTCTA
CATTGCCCTGTTTGACAAGGTGTCCTCCCTGGGCAGCGGCAGTGACCACGTCATGGACGCC
ATCTCCCAGTGCGAGCAGTACGCCAAGGAGCAGGGCGCCCAGGAGCGCAACGCCCCCTGG
AGGCTCTTCTTCCGCAAAGAGGTCTTCACGCCCTGGCACAGCCCCTCCGAGGACAACGTGG
CCACCAACCTCATCTACCAGCAGGTGGTGCGAGGAGTCAAGTTTGGGGAGTACAGGTGTGA
GAAGGAGGACGACCTGGCTGAGCTGGCCTCCCAGCAGTACTTTGTAGACTATGGCTCTGAG
ATGATCCTGGAGCGCCTCCTGAACCTCGTGCCCACCTACATCCCCGACCGCGAGATCACGC
CCCTGAAGACGCTGGAGAAGTGGGCCCAGCTGGCCATCGCCGCCCACAAGAAGGGGATTT
ATGCCCAGAGGAGAACTGATGCCCAGAAGGTCAAAGAGGATGTGGTCAGTTATGCCCGCTT
CAAGTGGCCCTTGCTCTTCTCCAGGTTTTATGAAGCCTACAAATTCTCAGGCCCCAGTCTCC
CCAAGAACGACGTCATCGTGGCCGTCAACTGGACGGGTGTGTACTTTGTGGATGAGCAGGA
GCAGGTACTTCTGGAGCTGTCCTTCCCAGAGATCATGGCCGTGTCCAGCAGCAGGGGAGCG
AAAACGACGGCCCCCAGCTTCACGCTGGCCACCATCAAGGGGGACGAATACACCTTCACCT
CCAGCAATGCTGAGGACATTCGTGACCTGGTGGTCACCTTCCTAGAGGGGCTCCGGAAGAG
ATCTAAGTATGTTGTGGCCCTGCAGGATAACCCCAACCCCGCAGGCGAGGAGTCAGGCTTC
CTCAGCTTTGCCAAGGGAGACCTCATCATCCTGGACCATGACACGGGCGAGCAGGTCATGA
ACTCGGGCTGGGCCAACGGCATCAATGAGAGGACCAAGCAGCGTGGGGACTTCCCCACCG
ACAGTGTGTACGTCATGCCCACTGTCACCATGCCACCGCGGGAGATTGTGGCCCTGGTCAC
CATGACTCCCGATCAGAGGCAGGACGTTGTCCGGCTCTTGCAGCTGCGAACGGCGGAGCCC
GAGGTGCGTGCCAAGCCCTACACGCTGGAGGAGTTTTCCTATGACTACTTCAGGCCCCCAC
CCAAGCACACGCTGAGCCGTGTCATGGTGTCCAAGGCCCGAGGCAAGGACCGGCTGTGGA
GCCACACGCGGGAACCGCTCAAGCAGGCGCTGCTCAAGAAGCTCCTGGGCAGTGAGGAGC
TCTCGCAGGAGGCCTGCCTGGCCTTCATTGCTGTGCTCAAGTACATGGGCGACTACCCGTCC
AAGAGGACACGCTCCGTCAACGAGCTCACCGACCAGATCTTTGAGGGTCCCCTGAAAGCCG
AGCCCCTGAAGGACGAGGCATATGTGCAGATCCTGAAGCAGCTGACCGACAACCACATCA
GGTACAGCGAGGAGCGGGGTTGGGAGCTGCTCTGGCTGTGCACGGGCCTTTTCCCACCCAG
CAACATCCTCCTGCCCCACGTGCAGCGCTTCCTGCAGTCCCGAAAGCACTGCCCACTCGCCA
TCGACTGCCTGCAACGGCTCCAGAAAGCCCTGAGAAACGGGTCCCGGAAGTACCCTCCGCA
CCTGGTGGAGGTGGAGGCCATCCAGCACAAGACCACCCAGATTTTCCACAAAGTCTACTTC
CCTGATGACACTGACGAGGCCTTCGAAGTGGAGTCCAGCACCAAGGCCAAGGACTTCTGCC
AGAACATCGCCACCAGGCTGCTCCTCAAGTCCTCAGAGGGATTCAGCCTCTTTGTCAAAATT
GCAGACAAGGTCATCAGCGTTCCTGAGAATGACTTCTTCTTTGACTTTGTTCGACACTTGAC
AGACTGGATAAAGAAAGCTCGGCCCATCAAGGACGGAATTGTGCCCTCACTCACCTACCAG
GTGTTCTTCATGAAGAAGCTGTGGACCACCACGGTGCCAGGGAAGGATCCCATGGCCGATT
CCATCTTCCACTATTACCAGGAGTTGCCCAAGTATCTCCGAGGCTACCACAAGTGCACGCG
GGAGGAGGTGCTGCAGCTGGGGGCGCTGATCTACAGGGTCAAGTTCGAGGAGGACAAGTC
CTACTTCCCCAGCATCCCCAAGCTGCTGCGGGAGCTGGTGCCCCAGGACCTTATCCGGCAG
GTCTCACCTGATGACTGGAAGCGGTCCATCGTCGCCTACTTCAACAAGCACGCAGGGAAGT
CCAAGGAGGAGGCCAAGCTGGCCTTCCTGAAGCTCATCTTCAAGTGGCCCACCTTTGGCTC
AGCCTTCTTCGAGGTGAAGCAAACTACGGAGCCAAACTTCCCTGAGATCCTCCTAATTGCC
ATCAACAAGTATGGGGTCAGCCTCATCGATCCCAAAACGAAGGATATCCTCACCACTCATC
CCTTCACCAAGATCTCCAACTGGAGCAGCGGCAACACCTACTTCCACATCACCATTGGGAA
CTTGGTGCGCGGGAGCAAACTGCTCTGCGAGACGTCACTGGGCTACAAGATGGATGACCTC
CTGACTTCCTACATTAGCCAGATGCTCACAGCCATGAGCAAACAGCGGGGCTCCAGGAGCG
GCAAGAGAGCTCGCTGATCAGCCTCGACTGTGCCTTCTAGTTGCCAGCCATCTGTTGTTTGC
CCCTCCCCCGTGCCTTCCTTGACCCTGGAAGGTGCCACTCCCACTGTCCTTTCCTAATAAAA
TGAGGAAATTGCATCGCATTGTCTGAGTAGGTGTCATTCTATTCTGGGGGGTGGGGTGGGG
CAGGACAGCAAGGGGGAGGATTGGGAAGACAATAGCAGGCATTTAATTAAGCATGCTGGG
GAGAGATCTGAGGAAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCGGGCGGCCTCAG
TGAGCGAGCGAGCGCGCAGAGAGGGAG
SEQ ID NO: 45 is the portion of overlapping sequence from the
original overlap vectors.
CAGGTCTAACTTTCTGAAGCTGAAGAACGCTGCCACACTGATCCAGAGGCACTGGCGGGGT
CACAACTGTAGGAAGAACTACGGGCTGATGCGTCTGGGCTTCCTGCGGCTGCAGGCCCTGC
ACCGCTCCCGGAAGCTGCACCAGCAGTACCGCCTGGCCCGCCAGCGCATCATCCAGTTCCA
GGCCCGCTGCCGCGCCTATCTGGTGCGCAAGGCCTTCCGCCACCGCCTCTGGGCTGTGCTCA
CCGTGCAGGCCTATGCCCGGGGCATGATCGCCCGCAGGCTGCACCAACGCCTCAGGGCTGA
GTATCTGTGGCGCCTCGAGGCTGAGAAAATGCGGCTGGCGGAGGAAGAGAAGCTTCGGAA
GGAGATGAGCGCCAAGAAGGCCAAGGAGGAGGCCGAGCGCAAGCATCAGGAGCGCCTGG
CCCAGCTGGCTCGTGAGGACGCTGAGCGGGAGCTGAAGGAGAAGGAGGCCGCTCGGCGGA
AGAAGGAGCTCCTGGAGCAGATGGAAAGGGCCCGCCATGAGCCTGTCAATCACTCAGACA
TGGTGGACAAGATGTTTGGCTTCCTGGGGACTTCAGGTGGCCTGCCAGGCCAGGAGGGCCA
GGCACCTAGTGGCTTTGAGGACCTGGAGCGAGGGCGGAGGGAGATGGTGGAGGAGGACCT
GGATGCAGCCCTGCCCCTGCCTGACGAGGATGAGGAGGACCTCTCTGAGTATAAATTTGCC
AAGTTCGCGGCCACCTACTTCCAGGGGACAACCACGCACTCCTACACCCGGCGGCCACTCA
AACAGCCACTGCTCTACCATGACGACGAGGGTGACCAGCTGGCAGCCCTGGCGGTCTGGAT
CACCATCCTCCGCTTCATGGGGGACCTCCCTGAGCCCAAGTACCACACAGCCATGAGTGAT
GGCAGTGAGAAGATCCCTGTGATGACCAAGATTTATGAGACCCTGGGCAAGAAGACGTAC
AAGAGGGAGCTGCAGGCCCTGCAGGGCGAGGGCGAGGCCCAGCTCCCCGAGGGCCAGAAG
AAGAGCAGTGTGAGGCACAAGCTGGTGCATTTGACTCTGAAAAAGAAGTCCAAGCTCACA
GAGGAGGTGACCAAGAGGCTGCATGACGGGGAGTCCACAGTGCAGGGCAACAGCATGCTG
GAGGACCGGCCCACCTCCAACCTGGAGAAGCTGCACTTCATCATCGGCAATGGCATCCTGC
GGCCAGCACTCCGGGACGAGATCTACTGCCAGATCAGCAAGCAGCTGACCCACAACCCCTC
CAAGAGCAGCTATGCCCGGGGCTGGATTCTCGTGTCTCTCTGCGTGGGCTGTTTCGCCCCCT
CCGAGAAGTTTGTCAAGTACCTGCGGAACTTC
SEQ ID NO: 46 is the polynucleotide sequence of the CMv3 hybrid
front half vector (i.e., AAV-smCBA-hMYO7A-NT-Ex21-APSD-APhead-CMv3)
(pairs with CMv2 back half vector).
CTCCCTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCC
ATCACTAGGGGTTCTCAGATCTGGCGCGCCCAATTCGGTACCCTAGTTATTAATAGTAATCA
ATTACGGGGTCATTAGTTCATAGCCCATATATGGAGTTCCGCGTTACATAACTTACGGTAAA
TGGCCCGCCTGGCTGACCGCCCAACGACCCCCGCCCATTGACGTCAATAATGACGTATGTTC
CCATAGTAACGCCAATAGGGACTTTCCATTGACGTCAATGGGTGGACTATTTACGGTAAAC
TGCCCACTTGGCAGTACATCAAGTGTATCATATGCCAAGTACGCCCCCTATTGACGTCAATG
ACGGTAAATGGCCCGCCTGGCATTATGCCCAGTACATGACCTTATGGGACTTTCCTACTTGG
CAGTACATCTACGTATTAGTCATCGCTATTACCATGGTCGAGGTGAGCCCCACGTTCTGCTT
CACTCTCCCCATCTCCCCCCCCTCCCCACCCCCAATTTTGTATTTATTTATTTTTTAATTATTT
TGTGCAGCGATGGGGGCGGGGGGGGGGGGGGGGCGCGCGCCAGGCGGGGCGGGGCGGGG
CGAGGGGCGGGGCGGGGCGAGGCGGAGAGGTGCGGCGGCAGCCAATCAGAGCGGCGCGC
TCCGAAAGTTTCCTTTTATGGCGAGGCGGCGGCGGCGGCGGCCCTATAAAAAGCGAAGCGC
GCGGCGGGCGGGAGTCGCTGCGCGCTGCCTTCGCCCCGTGCCCCGCTCCGCCGCCGCCTCG
CGCCGCCCGCCCCGGCTCTGACTGACCGCGTTACTCCCACAGGTGAGCGGGCGGGACGGCC
CTTCTCCTCCGGGCTGTAATTAGCGCTTGGTTTAATGACGGCTTGTTTCTTTTCTGTGGCTGC
GTGAAAGCCTTGAGGGGCTCCGGGAGCTAGAGCCTCTGCTAACCATGTTCATGCCTTCTTCT
TTTTCCTACAGCTCCTGGGCAACGTGCTGGTTATTGTGCTGTCTCATCATTTTGGCAAAGAA
TTCTAGCGGCCGCCACCATGGTGATTCTTCAGCAGGGGGACCATGTGTGGATGGACCTGAG
ATTGGGGCAGGAGTTCGACGTGCCCATCGGGGCGGTGGTGAAGCTCTGCGACTCTGGGCAG
GTCCAGGTGGTGGATGATGAAGACAATGAACACTGGATCTCTCCGCAGAACGCAACGCACA
TCAAGCCTATGCACCCCACGTCGGTCCACGGCGTGGAGGACATGATCCGCCTGGGGGACCT
CAACGAGGCGGGCATCTTGCGCAACCTGCTTATCCGCTACCGGGACCACCTCATCTACACG
TATACGGGCTCCATCCTGGTGGCTGTGAACCCCTACCAGCTGCTCTCCATCTACTCGCCAGA
GCACATCCGCCAGTATACCAACAAGAAGATTGGGGAGATGCCCCCCCACATCTTTGCCATT
GCTGACAACTGCTACTTCAACATGAAACGCAACAGCCGAGACCAGTGCTGCATCATCAGTG
GGGAATCTGGGGCCGGGAAGACGGAGAGCACAAAGCTGATCCTGCAGTTCCTGGCAGCCA
TCAGTGGGCAGCACTCGTGGATTGAGCAGCAGGTCTTGGAGGCCACCCCCATTCTGGAAGC
ATTTGGGAATGCCAAGACCATCCGCAATGACAACTCAAGCCGTTTCGGAAAGTACATCGAC
ATCCACTTCAACAAGCGGGGCGCCATCGAGGGCGCGAAGATTGAGCAGTACCTGCTGGAA
AAGTCACGTGTCTGTCGCCAGGCCCTGGATGAAAGGAACTACCACGTGTTCTACTGCATGC
TGGAGGGTATGAGTGAGGATCAGAAGAAGAAGCTGGGCTTGGGCCAGGCCTCTGACTACA
ACTACTTGGCCATGGGTAACTGCATAACCTGTGAGGGCCGGGTGGACAGCCAGGAGTACGC
CAACATCCGCTCCGCCATGAAGGTGCTCATGTTCACTGACACCGAGAACTGGGAGATCTCG
AAGCTCCTGGCTGCCATCCTGCACCTGGGCAACCTGCAGTATGAGGCACGCACATTTGAAA
ACCTGGATGCCTGTGAGGTTCTCTTCTCCCCATCGCTGGCCACAGCTGCATCCCTGCTTGAG
GTGAACCCCCCAGACCTGATGAGCTGCCTGACTAGCCGCACCCTCATCACCCGCGGGGAGA
CGGTGTCCACCCCACTGAGCAGGGAACAGGCACTGGACGTGCGCGACGCCTTCGTAAAGGG
GATCTACGGGCGGCTGTTCGTGTGGATTGTGGACAAGATCAACGCAGCAATTTACAAGCCT
CCCTCCCAGGATGTGAAGAACTCTCGCAGGTCCATCGGCCTCCTGGACATCTTTGGGTTTGA
GAACTTTGCTGTGAACAGCTTTGAGCAGCTCTGCATCAACTTCGCCAATGAGCACCTGCAGC
AGTTCTTTGTGCGGCACGTGTTCAAGCTGGAGCAGGAGGAATATGACCTGGAGAGCATTGA
CTGGCTGCACATCGAGTTCACTGACAACCAGGATGCCCTGGACATGATTGCCAACAAGCCC
ATGAACATCATCTCCCTCATCGATGAGGAGAGCAAGTTCCCCAAGGGCACAGACACCACCA
TGTTACACAAGCTGAACTCCCAGCACAAGCTCAACGCCAACTACATCCCCCCCAAGAACAA
CCATGAGACCCAGTTTGGCATCAACCATTTTGCAGGCATCGTCTACTATGAGACCCAAGGCT
TCCTGGAGAAGAACCGAGACACCCTGCATGGGGACATTATCCAGCTGGTCCACTCCTCCAG
GAACAAGTTCATCAAGCAGATCTTCCAGGCCGATGTCGCCATGGGCGCCGAGACCAGGAAG
CGCTCGCCCACACTTAGCAGCCAGTTCAAGCGGTCACTGGAGCTGCTGATGCGCACGCTGG
GTGCCTGCCAGCCCTTCTTTGTGCGATGCATCAAGCCCAATGAGTTCAAGAAGCCCATGCTG
TTCGACCGGCACCTGTGCGTGCGCCAGCTGCGGTACTCAGGAATGATGGAGACCATCCGAA
TCCGCCGAGCTGGCTACCCCATCCGCTACAGCTTCGTAGAGTTTGTGGAGCGGTACCGTGTG
CTGCTGCCAGGTGTGAAGCCGGCCTACAAGCAGGGCGACCTCCGCGGGACTTGCCAGCGCA
TGGCTGAGGCTGTGCTGGGCACCCACGATGACTGGCAGATAGGCAAAACCAAGATCTTTCT
GAAGGACCACCATGACATGCTGCTGGAAGTGGAGCGGGACAAAGCCATCACCGACAGAGT
CATCCTCCTTCAGAAAGTCATCCGGGGATTCAAAGACAGGTCTAACTTTCTGAAGCTGAAG
AACGCTGCCACACTGATCCAGAGGCACTGGCGGGGTCACAACTGTAGGAAGAACTACGGG
CTGATGCGTCTGGGCTTCCTGCGGCTGCAGGCCCTGCACCGCTCCCGGAAGCTGCACCAGC
AGTACCGCCTGGCCCGCCAGCGCATCATCCAGTTCCAGGCCCGCTGCCGCGCCTATCTGGTG
CGCAAGGCCTTCCGCCACCGCCTCTGGGCTGTGCTCACCGTGCAGGCCTATGCCCGGGGCA
TGATCGCCCGCAGGCTGCACCAACGCCTCAGGGCTGAGGTAAGTATCAAGGTTACAAGACA
GGTTAACGGAGACCAATTGAAACTGGGCTTGTCGAGACAGAGAAGACTCTTGCGTTTCAGC
GCTAGCCCCCGGGTGCGCGGCGTCGGTGGTGCCGGCGGGGGGCGCCAGGTCGCAGGCGGT
GTAGGGCTCCAGGCAGGCGGCGAAGGCCATGACGTGCGCTATGAAGGTCTGCTCCTGCACG
CCGTGAACCAGGTGCGCCTGCGGGCCGCGCGCGAACACCGCCACGTCCTCGCCTGCGTGGG
TCTCTTCGTCCAGGGGCACTGCGCACTGCTGCCGATACTCGGGGCTCCCGCTCTCGCTCTCG
GTAACATCCGGCCGGGCGCCGTCCTTGAGCACATAGCCTGGACCGTTTCGTCGACGGATCC
GCATGCTGGGGAGAGATCTGAGGAAACCCCTAGTGATGGAGTTGGCCACTCCCTCTCTGCG
CGCTCGCTCGCTCACTGAGGCCGGGCGACCAAAGGTCGCCCGACGCCCGGGCTTTGCCCGG
GCGGCCTCAGTGAGCGAGCGAGCGCGCAGAGAGGGAG
SEQ ID NO: 47 is the polynucleotide sequence of the CMv2.1
hybrid back half vector, containing the HA tag (i.e., AAV-
APhead-APSA-hMYO7ACTex22-CMv2.1.HA)
(pairs with CMv2 front half vector).
CTCCCTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCC
ATCACTAGGGGTTCTCAGATCTGGCGCGCCCCCCGGGTGCGCGGCGTCGGTGGTGCCGGCG
GGGGGCGCCAGGTCGCAGGCGGTGTAGGGCTCCAGGCAGGCGGCGAAGGCCATGACGTGC
GCTATGAAGGTCTGCTCCTGCACGCCGTGAACCAGGTGCGCCTGCGGGCCGCGCGCGAACA
CCGCCACGTCCTCGCCTGCGTGGGTCTCTTCGTCCAGGGGCACTGCGCACTGCTGCCGATAC
TCGGGGCTCCCGCTCTCGCTCTCGGTAACATCCGGCCGGGCGCCGTCCTTGAGCACATAGCC
TGGACCGTTTCTCTTAAGCGACGCATGCTCGCGATAGGCACCTATTGGTCTTACTGACATCC
ACTTTGCCTTTCTCTCCACAGTATCTGTGGCGCCTCGAGGCTGAGAAAATGCGGCTGGCGGA
GGAAGAGAAGCTTCGGAAGGAGATGAGCGCCAAGAAGGCCAAGGAGGAGGCCGAGCGCA
AGCATCAGGAGCGCCTGGCCCAGCTGGCTCGTGAGGACGCTGAGCGGGAGCTGAAGGAGA
AGGAGGCCGCTCGGCGGAAGAAGGAGCTCCTGGAGCAGATGGAAAGGGCCCGCCATGAGC
CTGTCAATCACTCAGACATGGTGGACAAGATGTTTGGCTTCCTGGGGACTTCAGGTGGCCTG
CCAGGCCAGGAGGGCCAGGCACCTAGTGGCTTTGAGGACCTGGAGCGAGGGCGGAGGGAG
ATGGTGGAGGAGGACCTGGATGCAGCCCTGCCCCTGCCTGACGAGGATGAGGAGGACCTCT
CTGAGTATAAATTTGCCAAGTTCGCGGCCACCTACTTCCAGGGGACAACCACGCACTCCTA
CACCCGGCGGCCACTCAAACAGCCACTGCTCTACCATGACGACGAGGGTGACCAGCTGGCA
GCCCTGGCGGTCTGGATCACCATCCTCCGCTTCATGGGGGACCTCCCTGAGCCCAAGTACCA
CACAGCCATGAGTGATGGCAGTGAGAAGATCCCTGTGATGACCAAGATTTATGAGACCCTG
GGCAAGAAGACGTACAAGAGGGAGCTGCAGGCCCTGCAGGGCGAGGGCGAGGCCCAGCTC
CCCGAGGGCCAGAAGAAGAGCAGTGTGAGGCACAAGCTGGTGCATTTGACTCTGAAAAAG
AAGTCCAAGCTCACAGAGGAGGTGACCAAGAGGCTGCATGACGGGGAGTCCACAGTGCAG
GGCAACAGCATGCTGGAGGACCGGCCCACCTCCAACCTGGAGAAGCTGCACTTCATCATCG
GCAATGGCATCCTGCGGCCAGCACTCCGGGACGAGATCTACTGCCAGATCAGCAAGCAGCT
GACCCACAACCCCTCCAAGAGCAGCTATGCCCGGGGCTGGATTCTCGTGTCTCTCTGCGTGG
GCTGTTTCGCCCCCTCCGAGAAGTTTGTCAAGTACCTGCGGAACTTCATCCACGGGGGCCCG
CCCGGCTACGCCCCGTACTGTGAGGAGCGCCTGAGAAGGACCTTTGTCAATGGGACACGGA
CACAGCCGCCCAGCTGGCTGGAGCTGCAGGCCACCAAGTCCAAGAAGCCAATCATGTTGCC
CGTGACATTCATGGATGGGACCACCAAGACCCTGCTGACGGACTCGGCAACCACGGCCAAG
GAGCTCTGCAACGCGCTGGCCGACAAGATCTCTCTCAAGGACCGGTTCGGGTTCTCCCTCTA
CATTGCCCTGTTTGACAAGGTGTCCTCCCTGGGCAGCGGCAGTGACCACGTCATGGACGCC
ATCTCCCAGTGCGAGCAGTACGCCAAGGAGCAGGGCGCCCAGGAGCGCAACGCCCCCTGG
AGGCTCTTCTTCCGCAAAGAGGTCTTCACGCCCTGGCACAGCCCCTCCGAGGACAACGTGG
CCACCAACCTCATCTACCAGCAGGTGGTGCGAGGAGTCAAGTTTGGGGAGTACAGGTGTGA
GAAGGAGGACGACCTGGCTGAGCTGGCCTCCCAGCAGTACTTTGTAGACTATGGCTCTGAG
ATGATCCTGGAGCGCCTCCTGAACCTCGTGCCCACCTACATCCCCGACCGCGAGATCACGC
CCCTGAAGACGCTGGAGAAGTGGGCCCAGCTGGCCATCGCCGCCCACAAGAAGGGGATTT
ATGCCCAGAGGAGAACTGATGCCCAGAAGGTCAAAGAGGATGTGGTCAGTTATGCCCGCTT
CAAGTGGCCCTTGCTCTTCTCCAGGTTTTATGAAGCCTACAAATTCTCAGGCCCCAGTCTCC
CCAAGAACGACGTCATCGTGGCCGTCAACTGGACGGGTGTGTACTTTGTGGATGAGCAGGA
GCAGGTACTTCTGGAGCTGTCCTTCCCAGAGATCATGGCCGTGTCCAGCAGCAGGGGAGCG
AAAACGACGGCCCCCAGCTTCACGCTGGCCACCATCAAGGGGGACGAATACACCTTCACCT
CCAGCAATGCTGAGGACATTCGTGACCTGGTGGTCACCTTCCTAGAGGGGCTCCGGAAGAG
ATCTAAGTATGTTGTGGCCCTGCAGGATAACCCCAACCCCGCAGGCGAGGAGTCAGGCTTC
CTCAGCTTTGCCAAGGGAGACCTCATCATCCTGGACCATGACACGGGCGAGCAGGTCATGA
ACTCGGGCTGGGCCAACGGCATCAATGAGAGGACCAAGCAGCGTGGGGACTTCCCCACCG
ACAGTGTGTACGTCATGCCCACTGTCACCATGCCACCGCGGGAGATTGTGGCCCTGGTCAC
CATGACTCCCGATCAGAGGCAGGACGTTGTCCGGCTCTTGCAGCTGCGAACGGCGGAGCCC
GAGGTGCGTGCCAAGCCCTACACGCTGGAGGAGTTTTCCTATGACTACTTCAGGCCCCCAC
CCAAGCACACGCTGAGCCGTGTCATGGTGTCCAAGGCCCGAGGCAAGGACCGGCTGTGGA
GCCACACGCGGGAACCGCTCAAGCAGGCGCTGCTCAAGAAGCTCCTGGGCAGTGAGGAGC
TCTCGCAGGAGGCCTGCCTGGCCTTCATTGCTGTGCTCAAGTACATGGGCGACTACCCGTCC
AAGAGGACACGCTCCGTCAACGAGCTCACCGACCAGATCTTTGAGGGTCCCCTGAAAGCCG
AGCCCCTGAAGGACGAGGCATATGTGCAGATCCTGAAGCAGCTGACCGACAACCACATCA
GGTACAGCGAGGAGCGGGGTTGGGAGCTGCTCTGGCTGTGCACGGGCCTTTTCCCACCCAG
CAACATCCTCCTGCCCCACGTGCAGCGCTTCCTGCAGTCCCGAAAGCACTGCCCACTCGCCA
TCGACTGCCTGCAACGGCTCCAGAAAGCCCTGAGAAACGGGTCCCGGAAGTACCCTCCGCA
CCTGGTGGAGGTGGAGGCCATCCAGCACAAGACCACCCAGATTTTCCACAAAGTCTACTTC
CCTGATGACACTGACGAGGCCTTCGAAGTGGAGTCCAGCACCAAGGCCAAGGACTTCTGCC
AGAACATCGCCACCAGGCTGCTCCTCAAGTCCTCAGAGGGATTCAGCCTCTTTGTCAAAATT
GCAGACAAGGTCATCAGCGTTCCTGAGAATGACTTCTTCTTTGACTTTGTTCGACACTTGAC
AGACTGGATAAAGAAAGCTCGGCCCATCAAGGACGGAATTGTGCCCTCACTCACCTACCAG
GTGTTCTTCATGAAGAAGCTGTGGACCACCACGGTGCCAGGGAAGGATCCCATGGCCGATT
CCATCTTCCACTATTACCAGGAGTTGCCCAAGTATCTCCGAGGCTACCACAAGTGCACGCG
GGAGGAGGTGCTGCAGCTGGGGGCGCTGATCTACAGGGTCAAGTTCGAGGAGGACAAGTC
CTACTTCCCCAGCATCCCCAAGCTGCTGCGGGAGCTGGTGCCCCAGGACCTTATCCGGCAG
GTCTCACCTGATGACTGGAAGCGGTCCATCGTCGCCTACTTCAACAAGCACGCAGGGAAGT
CCAAGGAGGAGGCCAAGCTGGCCTTCCTGAAGCTCATCTTCAAGTGGCCCACCTTTGGCTC
AGCCTTCTTCGAGGTGAAGCAAACTACGGAGCCAAACTTCCCTGAGATCCTCCTAATTGCC
ATCAACAAGTATGGGGTCAGCCTCATCGATCCCAAAACGAAGGATATCCTCACCACTCATC
CCTTCACCAAGATCTCCAACTGGAGCAGCGGCAACACCTACTTCCACATCACCATTGGGAA
CTTGGTGCGCGGGAGCAAACTGCTCTGCGAGACGTCACTGGGCTACAAGATGGATGACCTC
CTGACTTCCTACATTAGCCAGATGCTCACAGCCATGAGCAAACAGCGGGGCTCCAGGAGCG
GCAAGTACCCTTACGATGTACCGGATTACGCATGAAGAGCTCGCTGATCAGCCTCGACTGT
GCCTTCTAGTTGCCAGCCATCTGTTGTTTGCCCCTCCCCCGTGCCTTCCTTGACCCTGGAAGG
TGCCACTCCCACTGTCCTTTCCTAATAAAATGAGGAAATTGCATCGCATTGTCTGAGTAGGT
GTCATTCTATTCTGGGGGGTGGGGTGGGGCAGGACAGCAAGGGGGAGGATTGGGAAGACA
ATAGCAGGCATTTAATTAAGCATGCTGGGGAGAGATCTGAGGAAACCCCTAGTGATGGAGT
TGGCCACTCCCTCTCTGCGCGCTCGCTCGCTCACTGAGGCCGGGCGACCAAAGGTCGCCCG
ACGCCCGGGCTTTGCCCGGGCGGCCTCAGTGAGCGAGCGAGCGCGCAGAGAGGGAG
SEQ ID NO: 48 is the polynucleotide sequence of the minimized
second-generation hybrid back half vector, containing the HA
tag (i.e., AAV-APhead-APSA-hMYO7ACTex22.HA-MIN)
(pairs with second generation front half hybrid vector).
CTCCCTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCC
ATCACTAGGGGTTCTCAGATCTGGCGCGCCCCCCGGGTGCGCGGCGTCGGTGGTGCCGGCG
GGGGGCGCCAGGTCGCAGGCGGTGTAGGGCTCCAGGCAGGCGGCGAAGGCCATGACGTGC
GCTATGAAGGTCTGCTCCTGCACGCCGTGAACCAGGTGCGCCTGCGGGCCGCGCGCGAACA
CCGCCACGTCCTCGCCTGCGTGGGTCTCTTCGTCCAGGGGCACTGCTGACTGCTGCCGATAC
TCGGGGCTCCCGCTCTCGCTCTCGGTAACATCCGGCCGGGCGCCGTCCTTGAGCACATAGCC
TGGACCGTTTCCTTAAGCGACGCATGCTCGCGATAGGCACCTATTGGTCTTACTGACATCCA
CTTTGCCTTTCTCTCCACAGTATCTGTGGCGCCTCGAGGCTGAGAAAATGCGGCTGGCGGAG
GAAGAGAAGCTTCGGAAGGAGATGAGCGCCAAGAAGGCCAAGGAGGAGGCCGAGCGCAA
GCATCAGGAGCGCCTGGCCCAGCTGGCTCGTGAGGACGCTGAGCGGGAGCTGAAGGAGAA
GGAGGCCGCTCGGCGGAAGAAGGAGCTCCTGGAGCAGATGGAAAGGGCCCGCCATGAGCC
TGTCAATCACTCAGACATGGTGGACAAGATGTTTGGCTTCCTGGGGACTTCAGGTGGCCTGC
CAGGCCAGGAGGGCCAGGCACCTAGTGGCTTTGAGGACCTGGAGCGAGGGCGGAGGGAGA
TGGTGGAGGAGGACCTGGATGCAGCCCTGCCCCTGCCTGACGAGGATGAGGAGGACCTCTC
TGAGTATAAATTTGCCAAGTTCGCGGCCACCTACTTCCAGGGGACAACCACGCACTCCTAC
ACCCGGCGGCCACTCAAACAGCCACTGCTCTACCATGACGACGAGGGTGACCAGCTGGCAG
CCCTGGCGGTCTGGATCACCATCCTCCGCTTCATGGGGGACCTCCCTGAGCCCAAGTACCAC
ACAGCCATGAGTGATGGCAGTGAGAAGATCCCTGTGATGACCAAGATTTATGAGACCCTGG
GCAAGAAGACGTACAAGAGGGAGCTGCAGGCCCTGCAGGGCGAGGGCGAGGCCCAGCTCC
CCGAGGGCCAGAAGAAGAGCAGTGTGAGGCACAAGCTGGTGCATTTGACTCTGAAAAAGA
AGTCCAAGCTCACAGAGGAGGTGACCAAGAGGCTGCATGACGGGGAGTCCACAGTGCAGG
GCAACAGCATGCTGGAGGACCGGCCCACCTCCAACCTGGAGAAGCTGCACTTCATCATCGG
CAATGGCATCCTGCGGCCAGCACTCCGGGACGAGATCTACTGCCAGATCAGCAAGCAGCTG
ACCCACAACCCCTCCAAGAGCAGCTATGCCCGGGGCTGGATTCTCGTGTCTCTCTGCGTGGG
CTGTTTCGCCCCCTCCGAGAAGTTTGTCAAGTACCTGCGGAACTTCATCCACGGGGGCCCGC
CCGGCTACGCCCCGTACTGTGAGGAGCGCCTGAGAAGGACCTTTGTCAATGGGACACGGAC
ACAGCCGCCCAGCTGGCTGGAGCTGCAGGCCACCAAGTCCAAGAAGCCAATCATGTTGCCC
GTGACATTCATGGATGGGACCACCAAGACCCTGCTGACGGACTCGGCAACCACGGCCAAGG
AGCTCTGCAACGCGCTGGCCGACAAGATCTCTCTCAAGGACCGGTTCGGGTTCTCCCTCTAC
ATTGCCCTGTTTGACAAGGTGTCCTCCCTGGGCAGCGGCAGTGACCACGTCATGGACGCCA
TCTCCCAGTGCGAGCAGTACGCCAAGGAGCAGGGCGCCCAGGAGCGCAACGCCCCCTGGA
GGCTCTTCTTCCGCAAAGAGGTCTTCACGCCCTGGCACAGCCCCTCCGAGGACAACGTGGC
CACCAACCTCATCTACCAGCAGGTGGTGCGAGGAGTCAAGTTTGGGGAGTACAGGTGTGAG
AAGGAGGACGACCTGGCTGAGCTGGCCTCCCAGCAGTACTTTGTAGACTATGGCTCTGAGA
TGATCCTGGAGCGCCTCCTGAACCTCGTGCCCACCTACATCCCCGACCGCGAGATCACGCCC
CTGAAGACGCTGGAGAAGTGGGCCCAGCTGGCCATCGCCGCCCACAAGAAGGGGATTTAT
GCCCAGAGGAGAACTGATGCCCAGAAGGTCAAAGAGGATGTGGTCAGTTATGCCCGCTTCA
AGTGGCCCTTGCTCTTCTCCAGGTTTTATGAAGCCTACAAATTCTCAGGCCCCAGTCTCCCC
AAGAACGACGTCATCGTGGCCGTCAACTGGACGGGTGTGTACTTTGTGGATGAGCAGGAGC
AGGTACTTCTGGAGCTGTCCTTCCCAGAGATCATGGCCGTGTCCAGCAGCAGGGGAGCGAA
AACGACGGCCCCCAGCTTCACGCTGGCCACCATCAAGGGGGACGAATACACCTTCACCTCC
AGCAATGCTGAGGACATTCGTGACCTGGTGGTCACCTTCCTAGAGGGGCTCCGGAAGAGAT
CTAAGTATGTTGTGGCCCTGCAGGATAACCCCAACCCCGCAGGCGAGGAGTCAGGCTTCCT
CAGCTTTGCCAAGGGAGACCTCATCATCCTGGACCATGACACGGGCGAGCAGGTCATGAAC
TCGGGCTGGGCCAACGGCATCAATGAGAGGACCAAGCAGCGTGGGGACTTCCCCACCGAC
AGTGTGTACGTCATGCCCACTGTCACCATGCCACCGCGGGAGATTGTGGCCCTGGTCACCAT
GACTCCCGATCAGAGGCAGGACGTTGTCCGGCTCTTGCAGCTGCGAACGGCGGAGCCCGAG
GTGCGTGCCAAGCCCTACACGCTGGAGGAGTTTTCCTATGACTACTTCAGGCCCCCACCCAA
GCACACGCTGAGCCGTGTCATGGTGTCCAAGGCCCGAGGCAAGGACCGGCTGTGGAGCCAC
ACGCGGGAACCGCTCAAGCAGGCGCTGCTCAAGAAGCTCCTGGGCAGTGAGGAGCTCTCGC
AGGAGGCCTGCCTGGCCTTCATTGCTGTGCTCAAGTACATGGGCGACTACCCGTCCAAGAG
GACACGCTCCGTCAACGAGCTCACCGACCAGATCTTTGAGGGTCCCCTGAAAGCCGAGCCC
CTGAAGGACGAGGCATATGTGCAGATCCTGAAGCAGCTGACCGACAACCACATCAGGTAC
AGCGAGGAGCGGGGTTGGGAGCTGCTCTGGCTGTGCACGGGCCTTTTCCCACCCAGCAACA
TCCTCCTGCCCCACGTGCAGCGCTTCCTGCAGTCCCGAAAGCACTGCCCACTCGCCATCGAC
TGCCTGCAACGGCTCCAGAAAGCCCTGAGAAACGGGTCCCGGAAGTACCCTCCGCACCTGG
TGGAGGTGGAGGCCATCCAGCACAAGACCACCCAGATTTTCCACAAAGTCTACTTCCCTGA
TGACACTGACGAGGCCTTCGAAGTGGAGTCCAGCACCAAGGCCAAGGACTTCTGCCAGAAC
ATCGCCACCAGGCTGCTCCTCAAGTCCTCAGAGGGATTCAGCCTCTTTGTCAAAATTGCAGA
CAAGGTCATCAGCGTTCCTGAGAATGACTTCTTCTTTGACTTTGTTCGACACTTGACAGACT
GGATAAAGAAAGCTCGGCCCATCAAGGACGGAATTGTGCCCTCACTCACCTACCAGGTGTT
CTTCATGAAGAAGCTGTGGACCACCACGGTGCCAGGGAAGGATCCCATGGCCGATTCCATC
TTCCACTATTACCAGGAGTTGCCCAAGTATCTCCGAGGCTACCACAAGTGCACGCGGGAGG
AGGTGCTGCAGCTGGGGGCGCTGATCTACAGGGTCAAGTTCGAGGAGGACAAGTCCTACTT
CCCCAGCATCCCCAAGCTGCTGCGGGAGCTGGTGCCCCAGGACCTTATCCGGCAGGTCTCA
CCTGATGACTGGAAGCGGTCCATCGTCGCCTACTTCAACAAGCACGCAGGGAAGTCCAAGG
AGGAGGCCAAGCTGGCCTTCCTGAAGCTCATCTTCAAGTGGCCCACCTTTGGCTCAGCCTTC
TTCGAGGTGAAGCAAACTACGGAGCCAAACTTCCCTGAGATCCTCCTAATTGCCATCAACA
AGTATGGGGTCAGCCTCATCGATCCCAAAACGAAGGATATCCTCACCACTCATCCCTTCACC
AAGATCTCCAACTGGAGCAGCGGCAACACCTACTTCCACATCACCATTGGGAACTTGGTGC
GCGGGAGCAAACTGCTCTGCGAGACGTCACTGGGCTACAAGATGGATGACCTCCTGACTTC
CTACATTAGCCAGATGCTCACAGCCATGAGCAAACAGCGGGGCTCCAGGAGCGGCAAGTA
CCCTTACGATGTACCGGATTACGCATGAAGAGCTCGCTGATCAGCCTCGACTGTGCCTTCTA
GTTGCCAGCCATCTGTTGTTTGCCCCTCCCCCGTGCCTTCCTTGACCCTGGAAGGTGCCACTC
CCACTGTCCTTTCCTAATAAAATGAGGAAATTGCATCGCATTGTCTGAGTAGGTGTCATTCT
ATTCTGGGGGGTGGGGTGGGGCAGGACAGCAAGGGGGAGGATTGGGAAGACAATAGCAGG
CATTTAATTAAGCATGCTGGGGAGAGATCTGAGGAAACCCCTAGTGATGGAGTTGGCCACT
CCCTCTCTGCGCGCTCGCTCGCTCACTGAGGCCGGGCGACCAAAGGTCGCCCGACGCCCGG
GCTTTGCCCGGGCGGCCTCAGTGAGCGAGCGAGCGCGCAGAGAGGGAG
SEQ ID NO: 49 is the polynucleotide sequence of the minimized
second-generation hybrid back half vector, not containing the
HA tag (i.e., AAV-APhead-APSA-hMYO7ACTex22-MIN)
(pairs with second generation front half hybrid vector).
CTCCCTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCC
ATCACTAGGGGTTCTCAGATCTGGCGCGCCCCCCGGGTGCGCGGCGTCGGTGGTGCCGGCG
GGGGGCGCCAGGTCGCAGGCGGTGTAGGGCTCCAGGCAGGCGGCGAAGGCCATGACGTGC
GCTATGAAGGTCTGCTCCTGCACGCCGTGAACCAGGTGCGCCTGCGGGCCGCGCGCGAACA
CCGCCACGTCCTCGCCTGCGTGGGTCTCTTCGTCCAGGGGCACTGCTGACTGCTGCCGATAC
TCGGGGCTCCCGCTCTCGCTCTCGGTAACATCCGGCCGGGCGCCGTCCTTGAGCACATAGCC
TGGACCGTTTCCTTAAGCGACGCATGCTCGCGATAGGCACCTATTGGTCTTACTGACATCCA
CTTTGCCTTTCTCTCCACAGTATCTGTGGCGCCTCGAGGCTGAGAAAATGCGGCTGGCGGAG
GAAGAGAAGCTTCGGAAGGAGATGAGCGCCAAGAAGGCCAAGGAGGAGGCCGAGCGCAA
GCATCAGGAGCGCCTGGCCCAGCTGGCTCGTGAGGACGCTGAGCGGGAGCTGAAGGAGAA
GGAGGCCGCTCGGCGGAAGAAGGAGCTCCTGGAGCAGATGGAAAGGGCCCGCCATGAGCC
TGTCAATCACTCAGACATGGTGGACAAGATGTTTGGCTTCCTGGGGACTTCAGGTGGCCTGC
CAGGCCAGGAGGGCCAGGCACCTAGTGGCTTTGAGGACCTGGAGCGAGGGCGGAGGGAGA
TGGTGGAGGAGGACCTGGATGCAGCCCTGCCCCTGCCTGACGAGGATGAGGAGGACCTCTC
TGAGTATAAATTTGCCAAGTTCGCGGCCACCTACTTCCAGGGGACAACCACGCACTCCTAC
ACCCGGCGGCCACTCAAACAGCCACTGCTCTACCATGACGACGAGGGTGACCAGCTGGCAG
CCCTGGCGGTCTGGATCACCATCCTCCGCTTCATGGGGGACCTCCCTGAGCCCAAGTACCAC
ACAGCCATGAGTGATGGCAGTGAGAAGATCCCTGTGATGACCAAGATTTATGAGACCCTGG
GCAAGAAGACGTACAAGAGGGAGCTGCAGGCCCTGCAGGGCGAGGGCGAGGCCCAGCTCC
CCGAGGGCCAGAAGAAGAGCAGTGTGAGGCACAAGCTGGTGCATTTGACTCTGAAAAAGA
AGTCCAAGCTCACAGAGGAGGTGACCAAGAGGCTGCATGACGGGGAGTCCACAGTGCAGG
GCAACAGCATGCTGGAGGACCGGCCCACCTCCAACCTGGAGAAGCTGCACTTCATCATCGG
CAATGGCATCCTGCGGCCAGCACTCCGGGACGAGATCTACTGCCAGATCAGCAAGCAGCTG
ACCCACAACCCCTCCAAGAGCAGCTATGCCCGGGGCTGGATTCTCGTGTCTCTCTGCGTGGG
CTGTTTCGCCCCCTCCGAGAAGTTTGTCAAGTACCTGCGGAACTTCATCCACGGGGGCCCGC
CCGGCTACGCCCCGTACTGTGAGGAGCGCCTGAGAAGGACCTTTGTCAATGGGACACGGAC
ACAGCCGCCCAGCTGGCTGGAGCTGCAGGCCACCAAGTCCAAGAAGCCAATCATGTTGCCC
GTGACATTCATGGATGGGACCACCAAGACCCTGCTGACGGACTCGGCAACCACGGCCAAGG
AGCTCTGCAACGCGCTGGCCGACAAGATCTCTCTCAAGGACCGGTTCGGGTTCTCCCTCTAC
ATTGCCCTGTTTGACAAGGTGTCCTCCCTGGGCAGCGGCAGTGACCACGTCATGGACGCCA
TCTCCCAGTGCGAGCAGTACGCCAAGGAGCAGGGCGCCCAGGAGCGCAACGCCCCCTGGA
GGCTCTTCTTCCGCAAAGAGGTCTTCACGCCCTGGCACAGCCCCTCCGAGGACAACGTGGC
CACCAACCTCATCTACCAGCAGGTGGTGCGAGGAGTCAAGTTTGGGGAGTACAGGTGTGAG
AAGGAGGACGACCTGGCTGAGCTGGCCTCCCAGCAGTACTTTGTAGACTATGGCTCTGAGA
TGATCCTGGAGCGCCTCCTGAACCTCGTGCCCACCTACATCCCCGACCGCGAGATCACGCCC
CTGAAGACGCTGGAGAAGTGGGCCCAGCTGGCCATCGCCGCCCACAAGAAGGGGATTTAT
GCCCAGAGGAGAACTGATGCCCAGAAGGTCAAAGAGGATGTGGTCAGTTATGCCCGCTTCA
AGTGGCCCTTGCTCTTCTCCAGGTTTTATGAAGCCTACAAATTCTCAGGCCCCAGTCTCCCC
AAGAACGACGTCATCGTGGCCGTCAACTGGACGGGTGTGTACTTTGTGGATGAGCAGGAGC
AGGTACTTCTGGAGCTGTCCTTCCCAGAGATCATGGCCGTGTCCAGCAGCAGGGGAGCGAA
AACGACGGCCCCCAGCTTCACGCTGGCCACCATCAAGGGGGACGAATACACCTTCACCTCC
AGCAATGCTGAGGACATTCGTGACCTGGTGGTCACCTTCCTAGAGGGGCTCCGGAAGAGAT
CTAAGTATGTTGTGGCCCTGCAGGATAACCCCAACCCCGCAGGCGAGGAGTCAGGCTTCCT
CAGCTTTGCCAAGGGAGACCTCATCATCCTGGACCATGACACGGGCGAGCAGGTCATGAAC
TCGGGCTGGGCCAACGGCATCAATGAGAGGACCAAGCAGCGTGGGGACTTCCCCACCGAC
AGTGTGTACGTCATGCCCACTGTCACCATGCCACCGCGGGAGATTGTGGCCCTGGTCACCAT
GACTCCCGATCAGAGGCAGGACGTTGTCCGGCTCTTGCAGCTGCGAACGGCGGAGCCCGAG
GTGCGTGCCAAGCCCTACACGCTGGAGGAGTTTTCCTATGACTACTTCAGGCCCCCACCCAA
GCACACGCTGAGCCGTGTCATGGTGTCCAAGGCCCGAGGCAAGGACCGGCTGTGGAGCCAC
ACGCGGGAACCGCTCAAGCAGGCGCTGCTCAAGAAGCTCCTGGGCAGTGAGGAGCTCTCGC
AGGAGGCCTGCCTGGCCTTCATTGCTGTGCTCAAGTACATGGGCGACTACCCGTCCAAGAG
GACACGCTCCGTCAACGAGCTCACCGACCAGATCTTTGAGGGTCCCCTGAAAGCCGAGCCC
CTGAAGGACGAGGCATATGTGCAGATCCTGAAGCAGCTGACCGACAACCACATCAGGTAC
AGCGAGGAGCGGGGTTGGGAGCTGCTCTGGCTGTGCACGGGCCTTTTCCCACCCAGCAACA
TCCTCCTGCCCCACGTGCAGCGCTTCCTGCAGTCCCGAAAGCACTGCCCACTCGCCATCGAC
TGCCTGCAACGGCTCCAGAAAGCCCTGAGAAACGGGTCCCGGAAGTACCCTCCGCACCTGG
TGGAGGTGGAGGCCATCCAGCACAAGACCACCCAGATTTTCCACAAAGTCTACTTCCCTGA
TGACACTGACGAGGCCTTCGAAGTGGAGTCCAGCACCAAGGCCAAGGACTTCTGCCAGAAC
ATCGCCACCAGGCTGCTCCTCAAGTCCTCAGAGGGATTCAGCCTCTTTGTCAAAATTGCAGA
CAAGGTCATCAGCGTTCCTGAGAATGACTTCTTCTTTGACTTTGTTCGACACTTGACAGACT
GGATAAAGAAAGCTCGGCCCATCAAGGACGGAATTGTGCCCTCACTCACCTACCAGGTGTT
CTTCATGAAGAAGCTGTGGACCACCACGGTGCCAGGGAAGGATCCCATGGCCGATTCCATC
TTCCACTATTACCAGGAGTTGCCCAAGTATCTCCGAGGCTACCACAAGTGCACGCGGGAGG
AGGTGCTGCAGCTGGGGGCGCTGATCTACAGGGTCAAGTTCGAGGAGGACAAGTCCTACTT
CCCCAGCATCCCCAAGCTGCTGCGGGAGCTGGTGCCCCAGGACCTTATCCGGCAGGTCTCA
CCTGATGACTGGAAGCGGTCCATCGTCGCCTACTTCAACAAGCACGCAGGGAAGTCCAAGG
AGGAGGCCAAGCTGGCCTTCCTGAAGCTCATCTTCAAGTGGCCCACCTTTGGCTCAGCCTTC
TTCGAGGTGAAGCAAACTACGGAGCCAAACTTCCCTGAGATCCTCCTAATTGCCATCAACA
AGTATGGGGTCAGCCTCATCGATCCCAAAACGAAGGATATCCTCACCACTCATCCCTTCACC
AAGATCTCCAACTGGAGCAGCGGCAACACCTACTTCCACATCACCATTGGGAACTTGGTGC
GCGGGAGCAAACTGCTCTGCGAGACGTCACTGGGCTACAAGATGGATGACCTCCTGACTTC
CTACATTAGCCAGATGCTCACAGCCATGAGCAAACAGCGGGGCTCCAGGAGCGGCAAGAG
AGCTCGCTGATCAGCCTCGACTGTGCCTTCTAGTTGCCAGCCATCTGTTGTTTGCCCCTCCCC
CGTGCCTTCCTTGACCCTGGAAGGTGCCACTCCCACTGTCCTTTCCTAATAAAATGAGGAAA
TTGCATCGCATTGTCTGAGTAGGTGTCATTCTATTCTGGGGGGTGGGGTGGGGCAGGACAG
CAAGGGGGAGGATTGGGAAGACAATAGCAGGCATTTAATTAAGCATGCTGGGGAGAGATC
TAGGAAACCCCTAGTGATGGAGTTGGCCACTCCCTCTCTGCGCGCTCGCTCGCTCACTGAGG
CCGGGCGACCAAAGGTCGCCCGACGCCCGGGCTTTGCCCGGGCGGCCTCAGTGAGCGAGCG
AGCGCGCAGAGAGGGAG
SEQ ID NO: 50 is the polynucleotide sequence of the third
generation overlap front half vector
(i.e., AAV-smCBA-hMYO7A-NTlong-v3).
CTCCCTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCC
ATCACTAGGGGTTCAGATCTGGCGCGCCCAATTCGGTACCCTAGTTATTAATAGTAATCAAT
TACGGGGTCATTAGTTCATAGCCCATATATGGAGTTCCGCGTTACATAACTTACGGTAAATG
GCCCGCCTGGCTGACCGCCCAACGACCCCCGCCCATTGACGTCAATAATGACGTATGTTCCC
ATAGTAACGCCAATAGGGACTTTCCATTGACGTCAATGGGTGGACTATTTACGGTAAACTG
CCCACTTGGCAGTACATCAAGTGTATCATATGCCAAGTACGCCCCCTATTGACGTCAATGAC
GGTAAATGGCCCGCCTGGCATTATGCCCAGTACATGACCTTATGGGACTTTCCTACTTGGCA
GTACATCTACGTATTAGTCATCGCTATTACCATGGTCGAGGTGAGCCCCACGTTCTGCTTCA
CTCTCCCCATCTCCCCCCCCTCCCCACCCCCAATTTTGTATTTATTTATTTTTTAATTATTTTG
TGCAGCGATGGGGGCGGGGGGGGGGGGGGGGCGCGCGCCAGGCGGGGCGGGGCGGGGCG
AGGGGCGGGGCGGGGCGAGGCGGAGAGGTGCGGCGGCAGCCAATCAGAGCGGCGCGCTCC
GAAAGTTTCCTTTTATGGCGAGGCGGCGGCGGCGGCGGCCCTATAAAAAGCGAAGCGCGCG
GCGGGCGGGAGTCGCTGCGCGCTGCCTTCGCCCCGTGCCCCGCTCCGCCGCCGCCTCGCGC
CGCCCGCCCCGGCTCTGACTGACCGCGTTACTCCCACAGGTGAGCGGGCGGGACGGCCCTT
CTCCTCCGGGCTGTAATTAGCGCTTGGTTTAATGACGGCTTGTTTCTTTTCTGTGGCTGCGTG
AAAGCCTTGAGGGGCTCCGGGAGCTAGAGCCTCTGCTAACCATGTTCATGCCTTCTTCTTTT
TCCTACAGCTCCTGGGCAACGTGCTGGTTATTGTGCTGTCTCATCATTTTGGCAAAGAATTC
TAGCGGCCGCCACCATGGTGATTCTTCAGCAGGGGGACCATGTGTGGATGGACCTGAGATT
GGGGCAGGAGTTCGACGTGCCCATCGGGGCGGTGGTGAAGCTCTGCGACTCTGGGCAGGTC
CAGGTGGTGGATGATGAAGACAATGAACACTGGATCTCTCCGCAGAACGCAACGCACATCA
AGCCTATGCACCCCACGTCGGTCCACGGCGTGGAGGACATGATCCGCCTGGGGGACCTCAA
CGAGGCGGGCATCTTGCGCAACCTGCTTATCCGCTACCGGGACCACCTCATCTACACGTATA
CGGGCTCCATCCTGGTGGCTGTGAACCCCTACCAGCTGCTCTCCATCTACTCGCCAGAGCAC
ATCCGCCAGTATACCAACAAGAAGATTGGGGAGATGCCCCCCCACATCTTTGCCATTGCTG
ACAACTGCTACTTCAACATGAAACGCAACAGCCGAGACCAGTGCTGCATCATCAGTGGGGA
ATCTGGGGCCGGGAAGACGGAGAGCACAAAGCTGATCCTGCAGTTCCTGGCAGCCATCAGT
GGGCAGCACTCGTGGATTGAGCAGCAGGTCTTGGAGGCCACCCCCATTCTGGAAGCATTTG
GGAATGCCAAGACCATCCGCAATGACAACTCAAGCCGTTTCGGAAAGTACATCGACATCCA
CTTCAACAAGCGGGGCGCCATCGAGGGCGCGAAGATTGAGCAGTACCTGCTGGAAAAGTC
ACGTGTCTGTCGCCAGGCCCTGGATGAAAGGAACTACCACGTGTTCTACTGCATGCTGGAG
GGTATGAGTGAGGATCAGAAGAAGAAGCTGGGCTTGGGCCAGGCCTCTGACTACAACTACT
TGGCCATGGGTAACTGCATAACCTGTGAGGGCCGGGTGGACAGCCAGGAGTACGCCAACAT
CCGCTCCGCCATGAAGGTGCTCATGTTCACTGACACCGAGAACTGGGAGATCTCGAAGCTC
CTGGCTGCCATCCTGCACCTGGGCAACCTGCAGTATGAGGCACGCACATTTGAAAACCTGG
ATGCCTGTGAGGTTCTCTTCTCCCCATCGCTGGCCACAGCTGCATCCCTGCTTGAGGTGAAC
CCCCCAGACCTGATGAGCTGCCTGACTAGCCGCACCCTCATCACCCGCGGGGAGACGGTGT
CCACCCCACTGAGCAGGGAACAGGCACTGGACGTGCGCGACGCCTTCGTAAAGGGGATCTA
CGGGCGGCTGTTCGTGTGGATTGTGGACAAGATCAACGCAGCAATTTACAAGCCTCCCTCC
CAGGATGTGAAGAACTCTCGCAGGTCCATCGGCCTCCTGGACATCTTTGGGTTTGAGAACTT
TGCTGTGAACAGCTTTGAGCAGCTCTGCATCAACTTCGCCAATGAGCACCTGCAGCAGTTCT
TTGTGCGGCACGTGTTCAAGCTGGAGCAGGAGGAATATGACCTGGAGAGCATTGACTGGCT
GCACATCGAGTTCACTGACAACCAGGATGCCCTGGACATGATTGCCAACAAGCCCATGAAC
ATCATCTCCCTCATCGATGAGGAGAGCAAGTTCCCCAAGGGCACAGACACCACCATGTTAC
ACAAGCTGAACTCCCAGCACAAGCTCAACGCCAACTACATCCCCCCCAAGAACAACCATGA
GACCCAGTTTGGCATCAACCATTTTGCAGGCATCGTCTACTATGAGACCCAAGGCTTCCTGG
AGAAGAACCGAGACACCCTGCATGGGGACATTATCCAGCTGGTCCACTCCTCCAGGAACAA
GTTCATCAAGCAGATCTTCCAGGCCGATGTCGCCATGGGCGCCGAGACCAGGAAGCGCTCG
CCCACACTTAGCAGCCAGTTCAAGCGGTCACTGGAGCTGCTGATGCGCACGCTGGGTGCCT
GCCAGCCCTTCTTTGTGCGATGCATCAAGCCCAATGAGTTCAAGAAGCCCATGCTGTTCGAC
CGGCACCTGTGCGTGCGCCAGCTGCGGTACTCAGGAATGATGGAGACCATCCGAATCCGCC
GAGCTGGCTACCCCATCCGCTACAGCTTCGTAGAGTTTGTGGAGCGGTACCGTGTGCTGCTG
CCAGGTGTGAAGCCGGCCTACAAGCAGGGCGACCTCCGCGGGACTTGCCAGCGCATGGCTG
AGGCTGTGCTGGGCACCCACGATGACTGGCAGATAGGCAAAACCAAGATCTTTCTGAAGGA
CCACCATGACATGCTGCTGGAAGTGGAGCGGGACAAAGCCATCACCGACAGAGTCATCCTC
CTTCAGAAAGTCATCCGGGGATTCAAAGACAGGTCTAACTTTCTGAAGCTGAAGAACGCTG
CCACACTGATCCAGAGGCACTGGCGGGGTCACAACTGTAGGAAGAACTACGGGCTGATGC
GTCTGGGCTTCCTGCGGCTGCAGGCCCTGCACCGCTCCCGGAAGCTGCACCAGCAGTACCG
CCTGGCCCGCCAGCGCATCATCCAGTTCCAGGCCCGCTGCCGCGCCTATCTGGTGCGCAAG
GCCTTCCGCCACCGCCTCTGGGCTGTGCTCACCGTGCAGGCCTATGCCCGGGGCATGATCGC
CCGCAGGCTGCACCAACGCCTCAGGGCTGAGTATCTGTGGCGCCTCGAGGCTGAGAAAATG
CGGCTGGCGGAGGAAGAGAAGCTTCGGAAGGAGATGAGCGCCAAGAAGGCCAAGGAGGA
GGCCGAGCGCAAGCATCAGGAGCGCCTGGCCCAGCTGGCTCGTGAGGACGCTGAGCGGGA
GCTGAAGGAGAAGGAGGCCGCTCGGCGGAAGAAGGAGCTCCTGGAGCAGATGGAAAGGG
CCCGCCATGAGCCTGTCAATCACTCAGACATGGTGGACAAGATGTTTGGCTTCCTGGGGAC
TTCAGGTGGCCTGCCAGGCCAGGAGGGCCAGGCACCTAGTGGCTTTGAGGACCTGGAGCGA
GGGCGGAGGGAGATGGTGGAGGAGGACCTGGATGCAGCCCTGCCCCTGCCTGACGAGGAT
GAGGAGGACCTCTCTGAGTATAAATTTGCCAAGTTCGCGGCCACCTACTTCCAGGGGACAA
CCACGCACTCCTACACCCGGCGGCCACTCAAACAGCCACTGCTCTACCATGACGACGAGGG
TGACCAGCTGGCAGCCCTGGCGGTCTGGATCACCATCCTCCGCTTCATGGGGGACCTCCCTG
AGCCCAAGTACCACACAGCCATGAGTGATGGCAGTGAGAAGATCCCTGTGATGACCAAGAT
TTATGAGACCCTGGGCAAGAAGACGTACAAGAGGGAGCTGCAGGCCCTGCAGGGCGAGGG
CGAGGCCCAGCTCCCCGAGGGCCAGTTAATTAAGCATGCTGGGGAGAGATCTGAACCCCTA
GTGATGGAGTTGGCCACTCCCTCTCTGCGCGCTCGCTCGCTCACTGAGGCCGGGCGACCAA
AGGTCGCCCGACGCCCGGGCTTTGCCCGGGCGGCCTCAGTGAGCGAGCGAGCGCGCAGAG
AGGGAG
SEQ ID NO: 66 (only the N-myosin7A portion of SEQ ID NO: 50)
ATGGTGATTCTTCAGCAGGGGGACCATGTGTGGATGGACCTGAGATTGGGGCAGGAGTTCG
ACGTGCCCATCGGGGCGGTGGTGAAGCTCTGCGACTCTGGGCAGGTCCAGGTGGTGGATGA
TGAAGACAATGAACACTGGATCTCTCCGCAGAACGCAACGCACATCAAGCCTATGCACCCC
ACGTCGGTCCACGGCGTGGAGGACATGATCCGCCTGGGGGACCTCAACGAGGCGGGCATCT
TGCGCAACCTGCTTATCCGCTACCGGGACCACCTCATCTACACGTATACGGGCTCCATCCTG
GTGGCTGTGAACCCCTACCAGCTGCTCTCCATCTACTCGCCAGAGCACATCCGCCAGTATAC
CAACAAGAAGATTGGGGAGATGCCCCCCCACATCTTTGCCATTGCTGACAACTGCTACTTC
AACATGAAACGCAACAGCCGAGACCAGTGCTGCATCATCAGTGGGGAATCTGGGGCCGGG
AAGACGGAGAGCACAAAGCTGATCCTGCAGTTCCTGGCAGCCATCAGTGGGCAGCACTCGT
GGATTGAGCAGCAGGTCTTGGAGGCCACCCCCATTCTGGAAGCATTTGGGAATGCCAAGAC
CATCCGCAATGACAACTCAAGCCGTTTCGGAAAGTACATCGACATCCACTTCAACAAGCGG
GGCGCCATCGAGGGCGCGAAGATTGAGCAGTACCTGCTGGAAAAGTCACGTGTCTGTCGCC
AGGCCCTGGATGAAAGGAACTACCACGTGTTCTACTGCATGCTGGAGGGTATGAGTGAGGA
TCAGAAGAAGAAGCTGGGCTTGGGCCAGGCCTCTGACTACAACTACTTGGCCATGGGTAAC
TGCATAACCTGTGAGGGCCGGGTGGACAGCCAGGAGTACGCCAACATCCGCTCCGCCATGA
AGGTGCTCATGTTCACTGACACCGAGAACTGGGAGATCTCGAAGCTCCTGGCTGCCATCCT
GCACCTGGGCAACCTGCAGTATGAGGCACGCACATTTGAAAACCTGGATGCCTGTGAGGTT
CTCTTCTCCCCATCGCTGGCCACAGCTGCATCCCTGCTTGAGGTGAACCCCCCAGACCTGAT
GAGCTGCCTGACTAGCCGCACCCTCATCACCCGCGGGGAGACGGTGTCCACCCCACTGAGC
AGGGAACAGGCACTGGACGTGCGCGACGCCTTCGTAAAGGGGATCTACGGGCGGCTGTTCG
TGTGGATTGTGGACAAGATCAACGCAGCAATTTACAAGCCTCCCTCCCAGGATGTGAAGAA
CTCTCGCAGGTCCATCGGCCTCCTGGACATCTTTGGGTTTGAGAACTTTGCTGTGAACAGCT
TTGAGCAGCTCTGCATCAACTTCGCCAATGAGCACCTGCAGCAGTTCTTTGTGCGGCACGTG
TTCAAGCTGGAGCAGGAGGAATATGACCTGGAGAGCATTGACTGGCTGCACATCGAGTTCA
CTGACAACCAGGATGCCCTGGACATGATTGCCAACAAGCCCATGAACATCATCTCCCTCAT
CGATGAGGAGAGCAAGTTCCCCAAGGGCACAGACACCACCATGTTACACAAGCTGAACTCC
CAGCACAAGCTCAACGCCAACTACATCCCCCCCAAGAACAACCATGAGACCCAGTTTGGCA
TCAACCATTTTGCAGGCATCGTCTACTATGAGACCCAAGGCTTCCTGGAGAAGAACCGAGA
CACCCTGCATGGGGACATTATCCAGCTGGTCCACTCCTCCAGGAACAAGTTCATCAAGCAG
ATCTTCCAGGCCGATGTCGCCATGGGCGCCGAGACCAGGAAGCGCTCGCCCACACTTAGCA
GCCAGTTCAAGCGGTCACTGGAGCTGCTGATGCGCACGCTGGGTGCCTGCCAGCCCTTCTTT
GTGCGATGCATCAAGCCCAATGAGTTCAAGAAGCCCATGCTGTTCGACCGGCACCTGTGCG
TGCGCCAGCTGCGGTACTCAGGAATGATGGAGACCATCCGAATCCGCCGAGCTGGCTACCC
CATCCGCTACAGCTTCGTAGAGTTTGTGGAGCGGTACCGTGTGCTGCTGCCAGGTGTGAAG
CCGGCCTACAAGCAGGGCGACCTCCGCGGGACTTGCCAGCGCATGGCTGAGGCTGTGCTGG
GCACCCACGATGACTGGCAGATAGGCAAAACCAAGATCTTTCTGAAGGACCACCATGACAT
GCTGCTGGAAGTGGAGCGGGACAAAGCCATCACCGACAGAGTCATCCTCCTTCAGAAAGTC
ATCCGGGGATTCAAAGACAGGTCTAACTTTCTGAAGCTGAAGAACGCTGCCACACTGATCC
AGAGGCACTGGCGGGGTCACAACTGTAGGAAGAACTACGGGCTGATGCGTCTGGGCTTCCT
GCGGCTGCAGGCCCTGCACCGCTCCCGGAAGCTGCACCAGCAGTACCGCCTGGCCCGCCAG
CGCATCATCCAGTTCCAGGCCCGCTGCCGCGCCTATCTGGTGCGCAAGGCCTTCCGCCACCG
CCTCTGGGCTGTGCTCACCGTGCAGGCCTATGCCCGGGGCATGATCGCCCGCAGGCTGCAC
CAACGCCTCAGGGCTGAGTATCTGTGGCGCCTCGAGGCTGAGAAAATGCGGCTGGCGGAGG
AAGAGAAGCTTCGGAAGGAGATGAGCGCCAAGAAGGCCAAGGAGGAGGCCGAGCGCAAG
CATCAGGAGCGCCTGGCCCAGCTGGCTCGTGAGGACGCTGAGCGGGAGCTGAAGGAGAAG
GAGGCCGCTCGGCGGAAGAAGGAGCTCCTGGAGCAGATGGAAAGGGCCCGCCATGAGCCT
GTCAATCACTCAGACATGGTGGACAAGATGTTTGGCTTCCTGGGGACTTCAGGTGGCCTGC
CAGGCCAGGAGGGCCAGGCACCTAGTGGCTTTGAGGACCTGGAGCGAGGGCGGAGGGAGA
TGGTGGAGGAGGACCTGGATGCAGCCCTGCCCCTGCCTGACGAGGATGAGGAGGACCTCTC
TGAGTATAAATTTGCCAAGTTCGCGGCCACCTACTTCCAGGGGACAACCACGCACTCCTAC
ACCCGGCGGCCACTCAAACAGCCACTGCTCTACCATGACGACGAGGGTGACCAGCTGGCAG
CCCTGGCGGTCTGGATCACCATCCTCCGCTTCATGGGGGACCTCCCTGAGCCCAAGTACCAC
ACAGCCATGAGTGATGGCAGTGAGAAGATCCCTGTGATGACCAAGATTTATGAGACCCTGG
GCAAGAAGACGTACAAGAGGGAGCTGCAGGCCCTGCAGGGCGAGGGCGAGGCCCAGCTCC
CCGAGGGCCAG
SEQ ID NO: 65
(peptide encoded by the N-myosin7A portion of SEQ ID NO: 50)
MVILQQGDHVWMDLRLGQEFDVPIGAVVKLCDSGQVQVVDDEDNEHWISPQNATHIKPMHPT
SVHGVEDMIRLGDLNEAGILRNLLIRYRDHLIYTYTGSILVAVNPYQLLSIYSPEHIRQYTNKKIG
EMPPHIFAIADNCYFNMKRNSRDQCCIISGESGAGKTESTKLILQFLAAISGQHSWIEQQVLEATP
ILEAFGNAKTIRNDNSSRFGKYIDIHFNKRGAIEGAKIEQYLLEKSRVCRQALDERNYHVFYCML
EGMSEDQKKKLGLGQASDYNYLAMGNCITCEGRVDSQEYANIRSAMKVLMFTDTENWEISKL
LAAILHLGNLQYEARTFENLDACEVLFSPSLATAASLLEVNPPDLMSCLTSRTLITRGETVSTPLS
REQALDVRDAFVKGIYGRLFVWIVDKINAAIYKPPSQDVKNSRRSIGLLDIFGFENFAVNSFEQL
CINFANEHLQQFFVRHVFKLEQEEYDLESIDWLHIEFTDNQDALDMIANKPMNIISLIDEESKFPK
GTDTTMLHKLNSQHKLNANYIPPKNNHETQFGINHFAGIVYYETQGFLEKNRDTLHGDIIQLVH
SSRNKFIKQIFQADVAMGAETRKRSPTLSSQFKRSLELLMRTLGACQPFFVRCIKPNEFKKPMLF
DRHLCVRQLRYSGMMETIRIRRAGYPIRYSFVEFVERYRVLLPGVKPAYKQGDLRGTCQRMAE
AVLGTHDDWQIGKTKIFLKDHHDMLLEVERDKAITDRVILLQKVIRGFKDRSNFLKLKNAATLI
QRHWRGHNCRKNYGLMRLGFLRLQALHRSRKLHQQYRLARQRIIQFQARCRAYLVRKAFRHR
LWAVLTVQAYARGMIARRLHQRLRAEYLWRLEAEKMRLAEEEKLRKEMSAKKAKEEAERKH
QERLAQLAREDAERELKEKEAARRKKELLEQMERARHEPVNHSDMVDKMFGFLGTSGGLPGQ
EGQAPSGFEDLERGRREMVEEDLDAALPLPDEDEEDLSEYKFAKFAATYFQGTTTHSYTRRPLK
QPLLYHDDEGDQLAALAVWITILRFMGDLPEPKYHTAMSDGSEKIPVMTKIYETLGKKTYKREL
QALQGEGEAQLPEGQ
SEQ ID NO: 51 is the polynucleotide sequence of the third
generation overlap back half vector
(i.e., AAV-hMYO7A-CTlong-v3.HA).
CTCCCTCTCTGCGCGCTCGCTCGCTCACTGAGGCCGCCCGGGCAAAGCCCGGGCGTCGGGC
GACCTTTGGTCGCCCGGCCTCAGTGAGCGAGCGAGCGCGCAGAGAGGGAGTGGCCAACTCC
ATCACTAGGGGTTCAGATCTGGCGCGCCCTGGCGGAGGAAGAGAAGCTTCGGAAGGAGAT
GAGCGCCAAGAAGGCCAAGGAGGAGGCCGAGCGCAAGCATCAGGAGCGCCTGGCCCAGCT
GGCTCGTGAGGACGCTGAGCGGGAGCTGAAGGAGAAGGAGGCCGCTCGGCGGAAGAAGG
AGCTCCTGGAGCAGATGGAAAGGGCCCGCCATGAGCCTGTCAATCACTCAGACATGGTGGA
CAAGATGTTTGGCTTCCTGGGGACTTCAGGTGGCCTGCCAGGCCAGGAGGGCCAGGCACCT
AGTGGCTTTGAGGACCTGGAGCGAGGGCGGAGGGAGATGGTGGAGGAGGACCTGGATGCA
GCCCTGCCCCTGCCTGACGAGGATGAGGAGGACCTCTCTGAGTATAAATTTGCCAAGTTCG
CGGCCACCTACTTCCAGGGGACAACCACGCACTCCTACACCCGGCGGCCACTCAAACAGCC
ACTGCTCTACCATGACGACGAGGGTGACCAGCTGGCAGCCCTGGCGGTCTGGATCACCATC
CTCCGCTTCATGGGGGACCTCCCTGAGCCCAAGTACCACACAGCCATGAGTGATGGCAGTG
AGAAGATCCCTGTGATGACCAAGATTTATGAGACCCTGGGCAAGAAGACGTACAAGAGGG
AGCTGCAGGCCCTGCAGGGCGAGGGCGAGGCCCAGCTCCCCGAGGGCCAGAAGAAGAGCA
GTGTGAGGCACAAGCTGGTGCATTTGACTCTGAAAAAGAAGTCCAAGCTCACAGAGGAGGT
GACCAAGAGGCTGCATGACGGGGAGTCCACAGTGCAGGGCAACAGCATGCTGGAGGACCG
GCCCACCTCCAACCTGGAGAAGCTGCACTTCATCATCGGCAATGGCATCCTGCGGCCAGCA
CTCCGGGACGAGATCTACTGCCAGATCAGCAAGCAGCTGACCCACAACCCCTCCAAGAGCA
GCTATGCCCGGGGCTGGATTCTCGTGTCTCTCTGCGTGGGCTGTTTCGCCCCCTCCGAGAAG
TTTGTCAAGTACCTGCGGAACTTCATCCACGGGGGCCCGCCCGGCTACGCCCCGTACTGTGA
GGAGCGCCTGAGAAGGACCTTTGTCAATGGGACACGGACACAGCCGCCCAGCTGGCTGGA
GCTGCAGGCCACCAAGTCCAAGAAGCCAATCATGTTGCCCGTGACATTCATGGATGGGACC
ACCAAGACCCTGCTGACGGACTCGGCAACCACGGCCAAGGAGCTCTGCAACGCGCTGGCCG
ACAAGATCTCTCTCAAGGACCGGTTCGGGTTCTCCCTCTACATTGCCCTGTTTGACAAGGTG
TCCTCCCTGGGCAGCGGCAGTGACCACGTCATGGACGCCATCTCCCAGTGCGAGCAGTACG
CCAAGGAGCAGGGCGCCCAGGAGCGCAACGCCCCCTGGAGGCTCTTCTTCCGCAAAGAGGT
CTTCACGCCCTGGCACAGCCCCTCCGAGGACAACGTGGCCACCAACCTCATCTACCAGCAG
GTGGTGCGAGGAGTCAAGTTTGGGGAGTACAGGTGTGAGAAGGAGGACGACCTGGCTGAG
CTGGCCTCCCAGCAGTACTTTGTAGACTATGGCTCTGAGATGATCCTGGAGCGCCTCCTGAA
CCTCGTGCCCACCTACATCCCCGACCGCGAGATCACGCCCCTGAAGACGCTGGAGAAGTGG
GCCCAGCTGGCCATCGCCGCCCACAAGAAGGGGATTTATGCCCAGAGGAGAACTGATGCCC
AGAAGGTCAAAGAGGATGTGGTCAGTTATGCCCGCTTCAAGTGGCCCTTGCTCTTCTCCAG
GTTTTATGAAGCCTACAAATTCTCAGGCCCCAGTCTCCCCAAGAACGACGTCATCGTGGCCG
TCAACTGGACGGGTGTGTACTTTGTGGATGAGCAGGAGCAGGTACTTCTGGAGCTGTCCTTC
CCAGAGATCATGGCCGTGTCCAGCAGCAGGGGAGCGAAAACGACGGCCCCCAGCTTCACG
CTGGCCACCATCAAGGGGGACGAATACACCTTCACCTCCAGCAATGCTGAGGACATTCGTG
ACCTGGTGGTCACCTTCCTAGAGGGGCTCCGGAAGAGATCTAAGTATGTTGTGGCCCTGCA
GGATAACCCCAACCCCGCAGGCGAGGAGTCAGGCTTCCTCAGCTTTGCCAAGGGAGACCTC
ATCATCCTGGACCATGACACGGGCGAGCAGGTCATGAACTCGGGCTGGGCCAACGGCATCA
ATGAGAGGACCAAGCAGCGTGGGGACTTCCCCACCGACAGTGTGTACGTCATGCCCACTGT
CACCATGCCACCGCGGGAGATTGTGGCCCTGGTCACCATGACTCCCGATCAGAGGCAGGAC
GTTGTCCGGCTCTTGCAGCTGCGAACGGCGGAGCCCGAGGTGCGTGCCAAGCCCTACACGC
TGGAGGAGTTTTCCTATGACTACTTCAGGCCCCCACCCAAGCACACGCTGAGCCGTGTCATG
GTGTCCAAGGCCCGAGGCAAGGACCGGCTGTGGAGCCACACGCGGGAACCGCTCAAGCAG
GCGCTGCTCAAGAAGCTCCTGGGCAGTGAGGAGCTCTCGCAGGAGGCCTGCCTGGCCTTCA
TTGCTGTGCTCAAGTACATGGGCGACTACCCGTCCAAGAGGACACGCTCCGTCAACGAGCT
CACCGACCAGATCTTTGAGGGTCCCCTGAAAGCCGAGCCCCTGAAGGACGAGGCATATGTG
CAGATCCTGAAGCAGCTGACCGACAACCACATCAGGTACAGCGAGGAGCGGGGTTGGGAG
CTGCTCTGGCTGTGCACGGGCCTTTTCCCACCCAGCAACATCCTCCTGCCCCACGTGCAGCG
CTTCCTGCAGTCCCGAAAGCACTGCCCACTCGCCATCGACTGCCTGCAACGGCTCCAGAAA
GCCCTGAGAAACGGGTCCCGGAAGTACCCTCCGCACCTGGTGGAGGTGGAGGCCATCCAGC
ACAAGACCACCCAGATTTTCCACAAAGTCTACTTCCCTGATGACACTGACGAGGCCTTCGA
AGTGGAGTCCAGCACCAAGGCCAAGGACTTCTGCCAGAACATCGCCACCAGGCTGCTCCTC
AAGTCCTCAGAGGGATTCAGCCTCTTTGTCAAAATTGCAGACAAGGTCATCAGCGTTCCTG
AGAATGACTTCTTCTTTGACTTTGTTCGACACTTGACAGACTGGATAAAGAAAGCTCGGCCC
ATCAAGGACGGAATTGTGCCCTCACTCACCTACCAGGTGTTCTTCATGAAGAAGCTGTGGA
CCACCACGGTGCCAGGGAAGGATCCCATGGCCGATTCCATCTTCCACTATTACCAGGAGTT
GCCCAAGTATCTCCGAGGCTACCACAAGTGCACGCGGGAGGAGGTGCTGCAGCTGGGGGC
GCTGATCTACAGGGTCAAGTTCGAGGAGGACAAGTCCTACTTCCCCAGCATCCCCAAGCTG
CTGCGGGAGCTGGTGCCCCAGGACCTTATCCGGCAGGTCTCACCTGATGACTGGAAGCGGT
CCATCGTCGCCTACTTCAACAAGCACGCAGGGAAGTCCAAGGAGGAGGCCAAGCTGGCCTT
CCTGAAGCTCATCTTCAAGTGGCCCACCTTTGGCTCAGCCTTCTTCGAGGTGAAGCAAACTA
CGGAGCCAAACTTCCCTGAGATCCTCCTAATTGCCATCAACAAGTATGGGGTCAGCCTCATC
GATCCCAAAACGAAGGATATCCTCACCACTCATCCCTTCACCAAGATCTCCAACTGGAGCA
GCGGCAACACCTACTTCCACATCACCATTGGGAACTTGGTGCGCGGGAGCAAACTGCTCTG
CGAGACGTCACTGGGCTACAAGATGGATGACCTCCTGACTTCCTACATTAGCCAGATGCTC
ACAGCCATGAGCAAACAGCGGGGCTCCAGGAGCGGCAAGTACCCTTACGATGTACCGGATT
ACGCATGAGGTACCAAGGGCGAATTCTGCAGTCGACTAGAGCTCGCTGATCAGCCTCGACT
GTGCCTTCTAGTTGCCAGCCATCTGTTGTTTGCCCCTCCCCCGTGCCTTCCTTGACCCTGGAA
GGTGCCACTCCCACTGTCCTTTCCTAATAAAATGAGGAAATTGCATCGCATTGTCTGAGTAG
GTGTCATTCTATTCTGGGGGGTGGGGTGGGGCAGGACAGCAAGGGGGAGGATTGGGAAGA
CAATAGCAGGCATGCTGGGGAGAGATCTGAGGACTAGTCCGTCGACTGTTAATTAAGCATG
CTGGGGAGAGATCTGAACCCCTAGTGATGGAGTTGGCCACTCCCTCTCTGCGCGCTCGCTCG
CTCACTGAGGCCGGGCGACCAAAGGTCGCCCGACGCCCGGGCTTTGCCCGGGCGGCCTCAG
TGAGCGAGCGAGCGCGCAGAGAGGGAG
SEQ ID NO: 80 (hMYO7A c-term (e.g., AAV-hMYO7A-CTlong-v3.HA)
CTGGCGGAGGAAGAGAAGCTTCGGAAGGAGATGAGCGCCAAGAAGGCCAAGGAGGAGGC
CGAGCGCAAGCATCAGGAGCGCCTGGCCCAGCTGGCTCGTGAGGACGCTGAGCGGGAGCT
GAAGGAGAAGGAGGCCGCTCGGCGGAAGAAGGAGCTCCTGGAGCAGATGGAAAGGGCCC
GCCATGAGCCTGTCAATCACTCAGACATGGTGGACAAGATGTTTGGCTTCCTGGGGACTTC
AGGTGGCCTGCCAGGCCAGGAGGGCCAGGCACCTAGTGGCTTTGAGGACCTGGAGCGAGG
GCGGAGGGAGATGGTGGAGGAGGACCTGGATGCAGCCCTGCCCCTGCCTGACGAGGATGA
GGAGGACCTCTCTGAGTATAAATTTGCCAAGTTCGCGGCCACCTACTTCCAGGGGACAACC
ACGCACTCCTACACCCGGCGGCCACTCAAACAGCCACTGCTCTACCATGACGACGAGGGTG
ACCAGCTGGCAGCCCTGGCGGTCTGGATCACCATCCTCCGCTTCATGGGGGACCTCCCTGA
GCCCAAGTACCACACAGCCATGAGTGATGGCAGTGAGAAGATCCCTGTGATGACCAAGATT
TATGAGACCCTGGGCAAGAAGACGTACAAGAGGGAGCTGCAGGCCCTGCAGGGCGAGGGC
GAGGCCCAGCTCCCCGAGGGCCAGAAGAAGAGCAGTGTGAGGCACAAGCTGGTGCATTTG
ACTCTGAAAAAGAAGTCCAAGCTCACAGAGGAGGTGACCAAGAGGCTGCATGACGGGGAG
TCCACAGTGCAGGGCAACAGCATGCTGGAGGACCGGCCCACCTCCAACCTGGAGAAGCTGC
ACTTCATCATCGGCAATGGCATCCTGCGGCCAGCACTCCGGGACGAGATCTACTGCCAGAT
CAGCAAGCAGCTGACCCACAACCCCTCCAAGAGCAGCTATGCCCGGGGCTGGATTCTCGTG
TCTCTCTGCGTGGGCTGTTTCGCCCCCTCCGAGAAGTTTGTCAAGTACCTGCGGAACTTCAT
CCACGGGGGCCCGCCCGGCTACGCCCCGTACTGTGAGGAGCGCCTGAGAAGGACCTTTGTC
AATGGGACACGGACACAGCCGCCCAGCTGGCTGGAGCTGCAGGCCACCAAGTCCAAGAAG
CCAATCATGTTGCCCGTGACATTCATGGATGGGACCACCAAGACCCTGCTGACGGACTCGG
CAACCACGGCCAAGGAGCTCTGCAACGCGCTGGCCGACAAGATCTCTCTCAAGGACCGGTT
CGGGTTCTCCCTCTACATTGCCCTGTTTGACAAGGTGTCCTCCCTGGGCAGCGGCAGTGACC
ACGTCATGGACGCCATCTCCCAGTGCGAGCAGTACGCCAAGGAGCAGGGCGCCCAGGAGC
GCAACGCCCCCTGGAGGCTCTTCTTCCGCAAAGAGGTCTTCACGCCCTGGCACAGCCCCTCC
GAGGACAACGTGGCCACCAACCTCATCTACCAGCAGGTGGTGCGAGGAGTCAAGTTTGGGG
AGTACAGGTGTGAGAAGGAGGACGACCTGGCTGAGCTGGCCTCCCAGCAGTACTTTGTAGA
CTATGGCTCTGAGATGATCCTGGAGCGCCTCCTGAACCTCGTGCCCACCTACATCCCCGACC
GCGAGATCACGCCCCTGAAGACGCTGGAGAAGTGGGCCCAGCTGGCCATCGCCGCCCACA
AGAAGGGGATTTATGCCCAGAGGAGAACTGATGCCCAGAAGGTCAAAGAGGATGTGGTCA
GTTATGCCCGCTTCAAGTGGCCCTTGCTCTTCTCCAGGTTTTATGAAGCCTACAAATTCTCA
GGCCCCAGTCTCCCCAAGAACGACGTCATCGTGGCCGTCAACTGGACGGGTGTGTACTTTG
TGGATGAGCAGGAGCAGGTACTTCTGGAGCTGTCCTTCCCAGAGATCATGGCCGTGTCCAG
CAGCAGGGGAGCGAAAACGACGGCCCCCAGCTTCACGCTGGCCACCATCAAGGGGGACGA
ATACACCTTCACCTCCAGCAATGCTGAGGACATTCGTGACCTGGTGGTCACCTTCCTAGAGG
GGCTCCGGAAGAGATCTAAGTATGTTGTGGCCCTGCAGGATAACCCCAACCCCGCAGGCGA
GGAGTCAGGCTTCCTCAGCTTTGCCAAGGGAGACCTCATCATCCTGGACCATGACACGGGC
GAGCAGGTCATGAACTCGGGCTGGGCCAACGGCATCAATGAGAGGACCAAGCAGCGTGGG
GACTTCCCCACCGACAGTGTGTACGTCATGCCCACTGTCACCATGCCACCGCGGGAGATTGT
GGCCCTGGTCACCATGACTCCCGATCAGAGGCAGGACGTTGTCCGGCTCTTGCAGCTGCGA
ACGGCGGAGCCCGAGGTGCGTGCCAAGCCCTACACGCTGGAGGAGTTTTCCTATGACTACT
TCAGGCCCCCACCCAAGCACACGCTGAGCCGTGTCATGGTGTCCAAGGCCCGAGGCAAGGA
CCGGCTGTGGAGCCACACGCGGGAACCGCTCAAGCAGGCGCTGCTCAAGAAGCTCCTGGGC
AGTGAGGAGCTCTCGCAGGAGGCCTGCCTGGCCTTCATTGCTGTGCTCAAGTACATGGGCG
ACTACCCGTCCAAGAGGACACGCTCCGTCAACGAGCTCACCGACCAGATCTTTGAGGGTCC
CCTGAAAGCCGAGCCCCTGAAGGACGAGGCATATGTGCAGATCCTGAAGCAGCTGACCGA
CAACCACATCAGGTACAGCGAGGAGCGGGGTTGGGAGCTGCTCTGGCTGTGCACGGGCCTT
TTCCCACCCAGCAACATCCTCCTGCCCCACGTGCAGCGCTTCCTGCAGTCCCGAAAGCACTG
CCCACTCGCCATCGACTGCCTGCAACGGCTCCAGAAAGCCCTGAGAAACGGGTCCCGGAAG
TACCCTCCGCACCTGGTGGAGGTGGAGGCCATCCAGCACAAGACCACCCAGATTTTCCACA
AAGTCTACTTCCCTGATGACACTGACGAGGCCTTCGAAGTGGAGTCCAGCACCAAGGCCAA
GGACTTCTGCCAGAACATCGCCACCAGGCTGCTCCTCAAGTCCTCAGAGGGATTCAGCCTCT
TTGTCAAAATTGCAGACAAGGTCATCAGCGTTCCTGAGAATGACTTCTTCTTTGACTTTGTT
CGACACTTGACAGACTGGATAAAGAAAGCTCGGCCCATCAAGGACGGAATTGTGCCCTCAC
TCACCTACCAGGTGTTCTTCATGAAGAAGCTGTGGACCACCACGGTGCCAGGGAAGGATCC
CATGGCCGATTCCATCTTCCACTATTACCAGGAGTTGCCCAAGTATCTCCGAGGCTACCACA
AGTGCACGCGGGAGGAGGTGCTGCAGCTGGGGGCGCTGATCTACAGGGTCAAGTTCGAGG
AGGACAAGTCCTACTTCCCCAGCATCCCCAAGCTGCTGCGGGAGCTGGTGCCCCAGGACCT
TATCCGGCAGGTCTCACCTGATGACTGGAAGCGGTCCATCGTCGCCTACTTCAACAAGCAC
GCAGGGAAGTCCAAGGAGGAGGCCAAGCTGGCCTTCCTGAAGCTCATCTTCAAGTGGCCCA
CCTTTGGCTCAGCCTTCTTCGAGGTGAAGCAAACTACGGAGCCAAACTTCCCTGAGATCCTC
CTAATTGCCATCAACAAGTATGGGGTCAGCCTCATCGATCCCAAAACGAAGGATATCCTCA
CCACTCATCCCTTCACCAAGATCTCCAACTGGAGCAGCGGCAACACCTACTTCCACATCACC
ATTGGGAACTTGGTGCGCGGGAGCAAACTGCTCTGCGAGACGTCACTGGGCTACAAGATGG
ATGACCTCCTGACTTCCTACATTAGCCAGATGCTCACAGCCATGAGCAAACAGCGGGGCTC
CAGGAGCGGCAAG
SEQ ID NO: 81 (hMY07A c-term (e.g., AAV-hMYO7A-CTlong-v3.HA)
LAEEEKLRKEMSAKKAKEEAERKHQERLAQLAREDAERELKEKEAARRKKELLEQMERARHE
PVNHSDMVDKMFGFLGTSGGLPGQEGQAPSGFEDLERGRREMVEEDLDAALPLPDEDEEDLSE
YKFAKFAATYFQGTTTHSYTRRPLKQPLLYHDDEGDQLAALAVWITILRFMGDLPEPKYHTAM
SDGSEKIPVMTKIYETLGKKTYKRELQALQGEGEAQLPEGQKKSSVRHKLVHLTLKKKSKLTEE
VTKRLHDGESTVQGNSMLEDRPTSNLEKLHFIIGNGILRPALRDEIYCQISKQLTHNPSKSSYARG
WILVSLCVGCFAPSEKFVKYLRNFIHGGPPGYAPYCEERLRRTFVNGTRTQPPSWLELQATKSK
KPIMLPVTFMDGTTKTLLTDSATTAKELCNALADKISLKDRFGFSLYIALFDKVSSLGSGSDHVM
DAISQCEQYAKEQGAQERNAPWRLFFRKEVFTPWHSPSEDNVATNLIYQQVVRGVKFGEYRCE
KEDDLAELASQQYFVDYGSEMILERLLNLVPTYIPDREITPLKTLEKWAQLAIAAHKKGIYAQR
RTDAQKVKEDVVSYARFKWPLLFSRFYEAYKFSGPSLPKNDVIVAVNWTGVYFVDEQEQVLLE
LSFPEIMAVSSSRGAKTTAPSFTLATIKGDEYTFTSSNAEDIRDLVVTFLEGLRKRSKYVVALQD
NPNPAGEESGFLSFAKGDLIILDHDTGEQVMNSGWANGINERTKQRGDFPTDSVYVMPTVTMP
PREIVALVTMTPDQRQDVVRLLQLRTAEPEVRAKPYTLEEFSYDYFRPPPKHTLSRVMVSKARG
KDRLWSHTREPLKQALLKKLLGSEELSQEACLAFIAVLKYMGDYPSKRTRSVNELTDQIFEGPL
KAEPLKDEAYVQILKQLTDNHIRYSEERGWELLWLCTGLFPPSNILLPHVQRFLQSRKHCPLAID
CLQRLQKALRNGSRKYPPHLVEVEAIQHKTTQIFHKVYFPDDTDEAFEVESSTKAKDFCQNIAT
RLLLKSSEGFSLFVKIADKVISVPENDFFFDFVRHLTDWIKKARPIKDGIVPSLTYQVFFMKKLW
TTTVPGKDPMADSIFHYYQELPKYLRGYHKCTREEVLQLGALIYRVKFEEDKSYFPSIPKLLREL
VPQDLIRQVSPDDWKRSIVAYFNKHAGKSKEEAKLAFLKLIFKWPTFGSAFFEVKQTTEPNFPEI
LLIAINKYGVSLIDPKTKDILTTHPFTKISNWSSGNTYFHITIGNLVRGSKLLCETSLGYKMDDLL
TSYISQMLTAMSKQRGSRSGK

In some embodiments, the overlap polynucleotide vectors provided herein comprise a region of overlap that comprises a nucleotide sequence having 80%, 85%, 90%, 92.5%, 95%, 98%, or 99% sequence identity to any of the overlap sequences provided herein. In some embodiments, the overlapping regions comprises a nucleotide sequence having 80%, 85%, 90%, 92.5%, 95%, 98%, or 99% sequence identity to any one of SEQ ID NOs: 39 and 52-59. In some embodiments, the overlapping regions comprises a nucleotide sequence having 80%, 85%, 90%, 92.5%, 95%, 98%, or 99% sequence identity to any one of SEQ ID NOs: 39 and 53-59. In some embodiments, the overlapping regions (or polynucleotide sequence that overlaps) comprises a nucleotide sequence having 80%, 85%, 90%, 92.5%, 95%, 98%, or 99% sequence identity to SEQ ID NO: 39, 56 or 57. In some embodiments, the polynucleotide sequence that overlaps comprises a nucleotide sequence selected from any of SEQ ID NOs: 39 and 52-59. The overlap vectors provided herein may comprise a polynucleotide sequence that overlaps that differs from any of SEQ ID NOs: 39 and 52-59 by 1, 2, 3, 4, 5, 6, 7, 8, 9, 10, 10-15, 15-20, 20-25, or more than 25 nucleotides. In some embodiments, these overlap vectors contain two overlapping sequences disclosed herein, e.g., the mutually exclusive sequences SEQ ID NOs: 39 and 56, or the mutually exclusive sequences SEQ ID NOs: 39 and 57.

In exemplary embodiments, the polynucleotide sequence that overlaps comprises SEQ ID NO: 56. In some embodiments, the polynucleotide sequence that overlaps comprises SEQ ID NO: 57. In some embodiments, the polynucleotide sequence that overlaps comprises SEQ ID NO: 39. In some embodiments, the polynucleotide sequence that overlaps does not comprise SEQ ID NO: 52.

In some embodiments, the overlap polynucleotide vectors provided herein comprise a region of overlap (polynucleotide sequence that overlaps) that encodes a protein having an amino acid sequence comprising 95%, 98%, or 99% or greater sequence identity to any of SEQ ID NOs: 79 and 82-89. In exemplary embodiments, these vectors comprise a region of overlap (polynucleotide sequence that overlaps) that encodes a protein having the amino acid sequence of any one of SEQ ID NOs: 79 and 82-89. In exemplary embodiments, these vectors comprise a region of overlap (polynucleotide sequence that overlaps) that encodes a protein having the amino acid sequence of any one of SEQ ID NOs: 79 and 83-89. In some embodiments, the polynucleotide sequence that overlaps does not encode the amino acid sequence of SEQ ID NO: 82.

In various embodiments, the overlap polynucleotide vectors provided herein contain a region of overlap in the polypeptide coding sequence having a length of about 1365 bp, 1284 bp, 1027 bp, 1026 bp, 945 bp, 687 bp, 361 bp, 279 bp, or 20 bp. In some embodiments, overlap vectors contain a region of overlap of less than 1000 bp in length. In some embodiments, overlap vectors contain a region of overlap of less than 1365 bp. In some embodiments, overlap vectors contain a region of overlap of less than 700 bp. In some embodiments, the region of overlap has a length of between about 20 to 100 nucleotides, about 100 to 500 nucleotides, about 100 to 200 nucleotides, about 200 to 300 nucleotides, or about 300 to 400 nucleotides.

In various embodiments, the overlap polynucleotide vectors provided herein contain a region of overlap having a length of exactly 1365 bp, 1284 bp, 1027 bp, 1026 bp, 945 bp, 687 bp, 361 bp, 279 bp, or 20 bp. It will be understood that these regions of overlap may be mutually exclusive of one another in the coding sequence. In some embodiments, the overlap polynucleotide vectors contain one or more regions of overlap, e.g., contain two regions of overlap. In some embodiments, the overlap vectors contain two regions of overlap having lengths of 361 bp and 687 bp.

In exemplary embodiments, the overlap polynucleotide vectors provided herein contain a region of overlap having a length of 687 or 945 bp. In some embodiments, these vectors contain a region of overlapping MYO7A sequence having a length of 687 or 945 bp. In some embodiments, overlap vectors contain a region of overlap having a length of 361 bp.

1365 bp Overlap (SEQ ID NO: 52).
CAGGTCTAACTTTCTGAAGCTGAAGAACGCTGCCACACTGATCCAGAGGCACTGGC
GGGGTCACAACTGTAGGAAGAACTACGGGCTGATGCGTCTGGGCTTCCTGCGGCTG
CAGGCCCTGCACCGCTCCCGGAAGCTGCACCAGCAGTACCGCCTGGCCCGCCAGCG
CATCATCCAGTTCCAGGCCCGCTGCCGCGCCTATCTGGTGCGCAAGGCCTTCCGCCA
CCGCCTCTGGGCTGTGCTCACCGTGCAGGCCTATGCCCGGGGCATGATCGCCCGCA
GGCTGCACCAACGCCTCAGGGCTGAGTATCTGTGGCGCCTCGAGGCTGAGAAAATG
CGGCTGGCGGAGGAAGAGAAGCTTCGGAAGGAGATGAGCGCCAAGAAGGCCAAG
GAGGAGGCCGAGCGCAAGCATCAGGAGCGCCTGGCCCAGCTGGCTCGTGAGGACG
CTGAGCGGGAGCTGAAGGAGAAGGAGGCCGCTCGGCGGAAGAAGGAGCTCCTGGA
GCAGATGGAAAGGGCCCGCCATGAGCCTGTCAATCACTCAGACATGGTGGACAAG
ATGTTTGGCTTCCTGGGGACTTCAGGTGGCCTGCCAGGCCAGGAGGGCCAGGCACC
TAGTGGCTTTGAGGACCTGGAGCGAGGGCGGAGGGAGATGGTGGAGGAGGACCTG
GATGCAGCCCTGCCCCTGCCTGACGAGGATGAGGAGGACCTCTCTGAGTATAAATT
TGCCAAGTTCGCGGCCACCTACTTCCAGGGGACAACCACGCACTCCTACACCCGGC
GGCCACTCAAACAGCCACTGCTCTACCATGACGACGAGGGTGACCAGCTGGCAGCC
CTGGCGGTCTGGATCACCATCCTCCGCTTCATGGGGGACCTCCCTGAGCCCAAGTA
CCACACAGCCATGAGTGATGGCAGTGAGAAGATCCCTGTGATGACCAAGATTTATG
AGACCCTGGGCAAGAAGACGTACAAGAGGGAGCTGCAGGCCCTGCAGGGCGAGGG
CGAGGCCCAGCTCCCCGAGGGCCAGAAGAAGAGCAGTGTGAGGCACAAGCTGGTG
CATTTGACTCTGAAAAAGAAGTCCAAGCTCACAGAGGAGGTGACCAAGAGGCTGC
ATGACGGGGAGTCCACAGTGCAGGGCAACAGCATGCTGGAGGACCGGCCCACCTC
CAACCTGGAGAAGCTGCACTTCATCATCGGCAATGGCATCCTGCGGCCAGCACTCC
GGGACGAGATCTACTGCCAGATCAGCAAGCAGCTGACCCACAACCCCTCCAAGAG
CAGCTATGCCCGGGGCTGGATTCTCGTGTCTCTCTGCGTGGGCTGTTTCGCCCCCTC
CGAGAAGTTTGTCAAGTACCTGCGGAACTTC
SEQ ID NO: 82
(protein sequence that corresponds to SEQ ID NO: 52)
RSNFLKLKNAATLIQRHWRGHNCRKNYGLMRLGFLRLQALHRSRKLHQQYRLARQRII
QFQARCRAYLVRKAFRHRLWAVLTVQAYARGMIARRLHQRLRAEYLWRLEAEKMRL
AEEEKLRKEMSAKKAKEEAERKHQERLAQLAREDAERELKEKEAARRKKELLEQMER
ARHEPVNHSDMVDKMFGFLGTSGGLPGQEGQAPSGFEDLERGRREMVEEDLDAALPL
PDEDEEDLSEYKFAKFAATYFQGTTTHSYTRRPLKQPLLYHDDEGDQLAALAVWITILR
FMGDLPEPKYHTAMSDGSEKIPVMTKIYETLGKKTYKRELQALQGEGEAQLPEGQKKS
SVRHKLVHLTLKKKSKLTEEVTKRLHDGESTVQGNSMLEDRPTSNLEKLHFIIGNGILR
PALRDEIYCQISKQLTHNPSKSSYARGWILVSLCVGCFAPSEKFVKYLRNF
1284 bp Overlap (SEQ ID NO: 53).
GGGCTGATGCGTCTGGGCTTCCTGCGGCTGCAGGCCCTGCACCGCTCCCGGAAGCT
GCACCAGCAGTACCGCCTGGCCCGCCAGCGCATCATCCAGTTCCAGGCCCGCTGCC
GCGCCTATCTGGTGCGCAAGGCCTTCCGCCACCGCCTCTGGGCTGTGCTCACCGTGC
AGGCCTATGCCCGGGGCATGATCGCCCGCAGGCTGCACCAACGCCTCAGGGCTGAG
TATCTGTGGCGCCTCGAGGCTGAGAAAATGCGGCTGGCGGAGGAAGAGAAGCTTC
GGAAGGAGATGAGCGCCAAGAAGGCCAAGGAGGAGGCCGAGCGCAAGCATCAGG
AGCGCCTGGCCCAGCTGGCTCGTGAGGACGCTGAGCGGGAGCTGAAGGAGAAGGA
GGCCGCTCGGCGGAAGAAGGAGCTCCTGGAGCAGATGGAAAGGGCCCGCCATGAG
CCTGTCAATCACTCAGACATGGTGGACAAGATGTTTGGCTTCCTGGGGACTTCAGG
TGGCCTGCCAGGCCAGGAGGGCCAGGCACCTAGTGGCTTTGAGGACCTGGAGCGA
GGGCGGAGGGAGATGGTGGAGGAGGACCTGGATGCAGCCCTGCCCCTGCCTGACG
AGGATGAGGAGGACCTCTCTGAGTATAAATTTGCCAAGTTCGCGGCCACCTACTTC
CAGGGGACAACCACGCACTCCTACACCCGGCGGCCACTCAAACAGCCACTGCTCTA
CCATGACGACGAGGGTGACCAGCTGGCAGCCCTGGCGGTCTGGATCACCATCCTCC
GCTTCATGGGGGACCTCCCTGAGCCCAAGTACCACACAGCCATGAGTGATGGCAGT
GAGAAGATCCCTGTGATGACCAAGATTTATGAGACCCTGGGCAAGAAGACGTACA
AGAGGGAGCTGCAGGCCCTGCAGGGCGAGGGCGAGGCCCAGCTCCCCGAGGGCCA
GAAGAAGAGCAGTGTGAGGCACAAGCTGGTGCATTTGACTCTGAAAAAGAAGTCC
AAGCTCACAGAGGAGGTGACCAAGAGGCTGCATGACGGGGAGTCCACAGTGCAGG
GCAACAGCATGCTGGAGGACCGGCCCACCTCCAACCTGGAGAAGCTGCACTTCATC
ATCGGCAATGGCATCCTGCGGCCAGCACTCCGGGACGAGATCTACTGCCAGATCAG
CAAGCAGCTGACCCACAACCCCTCCAAGAGCAGCTATGCCCGGGGCTGGATTCTCG
TGTCTCTCTGCGTGGGCTGTTTCGCCCCCTCCGAGAAGTTTGTCAAGTACCTGCGGA
ACTTC
SEQ ID NO: 83
(protein sequence that corresponds to SEQ ID NO: 53)
GLMRLGFLRLQALHRSRKLHQQYRLARQRIIQFQARCRAYLVRKAFRHRLWAVLTVQ
AYARGMIARRLHQRLRAEYLWRLEAEKMRLAEEEKLRKEMSAKKAKEEAERKHQER
LAQLAREDAERELKEKEAARRKKELLEQMERARHEPVNHSDMVDKMFGFLGTSGGLP
GQEGQAPSGFEDLERGRREMVEEDLDAALPLPDEDEEDLSEYKFAKFAATYFQGTTTH
SYTRRPLKQPLLYHDDEGDQLAALAVWITILRFMGDLPEPKYHTAMSDGSEKIPVMTKI
YETLGKKTYKRELQALQGEGEAQLPEGQKKSSVRHKLVHLTLKKKSKLTEEVTKRLH
DGESTVQGNSMLEDRPTSNLEKLHFIIGNGILRPALRDEIYCQISKQLTHNPSKSSYARG
WILVSLCVGCFAPSEKFVKYLRNF
1027 bp Overlap (SEQ ID NO: 54).
CAGGTCTAACTTTCTGAAGCTGAAGAACGCTGCCACACTGATCCAGAGGCACTGGC
GGGGTCACAACTGTAGGAAGAACTACGGGCTGATGCGTCTGGGCTTCCTGCGGCTG
CAGGCCCTGCACCGCTCCCGGAAGCTGCACCAGCAGTACCGCCTGGCCCGCCAGCG
CATCATCCAGTTCCAGGCCCGCTGCCGCGCCTATCTGGTGCGCAAGGCCTTCCGCCA
CCGCCTCTGGGCTGTGCTCACCGTGCAGGCCTATGCCCGGGGCATGATCGCCCGCA
GGCTGCACCAACGCCTCAGGGCTGAGTATCTGTGGCGCCTCGAGGCTGAGAAAATG
CGGCTGGCGGAGGAAGAGAAGCTTCGGAAGGAGATGAGCGCCAAGAAGGCCAAG
GAGGAGGCCGAGCGCAAGCATCAGGAGCGCCTGGCCCAGCTGGCTCGTGAGGACG
CTGAGCGGGAGCTGAAGGAGAAGGAGGCCGCTCGGCGGAAGAAGGAGCTCCTGGA
GCAGATGGAAAGGGCCCGCCATGAGCCTGTCAATCACTCAGACATGGTGGACAAG
ATGTTTGGCTTCCTGGGGACTTCAGGTGGCCTGCCAGGCCAGGAGGGCCAGGCACC
TAGTGGCTTTGAGGACCTGGAGCGAGGGCGGAGGGAGATGGTGGAGGAGGACCTG
GATGCAGCCCTGCCCCTGCCTGACGAGGATGAGGAGGACCTCTCTGAGTATAAATT
TGCCAAGTTCGCGGCCACCTACTTCCAGGGGACAACCACGCACTCCTACACCCGGC
GGCCACTCAAACAGCCACTGCTCTACCATGACGACGAGGGTGACCAGCTGGCAGCC
CTGGCGGTCTGGATCACCATCCTCCGCTTCATGGGGGACCTCCCTGAGCCCAAGTA
CCACACAGCCATGAGTGATGGCAGTGAGAAGATCCCTGTGATGACCAAGATTTATG
AGACCCTGGGCAAGAAGACGTACAAGAGGGAGCTGCAGGCCCTGCAGGGCGAGGG
CGAGGCCCAGCTCCCCGAGGGCCAG
SEQ ID NO: 84
(protein sequence that corresponds to SEQ ID NO: 54)
RSNFLKLKNAATLIQRHWRGHNCRKNYGLMRLGFLRLQALHRSRKLHQQYRLARQRII
QFQARCRAYLVRKAFRHRLWAVLTVQAYARGMIARRLHQRLRAEYLWRLEAEKMRL
AEEEKLRKEMSAKKAKEEAERKHQERLAQLAREDAERELKEKEAARRKKELLEQMER
ARHEPVNHSDMVDKMFGFLGTSGGLPGQEGQAPSGFEDLERGRREMVEEDLDAALPL
PDEDEEDLSEYKFAKFAATYFQGTTTHSYTRRPLKQPLLYHDDEGDQLAALAVWITILR
FMGDLPEPKYHTAMSDGSEKIPVMTKIYETLGKKTYKRELQALQGEGEAQLPEGQ
1026 bp Overlap (SEQ ID NO: 55).
CTGGCGGAGGAAGAGAAGCTTCGGAAGGAGATGAGCGCCAAGAAGGCCAAGGAG
GAGGCCGAGCGCAAGCATCAGGAGCGCCTGGCCCAGCTGGCTCGTGAGGACGCTG
AGCGGGAGCTGAAGGAGAAGGAGGCCGCTCGGCGGAAGAAGGAGCTCCTGGAGC
AGATGGAAAGGGCCCGCCATGAGCCTGTCAATCACTCAGACATGGTGGACAAGAT
GTTTGGCTTCCTGGGGACTTCAGGTGGCCTGCCAGGCCAGGAGGGCCAGGCACCTA
GTGGCTTTGAGGACCTGGAGCGAGGGCGGAGGGAGATGGTGGAGGAGGACCTGGA
TGCAGCCCTGCCCCTGCCTGACGAGGATGAGGAGGACCTCTCTGAGTATAAATTTG
CCAAGTTCGCGGCCACCTACTTCCAGGGGACAACCACGCACTCCTACACCCGGCGG
CCACTCAAACAGCCACTGCTCTACCATGACGACGAGGGTGACCAGCTGGCAGCCCT
GGCGGTCTGGATCACCATCCTCCGCTTCATGGGGGACCTCCCTGAGCCCAAGTACC
ACACAGCCATGAGTGATGGCAGTGAGAAGATCCCTGTGATGACCAAGATTTATGAG
ACCCTGGGCAAGAAGACGTACAAGAGGGAGCTGCAGGCCCTGCAGGGCGAGGGCG
AGGCCCAGCTCCCCGAGGGCCAGAAGAAGAGCAGTGTGAGGCACAAGCTGGTGCA
TTTGACTCTGAAAAAGAAGTCCAAGCTCACAGAGGAGGTGACCAAGAGGCTGCAT
GACGGGGAGTCCACAGTGCAGGGCAACAGCATGCTGGAGGACCGGCCCACCTCCA
ACCTGGAGAAGCTGCACTTCATCATCGGCAATGGCATCCTGCGGCCAGCACTCCGG
GACGAGATCTACTGCCAGATCAGCAAGCAGCTGACCCACAACCCCTCCAAGAGCA
GCTATGCCCGGGGCTGGATTCTCGTGTCTCTCTGCGTGGGCTGTTTCGCCCCCTCCG
AGAAGTTTGTCAAGTACCTGCGGAACTTC
SEQ ID NO: 85
(protein sequence that corresponds to SEQ ID NO: 55)
LAEEEKLRKEMSAKKAKEEAERKHQERLAQLAREDAERELKEKEAARRKKELLEQME
RARHEPVNHSDMVDKMFGFLGTSGGLPGQEGQAPSGFEDLERGRREMVEEDLDAALP
LPDEDEEDLSEYKFAKFAATYFQGTTTHSYTRRPLKQPLLYHDDEGDQLAALAVWITIL
RFMGDLPEPKYHTAMSDGSEKIPVMTKIYETLGKKTYKRELQALQGEGEAQLPEGQKK
SSVRHKLVHLTLKKKSKLTEEVTKRLHDGESTVQGNSMLEDRPTSNLEKLHFIIGNGIL
RPALRDEIYCQISKQLTHNPSKSSYARGWILVSLCVGCFAPSEKFVKYLRNF
945 bp Overlap (SEQ ID NO: 56).
GGGCTGATGCGTCTGGGCTTCCTGCGGCTGCAGGCCCTGCACCGCTCCCGGAAGCT
GCACCAGCAGTACCGCCTGGCCCGCCAGCGCATCATCCAGTTCCAGGCCCGCTGCC
GCGCCTATCTGGTGCGCAAGGCCTTCCGCCACCGCCTCTGGGCTGTGCTCACCGTGC
AGGCCTATGCCCGGGGCATGATCGCCCGCAGGCTGCACCAACGCCTCAGGGCTGAG
TATCTGTGGCGCCTCGAGGCTGAGAAAATGCGGCTGGCGGAGGAAGAGAAGCTTC
GGAAGGAGATGAGCGCCAAGAAGGCCAAGGAGGAGGCCGAGCGCAAGCATCAGG
AGCGCCTGGCCCAGCTGGCTCGTGAGGACGCTGAGCGGGAGCTGAAGGAGAAGGA
GGCCGCTCGGCGGAAGAAGGAGCTCCTGGAGCAGATGGAAAGGGCCCGCCATGAG
CCTGTCAATCACTCAGACATGGTGGACAAGATGTTTGGCTTCCTGGGGACTTCAGG
TGGCCTGCCAGGCCAGGAGGGCCAGGCACCTAGTGGCTTTGAGGACCTGGAGCGA
GGGCGGAGGGAGATGGTGGAGGAGGACCTGGATGCAGCCCTGCCCCTGCCTGACG
AGGATGAGGAGGACCTCTCTGAGTATAAATTTGCCAAGTTCGCGGCCACCTACTTC
CAGGGGACAACCACGCACTCCTACACCCGGCGGCCACTCAAACAGCCACTGCTCTA
CCATGACGACGAGGGTGACCAGCTGGCAGCCCTGGCGGTCTGGATCACCATCCTCC
GCTTCATGGGGGACCTCCCTGAGCCCAAGTACCACACAGCCATGAGTGATGGCAGT
GAGAAGATCCCTGTGATGACCAAGATTTATGAGACCCTGGGCAAGAAGACGTACA
AGAGGGAGCTGCAGGCCCTGCAGGGCGAGGGCGAGGCCCAGCTCCCCGAGGGCCA
G
SEQ ID NO: 86
(protein sequence that corresponds to SEQ ID NO: 56)
GLMRLGFLRLQALHRSRKLHQQYRLARQRIIQFQARCRAYLVRKAFRHRLWAVLTVQ
AYARGMIARRLHQRLRAEYLWRLEAEKMRLAEEEKLRKEMSAKKAKEEAERKHQER
LAQLAREDAERELKEKEAARRKKELLEQMERARHEPVNHSDMVDKMFGFLGTSGGLP
GQEGQAPSGFEDLERGRREMVEEDLDAALPLPDEDEEDLSEYKFAKFAATYFQGTTTH
SYTRRPLKQPLLYHDDEGDQLAALAVWITILRFMGDLPEPKYHTAMSDGSEKIPVMTKI
YETLGKKTYKRELQALQGEGEAQLPEGQ
687 bp Overlap (SEQ ID NO: 57).
CTGGCGGAGGAAGAGAAGCTTCGGAAGGAGATGAGCGCCAAGAAGGCCAAGGAG
GAGGCCGAGCGCAAGCATCAGGAGCGCCTGGCCCAGCTGGCTCGTGAGGACGCTG
AGCGGGAGCTGAAGGAGAAGGAGGCCGCTCGGCGGAAGAAGGAGCTCCTGGAGC
AGATGGAAAGGGCCCGCCATGAGCCTGTCAATCACTCAGACATGGTGGACAAGAT
GTTTGGCTTCCTGGGGACTTCAGGTGGCCTGCCAGGCCAGGAGGGCCAGGCACCTA
GTGGCTTTGAGGACCTGGAGCGAGGGCGGAGGGAGATGGTGGAGGAGGACCTGGA
TGCAGCCCTGCCCCTGCCTGACGAGGATGAGGAGGACCTCTCTGAGTATAAATTTG
CCAAGTTCGCGGCCACCTACTTCCAGGGGACAACCACGCACTCCTACACCCGGCGG
CCACTCAAACAGCCACTGCTCTACCATGACGACGAGGGTGACCAGCTGGCAGCCCT
GGCGGTCTGGATCACCATCCTCCGCTTCATGGGGGACCTCCCTGAGCCCAAGTACC
ACACAGCCATGAGTGATGGCAGTGAGAAGATCCCTGTGATGACCAAGATTTATGAG
ACCCTGGGCAAGAAGACGTACAAGAGGGAGCTGCAGGCCCTGCAGGGCGAGGGCG
AGGCCCAGCTCCCCGAGGGCCAG
SEQ ID NO: 87
(protein sequence that corresponds to SEQ ID NO: 57)
LAEEEKLRKEMSAKKAKEEAERKHQERLAQLAREDAERELKEKEAARRKKELLEQME
RARHEPVNHSDMVDKMFGFLGTSGGLPGQEGQAPSGFEDLERGRREMVEEDLDAALP
LPDEDEEDLSEYKFAKFAATYFQGTTTHSYTRRPLKQPLLYHDDEGDQLAALAVWITIL
RFMGDLPEPKYHTAMSDGSEKIPVMTKIYETLGKKTYKRELQALQGEGEAQLPEGQ
361 bp Overlap (same as SEQ ID NO: 39)
CAGGTCTAACTTTCTGAAGCTGAAGAACGCTGCCACACTGATCCAGAGGCACTGGC
GGGGTCACAACTGTAGGAAGAACTACGGGCTGATGCGTCTGGGCTTCCTGCGGCTG
CAGGCCCTGCACCGCTCCCGGAAGCTGCACCAGCAGTACCGCCTGGCCCGCCAGCG
CATCATCCAGTTCCAGGCCCGCTGCCGCGCCTATCTGGTGCGCAAGGCCTTCCGCCA
CCGCCTCTGGGCTGTGCTCACCGTGCAGGCCTATGCCCGGGGCATGATCGCCCGCA
GGCTGCACCAACGCCTCAGGGCTGAGTATCTGTGGCGCCTCGAGGCTGAGAAAATG
CGGCTGGCGGAGGAAGAGAAGCTT
279 bp Overlap (SEQ ID NO: 58)
GGGCTGATGCGTCTGGGCTTCCTGCGGCTGCAGGCCCTGCACCGCTCCCGGAAGCT
GCACCAGCAGTACCGCCTGGCCCGCCAGCGCATCATCCAGTTCCAGGCCCGCTGCC
GCGCCTATCTGGTGCGCAAGGCCTTCCGCCACCGCCTCTGGGCTGTGCTCACCGTGC
AGGCCTATGCCCGGGGCATGATCGCCCGCAGGCTGCACCAACGCCTCAGGGCTGAG
TATCTGTGGCGCCTCGAGGCTGAGAAAATGCGGCTGGCGGAGGAAGAGAAGCTT
SEQ ID NO: 88
(protein sequence that corresponds to SEQ ID NO: 58)
GLMRLGFLRLQALHRSRKLHQQYRLARQRIIQFQARCRAYLVRKAFRHRLWAVLTVQ
AYARGMIARRLHQRLRAEYLWRLEAEKMRLAEEEKL
20 bp Overlap (SEQ ID NO: 59):
TGGCGGAGGAAGAGAAGCTT
SEQ ID NO: 89
(protein sequence that corresponds to SEQ ID NO: 59):
AEEEKL

In some embodiments of the disclosed hybrid and overlap vectors, any of the disclosed front half vectors (5′ AAV) comprise a left inverted terminal repeat sequence that comprises a nucleotide sequence having at least 95% or 98% identity to SEQ ID NO: 60. In some embodiments of the disclosed hybrid and overlap vectors, any of the disclosed front half vectors comprise a left inverted terminal repeat sequence that comprises SEQ ID NO: 60. In some embodiments of the disclosed hybrid and overlap vectors, any of the disclosed back half vectors (3′ AAV) comprise a right inverted terminal repeat sequence that comprises SEQ ID NO: 61. In some embodiments of the disclosed hybrid and overlap vectors, any of the disclosed back half vectors comprise a right inverted terminal repeat sequence that comprises a nucleotide sequence having at least 95% or 98% identity to SEQ ID NO: 61. In various embodiments, any of the disclosed dual hybrid and overlap vector pairs comprise a left ITR sequence comprising SEQ ID NO: 60 and a right ITR sequence comprising SEQ ID NO: 61.

Left ITR Sequence(SEQ ID NO: 60).
CTCCCTCTCTGCGCGCTCGCTCGCTCACTGAGGCCGCCCGGGCAAAGCCC
GGGCGTCGGGCGACCTTTGGTCGCCCGGCCTCAGTGAGCGAGCGAGCGCG
CAGAGAGGGAGTGGCCAACTCCATCACTAGGGGTT
Right ITR Sequence(SEQ ID NO: 61).
AACCCCTAGTGATGGAGTTGGCCACTCCCTCTCTGCGCGCTCGCTCGCTC
ACTGAGGCCGGGCGACCAAAGGTCGCCCGACGCCCGGGCTTTGCCCGGGC
GGCCTCAGTGAGCGAGCGAGCGCGCAGAGAGGGAG

TABLE 2
Overlap Region Information & Sequences

| | |
|---|---|
| Overlap Fragment | Position in hMYO7A cDNA |

| Name/Length: | Start: | End: |
|---|---|---|
| 1365 | 2281 | 3645 |
| 1284 | 2362 | 3645 |
| 1027 | 2281 | 3306 |
| 1026 | 2620 | 3645 |
| 945 | 2362 | 3306 |
| 687 | 2620 | 3306 |
| 361 | 2280 | 2640 |
| 279 | 2362 | 2640 |
| 20 | 2621 | 2640 |

TABLE 3
Select Examples 6 and 7 AAV Sequences and Components (amino acid (aa),
nucleic acid (nt)

| | |
|---|---|
| SEQUENCE | FULL-LENGTH AAV SEQUENCE |

Myosin7A N-terminus

| | |
|---|---|
| SEQ ID NO: 62 (aa) | AAV-smCBA-hMYO7A-noDimNT-CMv1 (SEQ ID NO: 36) |
| SEQ ID NO: 63 (nt) | |
| SEQ ID NO: 91 (aa) | AAV-smCBA-hMYO7A-noDIM-NTlong (SEQ ID NO: 37) |
| SEQ ID NO: 90 (nt) | |
| SEQ ID NO: 65 (aa) | AAV-smCBA-hMYO7A-NTlong-v3 (SEQ ID NO: 50) |
| SEQ ID NO: 66 (nt) | |
| SEQ ID NO: 74 (aa) | AAV-smCBA-hMYO7A-NT-Ex21-APSD-APhead (SEQ ID NO: 31) |
| SEQ ID NO: 73 (nt) | AAV-smCBA-hMYO7A-NT-Ex21-APSD-APhead CMv1 (SEQ ID NO: 33) |
| | AAV-smCBA-hMYO7A-NT-Ex21-APSD-APhead CMv2 (SEQ ID NO: 34) |
| | AAV-smCBA-hMYO7A-NT-Ex21-APSD-APhead-CMv3 (SEQ ID NO: |
| | 46) |

Myosin7A C-terminus

| | |
|---|---|
| SEQ ID NO: 78 (aa) | AAV-hMYO7A-CTlong-v2.HA (SEQ ID NO: 38) |
| SEQ ID NO: 77 (nt) | |
| SEQ ID NO: 81 (aa) | AAV-hMYO7A-CTlong-v3.HA (SEQ ID NO: 51) |
| SEQ ID NO: 80 (nt) | |
| SEQ ID NO: 76 (aa) | AAV-APhead-APSA-ex22hMYO7A-CT.HA (SEQ ID NO: 32) |
| SEQ ID NO: 75 (nt) | AAV-APhead-APSA-ex22hMYO7A-CT.HA-CMv2 (SEQ ID NO: 35) |
| | AAV-APhead-APSA-hMYO7ACTex22-CMv2.1 (SEQ ID NO: 44) |
| | AAV-APhead-APSA-hMYO7ACTex22-CMv2.1.HA (SEQ ID NO: 47) |
| | AAV-APhead-APSA-hMYO7ACTex 22.HA-MIN (SEQ ID NO: 48) |
| | AAV-APhead-APSA-hMYO7ACTex22-MIN (SEQ ID NO: 49) |

Myosin7A overlap

| | |
|---|---|
| SEQ ID NO: 79 (aa) | AAV-smCBA-hMYO7A-noDIM-NTlong (SEQ ID NO: 37) + AAV- |
| SEQ ID NO: 39 (nt) | hMYO7A-CTlong.HA (SEQ ID NO: 2) |
| | AAV-smCBA-hMYO7A-noDIM-NTlong-CMv1 (SEQ ID NO: 36) + AAV- |
| | hMYO7A-CTlong.HA (SEQ ID NO: 2) |
| SEQ ID NO: 82 (aa) | AAV-smCBA-hMYO7A-NTlong (SEQ ID NO: 1) + AAV-hMYO7A- |
| SEQ ID NO: 52 (nt) | CTlong.HA (SEQ ID NO: 2) |
| SEQ ID NO: 83 (aa) | AAV-smCBA-hMYO7A-NTlong (SEQ ID NO: 1) + AAV-hMYO7A- |
| SEQ ID NO: 53 (nt) | CTlong-v2.HA (SEQ ID NO: 38) |
| SEQ ID NO: 84 (aa) | AAV-smCBA-hMYO7A-NTlong-v3 (SEQ ID NO: 50) + AAV-hMYO7A- |
| SEQ ID NO: 54 (nt) | CTlong.HA (SEQ ID NO: 2) |
| SEQ ID NO: 85 (aa) | AAV-smCBA-hMYO7A-NTlong (SEQ ID NO: 1) + AAV-hMYO7A- |
| SEQ ID NO: 55 (nt) | CTlong-v3.HA (SEQ ID NO: 51) |
| SEQ ID NO: 86 (aa) | AAV-smCBA-hMYO7A-NTlong-v3 (SEQ ID NO: 50) + AAV-hMYO7A- |
| SEQ ID NO: 56 (nt) | CTlong-v2.HA (SEQ ID NO: 38) |
| SEQ ID NO: 87 (aa) | AAV-smCBA-hMYO7A-NTlong-v3 (SEQ ID NO: 50) + AAV-hMYO7A- |
| SEQ ID NO: 57 (nt) | CTlong-v3.HA (SEQ ID NO: 51) |
| SEQ ID NO: 88 (aa) | AAV-smCBA-hMYO7A-noDIM-NTlong (SEQ ID NO: 37) + AAV- |
| SEQ ID NO: 58 (nt) | hMYO7A-CTlong-v2.HA (SEQ ID NO: 38) |
| | AAV-smCBA-hMYO7A-noDimNT-CMv1 (SEQ ID NO: 36) + AAV- |
| | hMYO7A-CTlong-v2.HA (SEQ ID NO: 38) |
| SEQ ID NO: 89 (aa) | AAV-smCBA-hMYO7A-noDIMNTlong (SEQ ID NO: 37) + (AAV- |
| SEQ ID NO: 59 (nt) | hMYO7A-CTlong-v3.HA (SEQ ID NO: 51) |
| | AAV-smCBA-hMYO7A-noDIMNTlong-CMv1 (SEQ ID NO: 36) + (AAV- |
| | hMYO7A-CTlong-v3.HA (SEQ ID NO: 51) |

smCBA Promoter

| | |
|---|---|
| SEQ ID NO: 64 (nt) | (SEQ ID NO: 1) |
| | AAV-smCBA-hMYO7A-NT-Ex21-APSD-APhead (SEQ ID NO: 31) |
| | (SEQ ID NO: 46) |
| | (SEQ ID NO: 34) |
| | (SEQ ID NO: 3) |
| | (SEQ ID NO: 33) |
| | (SEQ ID NO: 37) |
| | (SEQ ID NO: 36) |
| | (SEQ ID NO: 50) |

HA tag

| | |
|---|---|
| SEQ ID NO: 72 (nt) | AAV-APhead-APSA-ex22hMYO7A-CT.HA (SEQ ID NO: 32) |
| | AAV-APhead-APSA-ex22hMYO7A-CT.HA-CMv2 (SEQ ID NO: 35) |
| | AAV-hMYO7A-CTlong-v2.HA (SEQ ID NO: 38) |
| | AAV-APhead-APSA-hMYO7ACTex22-CMv2.1 (SEQ ID NO: 44) |
| | AAV-APhead-APSA-hMYO7ACT ex22-CMv2.1.HA (SEQ ID NO: 47) |
| | AAV-APhead-APSA-hMYO7ACTex 22.HA-MIN (SEQ ID NO: 48) |

### REFERENCES

The following references, to the extent that they provide exemplary procedural or other details supplementary to those set forth herein, are specifically incorporated herein by reference:
Allocca, M et al., “Novel adeno-associated virus serotypes efficiently transduce murine photoreceptors,” J. Virol., 81(20):11372-80 (2007).
Allocca, M et al., “Serotype-dependent packaging of large genes in adeno-associated viral vectors results in effective gene delivery in mice,” J. Clin. Invest., 118(5):1955-1964 (2008).
Altschul, S F et al., “Basic local alignment search tool,” J. Mol. Biol., 215:403-410 (1990).
Altschul, S F et al., “Gapped BLAST and PSI-BLAST: A new generation of protein database search programs,” Nucl. Acids Res., 25:3389-3402 (1997).
Astuto, L M et al., “Genetic heterogeneity of Usher syndrome: analysis of 151 families with Usher type I,” Am. J. Hum. Genet., 67:1569-1574 (2000).
Bainbridge, J W et al., “Effect of gene therapy on visual function in Leber's congenital amaurosis,” N. Engl. J. Med., 358:2231-2239 (2008).
Beltz, G A et al., “Isolation of multigene families and determination of homologies by filter hybridization methods,” Meth. Enzymol., 100:266-285 (1983).
Bharadwaj, A K et al., “Evaluation of the myosin VIIA gene and visual function in patients with Usher syndrome type I,” Exp. Eye Res., 71:173-181 (2000).
Bowles, D E et al., “Phase I gene therapy for Duchenne muscular dystrophy using a translational optimized AAV vector,” Mol. Ther., 20:443-455 (2012).
Boye, S E et al., “A comprehensive review of retinal gene therapy,” Mol. Ther., 21:509-519 (2013).
Boye, S L et al., “AAV-mediated gene therapy in the guanylate cyclase (RetGC1/RetGC2) double knockout mouse model of Leber congenital amaurosis,” Hum. Gene Ther., 24:189-202 (2012).
Boye, S L et al., “Long-term preservation of cone photoreceptors and restoration of cone function by gene therapy in the guanylate cyclase-1 knockout (GC1KO) mouse,” Invest. Ophthalmol. Vis. Sci., 52:7098-7108 (2011).
Chen, Z Y et al., “Molecular cloning and domain structure of human myosin-VIIa, the gene product defective in Usher syndrome 1B,” Genomics, 36:440-448 (1996).
Cideciyan, A V et al., “Human RPE65 gene therapy for Leber congenital amaurosis: persistence of early visual improvements and safety at 1 year,” Hum. Gene Ther., 20:999-1004 (2009).
Dong, B et al., “Characterization of genome integrity for oversized recombinant AAV vector,” Mol. Ther., 18(1):87-92 (2010).
Duan, D et al., “Circular intermediates of recombinant adeno-associated virus have defined structural characteristics responsible for long-term episomal persistence in muscle tissue,” J. Virol., 72:8568-8577 (1998).
Duan, D et al., “Expanding AAV packaging capacity with trans-splicing or overlapping vectors: a quantitative comparison,” Mol. Ther., 4:383-391 (2001).
Duan, D et al., “Trans-splicing vectors expand the packaging limits of adeno-associated virus for gene therapy applications,” Methods Mol. Med., 76:287-307 (2003).
Dyka, F M et al., “Dual adeno-associated virus vectors result in efficient in vitro and in vivo expression of an oversized gene, MYO7A,” Hum. Gene Ther. Methods, 25(2):166-177 (2014).
Esumi, N et al., “Analysis of the VMD2 promoter and implication of E-box binding factors in its regulation,” J. Biol. Chem., 279:19064-19073 (2004).
Felgner, P L et al., “Lipofection: a highly efficient, lipid-mediated DNA-transfection procedure,” Proc. Nat'l. Acad. Sci. USA, 84(21):7413-7417 (1987).
Flotte, T R et al., “Phase 2 clinical trial of a recombinant adeno-associated viral vector expressing alpha1-antitrypsin: interim results,” Hum. Gene Ther., 22:1239-1247 (2011).
Gene Therapy: Principles and Applications, Blankenstein, T. (Ed.), Birkhauser-Verlag, Basel, Switzerland (1999).
Ghosh, A et al., “A hybrid vector system expands adeno-associated viral vector packaging capacity in a transgene-independent manner,” Mol. Ther., 16:124-130 (2008).
Ghosh, A et al., “Efficient transgene reconstitution with hybrid dual AAV vectors carrying the minimized bridging sequences,” Hum. Gene Ther., 22:77-83 (2011).
Gibbs, D and Williams, D S, “Isolation and culture of primary mouse retinal pigmented epithelial cells,” Adv. Exp. Med. Biol., 533:347-352 (2003b).
Gibbs, D et al., “Abnormal phagocytosis by retinal pigmented epithelium that lacks myosin VIIa, the Usher syndrome 1B protein,” Proc. Nat'l. Acad. Sci. USA, 100:6481-6486 (2003a).
Gibbs, D et al., “Role of myosin VIIa and Rab27a in the motility and localization of RPE melanosomes,” J. Cell Sci., 117:6473-6483 (2004).
Gibson, F et al., “A type VII myosin encoded by mouse deafness gene shaker-1,” Nature, 374:62-64 (1995).
Grieger, J C and Samulski, R J, “Packaging capacity of adeno-associated virus serotypes: impact of larger genomes on infectivity and postentry steps,” J. Virol., 79:9933-9944 (2005).
Haire, S E et al., “Light-driven cone arrestin translocation in cones of postnatal guanylate cyclase-1 knockout mouse retina treated with AAV-GC1,” Invest. Ophthalmol. Vis. Sci., 47:3745-3753 (2006).
Halbert, C L et al., “Efficient mouse airway transduction following recombination between AAV vectors carrying parts of a larger gene,” Nat. Biotechnol., 20:697-701 (2002).
Hashimoto, T et al., “Lentiviral gene replacement therapy of retinas in a mouse model for Usher syndrome type 1B,” Gene Ther., 14(7):584-594 (2007).
Hasson, T et al., “Effects of shaker-1 mutations on myosin-VIIa protein and mRNA expression,” Cell Motil. Cytoskeleton, 37:127-138 (1997).
Hasson, T et al., “Expression in cochlea and retina of myosin VIIa, the gene product defective in Usher syndrome type 1B,” Proc. Nat'l. Acad. Sci. USA, 92:9815-9819 (1995).
Hauswirth, W W et al., “Treatment of leber congenital amaurosis due to RPE65 mutations by ocular subretinal injection of adeno-associated virus gene vector: short-term results of a phase I trial,” Hum. Gene Ther., 19:979-990 (2008).
Hirsch, M L et al., “AAV recombineering with single strand oligonucleotides,” PLoS One 4:e7705 (2009).
Hirsch, M L et al., “Little vector, big gene transduction: fragmented genome reassembly of adeno-associated virus,” Mol. Ther., 18(1):6-8 (2010).
Hirsch, M L et al., “Oversized AAV transduction is mediated via a DNA-PKcs-independent, Rad51C-dependent repair pathway,” Mol. Ther., 21:2205-2216 (2013).
Jacobson, S G et al., “Retinal disease course in Usher syndrome 1B due to MYO7A mutations,” Invest. Ophthalmol. Vis. Sci., 52:7924-7936 (2011).
Jacobson, S G et al., “Safety of recombinant adeno-associated virus type 2-RPE65 vector delivered by ocular subretinal injection,” Molec. Ther., 13:1074-1084 (2006).
Jacobson, S G et al., “Usher syndromes due to MYO7A, PCDH15, USH2A or GPR98 mutations share retinal disease mechanism,” Hum. Mol. Genet., 17:2405-2415 (2008).
Kapranov, P et al., “Native molecular state of adeno-associated viral vectors revealed by single-molecule sequencing,” Hum. Gene Ther., 23:46-55 (2012).
Karlin, S and Altschul, S F “Applications and statistics for multiple high-scoring segments in molecular sequences,” Proc. Nat'l. Acad. Sci. USA, 90:5873-5877 (1993).
Karlin, S and Altschul, S F, “Methods for assessing the statistical significance of molecular sequence features by using general scoring schemes,” Proc. Nat'l. Acad. Sci. USA, 87:2264-2268 (1990).
Keats, B J and Corey, D P “The usher syndromes,” Am. J. Med. Genet., 89:158-166 (1999). Klomp, A E et al., “Analysis of the linkage of MYRIP and MYO7A to melanosomes by RAB27A in retinal pigment epithelial cells,” Cell Motil. Cytoskeleton, 64:474-487 (2007).
Lai, Y et al., “Design of trans-splicing adeno-associated viral vectors for Duchenne muscular dystrophy gene therapy,” Methods Mol. Biol., 433:259-275 (2008).
Lai, Y et al., “Efficient in vivo gene expression by trans-splicing adeno-associated viral vectors,” Nat. Biotechnol., 23:1435-1439 (2005).
Lai, Y et al., “Evidence for the failure of adeno-associated virus serotype 5 to package a viral genome >8.2 kb,” Mol. Ther., 18(1):75-79 (2010).
Lai, Y et al., “Synthetic intron improves transduction efficiency of trans-splicing adeno-associated viral vectors,” Hum. Gene Ther., 17:1036-1042 (2006).
Li, M et al., “High-efficiency transduction of fibroblasts and mesenchymal stem cells by tyrosine-mutant AAV2 vectors for their potential use in cellular therapy,” Hum. Gene Ther., 21:1527-1543 (2010).
Liu, X et al., “Mutant myosin VIIa causes defective melanosome distribution in the RPE of shaker-1 mice,” Nat. Genet., 19:117-118 (1998).
Liu, X et al., “Myosin VIIa participates in opsin transport through the photoreceptor cilium,” J. Neurosci., 19:6267-6274 (1999).
Liu, X et al., “Myosin VIIa, the product of the Usher 1B syndrome gene, is concentrated in the connecting cilia of photoreceptor cells,” Cell Motil. Cytoskel., 37:240-252 (1997).
Liu, X Z et al., “Mutations in the myosin VIIA gene cause non-syndromic recessive deafness,” Nat. Genet., 16:188-190 (1997).
Lopes, V S et al., “Retinal gene therapy with a large MYO7A cDNA using adeno-associated virus,” Gene Ther., 20:824-833 (2013).
Lopes, V S et al., “The ternary Rab27a-Myrip-Myosin VIIa complex regulates melanosome motility in the retinal pigment epithelium,” Traffic, 8:486-499 (2007).
Lopes, V S et al., “The Usher 1B protein, MYO7A, is required for normal localization and function of the visual retinoid cycle enzyme, RPE65,” Hum. Mol. Genet., 20(13):2560-2570 (2011).
Lostal, W et al., “Efficient recovery of dysferlin deficiency by dual adeno-associated vector-mediated gene transfer,” Hum. Mol. Genet., 19:1897-1907 (2010).
Maguire, A M et al., “Age-dependent effects of RPE65 gene therapy for Leber's congenital amaurosis: a phase 1 dose-escalation trial,” Lancet, 374:1597-1605 (2009).
Markusic, D M et al., “High-efficiency transduction and correction of murine hemophilia B using AAV2 vectors devoid of multiple surface-exposed tyrosines,” Mol. Ther., 18:2048-2056 (2010). Molecular Cloning: A Laboratory Manual, (Maniatis, T, Fritsch, E F, and Sambrook, J), Cold Spring Harbor Laboratory, Cold Spring Harbor, N Y (1982).
Nathwani, A C et al., “Adenovirus-associated virus vector-mediated gene transfer in hemophilia B,” N. Engl. J. Med., 365:2357-2365 (2011).
Ouyang, X M et al., “Characterization of Usher syndrome type I gene mutations in an Usher syndrome patient population,” Hum. Genet., 116:292-299 (2005).
Pang, J J et al., “Comparative analysis of in vivo and in vitro AAV vector transduction in the neonatal mouse retina: effects of serotype and site of administration,” Vision Res., 48:377-385 (2008).
Petrs-Silva, H et al., “High-efficiency transduction of the mouse retina by tyrosine-mutant AAV serotype vectors.” Mol. Ther. 17:463-471 (2009).
Petrs-Silva, H et al., “Novel properties of tyrosine-mutant AAV2 vectors in the mouse retina,” Mol. Ther., 19:293-301 (2011).
Ryals, R C et al., “Quantifying transduction efficiencies of unmodified and tyrosine capsid mutant AAV vectors in vitro using two ocular cell lines,” Mol. Vis., 17:1090-1102 (2011).
Sahly, I et al., “Localization of Usher 1 proteins to the photoreceptor calyceal processes, which are absent from mice,” J. Cell Biol., 199:381-399 (2012).
Saihan, Z et al., “Update on Usher syndrome,” Curr. Opin. Neurol., 22:19-27 (2009).
Simonelli, F et al., “Gene therapy for Leber's congenital amaurosis is safe and effective through 1.5 years after vector administration,” Mol. Ther., 18:643-650 (2010).
Smith, R J et al., “Clinical diagnosis of the Usher syndromes,” Usher Syndrome Consortium. Am. J. Med. Genet., 50:32-38 (1994).
Soni, L E et al., “The unconventional myosin-VIIa associates with lysosomes,” Cell Motil. Cytoskeleton, 62:13-26 (2005).
Timmers, A M et al., “Subretinal injections in rodent eyes: effects on electrophysiology and histology of rat retina,” Mol. Vis., 7:131-137 (2001).
Trapani, I et al., “Effective delivery of large genes to the retina by dual AAV vectors,” EMBO Mol. Med., 6:194-211 (2014).
Trapani, I, et al., “Improved dual AAV vectors with reduced expression of truncated proteins are safe and effective in the retina of a mouse model of Stargardt disease,” Hum. Mol. Gen., 24(23): 8811-6825 (2015).
Weil, D et al., “Defective myosin VIIA gene responsible for Usher syndrome type 1B,” Nature, 374:60-61 (1995).
Weil, D et al., “Human myosin VIIA responsible for the Usher 1B syndrome: a predicted membrane-associated motor protein expressed in developing sensory epithelia,” Proc. Nat'l. Acad. Sci. USA, 93:3232-3237 (1996).
Williams, D S and Lopes, V S “The many different cellular functions of MYO7A in the retina,” Biochem. Soc. Trans., 39:1207-1210 (2011).
Williams, D S, “Usher syndrome: Animal models, retinal function of Usher proteins, and prospects for gene therapy,” Vision Res., 48:433-441 (2008).
Wolfrum, U et al., “Myosin VIIa as a common component of cilia and microvilli,” Cell Motil. Cytoskeleton, 40:261-271 (1998).
Wu, Z et al., “Effect of genome size on AAV vector packaging,” Mol. Ther., 18(1):80-86 (2010).
Yan, Z et al., “Inverted terminal repeat sequences are important for intermolecular recombination and circularization of adeno-associated virus genomes,” J. Virol., 79:364-379 (2005).
Yan, Z et al., “Recombinant AAV-mediated gene delivery using dual vector heterodimerization,” Methods Enzymol., 346:334-357 (2002).

Yan, Z et al., “Trans-splicing vectors expand the utility of adeno-associated virus for gene therapy,” Proc. Nat'l. Acad. Sci. USA, 97:6716-6721 (2000).
Yang, J et al., “Concatemerization of adeno-associated virus circular genomes occurs through intermolecular recombination,” J. Virol., 73:9468-9477 (1999).
Zhang, Y and Duan, D “Novel mini-dystrophin gene dual adeno-associated virus vectors restore neuronal nitric oxide synthase expression at the sarcolemma,” Hum. Gene Ther., 23:98-103 (2012).
Zhong, L et al., “Next generation of adeno-associated virus 2 vectors: point mutations in tyrosines lead to high-efficiency transduction at lower doses,” Proc. Nat'l. Acad. Sci. USA, 105(22):7827-7832 (2008).
Zolotukhin, S et al., “Production and purification of serotype 1, 2, and 5 recombinant adeno-associated viral vectors,” Methods, 28:158-167 (2002).

### EQUIVALENTS

It should be understood that the examples and embodiments described herein are for illustrative purposes only and that various modifications or changes in light thereof will be suggested to persons skilled in the art and are to be included within the spirit and purview of this application and the scope of the appended claims.

All references cited herein (including publications, patent applications and patents) are incorporated by reference to the same extent as if each reference was individually and specifically incorporated by reference, and was set forth in its entirety herein.

Recitation of ranges of values herein are merely intended to serve as a shorthand method of referring individually to each separate value falling within the range, unless otherwise indicated herein, and each separate value is incorporated into the specification as if it were individually recited herein.

All methods described herein can be performed in any suitable order, unless otherwise indicated herein, or unless otherwise clearly contradicted by context.

The use of any examples, or exemplary language (e.g., “such as”) provided herein, is intended merely to better illustrate the disclosure and does not pose a limitation on the scope of the disclosure unless otherwise indicated. No language in the specification should be construed as indicating any element is essential to the practice of the disclosure unless as much is explicitly stated.

The description herein of any aspect or embodiment of the disclosure using terms such as “comprising”, “having”, “including” or “containing” with reference to an element or elements is intended to provide support for a similar aspect or embodiment of the disclosure that “consists of”, “consists essentially of”, or “substantially comprises” that particular element or elements, unless otherwise stated or clearly contradicted by context (e.g., a composition described herein as comprising a particular element should be understood as also describing a composition consisting of that element, unless otherwise stated or clearly contradicted by context). For example, a nucleotide sequence described herein as comprising a particular element should be understood as also describing a nucleotide sequence consisting of that element, unless otherwise stated or clearly contradicted by context.

All of the compositions and methods disclosed and claimed herein can be made and executed without undue experimentation in light of the present disclosure. While the compositions and methods of this invention have been described in terms of preferred embodiments, it will be apparent to those of ordinary skill in the art that variations may be applied to the compositions and/or methods disclosed herein, and/or to the steps or the sequence of steps of the methods described herein without departing from the concept, spirit and/or scope of the disclosure. More specifically, it will be apparent that certain agents that are chemically- and/or physiologically-related may be substituted for the agents described herein while the same or similar results would be achieved. All such similar substitutes and modifications apparent to those skilled in the art are deemed to be within the spirit, scope and concept of the disclosure as defined by the appended claims.

1-151. (canceled)

152. A polynucleotide vector system comprising:
a first AAV vector polynucleotide comprising an inverted terminal repeat at each end of a polynucleotide comprising a promoter followed by a first partial coding sequence that encodes an N-terminal part of a full-length myosin polypeptide, and
a second AAV vector polynucleotide comprising an inverted terminal repeat at each end of a polynucleotide comprising a second partial coding sequence that encodes a C-terminal part of the full-length myosin polypeptide, and
wherein the N-terminal part of the full-length myosin polypeptide comprises an amino acid sequence at least about 95% identity to the amino acid sequence of SEQ ID NO: 74; and the C-terminal part of the full-length myosin polypeptide comprises an amino acid sequence at least about 95% identical to the amino acid sequence of SEQ ID NO: 76,
wherein the first AAV vector polynucleotide does not encode the single-alpha helix (SAH) domain of the full-length myosin polypeptide, and the first partial coding sequence and the second partial coding sequence encode the full-length myosin polypeptide.

153. The polynucleotide vector system of claim 152, wherein the promoter is selected from the group consisting of: a CMV promoter, an EF-1 alpha promoter, a cone arrestin promoter, a smCBA promoter, a human myosin 7a gene-derived promoter, a TaC gene-derived promoter, a rhodopsin promoter, a cGMP-phosphodiesterase β-subunit promoter, human or mouse rhodopsin promoter, a hGRK1 promoter, a rod specific IRBP promoter, a VMD2 promoter, a synapsin promoter, a glial fibrillary acidic protein (GFAP) promoter, and combinations thereof.

154. The polynucleotide vector system of claim 153, wherein the promoter is the smCBA promoter, and the smCBA promoter is at least 95% identical to SEQ ID NO: 64.

155. The polynucleotide vector system of claim 152, wherein the first AAV vector polynucleotide comprises an overlap sequence and the second AAV vector polynucleotide comprises the overlap sequence, wherein the overlap sequence is an intron of a gene encoding the full-length myosin polypeptide, the AK sequence of the F1 phage, or a synthetic alkaline phosphatase (AP) intron.

156. The polynucleotide vector system of claim 155, wherein the overlap sequence is the alkaline phosphatase (AP) intron, and the AP intron comprises SEQ ID NO: 70.

157. The polynucleotide vector system of claim 152, wherein the first AAV vector polynucleotide comprises a splice donor site and the second AAV vector polynucleotide comprises a splice acceptor site.

158. The polynucleotide vector system of claim 152, wherein the amino acid sequence at least 95% identical to the amino acid sequence of SEQ ID NO: 74 is SEQ ID NO: 74.

159. The polynucleotide vector system of claim 158, wherein the amino acid sequence at least 95% identical to the amino acid sequence of SEQ ID NO: 76 is SEQ ID NO: 76.

160. The polynucleotide vector system of claim 159, wherein the promoter is a smCBA promoter at least 95% identical to SEQ ID NO: 64.

161. A viral particle comprising the polynucleotide vector system of claim 152, wherein the viral particle comprises an AAV2, AAV5, AAV7, AAV8, AAV44.9, AAV44.9(E531D), AAV9-PHP.B, or AAV44.9(Y733F) capsid.

162. An isolated host cell comprising the polynucleotide vector system of claim 152.

163. A method for treating or ameliorating a disease or condition in a human or animal, comprising administering to one or more cells of the human or animal, the polynucleotide vector system of claim 152, wherein the full-length myosin polypeptide provides for treatment or amelioration of the disease or condition and is expressed in the one or more cells.

164. The method of claim 163, wherein the disease or condition is Usher syndrome or autosomal recessive isolated deafness (DFNB2).

165. The method of claim 163, wherein administration of the polynucleotide vector system provides a partial or complete restoration of vision loss.

166. The method of claim 163, wherein administration of the polynucleotide vector system provides a partial or complete restoration of hearing loss.

167. The method of claim 163, wherein administration of the polynucleotide vector system provides a partial or complete restoration of vestibular function.

168. A polynucleotide vector system comprising:
a first AAV vector polynucleotide comprising an inverted terminal repeat at each end of a polynucleotide comprising a promoter followed by a first partial coding sequence that encodes an N-terminal part of a full-length myosin polypeptide, and
a second AAV vector polynucleotide comprising an inverted terminal repeat at each end of a polynucleotide comprising a second partial coding sequence that encodes a C-terminal part of the full-length myosin polypeptide, and
wherein the sequence that encodes the N-terminal part of the full-length myosin polypeptide is at least 95% identical to the nucleotide sequence of SEQ ID NO: 73 and the sequence that encodes the C-terminal part of the full-length myosin polypeptide is at least 95% identical to the nucleotide sequence of SEQ ID NO: 75,
wherein the first AAV vector polynucleotide does not encode the single-alpha helix (SAH) domain of the full-length myosin polypeptide, and the first partial coding sequence and the second partial coding sequence encode the full-length myosin polypeptide.

169. A method for treating or ameliorating a disease or condition in a human or animal, comprising administering to one or more cells of the human or animal, the polynucleotide vector system of claim 168, wherein the full-length myosin polypeptide provides for treatment or amelioration of the disease or condition and is expressed in the one or more cells.

170. A polynucleotide vector system comprising:
a first AAV vector polynucleotide comprising an inverted terminal repeat at each end of a polynucleotide comprising a promoter followed by a first partial coding sequence that encodes an N-terminal part of a full-length myosin polypeptide, and
a second AAV vector polynucleotide comprising an inverted terminal repeat at each end of a polynucleotide comprising a second partial coding sequence that encodes a C-terminal part of the full-length myosin polypeptide, and
wherein the first AAV vector polynucleotide is at least 95% identical to the nucleotide sequence of SEQ ID NO: 90 and the second AAV vector polynucleotide is at least 95% identical to the nucleotide sequence of SEQ ID NO: 80,
wherein the first AAV vector polynucleotide does not encode the single-alpha helix (SAH) domain of the full-length myosin polypeptide, and the first partial coding sequence and the second partial coding sequence encode the full-length myosin polypeptide.

171. A method for treating or ameliorating a disease or condition in a human or animal, comprising administering to one or more cells of the human or animal, the polynucleotide vector system of claim 170, wherein the full-length myosin polypeptide provides for treatment or amelioration of the disease or condition and is expressed in the one or more cells.


SEQUENCE LISTING (91 sequences)

SEQ ID NO: 1 - DNA, 4961 bp, synthetic construct
  misc_feature 1..4961: note = Synthetic
  source 1..4961: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttctcagatctggcgcgcccaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcgctagcgggcactagtccgtcgactgttaattaagcatgctggggagagatctaggaaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 2 - DNA, 4915 bp, synthetic construct
  misc_feature 1..4915: note = Synthetic
  source 1..4915: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcagatctggcgcgcccaattggcttcgaattctagcggccgctgcttaagcaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtacccttacgatgtaccggattacgcatgaggtaccaagggcgaattctgcagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggactagtccgtcgactgttaattaagcatgctggggagagatctaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 3 - DNA, 4578 bp, synthetic construct
  misc_feature 1..4578: note = Synthetic
  source 1..4578: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttctcagatctggcgcgcccaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgactgttaattaagcatgctggggagagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 4 - DNA, 4646 bp, synthetic construct
  misc_feature 1..4646: note = Synthetic
  source 1..4646: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttctcagatctggcgcgcccaattggcttcgaattctagcggccgcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccttaagcgacgcatgctcgcgataggcacctattggtcttactgacatccactttgcctttctctccacaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtacccttacgatgtaccggattacgcatgaggtaccaagggcgaattctgcagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggactagtccgtcgactgttaattaagcatgctggggagagatctaggaaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 5 - DNA, 7465 bp, Homo sapiens
  source 1..7465: mol_type = genomic DNA
gctctgggcaggagagagagtgagagacaagagacacacacagagagacggcgaggaagggaaagacccagagggacgcctagaacgagacttggagccagacagaggaagaggggacgtgtgtttgcagactggctgggcccgtgacccagcttcctgagtcctccgtgcaggtggcagctgtaccaggctggcaggtcactgagagtgggcagctgggccccagaactgtgcctggcccagtgggcagcaggagctcctgacttgggaccatggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcagggagtgccgtgtctggctctcactgggctgctctgatcttggctgtgctgcgcctcactcaggctgggcaggactgaccccggcggggccctgttctccgtgttggtcctgcaggggagcgaaaacgacggcccccagcttcacgctggccaccatcaagggggacgaatacaccttcacctccagtaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgactgtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaggtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtgaacagtcacggggaggtgctggttccatgcctgctctcgaggcagcagtgggttcaggcccatcagctacccctgcagctggggaagacttatgccatcccggcagcgaggctgggctggccagccaccactgactataccaactgggcctctgatgttcttccagtgaggcatctctctgggatgcagaacttccctccatccacccctctggcacctgggttggtctaatcctagtttgctgtggccttcccggttgtgagagcctgtgatccttagatgtgtctcctgtttcagaccagccccaccatgcaacttcctttgactttctgtgtaccactgggatagaggaatcaagaggacaatctagctctccatactttgaacaaccaaatgtgcattgaatactctgaaaccgaagggactggatctgcaggtgggatgagggagacagaccacttttctatattgcagtgtgaatgctgggcccctgctcaagtctaccctgatcacctcagggcataaagcatgtttcattctctgaaa

SEQ ID NO: 6 - protein, 2215 aa, Homo sapiens
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRECRVWLSLGCSDLGCAAPHSGWAGLTPAGPCSPCWSCRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDCVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVISVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 7 - DNA, 6648 bp, Homo sapiens
  source 1..6648: mol_type = genomic DNA
atggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcagggagtgccgtgtctggctctcactgggctgctctgatcttggctgtgctgcgcctcactcaggctgggcaggactgaccccggcggggccctgttctccgtgttggtcctgcaggggagcgaaaacgacggcccccagcttcacgctggccaccatcaagggggacgaatacaccttcacctccagtaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgactgtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaggtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtga

SEQ ID NO: 8 - protein, 2175 aa, Homo sapiens
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVLSVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 9 - DNA, 39 bp, synthetic construct
  misc_feature 1..39: note = Synthetic
  source 1..39: mol_type = other DNA
gcggcggccgccaccatggtgattcttcagcagggggac

SEQ ID NO: 10 - DNA, 30 bp, synthetic construct
  misc_feature 1..30: note = Synthetic
  source 1..30: mol_type = other DNA
gcggctagcgaagttccgcaggtacttgac

SEQ ID NO: 11 - DNA, 31 bp, synthetic construct
  misc_feature 1..31: note = Synthetic
  source 1..31: mol_type = other DNA
gcgcttaagcaggtctaactttctgaagctg

SEQ ID NO: 12 - DNA, 30 bp, synthetic construct
  misc_feature 1..30: note = Synthetic
  source 1..30: mol_type = other DNA
gcgggtacctcacttgccgctcctggagcc

SEQ ID NO: 13 - DNA, 41 bp, synthetic construct
  misc_feature 1..41: note = Synthetic
  source 1..41: mol_type = other DNA
ggcacctagtggctttgaggtaagtatcaaggttacaagac

SEQ ID NO: 14 - DNA, 29 bp, synthetic construct
  misc_feature 1..29: note = Synthetic
  source 1..29: mol_type = other DNA
gcggctagctcagaaacgcaagagtcttc

SEQ ID NO: 15 - DNA, 22 bp, synthetic construct
  misc_feature 1..22: note = Synthetic
  source 1..22: mol_type = other DNA
cttctttgtgcgatgcatcaag

SEQ ID NO: 16 - DNA, 28 bp, synthetic construct
  misc_feature 1..28: note = Synthetic
  source 1..28: mol_type = other DNA
gcgcttaagcgacgcatgctcgcgatag

SEQ ID NO: 17 - DNA, 37 bp, synthetic construct
  misc_feature 1..37: note = Synthetic
  source 1..37: mol_type = other DNA
cgccctcgctccaggtcctgtggagagaaaggcaaag

SEQ ID NO: 18 - DNA, 19 bp, synthetic construct
  misc_feature 1..19: note = Synthetic
  source 1..19: mol_type = other DNA
gaacccgaaccggtccttg

SEQ ID NO: 19 - DNA, 24 bp, synthetic construct
  misc_feature 1..24: note = Synthetic
  source 1..24: mol_type = other DNA
gcggctagcccccgggtgcgcggc

SEQ ID NO: 20 - DNA, 29 bp, synthetic construct
  misc_feature 1..29: note = Synthetic
  source 1..29: mol_type = other DNA
gcggtcgacgaaacggtccaggctatgtg

SEQ ID NO: 21 - DNA, 27 bp, synthetic construct
  misc_feature 1..27: note = Synthetic
  source 1..27: mol_type = other DNA
gcggcggccgcccccgggtgcgcggcg

SEQ ID NO: 22 - DNA, 29 bp, synthetic construct
  misc_feature 1..29: note = Synthetic
  source 1..29: mol_type = other DNA
gcgcttaaggaaacggtccaggctatgtg

SEQ ID NO: 23 - DNA, 40 bp, synthetic construct
  misc_feature 1..40: note = Synthetic
  source 1..40: mol_type = other DNA
caggcacctagtggctttgaggtaccaggctagggacagg

SEQ ID NO: 24 - DNA, 28 bp, synthetic construct
  misc_feature 1..28: note = Synthetic
  source 1..28: mol_type = other DNA
gcggctagccgcctgagcccagaagttc

SEQ ID NO: 25 - DNA, 38 bp, synthetic construct
  misc_feature 1..38: note = Synthetic
  source 1..38: mol_type = other DNA
cgccctcgctccaggtcctgaaggagacaagaggtatg

SEQ ID NO: 26 - DNA, 29 bp, synthetic construct
  misc_feature 1..29: note = Synthetic
  source 1..29: mol_type = other DNA
gcgcttaagcaccgcttgtgttgatcctc

SEQ ID NO: 27 - DNA, 19 bp, synthetic construct
  misc_feature 1..19: note = Synthetic
  source 1..19: mol_type = other DNA
gccagggaaggatcccatg

SEQ ID NO: 28 - DNA, 57 bp, synthetic construct
  misc_feature 1..57: note = Synthetic
  source 1..57: mol_type = other DNA
gcgggtacctcatgcgtaatccggtacatcgtaagggtacttgccgctcctggagcc

SEQ ID NO: 29 - DNA, 24 bp, synthetic construct
  misc_feature 1..24: note = Synthetic
  source 1..24: mol_type = other DNA
agcttcgtagagtttgtggagcgg

SEQ ID NO: 30 - DNA, 19 bp, synthetic construct
  misc_feature 1..19: note = Synthetic
  source 1..19: mol_type = other DNA
gaggggcaaacaacagatg

SEQ ID NO: 31 - DNA, 4262 bp, synthetic construct
  misc_feature 1..4262: note = Synthetic
  source 1..4262: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttctcagatctggcgcgcccaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgaggtaagtatcaaggttacaagacaggtttaaggagaccaatagaaactgggcttgtcgagacagagaagactcttgcgtttctgagctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgactgttaattaagcatgctggggagagatctaggaaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 32 - DNA, 4965 bp, synthetic construct
  misc_feature 1..4965: note = Synthetic
  source 1..4965: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttctcagatctggcgcgcccaattggcttcgaattctagcggccgcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccttaagcgacgcatgctcgcgataggcacctattggtcttactgacatccactttgcctttctctccacagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtacccttacgatgtaccggattacgcatgaggtaccaagggcgaattctgcagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctggaggactagtccgtcgactgttaattaagcatgctggggagagatctaggaaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 33 - DNA, 4262 bp, synthetic construct
  misc_feature 1..4262: note = Synthetic
  source 1..4262: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttctcagatctggcgcgcccaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgaggtaagtatcaaggttacaagacaggttaacggagaccaattgaaactgggcttgtcgagacagagaagactcttgcgtttcagcgctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttcgtcgactgttaattaagcatgctggggagagatctgaggaaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 34 - DNA, 4259 bp, synthetic construct
  misc_feature 1..4259: note = Synthetic
  source 1..4259: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttctcagatctggcgcgcccaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgaggtaagtatcaaggttacaagacaggttaacggagaccaattgaaactgggcttgtcgagacagagaagactcttgcgtttcagcgctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgcgcactgctgccgatactcggggctcccgctctcgctctcggtaacatccggccgggcgccgtccttgagcacatagcctggaccgtttcgtcgactgttaattaagcatgctggggagagatctgtaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 35 - DNA, 4964 bp, synthetic construct
  misc_feature 1..4964: note = Synthetic
  source 1..4964: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcagatctggcgcgcccaattggcttcgaattctagcggccgcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgcgcactgctgccgatactcggggctcccgctctcgctctcggtaacatccggccgggcgccgtccttgagcacatagcctggaccgtttctcttaagcgacgcatgctcgcgataggcacctattggtcttactgacatccactttgcctttctctccacagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtacccttacgatgtaccggattacgcatgaggtaccaagggcgaattctgcagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctggaggactagtccgtcgactgttaattaagcatgctggggagagatctaggaaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 36 - DNA, 3949 bp, synthetic construct
  misc_feature 1..3949: note = Synthetic
  source 1..3949: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttctcagatctggcgcgcccaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttagaggatcctcccgtcgactgtttaagcatgctggggagagatctgaggaaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 37 - DNA, 3960 bp, synthetic construct
  misc_feature 1..3960: note = Synthetic
  source 1..3960: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttctcagatctggcgcgcccaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagctttgaaagtgacattaggctcccgtcgactgttaattaagcatgctggggagagatctgaggaaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 38 - DNA, 4793 bp, synthetic construct
  misc_feature 1..4793: note = Synthetic
  source 1..4793: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcagatctggcgcgccggatccgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtacccttacgatgtaccggattacgcatgaggtaccaagggcgaattctgcagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggatccttaattaagcatgctggggagagatctgaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 39 - DNA, 361 bp, synthetic construct
  misc_feature 1..361: note = Synthetic
  source 1..361: mol_type = other DNA
caggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagctt

SEQ ID NO: 40 - DNA, 111 bp, synthetic construct
  misc_feature 1..111: note = Synthetic
  source 1..111: mol_type = other DNA
caacgcctcagggctgaggtaagtatcaaggttacaagacaggtttaaggagaccaatagaaactgggcttgtcgagacagagaagactcttgcgtttctgagctagcccc

SEQ ID NO: 41 - DNA, 111 bp, synthetic construct
  misc_feature 1..111: note = Synthetic
  source 1..111: mol_type = other DNA
caacgcctcagggctgaggtaagtatcaaggttacaagacaggttaacggagaccaattgaaactgggcttgtcgagacagagaagactcttgcgtttcagcgctagcccc

SEQ ID NO: 42 - DNA, 78 bp, synthetic construct
  misc_feature 1..78: note = Synthetic
  source 1..78: mol_type = other DNA
ccgcaggctgcaccaacgcctcagggctgaggtaagtatcaaggttacaagacaggttaacggagaccaattgaaact

SEQ ID NO: 43 - DNA, 79 bp, synthetic construct
  misc_feature 1..79: note = Synthetic
  source 1..79: mol_type = other DNA
aacggagaccaattgaaactgggcttgtcgagacagagaagactcttgcgtttcagcgctagcccccgggtgcgcggcg

SEQ ID NO: 44 - DNA, 4845 bp, synthetic construct
  misc_feature 1..4845: note = Synthetic
  source 1..4845: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttctcagatctggcgcgccccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgcgcactgctgccgatactcggggctcccgctctcgctctcggtaacatccggccgggcgccgtccttgagcacatagcctggaccgtttctcttaagcgacgcatgctcgcgataggcacctattggtcttactgacatccactttgcctttctctccacagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatttaattaagcatgctggggagagatctgaggaaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 45 - DNA, 1366 bp, synthetic construct
  misc_feature 1..1366: note = Synthetic
  source 1..1366: mol_type = other DNA
caggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttc

SEQ ID NO: 46 - DNA, 4259 bp, synthetic construct
  misc_feature 1..4259: note = Synthetic
  source 1..4259: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttctcagatctggcgcgcccaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgacgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgaggtaagtatcaaggttacaagacaggttaacggagaccaattgaaactgggcttgtcgagacagagaagactcttgcgtttcagcgctagcccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgcgcactgctgccgatactcggggctcccgctctcgctctcggtaacatccggccgggcgccgtccttgagcacatagcctggaccgtttcgtcgacggatccgcatgctggggagagatctgaggaaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 47 - DNA, 4875 bp, synthetic construct
  misc_feature 1..4875: note = Synthetic
  source 1..4875: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttctcagatctggcgcgccccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgcgcactgctgccgatactcggggctcccgctctcgctctcggtaacatccggccgggcgccgtccttgagcacatagcctggaccgtttctcttaagcgacgcatgctcgcgataggcacctattggtcttactgacatccactttgcctttctctccacagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtacccttacgatgtaccggattacgcatgaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatttaattaagcatgctggggagagatctgaggaaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 48 - DNA, 4874 bp, synthetic construct
  misc_feature 1..4874: note = Synthetic
  source 1..4874: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttctcagatctggcgcgccccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccttaagcgacgcatgctcgcgataggcacctattggtcttactgacatccactttgcctttctctccacagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtacccttacgatgtaccggattacgcatgaagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatttaattaagcatgctggggagagatctgaggaaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 49 - DNA, 4843 bp, synthetic construct
  misc_feature 1..4843: note = Synthetic
  source 1..4843: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttctcagatctggcgcgccccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttccttaagcgacgcatgctcgcgataggcacctattggtcttactgacatccactttgcctttctctccacagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatttaattaagcatgctggggagagatctaggaaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 50 - DNA, 4591 bp, synthetic construct
  misc_feature 1..4591: note = Synthetic
  source 1..4591: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcagatctggcgcgcccaattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaagaattctagcggccgccaccatggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagttaattaagcatgctggggagagatctgaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 51 - DNA, 4541 bp, synthetic construct
  misc_feature 1..4541: note = Synthetic
  source 1..4541: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggttcagatctggcgcgccctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaagtacccttacgatgtaccggattacgcatgaggtaccaagggcgaattctgcagtcgactagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggagagatctgaggactagtccgtcgactgttaattaagcatgctggggagagatctgaacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 52 - DNA, 1366 bp, synthetic construct; identical to SEQ ID NO: 45
  misc_feature 1..1366: note = Synthetic
  source 1..1366: mol_type = other DNA

SEQ ID NO: 53 - DNA, 1284 bp, synthetic construct
  misc_feature 1..1284: note = Synthetic
  source 1..1284: mol_type = other DNA
gggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttc

SEQ ID NO: 54 - DNA, 1027 bp, synthetic construct
  misc_feature 1..1027: note = Synthetic
  source 1..1027: mol_type = other DNA
caggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccag

SEQ ID NO: 55 - DNA, 1026 bp, synthetic construct
  misc_feature 1..1026: note = Synthetic
  source 1..1026: mol_type = other DNA
ctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttc

SEQ ID NO: 56 - DNA, 945 bp, synthetic construct
  misc_feature 1..945: note = Synthetic
  source 1..945: mol_type = other DNA
gggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccag

SEQ ID NO: 57 - DNA, 687 bp, synthetic construct
  misc_feature 1..687: note = Synthetic
  source 1..687: mol_type = other DNA
ctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccag

SEQ ID NO: 58 - DNA, 279 bp, synthetic construct
  misc_feature 1..279: note = Synthetic
  source 1..279: mol_type = other DNA
gggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagctt

SEQ ID NO: 59 - DNA, 20 bp, synthetic construct
  misc_feature 1..20: note = Synthetic
  source 1..20: mol_type = other DNA
tggcggaggaagagaagctt

SEQ ID NO: 60 - DNA, 135 bp, synthetic construct
  misc_feature 1..135: note = Synthetic
  source 1..135: mol_type = other DNA
ctccctctctgcgcgctcgctcgctcactgaggccgcccgggcaaagcccgggcgtcgggcgacctttggtcgcccggcctcagtgagcgagcgagcgcgcagagagggagtggccaactccatcactaggggtt

SEQ ID NO: 61 - DNA, 135 bp, synthetic construct
  misc_feature 1..135: note = Synthetic
  source 1..135: mol_type = other DNA
aacccctagtgatggagttggccactccctctctgcgcgctcgctcgctcactgaggccgggcgaccaaaggtcgcccgacgcccgggctttgcccgggcggcctcagtgagcgagcgagcgcgcagagagggag

SEQ ID NO: 62 - protein, 880 aa, synthetic construct
  REGION 1..880: note = Synthetic
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKL

SEQ ID NO: 63 - DNA, 2640 bp, synthetic construct
  misc_feature 1..2640: note = Synthetic
  source 1..2640: mol_type = other DNA
atggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagctt

SEQ ID NO: 64 - DNA, 952 bp, synthetic construct
  misc_feature 1..952: note = Synthetic
  source 1..952: mol_type = other DNA
aattcggtaccctagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagctagagcctctgctaaccatgttcatgccttcttctttttcctacagctcctgggcaacgtgctggttattgtgctgtctcatcattttggcaaag

SEQ ID NO: 65 - protein, 1102 aa, synthetic construct
  REGION 1..1102: note = Synthetic
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQ

SEQ ID NO: 66 - DNA, 3306 bp, synthetic construct
  misc_feature 1..3306: note = Synthetic
  source 1..3306: mol_type = other DNA
atggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccag

SEQ ID NO: 67 - protein, 2905 aa, synthetic construct
  REGION 1..2905: note = Synthetic
ATGGTGATTCTTCAGCAGGGGGACCATGTGTGGATGGACCTGAGATTGGGGCAGGAGTTCGACGTGCCCATCGGGGCGGTGGTGAAGCTCTGCGACTCTGGGCAGGTCCAGGTGGTGGATGATGAAGACAATGAACACTGGATCTCTCCGCAGAACGCAACGCACATCAAGCCTATGCACCCCACGTCGGTCCACGGCGTGGAGGACATGATCCGCCTGGGGGACCTCAACGAGGCGGGCATCTTGCGCAACCTGCTTATCCGCTACCGGGACCACCTCATCTACACGTATACGGGCTCCATCCTGGTGGCTGTGAACCCCTACCAGCTGCTCTCCATCTACTCGCCAGAGCACATCCGCCAGTATACCAACAAGAAGATTGGGGAGATGCCCCCCCACATCTTTGCCATTGCTGACAACTGCTACTTCAACATGAAACGCAACAGCCGAGACCAGTGCTGCATCATCAGTGGGGAATCTGGGGCCGGGAAGACGGAGAGCACAAAGCTGATCCTGCAGTTCCTGGCAGCCATCAGTGGGCAGCACTCGTGGATTGAGCAGCAGGTCTTGGAGGCCACCCCCATTCTGGAAGCATTTGGGAATGCCAAGACCATCCGCAATGACAACTCAAGCCGTTTCGGAAAGTACATCGACATCCACTTCAACAAGCGGGGCGCCATCGAGGGCGCGAAGATTGAGCAGTACCTGCTGGAAAAGTCACGTGTCTGTCGCCAGGCCCTGGATGAAAGGAACTACCACGTGTTCTACTGCATGCTGGAGGGTATGAGTGAGGATCAGAAGAAGAAGCTGGGCTTGGGCCAGGCCTCTGACTACAACTACTTGGCCATGGGTAACTGCATAACCTGTGAGGGCCGGGTGGACAGCCAGGAGTACGCCAACATCCGCTCCGCCATGAAGGTGCTCATGTTCACTGACACCGAGAACTGGGAGATCTCGAAGCTCCTGGCTGCCATCCTGCACCTGGGCAACCTGCAGTATGAGGCACGCACATTTGAAAACCTGGATGCCTGTGAGGTTCTCTTCTCCCCATCGCTGGCCACAGCTGCATCCCTGCTTGAGGTGAACCCCCCAGACCTGATGAGCTGCCTGACTAGCCGCACCCTCATCACCCGCGGGGAGACGGTGTCCACCCCACTGAGCAGGGAACAGGCACTGGACGTGCGCGACGCCTTCGTAAAGGGGATCTACGGGCGGCTGTTCGTGTGGATTGTGGACAAGATCAACGCAGCAATTTACAAGCCTCCCTCCCAGGATGTGAAGAACTCTCGCAGGTCCATCGGCCTCCTGGACATCTTTGGGTTTGAGAACTTTGCTGTGAACAGCTTTGAGCAGCTCTGCATCAACTTCGCCAATGAGCACCTGCAGCAGTTCTTTGTGCGGCACGTGTTCAAGCTGGAGCAGGAGGAATATGACCTGGAGAGCATTGACTGGCTGCACATCGAGTTCACTGACAACCAGGATGCCCTGGACATGATTGCCAACAAGCCCATGAACATCATCTCCCTCATCGATGAGGAGAGCAAGTTCCCCAAGGGCACAGACACCACCATGTTACACAAGCTGAACTCCCAGCACAAGCTCAACGCCAACTACATCCCCCCCAAGAACAACCATGAGACCCAGTTTGGCATCAACCATTTTGCAGGCATCGTCTACTATGAGACCCAAGGCTTCCTGGAGAAGAACCGAGACACCCTGCATGGGGACATTATCCAGCTGGTCCACTCCTCCAGGAACAAGTTCATCAAGCAGATCTTCCAGGCCGATGTCGCCATGGGCGCCGAGACCAGGAAGCGCTCGCCCACACTTAGCAGCCAGTTCAAGCGGTCACTGGAGCTGCTGATGCGCACGCTGGGTGCCTGCCAGCCCTTCTTTGTGCGATGCATCAAGCCCAATGAGTTCAAGAAGCCCATGCTGTTCGACCGGCACCTGTGCGTGCGCCAGCTGCGGTACTCAGGAATGATGGAGACCATCCGAATCCGCCGAGCTGGCTACCCCATCCGCTACAGCTTCGTAGAGTTTGTGGAGCGGTACCGTGTGCTGCTGCCAGGTGTGAAGCCGGCCTACAAGCAGGGCGACCTCCGCGGGACTTGCCAGCGCATGGCTGAGGCTGTGCTGGGCACCCACGATGACTGGCAGATAGGCAAAACCAAGATCTTTCTGAAGGACCACCATGACATGCTGCTGGAAGTGGAGCGGGACAAAGCCATCACCGACAGAGTCATCCTCCTTCAGAAAGTCATCCGGGGATTCAAAGACAGGTCTAACTTTCTGAAGCTGAAGAACGCTGCCACACTGATCCAGAGGCACTGGCGGGGTCACAACTGTAGGAAGAACTACGGGCTGATGCGTCTGGGCTTCCTGCGGCTGCAGGCCCTGCACCGCTCCCGGAAGCTGCACCAGCAGTACCGCCTGGCCCGCCAGCGCATCATCCAGTTCCAGGCCCGCTGCCGCGCCTATCTGGTGCGCAAGGCCTTCCGCCACCGCCTCTGGGCTGTGCTCACCGTGCAGGCCTATGCCCGGGGCATGATCGCCCGCAGGCTGCACCAACGCCTCAGGGCTGAGTATCTGTGGCGCCTCGAGGCTGAGAAAATGCGGCTGGCGGAGGAAGAGAAGCTTCGGAAGGAGATGAGCGCCAAGAAGGCCAAGGAGGAGGCCGAGCGCAAGCATCAGGAGCGCCTGGCCCAGCTGGCTCGTGAGGACGCTGAGCGGGAGCTGAAGGAGAAGGAGGCCGCTCGGCGGAAGAAGGAGCTCCTGGAGCAGATGGAAAGGGCCCGCCATGAGCCTGTCAATCACTCAGACATGGTGGACAAGATGTTTGGCTTCCTGGGGACTTCAGGTGGCCTGCCAGGCCAGGAGGGCCAGGCACCTAGTGGCTTTGAGG

SEQ ID NO: 68 - protein, 968 aa, synthetic construct
  REGION 1..968: note = Synthetic
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFE

SEQ ID NO: 69 - DNA, 287 bp, synthetic construct
  misc_feature 1..287: note = Synthetic
  source 1..287: mol_type = other DNA
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgctgactgctgccgatactcggggctcccgctctcgctctcggtaacatccggccgggcgccgtccttgagcacatagcctggaccgtttc

SEQ ID NO: 70 - DNA, 287 bp, synthetic construct
  misc_feature 1..287: note = Synthetic
  source 1..287: mol_type = other DNA
ccccgggtgcgcggcgtcggtggtgccggcggggggcgccaggtcgcaggcggtgtagggctccaggcaggcggcgaaggccatgacgtgcgctatgaaggtctgctcctgcacgccgtgaaccaggtgcgcctgcgggccgcgcgcgaacaccgccacgtcctcgcctgcgtgggtctcttcgtccaggggcactgcgcactgctgccgatactcggggctcccgctctcgctctcggtaacatccggccgggcgccgtccttgagcacatagcctggaccgtttc

SEQ ID NO: 71 - DNA, 3627 bp, synthetic construct
  misc_feature 1..3627: note = 72
  source 1..3627: mol_type = other DNA
gacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaag

SEQ ID NO: 72 - DNA, 30 bp, synthetic construct
  misc_feature 1..30: note = Synthetic
  source 1..30: mol_type = other DNA
tacccttacgatgtaccggattacgcatga

SEQ ID NO: 73 - DNA, 2586 bp, synthetic construct
  misc_feature 1..2586: note = Synthetic
  source 1..2586: mol_type = other DNA
atggtgattcttcagcagggggaccatgtgtggatggacctgagattggggcaggagttcgacgtgcccatcggggcggtggtgaagctctgcgactctgggcaggtccaggtggtggatgatgaagacaatgaacactggatctctccgcagaacgcaacgcacatcaagcctatgcaccccacgtcggtccacggcgtggaggacatgatccgcctgggggacctcaacgaggcgggcatcttgcgcaacctgcttatccgctaccgggaccacctcatctacacgtatacgggctccatcctggtggctgtgaacccctaccagctgctctccatctactcgccagagcacatccgccagtataccaacaagaagattggggagatgcccccccacatctttgccattgctgacaactgctacttcaacatgaaacgcaacagccgagaccagtgctgcatcatcagtggggaatctggggccgggaagacggagagcacaaagctgatcctgcagttcctggcagccatcagtgggcagcactcgtggattgagcagcaggtcttggaggccacccccattctggaagcatttgggaatgccaagaccatccgcaatgacaactcaagccgtttcggaaagtacatcgacatccacttcaacaagcggggcgccatcgagggcgcgaagattgagcagtacctgctggaaaagtcacgtgtctgtcgccaggccctggatgaaaggaactaccacgtgttctactgcatgctggagggtatgagtgaggatcagaagaagaagctgggcttgggccaggcctctgactacaactacttggccatgggtaactgcataacctgtgagggccgggtggacagccaggagtacgccaacatccgctccgccatgaaggtgctcatgttcactgacaccgagaactgggagatctcgaagctcctggctgccatcctgcacctgggcaacctgcagtatgaggcacgcacatttgaaaacctggatgcctgtgaggttctcttctccccatcgctggccacagctgcatccctgcttgaggtgaaccccccagacctgatgagctgcctgactagccgcaccctcatcacccgcggggagacggtgtccaccccactgagcagggaacaggcactggacgtgcgcgacgccttcgtaaaggggatctacgggcggctgttcgtgtggattgtggacaagatcaacgcagcaatttacaagcctccctcccaggatgtgaagaactctcgcaggtccatcggcctcctggacatctttgggtttgagaactttgctgtgaacagctttgagcagctctgcatcaacttcgccaatgagcacctgcagcagttctttgtgcggcacgtgttcaagctggagcaggaggaatatgacctggagagcattgactggctgcacatcgagttcactgacaaccaggatgccctggacatgattgccaacaagcccatgaacatcatctccctcatcgatgaggagagcaagttccccaagggcacagacaccaccatgttacacaagctgaactcccagcacaagctcaacgccaactacatcccccccaagaacaaccatgagacccagtttggcatcaaccattttgcaggcatcgtctactatgagacccaaggcttcctggagaagaaccgagacaccctgcatggggacattatccagctggtccactcctccaggaacaagttcatcaagcagatcttccaggccgatgtcgccatgggcgccgagaccaggaagcgctcgcccacacttagcagccagttcaagcggtcactggagctgctgatgcgcacgctgggtgcctgccagcccttctttgtgcgatgcatcaagcccaatgagttcaagaagcccatgctgttcgaccggcacctgtgcgtgcgccagctgcggtactcaggaatgatggagaccatccgaatccgccgagctggctaccccatccgctacagcttcgtagagtttgtggagcggtaccgtgtgctgctgccaggtgtgaagccggcctacaagcagggcgacctccgcgggacttgccagcgcatggctgaggctgtgctgggcacccacgatgactggcagataggcaaaaccaagatctttctgaaggaccaccatgacatgctgctggaagtggagcgggacaaagccatcaccgacagagtcatcctccttcagaaagtcatccggggattcaaagacaggtctaactttctgaagctgaagaacgctgccacactgatccagaggcactggcggggtcacaactgtaggaagaactacgggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgag

SEQ ID NO: 74 - protein, 862 aa, synthetic construct
  REGION 1..862: note = Synthetic
MVILQQGDHVWMDLRLGQEFDVPIGAVVKLCDSGQVQVVDDEDNEHWISPQNATHIKPMHPTSVHGVEDMIRLGDLNEAGILRNLLIRYRDHLIYTYTGSILVAVNPYQLLSIYSPEHIRQYTNKKIGEMPPHIFAIADNCYFNMKRNSRDQCCIISGESGAGKTESTKLILQFLAAISGQHSWIEQQVLEATPILEAFGNAKTIRNDNSSRFGKYIDIHFNKRGAIEGAKIEQYLLEKSRVCRQALDERNYHVFYCMLEGMSEDQKKKLGLGQASDYNYLAMGNCITCEGRVDSQEYANIRSAMKVLMFTDTENWEISKLLAAILHLGNLQYEARTFENLDACEVLFSPSLATAASLLEVNPPDLMSCLTSRTLITRGETVSTPLSREQALDVRDAFVKGIYGRLFVWIVDKINAAIYKPPSQDVKNSRRSIGLLDIFGFENFAVNSFEQLCINFANEHLQQFFVRHVFKLEQEEYDLESIDWLHIEFTDNQDALDMIANKPMNIISLIDEESKFPKGTDTTMLHKLNSQHKLNANYIPPKNNHETQFGINHFAGIVYYETQGFLEKNRDTLHGDIIQLVHSSRNKFIKQIFQADVAMGAETRKRSPTLSSQFKRSLELLMRTLGACQPFFVRCIKPNEFKKPMLFDRHLCVRQLRYSGMMETIRIRRAGYPIRYSFVEFVERYRVLLPGVKPAYKQGDLRGTCQRMAEAVLGTHDDWQIGKTKIFLKDHHDMLLEVERDKAITDRVILLQKVIRGFKDRSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAE

SEQ ID NO: 75 - DNA, 3945 bp, synthetic construct
  misc_feature 1..3945: note = Synthetic
  source 1..3945: mol_type = other DNA
tatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaag

SEQ ID NO: 76 - protein, 1315 aa, synthetic construct
  REGION 1..1315: note = Synthetic
YLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVISVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 77 - DNA, 4170 bp, synthetic construct
  misc_feature 1..4170: note = Synthetic
  source 1..4170: mol_type = other DNA
gggctgatgcgtctgggcttcctgcggctgcaggccctgcaccgctcccggaagctgcaccagcagtaccgcctggcccgccagcgcatcatccagttccaggcccgctgccgcgcctatctggtgcgcaaggccttccgccaccgcctctgggctgtgctcaccgtgcaggcctatgcccggggcatgatcgcccgcaggctgcaccaacgcctcagggctgagtatctgtggcgcctcgaggctgagaaaatgcggctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaag

SEQ ID NO: 78 - protein, 1390 aa, synthetic construct
  REGION 1..1390: note = Synthetic
GLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVISVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 79 - protein, 120 aa, synthetic construct
  REGION 1..120: note = Synthetic
RSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKL

SEQ ID NO: 80 - DNA, 3912 bp, synthetic construct
  misc_feature 1..3912: note = 81
  source 1..3912: mol_type = other DNA
ctggcggaggaagagaagcttcggaaggagatgagcgccaagaaggccaaggaggaggccgagcgcaagcatcaggagcgcctggcccagctggctcgtgaggacgctgagcgggagctgaaggagaaggaggccgctcggcggaagaaggagctcctggagcagatggaaagggcccgccatgagcctgtcaatcactcagacatggtggacaagatgtttggcttcctggggacttcaggtggcctgccaggccaggagggccaggcacctagtggctttgaggacctggagcgagggcggagggagatggtggaggaggacctggatgcagccctgcccctgcctgacgaggatgaggaggacctctctgagtataaatttgccaagttcgcggccacctacttccaggggacaaccacgcactcctacacccggcggccactcaaacagccactgctctaccatgacgacgagggtgaccagctggcagccctggcggtctggatcaccatcctccgcttcatgggggacctccctgagcccaagtaccacacagccatgagtgatggcagtgagaagatccctgtgatgaccaagatttatgagaccctgggcaagaagacgtacaagagggagctgcaggccctgcagggcgagggcgaggcccagctccccgagggccagaagaagagcagtgtgaggcacaagctggtgcatttgactctgaaaaagaagtccaagctcacagaggaggtgaccaagaggctgcatgacggggagtccacagtgcagggcaacagcatgctggaggaccggcccacctccaacctggagaagctgcacttcatcatcggcaatggcatcctgcggccagcactccgggacgagatctactgccagatcagcaagcagctgacccacaacccctccaagagcagctatgcccggggctggattctcgtgtctctctgcgtgggctgtttcgccccctccgagaagtttgtcaagtacctgcggaacttcatccacgggggcccgcccggctacgccccgtactgtgaggagcgcctgagaaggacctttgtcaatgggacacggacacagccgcccagctggctggagctgcaggccaccaagtccaagaagccaatcatgttgcccgtgacattcatggatgggaccaccaagaccctgctgacggactcggcaaccacggccaaggagctctgcaacgcgctggccgacaagatctctctcaaggaccggttcgggttctccctctacattgccctgtttgacaaggtgtcctccctgggcagcggcagtgaccacgtcatggacgccatctcccagtgcgagcagtacgccaaggagcagggcgcccaggagcgcaacgccccctggaggctcttcttccgcaaagaggtcttcacgccctggcacagcccctccgaggacaacgtggccaccaacctcatctaccagcaggtggtgcgaggagtcaagtttggggagtacaggtgtgagaaggaggacgacctggctgagctggcctcccagcagtactttgtagactatggctctgagatgatcctggagcgcctcctgaacctcgtgcccacctacatccccgaccgcgagatcacgcccctgaagacgctggagaagtgggcccagctggccatcgccgcccacaagaaggggatttatgcccagaggagaactgatgcccagaaggtcaaagaggatgtggtcagttatgcccgcttcaagtggcccttgctcttctccaggttttatgaagcctacaaattctcaggccccagtctccccaagaacgacgtcatcgtggccgtcaactggacgggtgtgtactttgtggatgagcaggagcaggtacttctggagctgtccttcccagagatcatggccgtgtccagcagcaggggagcgaaaacgacggcccccagcttcacgctggccaccatcaagggggacgaatacaccttcacctccagcaatgctgaggacattcgtgacctggtggtcaccttcctagaggggctccggaagagatctaagtatgttgtggccctgcaggataaccccaaccccgcaggcgaggagtcaggcttcctcagctttgccaagggagacctcatcatcctggaccatgacacgggcgagcaggtcatgaactcgggctgggccaacggcatcaatgagaggaccaagcagcgtggggacttccccaccgacagtgtgtacgtcatgcccactgtcaccatgccaccgcgggagattgtggccctggtcaccatgactcccgatcagaggcaggacgttgtccggctcttgcagctgcgaacggcggagcccgaggtgcgtgccaagccctacacgctggaggagttttcctatgactacttcaggcccccacccaagcacacgctgagccgtgtcatggtgtccaaggcccgaggcaaggaccggctgtggagccacacgcgggaaccgctcaagcaggcgctgctcaagaagctcctgggcagtgaggagctctcgcaggaggcctgcctggccttcattgctgtgctcaagtacatgggcgactacccgtccaagaggacacgctccgtcaacgagctcaccgaccagatctttgagggtcccctgaaagccgagcccctgaaggacgaggcatatgtgcagatcctgaagcagctgaccgacaaccacatcaggtacagcgaggagcggggttgggagctgctctggctgtgcacgggccttttcccacccagcaacatcctcctgccccacgtgcagcgcttcctgcagtcccgaaagcactgcccactcgccatcgactgcctgcaacggctccagaaagccctgagaaacgggtcccggaagtaccctccgcacctggtggaggtggaggccatccagcacaagaccacccagattttccacaaagtctacttccctgatgacactgacgaggccttcgaagtggagtccagcaccaaggccaaggacttctgccagaacatcgccaccaggctgctcctcaagtcctcagagggattcagcctctttgtcaaaattgcagacaaggtcatcagcgttcctgagaatgacttcttctttgactttgttcgacacttgacagactggataaagaaagctcggcccatcaaggacggaattgtgccctcactcacctaccaggtgttcttcatgaagaagctgtggaccaccacggtgccagggaaggatcccatggccgattccatcttccactattaccaggagttgcccaagtatctccgaggctaccacaagtgcacgcgggaggaggtgctgcagctgggggcgctgatctacagggtcaagttcgaggaggacaagtcctacttccccagcatccccaagctgctgcgggagctggtgccccaggaccttatccggcaggtctcacctgatgactggaagcggtccatcgtcgcctacttcaacaagcacgcagggaagtccaaggaggaggccaagctggccttcctgaagctcatcttcaagtggcccacctttggctcagccttcttcgaggtgaagcaaactacggagccaaacttccctgagatcctcctaattgccatcaacaagtatggggtcagcctcatcgatcccaaaacgaaggatatcctcaccactcatcccttcaccaagatctccaactggagcagcggcaacacctacttccacatcaccattgggaacttggtgcgcgggagcaaactgctctgcgagacgtcactgggctacaagatggatgacctcctgacttcctacattagccagatgctcacagccatgagcaaacagcggggctccaggagcggcaag

SEQ ID NO: 81 - protein, 1304 aa, synthetic construct
  REGION 1..1304: note = Synthetic
LAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNFIHGGPPGYAPYCEERLRRTFVNGTRTQPPSWLELQATKSKKPIMLPVTFMDGTTKTLLTDSATTAKELCNALADKISLKDRFGFSLYIALFDKVSSLGSGSDHVMDAISQCEQYAKEQGAQERNAPWRLFFRKEVFTPWHSPSEDNVATNLIYQQVVRGVKFGEYRCEKEDDLAELASQQYFVDYGSEMILERLLNLVPTYIPDREITPLKTLEKWAQLAIAAHKKGIYAQRRTDAQKVKEDVVSYARFKWPLLFSRFYEAYKFSGPSLPKNDVIVAVNWTGVYFVDEQEQVLLELSFPEIMAVSSSRGAKTTAPSFTLATIKGDEYTFTSSNAEDIRDLVVTFLEGLRKRSKYVVALQDNPNPAGEESGFLSFAKGDLIILDHDTGEQVMNSGWANGINERTKQRGDFPTDSVYVMPTVTMPPREIVALVTMTPDQRQDVVRLLQLRTAEPEVRAKPYTLEEFSYDYFRPPPKHTLSRVMVSKARGKDRLWSHTREPLKQALLKKLLGSEELSQEACLAFIAVLKYMGDYPSKRTRSVNELTDQIFEGPLKAEPLKDEAYVQILKQLTDNHIRYSEERGWELLWLCTGLFPPSNILLPHVQRFLQSRKHCPLAIDCLQRLQKALRNGSRKYPPHLVEVEAIQHKTTQIFHKVYFPDDTDEAFEVESSTKAKDFCQNIATRLLLKSSEGFSLFVKIADKVISVPENDFFFDFVRHLTDWIKKARPIKDGIVPSLTYQVFFMKKLWTTTVPGKDPMADSIFHYYQELPKYLRGYHKCTREEVLQLGALIYRVKFEEDKSYFPSIPKLLRELVPQDLIRQVSPDDWKRSIVAYFNKHAGKSKEEAKLAFLKLIFKWPTFGSAFFEVKQTTEPNFPEILLIAINKYGVSLIDPKTKDILTTHPFTKISNWSSGNTYFHITIGNLVRGSKLLCETSLGYKMDDLLTSYISQMLTAMSKQRGSRSGK

SEQ ID NO: 82 - protein, 455 aa, synthetic construct
  REGION 1..455: note = Synthetic
RSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNF

SEQ ID NO: 83 - protein, 428 aa, synthetic construct
  REGION 1..428: note = Synthetic
GLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNF

SEQ ID NO: 84 - protein, 342 aa, synthetic construct
  REGION 1..342: note = Synthetic
RSNFLKLKNAATLIQRHWRGHNCRKNYGLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQ

SEQ ID NO: 85 - protein, 342 aa, synthetic construct
  REGION 1..342: note = Synthetic
LAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQKKSSVRHKLVHLTLKKKSKLTEEVTKRLHDGESTVQGNSMLEDRPTSNLEKLHFIIGNGILRPALRDEIYCQISKQLTHNPSKSSYARGWILVSLCVGCFAPSEKFVKYLRNF

SEQ ID NO: 86 - protein, 315 aa, synthetic construct
  REGION 1..315: note = Synthetic
GLMRLGFLRLQALHRSRKLHQQYRLARQRIIQFQARCRAYLVRKAFRHRLWAVLTVQAYARGMIARRLHQRLRAEYLWRLEAEKMRLAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQ

SEQ ID NO: 87 - protein, 229 aa, synthetic construct
  REGION 1..229: note = Synthetic
LAEEEKLRKEMSAKKAKEEAERKHQERLAQLAREDAERELKEKEAARRKKELLEQMERARHEPVNHSDMVDKMFGFLGTSGGLPGQEGQAPSGFEDLERGRREMVEEDLDAALPLPDEDEEDLSEYKFAKFAATYFQGTTTHSYTRRPLKQPLLYHDDEGDQLAALAVWITILRFMGDLPEPKYHTAMSDGSEKIPVMTKIYETLGKKTYKRELQALQGEGEAQLPEGQ

SEQ ID NO: 88 - protein, 93 aa, synthetic construct
  REGION 1..93: note = Synthetic
GLMRLGFLRLQALHRSRKLHQQYRLARQRIIQFQARCRAYLVRKAFRHRLWAVLTVQAYARGMIARRLHQRLRAEYLWRLEAEKMRLAEEEKL

SEQ ID NO: 89 - DNA, 20 bp, synthetic construct
  misc_feature 1..20: note = Synthetic
  source 1..20: mol_type = other DNA
tggcggaggaagagaagctt

SEQ ID NO: 90 - DNA, 2640 bp, synthetic construct; identical to SEQ ID NO: 63
  misc_feature 1..2640: note = Synthetic
  source 1..2640: mol_type = other DNA

SEQ ID NO: 91 - protein, 880 aa, synthetic construct; identical to SEQ ID NO: 62
  REGION 1..880: note = Synthetic